(19) United States
(12) Patent Application Publication
(10) Pub. No.: US 2022/0265715 A1
(43) Pub. Date: Aug. 25, 2022

(54) CHIMERIC ANTIGEN RECEPTOR T CELLS AND USES THEREOF

(71) Applicant: Eureka Therapeutics, Inc., Emeryville, CA (US)

(72) Inventors: Hongruo YUN, Emeryville, CA (US); Jun Cui, Emeryville, CA (US); Pengbo Zhang, Emeryville, CA (US); Yiyang Xu, Emeryville, CA (US); Lucas Horan, Emeryville, CA (US); Shaohua Xu, Emeryville, CA (US); Guangyan Xiong, Emeryville, CA (US); Shan LI, Emeryville, CA (US); Yixiang Xu, Emeryville, CA (US); Hong Liu, Emeryville, CA (US)

(21) Appl. No.: 17/628,118

(22) PCT Filed: Jul. 24, 2020
(86) PCT No.: PCT/US2020/043626

§ 371 Date: Jan. 18, 2022

Related U.S. Application Data
(60) Provisional application No. 62/878,182, filed on Jul. 24, 2019.

(51) Int. Cl. A61K 35/17; A61P 35/00; C07K 14/725; C07K 14/705

(52) U.S. Cl. CPC A61K 35/17; C07K 14/70578; C07K 14/7051; A61P 35/00

(57) ABSTRACT

Described herein are chimeric antigen receptors (CARs) comprising an extracellular target-binding domain comprising an antibody moiety (e.g., a single chain variable fragment (scFv) antibody), a transmembrane domain, a CD30 costimulatory domain, and a primary signaling domain. Also provided herein are methods of using the same or compositions thereof for the therapeutic treatment of cancers (e.g., hematological cancers or solid tumor cancers).

### CROSS-REFERENCES TO RELATED APPLICATIONS

This application claims priority to U.S. Provisional Application No. 62/878,182, filed Jul. 24, 2019, the disclosure of which is hereby incorporated by reference in its entirety for all purposes.

### BACKGROUND

Adoptive T cell immunotherapy, in which a patient's own T lymphocytes are engineered to express chimeric antigen receptors (CARs), has shown great promise in treating hematological malignancies. CARs commonly contain 3 modules: an extracellular target-binding module, a transmembrane domain (TM domain), and an intracellular signaling domain (ICD) that transmits activation signals. TM domains are primarily considered a structural requirement, anchoring the CAR in the cell membrane, and are most commonly derived from molecules regulating T cell function, such as CD8 and CD28. The intracellular module typically consists of the T cell receptor CD3ζ chain and one or more costimulatory domains from either the Ig (CD28-like) or TNF receptor (TNFR) superfamilies. CARs containing either CD28 or 4-1BB costimulatory domains have been the most widely used, to date, and both of them have yielded dramatic responses in clinical trials.

Most of the homology between TNF receptor family members occurs in the extracellular domain, with little homology in the cytoplasmic domain. This suggested that different members of the TNF receptor family might utilize distinct signaling pathways. Consistent with this hypothesis, the TNF receptor type 1 and Fas have been shown to interact with a set of intracellular signaling molecules through a 65-amino acid domain termed a death domain, whereas the TNF receptor type 2 and CD40 have been found to associate with members of the tumor necrosis factor receptor-associated factor (TRAF) family of signal transducing molecules.

CD30 is a member of the TNF1 receptor superfamily of receptor proteins. The membrane bound form of CD30 is a 120-kDa, 595-amino acid glycoprotein with a 188-amino acid cytoplasmic domain. Cross-linking of CD30 with either antibodies or with CD30 ligand produces a variety of effects in cells, including augmenting the proliferation of primary T cells following T cell receptor engagement and induction of the NF-kB transcription factor. CD30 was originally identified as an antigen expressed on the surface of Hodgkin's lymphoma cells. Subsequently, CD30 was shown to be expressed by lymphocytes with an activated phenotype, cells on the periphery of germinal centers, and CD45RO1 (memory) T cells. CD30 may also play a role in the development of T helper 2 type cells. The T cell activation properties of the TNF receptor family member 4-1BB have been shown to involve the specific ability of its cytoplasmic domain to associate with the tyrosine kinase p56lck. The sequence of the cytoplasmic domain of CD30 shows little sequence similarity to any of these receptors; CD30 lacks an obvious death domain or a p56lck-binding site.

### SUMMARY

The present invention provides, among other things, CARs that use a costimulatory domain from CD30 (also referred to herein as a CD30 costimulatory domain). As described in detail and demonstrated herein, T cells with CARs containing a costimulatory domain from CD30 express far less PD-1, an inhibitor of T cell activation, than T cells with CARs containing a costimulatory domain from, e.g., CD28 or 4-1BB, and at the same time demonstrate equal cytotoxic potential. The data suggests that the costimulatory domain from CD30 ameliorates the functional unresponsiveness that leads to T cell exhaustion, also called anergy, and subsequently, provides superior persistence of tumor cell killing. It is unexpected since CD30 lacks a p56lck-binding site that is thought to be crucial for CAR costimulation.

In one aspect, the invention features a chimeric antigen receptor (CAR) comprising: (a) an extracellular target-binding domain comprising an antibody moiety; (b) a transmembrane domain; (c) a CD30 costimulatory domain; and (d) a primary signaling domain. In some embodiments, the CD30 costimulatory domain comprises a sequence that can bind to an intracellular TRAF signaling protein. In some embodiments, the sequence that can bind to an intracellular TRAF signaling protein corresponds to residues 561-573 or 578-586 of a full-length CD30 having the sequence of SEQ ID NO:11. In some embodiments, the CD30 costimulatory domain comprises a sequence that is at least 80%, 85%, 90%, 95%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to residues 561-573 or 578-586 of SEQ ID NO:11. In some embodiments, the CD30 costimulatory domain comprises a sequence that is at least 70%, 75%, 80%, 85%, 90%, 95%, or 100% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) identical to the sequence of SEQ ID NO:35.

In some embodiments, the CAR comprises more than one CD30 costimulatory domain. In some embodiments, in addition to the CD30 costimulatory domain, the CAR further comprises at least one costimulatory domain which comprises the intracellular sequence of a costimulatory molecule that is different from CD30. In some embodiments, the costimulatory molecule that is different from CD30 is selected from the group consisting of CD27, CD28, 4-1BB (CD137), OX40, CD40, PD-1, ICOS, lymphocyte function-associated antigen-1 (LFA-1), CD2, CD7, LIGHT, NKG2C, B7-H3, and a ligand that specifically binds with CD83.

In some embodiments, the antibody moiety of the CAR is a single chain antibody fragment. The antibody moiety can be a single chain Fv (scFv), a single chain Fab, a single chain Fab′, a single domain antibody fragment, a single domain multispecific antibody, an intrabody, a nanobody, or a single chain immunokine. In certain embodiments, the antibody moiety is a single domain multispecific antibody (e.g., a single domain bispecific antibody). In certain embodiments, the antibody moiety is a single chain Fv (scFv), e.g., a tandem scFv.

In some embodiments, the transmembrane domain of the CAR is derived from the transmembrane domain of a TCR co-receptor or a T cell costimulatory molecule. The TCR co-receptor or T cell costimulatory molecule can be selected from the group consisting of CD8, 4-1BB, CD27, CD28, CD30, OX40, CD3ε, CD3ζ, CD45, CD4, CD5, CD9, CD16, CD22, CD33, CD37, CD64, CD80, CD86, CD134, CD137, and CD154. In certain embodiments, the TCR co-receptor or T cell costimulatory molecule is CD30 or CD8. In certain embodiments, the T cell costimulatory molecule is CD30. In certain embodiments, the TCR co-receptor is CD8.

In some embodiments, the transmembrane domain of the CAR is the transmembrane domain of CD8, 4-1BB, CD27, CD28, CD30, OX40, CD3ε, CD3ζ, CD45, CD4, CD5, CD9, CD16, CD22, CD33, CD37, CD64, CD80, CD86, CD134, CD137, or CD154. In certain embodiments, the transmembrane domain of the CAR is the transmembrane domain of CD30 or CD8. In certain embodiments, the transmembrane domain of the CAR is the transmembrane domain of CD30. In certain embodiments, the transmembrane domain of the CAR is the transmembrane domain of CD8. In certain embodiments, the transmembrane domain of the CAR comprises an amino acid sequence selected from the group consisting of SEQ ID NOS:26-31.

In some embodiments, the primary signaling domain comprises a sequence derived from the intracellular signaling sequence of a molecule selected from the group consisting of CD3ζ, TCRζ, FcRγ, FcRβ, CD3γ, CD3δ, CD3ε, CD5, CD22, CD79a, CD79b, and CD66d.

The primary signaling domain can comprise a sequence derived from the intracellular signaling sequence of CD3ζ. The primary signaling domain can comprise the intracellular signaling sequence of CD3ζ. In certain embodiments, the primary signaling domain comprises a sequence that is at least 80%, 85%, 90%, 95%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to the sequence of SEQ ID NO:37.

In some embodiments, the CAR described herein further comprises a peptide linker between the extracellular target-binding domain and the transmembrane domain. In some embodiments, the CAR described herein further comprises a peptide linker between the transmembrane domain and the CD30 costimulatory domain. In some embodiments, the CAR described herein further comprises a peptide linker between the CD30 costimulatory domain and the primary signaling domain.

In some embodiments, the antibody moiety specifically binds to a disease-related antigen, e.g., a cancer-related antigen or a virus-related antigen. In certain embodiments, the antibody moiety specifically binds to a cell surface antigen. The cell surface antigen can be selected from the group consisting of protein, carbohydrate, and lipid. The cell surface antigen can be CD19, CD20, CD22, CD47, CD158e, GPC3, ROR1, ROR2, BCMA, GPRC5D, FcRL5, MUC16, MCT4, PSMA, or a variant or mutant thereof.

In some embodiments, the antibody moiety specifically binds to human CD19. In some embodiments, the antibody moiety specifically binds to human CD22. In some embodiments, the antibody moiety specifically binds to human CD20. In some embodiments, the antibody moiety specifically binds to both human CD19 and human CD22. In some embodiments, the antibody moiety specifically binds to both human CD19 and human CD20. In some embodiments, the antibody moiety specifically binds to both human CD20 and human CD22. In some embodiments, the antibody moiety specifically binds to human CD19, human CD20, and human CD22.

In some embodiments, the antibody moiety specifically binds to a MHC-restricted antigen. For example, the antibody moiety can specifically bind to a complex comprising an alpha-fetoprotein (AFP) peptide and a MHC class I protein. In some embodiments, the AFP peptide comprises a sequence of any one of SEQ ID NOS:72-82. In some embodiments, the antibody moiety comprises sequences of HCDR1, HCDR2, and HCDR3 of SEQ ID NOS:83-85, respectively, and optionally a heavy chain variable region having the sequence of SEQ ID NO:86. In some embodiments, the antibody moiety comprises sequences of LCDR1, LCDR2, and LCDR3 of SEQ ID NOS:87-89, respectively, and optionally a light chain variable region having the sequence of SEQ ID NO:90. In some embodiments, the antibody moiety comprises sequences of HCDR1, HCDR2, and HCDR3 of SEQ ID NOS:91-93, respectively, and optionally a heavy chain variable region having the sequence of SEQ ID NO:94. In some embodiments, the antibody moiety comprises sequences of LCDR1, LCDR2, and LCDR3 of SEQ ID NOS:95-97, respectively, and optionally a light chain variable region having the sequence of SEQ ID NO:98. In some embodiments, the antibody moiety comprises sequences of HCDR1, HCDR2, and HCDR3 of SEQ ID NOS:99-101, respectively, and optionally a heavy chain variable region having the sequence of SEQ ID NO:102. In some embodiments, the antibody moiety comprises sequences of LCDR1, LCDR2, and LCDR3 of SEQ ID NOS:103-105, respectively, and optionally a light chain variable region having the sequence of SEQ ID NO:106. In some embodiments, the antibody moiety comprises sequences of HCDR1, HCDR2, and HCDR3 of SEQ ID NOS:107-109, respectively, and optionally a heavy chain variable region having the sequence of SEQ ID NO:110. In some embodiments, the antibody moiety comprises sequences of LCDR1, LCDR2, and LCDR3 of SEQ ID NOS:111-113, respectively, and optionally a light chain variable region having the sequence of SEQ ID NO:114. In some embodiments, the antibody moiety comprises sequences of HCDR1, HCDR2, and HCDR3 of SEQ ID NOS:115-117, respectively, and optionally a heavy chain variable region having the sequence of SEQ ID NO:118. In some embodiments, the antibody moiety comprises sequences of LCDR1, LCDR2, and LCDR3 of SEQ ID NOS:119-121, respectively, and optionally a light chain variable region having the sequence of SEQ ID NO:122.

In some embodiments, the antibody moiety specifically binds to a glypican 3 (GPC3) peptide. In some embodiments, the antibody moiety comprises sequences of HCDR1, HCDR2, and HCDR3 of SEQ ID NOS:123-125, respectively, and optionally a heavy chain variable region having the sequence of SEQ ID NO:126. In some embodiments, the antibody moiety comprises sequences of LCDR1, LCDR2, and LCDR3 of SEQ ID NOS:127-129, respectively, and optionally a light chain variable region having the sequence of SEQ ID NO:130. In some embodiments, the antibody moiety comprises sequences of HCDR1, HCDR2, and HCDR3 of SEQ ID NOS:131-133, respectively, and optionally a heavy chain variable region having the sequence of SEQ ID NO:134. In some embodiments, the antibody moiety comprises sequences of LCDR1, LCDR2, and LCDR3 of SEQ ID NOS:135-137, respectively, and optionally a light chain variable region having the sequence of SEQ ID NO:138. In some embodiments, the antibody moiety comprises sequences of HCDR1, HCDR2, and HCDR3 of SEQ ID NOS:139-141, respectively, and optionally a heavy chain variable region having the sequence of SEQ ID NO:142. In some embodiments, the antibody moiety comprises sequences of LCDR1, LCDR2, and LCDR3 of SEQ ID NOS:143-145, respectively, and optionally a light chain variable region having the sequence of SEQ ID NO:146. In some embodiments, the antibody moiety comprises sequences of HCDR1, HCDR2, and HCDR3 of SEQ ID NOS:147-149, respectively, and optionally a heavy chain variable region having the sequence of SEQ ID NO:150. In some embodiments, the antibody moiety comprises sequences of LCDR1, LCDR2, and LCDR3 of SEQ ID NOS:151-153, respectively, and optionally a light chain variable region having the sequence of SEQ ID NO:154. In some embodiments, the antibody moiety comprises sequences of HCDR1, HCDR2, and HCDR3 of SEQ ID NOS:155-157, respectively, and optionally a heavy chain variable region having the sequence of SEQ ID NO:158. In some embodiments, the antibody moiety comprises sequences of LCDR1, LCDR2, and LCDR3 of SEQ ID NOS:159-161, respectively, and optionally a light chain variable region having the sequence of SEQ ID NO:162. In some embodiments, the antibody moiety comprises sequences of HCDR1, HCDR2, and HCDR3 of SEQ ID NOS:163-165, respectively, and optionally a heavy chain variable region having the sequence of SEQ ID NO:68. In some embodiments, the antibody moiety comprises sequences of LCDR1, LCDR2, and LCDR3 of SEQ ID NOS:166-168, respectively, and optionally a light chain variable region having the sequence of SEQ ID NO:69. In some embodiments, the antibody moiety comprises sequences of HCDR1, HCDR2, and HCDR3 of SEQ ID NOS:169-171, respectively, and optionally a heavy chain variable region having the sequence of SEQ ID NO:70. In some embodiments, the antibody moiety comprises sequences of LCDR1, LCDR2, and LCDR3 of SEQ ID NOS:172-174, respectively, and optionally a light chain variable region having the sequence of SEQ ID NO:71. In some embodiments, the antibody moiety comprises a sequence of SEQ ID NO:12 or 13.

In some embodiments, the antibody moiety specifically binds to a KRAS peptide, e.g., a complex comprising a KRAS peptide and a MHC class I protein. In some embodiments, the KRAS peptide comprises a sequence of any one of SEQ ID NOS:175-183. In some embodiments, the antibody moiety comprises sequences of HCDR1, HCDR2, and HCDR3 of SEQ ID NOS:184-186, respectively, and optionally a heavy chain variable region having the sequence of SEQ ID NO:187. In some embodiments, the antibody moiety comprises sequences of LCDR1, LCDR2, and LCDR3 of SEQ ID NOS:188-190, respectively, and optionally a light chain variable region having the sequence of SEQ ID NO:191. In some embodiments, the antibody moiety comprises a sequence of SEQ ID NO:192. In some embodiments, the antibody moiety comprises sequences of HCDR1, HCDR2, and HCDR3 of SEQ ID NOS:193-195, respectively, and optionally a heavy chain variable region having the sequence of SEQ ID NO:196. In some embodiments, the antibody moiety comprises sequences of LCDR1, LCDR2, and LCDR3 of SEQ ID NOS:197-199, respectively, and optionally a light chain variable region having the sequence of SEQ ID NO:200. In some embodiments, the antibody moiety comprises a sequence of SEQ ID NO:201. In some embodiments, the antibody moiety comprises sequences of HCDR1, HCDR2, and HCDR3 of SEQ ID NOS:202-204, respectively, and optionally a heavy chain variable region having the sequence of SEQ ID NO:205. In some embodiments, the antibody moiety comprises sequences of LCDR1, LCDR2, and LCDR3 of SEQ ID NOS:206-208, respectively, and optionally a light chain variable region having the sequence of SEQ ID NO:209. In some embodiments, the antibody moiety comprises a sequence of SEQ ID NO:210. In some embodiments, the antibody moiety comprises sequences of HCDR1, HCDR2, and HCDR3 of SEQ ID NOS:211-213, respectively, and optionally a heavy chain variable region having the sequence of SEQ ID NO:214. In some embodiments, the antibody moiety comprises sequences of LCDR1, LCDR2, and LCDR3 of SEQ ID NOS:215-217, respectively, and optionally a light chain variable region having the sequence of SEQ ID NO:218. In some embodiments, the antibody moiety comprises a sequence of SEQ ID NO:219. In some embodiments, the antibody moiety comprises sequences of HCDR1, HCDR2, and HCDR3 of SEQ ID NOS:220-222, respectively, and optionally a heavy chain variable region having the sequence of SEQ ID NO:223. In some embodiments, the antibody moiety comprises sequences of LCDR1, LCDR2, and LCDR3 of SEQ ID NOS:224-226, respectively, and optionally a light chain variable region having the sequence of SEQ ID NO:227. In some embodiments, the antibody moiety comprises a sequence of SEQ ID NO:228. In some embodiments, the antibody moiety comprises sequences of HCDR1, HCDR2, and HCDR3 of SEQ ID NOS:229-231, respectively, and optionally a heavy chain variable region having the sequence of SEQ ID NO:232. In some embodiments, the antibody moiety comprises sequences of LCDR1, LCDR2, and LCDR3 of SEQ ID NOS:233-235, respectively, and optionally a light chain variable region having the sequence of SEQ ID NO:236. In some embodiments, the antibody moiety comprises a sequence of SEQ ID NO:237. In some embodiments, the antibody moiety comprises sequences of HCDR1, HCDR2, and HCDR3 of SEQ ID NOS:238-240, respectively, and optionally a heavy chain variable region having the sequence of SEQ ID NO:241. In some embodiments, the antibody moiety comprises sequences of LCDR1, LCDR2, and LCDR3 of SEQ ID NOS:242-244, respectively, and optionally a light chain variable region having the sequence of SEQ ID NO:245. In some embodiments, the antibody moiety comprises a sequence of SEQ ID NO:246. In some embodiments, the antibody moiety comprises sequences of HCDR1, HCDR2, and HCDR3 of SEQ ID NOS:247-249, respectively, and optionally a heavy chain variable region having the sequence of SEQ ID NO:250. In some embodiments, the antibody moiety comprises sequences of LCDR1, LCDR2, and LCDR3 of SEQ ID NOS:251-253, respectively, and optionally a light chain variable region having the sequence of SEQ ID NO:254. In some embodiments, the antibody moiety comprises a sequence of SEQ ID NO:255.

In some embodiments, the antibody moiety specifically binds to a NY-ESO-1 peptide, e.g., a complex comprising a NY-ESO-1 peptide and a MHC class I protein. In some embodiments, the NY-ESO-1 peptide comprises a sequence of any one of SEQ ID NOS:256-266. In some embodiments, the antibody moiety comprises sequences of HCDR1, HCDR2, and HCDR3 of SEQ ID NOS:267-269, respectively, and optionally a heavy chain variable region having the sequence of SEQ ID NO:270. In some embodiments, the antibody moiety comprises sequences of LCDR1, LCDR2, and LCDR3 of SEQ ID NOS:271-273, respectively, and optionally a light chain variable region having the sequence of SEQ ID NO:274. In some embodiments, the antibody moiety comprises sequences of HCDR1, HCDR2, and HCDR3 of SEQ ID NOS:275-277, respectively, and optionally a heavy chain variable region having the sequence of SEQ ID NO:278. In some embodiments, the antibody moiety comprises sequences of LCDR1, LCDR2, and LCDR3 of SEQ ID NOS:279-281, respectively, and optionally a light chain variable region having the sequence of SEQ ID NO:282. In some embodiments, the antibody moiety comprises sequences of HCDR1, HCDR2, and HCDR3 of SEQ ID NOS:283-285, respectively, and optionally a heavy chain variable region having the sequence of SEQ ID NO:286. In some embodiments, the antibody moiety comprises sequences of LCDR1, LCDR2, and LCDR3 of SEQ ID NOS:287-289, respectively, and optionally a light chain variable region having the sequence of SEQ ID NO:290. In some embodiments, the antibody moiety comprises sequences of HCDR1, HCDR2, and HCDR3 of SEQ ID NOS:291-293, respectively, and optionally a heavy chain variable region having the sequence of SEQ ID NO:294. In some embodiments, the antibody moiety comprises sequences of LCDR1, LCDR2, and LCDR3 of SEQ ID NOS:295-297, respectively, and optionally a light chain variable region having the sequence of SEQ ID NO:298. In some embodiments, the antibody moiety comprises sequences of HCDR1, HCDR2, and HCDR3 of SEQ ID NOS:299-301, respectively, and optionally a heavy chain variable region having the sequence of SEQ ID NO:302. In some embodiments, the antibody moiety comprises sequences of LCDR1, LCDR2, and LCDR3 of SEQ ID NOS:303-305, respectively, and optionally a light chain variable region having the sequence of SEQ ID NO:306. In some embodiments, the antibody moiety comprises sequences of HCDR1, HCDR2, and HCDR3 of SEQ ID NOS:307-309, respectively, and optionally a heavy chain variable region having the sequence of SEQ ID NO:310. In some embodiments, the antibody moiety comprises sequences of LCDR1, LCDR2, and LCDR3 of SEQ ID NOS:311-313, respectively, and optionally a light chain variable region having the sequence of SEQ ID NO:314. In some embodiments, the antibody moiety comprises sequences of HCDR1, HCDR2, and HCDR3 of SEQ ID NOS:315-317, respectively, and optionally a heavy chain variable region having the sequence of SEQ ID NO:318. In some embodiments, the antibody moiety comprises sequences of LCDR1, LCDR2, and LCDR3 of SEQ ID NOS:319-321, respectively, and optionally a light chain variable region having the sequence of SEQ ID NO:322.

In some embodiments, the antibody moiety specifically binds to a PRAME peptide, e.g., a complex comprising a PRAME peptide and a MHC class I protein. In some embodiments, the PRAME peptide comprises a sequence of any one of SEQ ID NOS:323-327. In some embodiments, the antibody moiety comprises sequences of HCDR1, HCDR2, and HCDR3 of SEQ ID NOS:328-330, respectively, and optionally a heavy chain variable region having the sequence of SEQ ID NO:331. In some embodiments, the antibody moiety comprises sequences of LCDR1, LCDR2, and LCDR3 of SEQ ID NOS:332-334, respectively, and optionally a light chain variable region having the sequence of SEQ ID NO:335. In some embodiments, the antibody moiety comprises sequences of HCDR1, HCDR2, and HCDR3 of SEQ ID NOS:336-338, respectively, and optionally a heavy chain variable region having the sequence of SEQ ID NO:339. In some embodiments, the antibody moiety comprises sequences of LCDR1, LCDR2, and LCDR3 of SEQ ID NOS:340-342, respectively, and optionally a light chain variable region having the sequence of SEQ ID NO:343. In some embodiments, the antibody moiety comprises sequences of HCDR1, HCDR2, and HCDR3 of SEQ ID NOS:344-346, respectively, and optionally a heavy chain variable region having the sequence of SEQ ID NO:347. In some embodiments, the antibody moiety comprises sequences of LCDR1, LCDR2, and LCDR3 of SEQ ID NOS:348-350, respectively, and optionally a light chain variable region having the sequence of SEQ ID NO:351. In some embodiments, the antibody moiety comprises sequences of HCDR1, HCDR2, and HCDR3 of SEQ ID NOS:352-354, respectively, and optionally a heavy chain variable region having the sequence of SEQ ID NO:355. In some embodiments, the antibody moiety comprises sequences of LCDR1, LCDR2, and LCDR3 of SEQ ID NOS:356-358, respectively, and optionally a light chain variable region having the sequence of SEQ ID NO:359. In some embodiments, the antibody moiety comprises sequences of HCDR1, HCDR2, and HCDR3 of SEQ ID NOS:360-362, respectively, and optionally a heavy chain variable region having the sequence of SEQ ID NO:363. In some embodiments, the antibody moiety comprises sequences of LCDR1, LCDR2, and LCDR3 of SEQ ID NOS:364-366, respectively, and optionally a light chain variable region having the sequence of SEQ ID NO:367. In some embodiments, the antibody moiety comprises sequences of HCDR1, HCDR2, and HCDR3 of SEQ ID NOS:368-370, respectively, and optionally a heavy chain variable region having the sequence of SEQ ID NO:371. In some embodiments, the antibody moiety comprises sequences of LCDR1, LCDR2, and LCDR3 of SEQ ID NOS:372-374, respectively, and optionally a light chain variable region having the sequence of SEQ ID NO:375. In some embodiments, the antibody moiety comprises sequences of HCDR1, HCDR2, and HCDR3 of SEQ ID NOS:376-378, respectively, and optionally a heavy chain variable region having the sequence of SEQ ID NO:379. In some embodiments, the antibody moiety comprises sequences of LCDR1, LCDR2, and LCDR3 of SEQ ID NOS:380-382, respectively, and optionally a light chain variable region having the sequence of SEQ ID NO:383.

In some embodiments, the antibody moiety specifically binds to a histone H3.3 peptide, e.g., a complex comprising a histone H3.3 peptide and a MHC class I protein. In some embodiments, the histone H3.3 peptide comprises a sequence of any one of SEQ ID NOS:384-403. In some embodiments, the antibody moiety comprises sequences of HCDR1, HCDR2, and HCDR3 of SEQ ID NOS:404-406, respectively, and optionally a heavy chain variable region having the sequence of SEQ ID NO:407. In some embodiments, the antibody moiety comprises sequences of LCDR1, LCDR2, and LCDR3 of SEQ ID NOS:408-410, respectively, and optionally a light chain variable region having the sequence of SEQ ID NO:411. In some embodiments, the antibody moiety comprises sequences of HCDR1, HCDR2, and HCDR3 of SEQ ID NOS:412-414, respectively, and optionally a heavy chain variable region having the sequence of SEQ ID NO:415. In some embodiments, the antibody moiety comprises sequences of LCDR1, LCDR2, and LCDR3 of SEQ ID NOS:416-418, respectively, and optionally a light chain variable region having the sequence of SEQ ID NO:419. In some embodiments, the antibody moiety comprises sequences of HCDR1, HCDR2, and HCDR3 of SEQ ID NOS:420-422, respectively, and optionally a heavy chain variable region having the sequence of SEQ ID NO:423. In some embodiments, the antibody moiety comprises sequences of LCDR1, LCDR2, and LCDR3 of SEQ ID NOS:424-426, respectively, and optionally a light chain variable region having the sequence of SEQ ID NO:427. In some embodiments, the antibody moiety comprises sequences of HCDR1, HCDR2, and HCDR3 of SEQ ID NOS:428-430, respectively, and optionally a heavy chain variable region having the sequence of SEQ ID NO:431. In some embodiments, the antibody moiety comprises sequences of LCDR1, LCDR2, and LCDR3 of SEQ ID NOS:432-434, respectively, and optionally a light chain variable region having the sequence of SEQ ID NO:435. In some embodiments, the antibody moiety comprises sequences of HCDR1, HCDR2, and HCDR3 of SEQ ID NOS:436-438, respectively, and optionally a heavy chain variable region having the sequence of SEQ ID NO:439. In some embodiments, the antibody moiety comprises sequences of LCDR1, LCDR2, and LCDR3 of SEQ ID NOS:440-442, respectively, and optionally a light chain variable region having the sequence of SEQ ID NO:443. In some embodiments, the antibody moiety comprises sequences of HCDR1, HCDR2, and HCDR3 of SEQ ID NOS:444-446, respectively, and optionally a heavy chain variable region having the sequence of SEQ ID NO:447. In some embodiments, the antibody moiety comprises sequences of LCDR1, LCDR2, and LCDR3 of SEQ ID NOS:448-450, respectively, and optionally a light chain variable region having the sequence of SEQ ID NO:451. In some embodiments, the antibody moiety comprises sequences of HCDR1, HCDR2, and HCDR3 of SEQ ID NOS:452-454, respectively, and optionally a heavy chain variable region having the sequence of SEQ ID NO:455. In some embodiments, the antibody moiety comprises sequences of LCDR1, LCDR2, and LCDR3 of SEQ ID NOS:456-458, respectively, and optionally a light chain variable region having the sequence of SEQ ID NO:459. In some embodiments, the antibody moiety comprises sequences of HCDR1, HCDR2, and HCDR3 of SEQ ID NOS:460-462, respectively, and optionally a heavy chain variable region having the sequence of SEQ ID NO:463. In some embodiments, the antibody moiety comprises sequences of LCDR1, LCDR2, and LCDR3 of SEQ ID NOS:464-466, respectively, and optionally a light chain variable region having the sequence of SEQ ID NO:467. In some embodiments, the antibody moiety comprises sequences of HCDR1, HCDR2, and HCDR3 of SEQ ID NOS:468-470, respectively, and optionally a heavy chain variable region having the sequence of SEQ ID NO:471. In some embodiments, the antibody moiety comprises sequences of LCDR1, LCDR2, and LCDR3 of SEQ ID NOS:472-474, respectively, and optionally a light chain variable region having the sequence of SEQ ID NO:475. In some embodiments, the antibody moiety comprises sequences of HCDR1, HCDR2, and HCDR3 of SEQ ID NOS:476-478, respectively, and optionally a heavy chain variable region having the sequence of SEQ ID NO:479. In some embodiments, the antibody moiety comprises sequences of LCDR1, LCDR2, and LCDR3 of SEQ ID NOS:480-482, respectively, and optionally a light chain variable region having the sequence of SEQ ID NO:483. In some embodiments, the antibody moiety comprises sequences of HCDR1, HCDR2, and HCDR3 of SEQ ID NOS:484-486, respectively, and optionally a heavy chain variable region having the sequence of SEQ ID NO:487. In some embodiments, the antibody moiety comprises sequences of LCDR1, LCDR2, and LCDR3 of SEQ ID NOS:488-490, respectively, and optionally a light chain variable region having the sequence of SEQ ID NO:491. In some embodiments, the antibody moiety comprises sequences of HCDR1, HCDR2, and HCDR3 of SEQ ID NOS:492-494, respectively, and optionally a heavy chain variable region having the sequence of SEQ ID NO:495. In some embodiments, the antibody moiety comprises sequences of LCDR1, LCDR2, and LCDR3 of SEQ ID NOS:496-498, respectively, and optionally a light chain variable region having the sequence of SEQ ID NO:499.

In some embodiments, the antibody moiety specifically binds to a WT1 peptide, e.g., a complex comprising a WT1 peptide and a MHC class I protein. In some embodiments, the WT1 peptide comprises a sequence of SEQ ID NO:500. In some embodiments, the antibody moiety comprises sequences of HCDR1, HCDR2, and HCDR3 of SEQ ID NOS:501-503, respectively, and optionally a heavy chain variable region having the sequence of SEQ ID NO:504. In some embodiments, the antibody moiety comprises sequences of LCDR1, LCDR2, and LCDR3 of SEQ ID NOS:505-507, respectively, and optionally a light chain variable region having the sequence of SEQ ID NO:508. In some embodiments, the antibody moiety comprises a sequence of SEQ ID NO:509. In some embodiments, the antibody moiety comprises sequences of HCDR1, HCDR2, and HCDR3 of SEQ ID NOS:510-512, respectively, and optionally a heavy chain variable region having the sequence of SEQ ID NO:513. In some embodiments, the antibody moiety comprises sequences of LCDR1, LCDR2, and LCDR3 of SEQ ID NOS:514-516, respectively, and optionally a light chain variable region having the sequence of SEQ ID NO:517. In some embodiments, the antibody moiety comprises a sequence of SEQ ID NO:518. In some embodiments, the antibody moiety comprises sequences of HCDR1, HCDR2, and HCDR3 of SEQ ID NOS:519-521, respectively, and optionally a heavy chain variable region having the sequence of SEQ ID NO:522. In some embodiments, the antibody moiety comprises sequences of LCDR1, LCDR2, and LCDR3 of SEQ ID NOS:523-525, respectively, and optionally a light chain variable region having the sequence of SEQ ID NO:526. In some embodiments, the antibody moiety comprises a sequence of SEQ ID NO:527. In some embodiments, the antibody moiety comprises sequences of HCDR1, HCDR2, and HCDR3 of SEQ ID NOS:528-530, respectively, and optionally a heavy chain variable region having the sequence of SEQ ID NO:531. In some embodiments, the antibody moiety comprises sequences of LCDR1, LCDR2, and LCDR3 of SEQ ID NOS:532-534, respectively, and optionally a light chain variable region having the sequence of SEQ ID NO:535. In some embodiments, the antibody moiety comprises a sequence of SEQ ID NO:536. In some embodiments, the antibody moiety comprises sequences of HCDR1, HCDR2, and HCDR3 of SEQ ID NOS:537-539, respectively, and optionally a heavy chain variable region having the sequence of SEQ ID NO:540. In some embodiments, the antibody moiety comprises sequences of LCDR1, LCDR2, and LCDR3 of SEQ ID NOS:541-543, respectively, and optionally a light chain variable region having the sequence of SEQ ID NO:544. In some embodiments, the antibody moiety comprises a sequence of SEQ ID NO:545. In some embodiments, the antibody moiety comprises sequences of HCDR1, HCDR2, and HCDR3 of SEQ ID NOS:546-548, respectively, and optionally a heavy chain variable region having the sequence of SEQ ID NO:549. In some embodiments, the antibody moiety comprises sequences of LCDR1, LCDR2, and LCDR3 of SEQ ID NOS:550-552, respectively, and optionally a light chain variable region having the sequence of SEQ ID NO:553. In some embodiments, the antibody moiety comprises a sequence of SEQ ID NO:554.

In some embodiments, the antibody moiety specifically binds to a PSA peptide, e.g., a complex comprising a PSA peptide and a MHC class I protein. In some embodiments, the PSA peptide comprises a sequence of any one of SEQ ID NOS:555-565. In some embodiments, the antibody moiety comprises an HCDR1 sequence of any one of SEQ ID NOS:566-580, an HCDR2 sequence of any one of SEQ ID NOS:581-594, and an HCDR3 sequence of any one of SEQ ID NOS:595-612, and optionally a heavy chain variable region having a sequence of any one of SEQ ID NOS:613-630. In some embodiments, the antibody moiety comprises a LCDR1 sequence of any one of SEQ ID NOS:631-647, a LCDR2 sequence of any one of SEQ ID NOS:648-660, and a LCDR3 sequence of any one of SEQ ID NOS:661-678, and optionally a light chain variable region having a sequence of any one of SEQ ID NOS:679-696.

In another aspect, the disclosure also features a nucleic acid molecule encoding, in whole or in part, any of the CARs described herein.

In another aspect, the disclosure also features a vector comprising the nucleic acid molecule described above.

In another aspect, the disclosure also features a CD30-CAR effector cell: (a) expressing any one the CAR described herein, or (b) comprising the nucleic acid molecule or the vector described above. In certain embodiments, the effector cell is a T cell.

In another aspect, the disclosure also features a pharmaceutical composition comprising any of the CARs described herein, the nucleic acid molecule described above, the vector described above, or the CD30-CAR effector cell described above, and a pharmaceutically acceptable carrier or diluent.

In another aspect, the disclosure also features a method of killing target cells, comprising: contacting one or more target cells with one or more CD30-CAR effector cells described herein under conditions and for a time sufficient so that the CD30-CAR effector cells mediate killing of the target cells, wherein the target cells express an antigen specific to the CD30-CAR effector cells, and wherein the CD30-CAR effector cells express a low cell exhaustion level upon contacting the target cells.

In some embodiments, the CD30-CAR effector T cells express a low level of an exhaustion marker selected from the group consisting of PD-1, TIM-3, and LAG-3. In some embodiments, the CD30-CAR effector cells are T cells. In some embodiments, the CD30-CAR effector T cells express a low level of PD-1. In some embodiments, the CD30-CAR effector T cells express a low level of TIM-3. In some embodiments, the CD30-CAR effector T cells express a low level of LAG-3. In some embodiments, the CD30-CAR effector cells express a lower level of PD-1, TIM-3, or LAG-3 than corresponding effector cells expressing a CAR comprising a CD28 costimulatory domain. For example, the CD30-CAR effector cells express a lower level of PD-1 than the corresponding CD28 CAR effector cells, and wherein the ratio of PD-1 expression level of the CD30-CAR effector cells to the corresponding CD28 CAR effector cells is 0.9, 0.8, 0.7, 0.6, 0.5, 0.4, 0.3, 0.2, 0.1 or lower. For example, the CD30-CAR effector cells express a lower level of TIM-3 than the corresponding CD28 CAR effector cells, and wherein the ratio of TIM-3 expression level of the CD30-CAR effector cells to the corresponding CD28 CAR effector cells is 0.9, 0.8, 0.7, 0.6, 0.5, 0.4, 0.3, 0.2, 0.1 or lower. For example, the CD30-CAR effector cells express a lower level of LAG-3 than the corresponding CD28 CAR effector cells, and wherein the ratio of LAG-3 expression level of the CD30-CAR effector cells to the corresponding CD28 CAR effector cells is 0.9, 0.8, 0.7, 0.6, 0.5, 0.4, 0.3, 0.2, 0.1 or lower.

In some embodiments, the CD30-CAR effector T cells express a lower level of PD-1, TIM-3, or LAG-3 than corresponding effector T cells expressing a CAR comprising a 4-1BB costimulatory domain. For example, the CD30-CAR effector T cells express a lower cell exhaustion level of PD-1 than the corresponding 4-1BB CAR effector cells, and wherein the ratio of PD-1 expression level of the CD30-CAR effector cells to the corresponding 4-1BB CAR effector cells is 0.9, 0.8, 0.7, 0.6, 0.5, 0.4, 0.3, 0.2, 0.1 or lower. For example, the CD30-CAR effector cells express a lower level of TIM-3 than the corresponding 4-1BB CAR effector cells, and wherein the ratio of TIM-3 expression level of the CD30-CAR effector cells to the corresponding 4-1BB CAR effector cells is 0.9, 0.8, 0.7, 0.6, 0.5, 0.4, 0.3, 0.2, 0.1 or lower. For example, the CD30-CAR effector cells express a lower level of LAG-3 than the corresponding 4-1BB CAR effector cells, and wherein the ratio of LAG-3 expression level of the CD30-CAR effector cells to the corresponding 4-1BB CAR effector cells is 0.9, 0.8, 0.7, 0.6, 0.5, 0.4, 0.3, 0.2, 0.1 or lower.

In some embodiments, a “corresponding effector cell” refers to a reference effector cell comprising a CAR which comprises the same extracellular target-binding domain and primary signaling domain as the CAR in a subject effector cell, but has a different costimulatory domain. The CAR in the subject effector cell has a CD30 costimulatory domain. The CAR in the corresponding effector cell (e.g., reference effector cell) does not have a CD30 costimulatory domain. In some embodiments, the CAR in the corresponding effector cell (e.g., reference effector cell) has a CD28 costimulatory domain or a 4-1BB costimulatory domain. The corresponding effector cell's CAR can have the same transmembrane domain as the CAR in the subject effector cell. It can also have a different transmembrane domain from the CAR in the subject effector cell. In some embodiments, the CAR in the corresponding effector cell has a CD28 transmembrane domain and CD28 costimulatory domain while the CAR in the subject effector cell has a CD30 or CD8 transmembrane domain and CD30 costimulatory domain. In some embodiments, the CAR in the corresponding effector cell has a CD8 transmembrane domain and 4-1BB costimulatory domain while the CAR in the subject effector cell has a CD8 or CD30 transmembrane domain and CD30 costimulatory domain. In some embodiments, an effector cell comprising a CD30 costimulatory domain can be compared to a corresponding effector cell under to same conditions to measure, for example, the level of exhaustion markers (e.g., PD-1, TIM-3, or LAG-3).

In some embodiments of this aspect, the target cells are cancer cells. In some embodiments, the cancer cells are from a cancer selected from the group consisting of adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, leukemia, lymphoma, lung cancer, melanoma, mesothelioma, multiple myeloma, pancreatic cancer, pheochromocytoma, plasmacytoma, neuroblastoma, ovarian cancer, prostate cancer, sarcoma, stomach cancer, uterine cancer, and thyroid cancer. In some embodiments, the cancer cells are hematological cancer cells. In some embodiments, the cancer cells are solid tumor cells. In some embodiments, the target cells are virus-infected cells, e.g., virus-infected cells from a viral infection caused by a virus selected from the group consisting of Cytomegalovirus (CMV), Epstein-Barr Virus (EBV), Hepatitis B Virus (HBV), Kaposi's Sarcoma associated herpesvirus (KSHV), Human papillomavirus (HPV), Molluscum contagiosum virus (MCV), Human T cell leukemia virus 1 (HTLV-1), HIV (Human immunodeficiency virus), and Hepatitis C Virus (HCV).

In another aspect, the disclosure features a method of treating a disease, the method comprising a step of administering to a subject any of the CARs described herein, the nucleic acid molecule described above, the vector described above, the CD30-CAR effector cell described above, or the pharmaceutical composition described above. In some embodiments, the disease is cancer. The cancer can be selected from the group consisting of adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, leukemia, lymphoma, lung cancer, melanoma, mesothelioma, multiple myeloma, pancreatic cancer, pheochromocytoma, plasmacytoma, neuroblastoma, ovarian cancer, prostate cancer, sarcoma, stomach cancer, uterine cancer, and thyroid cancer. In some embodiments, the cancer is a hematological cancer. In some embodiments, the cancer is a solid tumor cancer. In some embodiments, the disease is a viral infection.

In another aspect, the disclosure features a method for preventing and/or reversing T cell exhaustion in a subject, comprising administering to the subject any of the CARs described herein, the nucleic acid molecule described above, the vector described above, the CD30-CAR effector cell described above, or the pharmaceutical composition described above. In some embodiments, the method decreases the expression of an exhaustion marker in a T cell, e.g., the exhaustion marker can be selected from the group consisting of PD-1, TIM-3, and LAG-3.

### Definitions

The scope of present invention is defined by the claims appended hereto and is not limited by particular embodiments described herein; those skilled in the art, reading the present disclosure, will be aware of various modifications that may be equivalent to such described embodiments, or otherwise within the scope of the claims.

In general, terminology used herein is in accordance with its understood meaning in the art, unless clearly indicated otherwise. Explicit definitions of certain terms are provided below; meanings of these and other terms in particular instances throughout this specification will be clear to those skilled in the art from context.

In order that the present invention may be more readily understood, certain terms are first defined below. Additional definitions for the following terms and other terms are set forth throughout the specification.

Administration: As used herein, the term “administration” refers to the administration of a composition to a subject or system (e.g., to a cell, organ, tissue, organism, or relevant component or set of components thereof). Those of ordinary skill will appreciate that route of administration may vary depending, for example, on the subject or system to which the composition is being administered, the nature of the composition, the purpose of the administration, etc. For example, in certain embodiments, administration to an animal subject (e.g., to a human) may be bronchial (including by bronchial instillation), buccal, enteral, interdermal, intra-arterial, intradermal, intragastric, intrahepatic, intramedullary, intramuscular, intranasal, intraperitoneal, intrathecal, intratumoral, intravenous, intraventricular, mucosal, nasal, oral, rectal, subcutaneous, sublingual, topical, tracheal (including by intratracheal instillation), transdermal, vaginal and/or vitreal. In some embodiments, administration may involve intermittent dosing. In some embodiments, administration may involve continuous dosing (e.g., perfusion) for at least a selected period of time.

Affinity: As is known in the art, “affinity” is a measure of the tightness with a particular ligand binds to its partner. Affinities can be measured in different ways. In some embodiments, affinity is measured by a quantitative assay. In some such embodiments, binding partner concentration may be fixed to be in excess of ligand concentration so as to mimic physiological conditions. Alternatively or additionally, in some embodiments, binding partner concentration and/or ligand concentration may be varied. In some such embodiments, affinity may be compared to a reference under comparable conditions (e.g., concentrations).

Affinity matured (or affinity matured antibody): As used herein, refers to an antibody with one or more alterations in one or more CDRs (or, in some embodiments, framework regions) thereof which result an improvement in the affinity of the antibody for antigen, compared to a parent antibody which does not possess those alteration(s). In some embodiments, affinity matured antibodies will have nanomolar or even picomolar affinities for a target antigen. Affinity matured antibodies may be produced by any of a variety of procedures known in the art. Marks et al., 1992, BioTechnology 10:779-783 describes affinity maturation by V_{H} and V_{L} domain shuffling. Random mutagenesis of CDR and/or framework residues is described by: Barbas et al., 1994, Proc. Nat. Acad. Sci., U.S.A. 91:3809-3813; Schier et al., 1995, Gene 169: 147-155; Yelton et al., 1995. J. Immunol. 155:1994-2004; Jackson et al., 1995, J. Immunol. 154(7):3310-9; and Hawkins et al., 1992, J. Mol. Biol. 226:889-896. Selection of binders with improved binding properties is described by Thie et al., 2009, Methods Mol. Bio. 525:309-22.

Agent: As used herein may refer to a compound or entity of any chemical class including, for example, polypeptides, nucleic acids, saccharides, lipids, small molecules, metals, or combinations thereof. In some embodiments, an agent is or comprises a natural product in that it is found in and/or is obtained from nature. In some embodiments, an agent is or comprises one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents are provided as collections or libraries, for example that may be screened to identify or characterize active agents within them. Some particular embodiments of agents that may be utilized in accordance with the present invention include small molecules, antibodies, aptamers, nucleic acids (e.g., siRNAs, shRNAs, DNA/RNA hybrids, antisense oligonucleotides, ribozymes), peptides, peptide mimetics, etc. In some embodiments, an agent is or comprises a polymer. In some embodiments, an agent is not a polymer and/or is substantially free of any polymer. In some embodiments, an agent contains at least one polymeric moiety. In some embodiments, an agent lacks or is substantially free of any polymeric moiety.

Amino acid: As used herein, term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an 1-amino acid. “Standard amino acid” refers to any of the twenty standard 1-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or post-translational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Animal: As used herein refers to any member of the animal kingdom. In some embodiments, “animal” refers to humans, of either sex and at any stage of development. In some embodiments, “animal” refers to non-human animals, at any stage of development. In certain embodiments, the non-human animal is a mammal (e.g., a mouse, a rat, a rabbit, a pig, a cow, a deer, a sheep, a goat, a cat, a dog, or a monkey). In some embodiments, animals include, but are not limited to, mammals, birds, reptiles, amphibians, fish, insects, and/or worms. In some embodiments, an animal may be a transgenic animal, genetically engineered animal, and/or a clone.

Antibody moiety: As used herein, this term encompasses full-length antibodies and antigen-binding fragments thereof. A full-length antibody comprises two heavy chains and two light chains. The variable regions of the light and heavy chains are responsible for antigen binding. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as igG1 (γ1 heavy chain), lgG2 (γ2 heavy chain), lgG3 (γ3 heavy chain), lgG4 (γ4 heavy chain), igA1 (α1 heavy chain), or lgA2 (α2 heavy chain).

Antigen-binding fragment or Antigen-binding portion: The term “antigen-binding fragment” or “antigen-binding portion,” as used herein, refers to an antibody fragment including, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain Fv (scFv), an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

Biological activity: As used herein, refers to an observable biological effect or result achieved by an agent or entity of interest. For example, in some embodiments, a specific binding interaction is a biological activity. In some embodiments, modulation (e.g., induction, enhancement, or inhibition) of a biological pathway or event is a biological activity. In some embodiments, presence or extent of a biological activity is assessed through detection of a direct or indirect product produced by a biological pathway or event of interest.

Bispecific antibody: As used herein, refers to a bispecific binding agent in which at least one, and typically both, of the binding moieties is or comprises an antibody moiety. A variety of different bispecific antibody structures are known in the art. In some embodiments, each binding moiety in a bispecific antibody that is or comprises an antibody moiety includes V_{H} and/or V_{L} regions; in some such embodiments, the V_{H} and/or V_{L} regions are those found in a particular monoclonal antibody. In some embodiments, where the bispecific antibody contains two antibody moieties, each includes V_{H} and/or V_{L} regions from different monoclonal antibodies.

The term “bispecific antibody” as used herein also refers to a polypeptide with two discrete binding moieties, each of which binds a distinct target. In some embodiments, a bispecific binding antibody is a single polypeptide; in some embodiments, a bispecific binding antibody is or comprises a plurality of peptides which, in some such embodiments may be covalently associated with one another, for example by cross-linking. In some embodiments, the two binding moieties of a bispecific binding antibody recognize different sites (e.g., epitopes) of the same target (e.g., antigen); in some embodiments, they recognize different targets. In some embodiments, a bispecific binding antibody is capable of binding simultaneously to two targets, which are of different structure.

Carrier: As used herein, refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

CDR: As used herein, the term “CDR” or “complementarity determining region” is intended to mean the non-contiguous antigen combining sites found within the variable region of both heavy and light chain polypeptides. There are three CDRs in each of the variable regions of the heavy chain and the light chain, which are designated CDR1, CDR2 and CDR3, for each of the variable regions. A “set of CDRs” or “CDR set” refers to a group of three or six CDRs that occur in either a single variable region capable of binding the antigen or the CDRs of cognate heavy and light chain variable regions capable of binding the antigen. These particular regions have been described by Kabat et al., J. Biol. Chem. 252:6609-6616 (1977); Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273: 927-948 (1997); MacCallum et al., J. Mol. Biol. 262:732-745 (1996); Abhinandan and Martin, Mol. Immunol., 45: 3832-3839 (2008); Lefranc M. P. et al., Dev. Comp. Immunol., 27: 55-77 (2003); and Honegger and Pluckthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison. CDR prediction algorithms and interfaces are known in the art, including, for example, Abhinandan and Martin, Mol. Immunol., 45: 3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present invention and for possible inclusion in one or more claims herein.

TABLE 1

| | Kabat¹ | Chothia² | MacCallum³ | IMGT⁴ | AHo⁵ |
|---|---|---|---|---|---|
| V_{H} CDR1 | 31-35 | 26-32 | 30-35 | 27-38 | 25-40 |
| V_{H} CDR2 | 50-65 | 53-55 | 47-58 | 56-65 | 58-77 |
| V_{H} CDR3 | 95-102 | 96-101 | 93-101 | 105-117 | 109-137 |
| V_{L} CDR1 | 24-34 | 26-32 | 30-36 | 27-38 | 25-40 |
| V_{L} CDR2 | 50-56 | 50-52 | 46-55 | 56-65 | 58-77 |
| V_{L} CDR3 | 89-97 | 91-96 | 89-96 | 105-117 | 109-137 |

¹Residue numbering follows the nomenclature of Kabat et al., supra
²Residue numbering follows the nomenclature of Chothia et al., supra
³Residue numbering follows the nomenclature of MacCallum et al., supra
⁴Residue numbering follows the nomenclature of Lefranc et al., supra
⁵Residue numbering follows the nomenclature of Honegger and Plückthun, supra

Chimeric antigen receptors (CARs): As used herein, refers to an artificially constructed hybrid single-chain protein or single-chain polypeptide containing an extracellular target-binding (e.g., antigen-binding) domain, linked directly or indirectly to a transmembrane domain (“TM domain”, e.g., the transmembrane domain of a costimulatory molecule), which is in turn linked directly or indirectly to an intracellular signaling domain (ISD) comprising a primary immune cell signaling domain (e.g., one involved in T cell or NK cell activation). The extracellular target-binding domain can be a single-chain variable fragment derived from an antibody (scFv). In addition to scFvs, other single chain antigen binding domains can be used in CAR, e.g., tandem scFvs, single-domain antibody fragments (VHHs or sdAbs), single domain bispecific antibodies (BsAbs), intrabodies, nanobodies, immunokines in a single chain format, and Fab, Fab′, or (Fab′)2 in single chain formats. The extracellular target-binding domain can be joined to the TM domain via a flexible hinge/spacer region. The intracellular signaling domain (ISD) comprises a primary signaling sequence, or primary immune cell signaling sequence, which can be from an antigen-dependent, TCR-associated T cell activation molecule, e.g., a portion of the intracellular domain of TCRζ, FcRγ, FcRβ, CD3γ, CD3δ, CD3ε, CD3ζ, CD5, CD22, CD79a, CD79b, or CD66d. The ISD can further comprise a costimulatory signaling sequence; e.g., a portion of the intracellular domain of an antigen-independent, costimulatory molecule such as CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds CD83, or the like. Characteristics of CARs include their ability to redirect immune cell (e.g., T cell or NK cell) specificity and reactivity toward a selected target in either MHC-restricted (in cases of TCR-mimic antibodies) or non-MHC-restricted (in cases of antibodies against cell surface proteins) manners, exploiting the antigen-binding properties of monoclonal antibodies. The non-MHC-restricted antigen recognition gives immune cells (e.g., T cells or NK cells) expressing CARs the ability to recognize antigen independent of antigen processing, thus bypassing a major mechanism of tumor escape.

There are currently three generations of CARs. The “first generation” CARs are typically single-chain polypeptides composed of a scFv as the antigen-binding domain fused to a transmembrane domain fused to the cytoplasmic/intracellular domain, which comprises a primary immune cell signaling sequence such as the intracellular domain from the CD3ζ chain, which is the primary transmitter of signals from endogenous TCRs. The “first generation” CARs can provide de novo antigen recognition and cause activation of both CD4⁺ and CD8⁺ T cells through their CD3ζ chain signaling domain in a single fusion molecule, independent of HLA-mediated antigen presentation. The “second generation” CARs add intracellular domains from various costimulatory molecules (e.g., CD28, 4-1BB, ICOS, OX40) to the primary immune cell signaling sequence of the CAR to provide additional signals to the T cell. Thus, the “second generation” CARs comprise fragments that provide costimulation (e.g., CD28 or 4-IBB) and activation (e.g., CD3ζ). Preclinical studies have indicated that the “second generation” CARs can improve the antitumor activity of T cells. For example, robust efficacy of the “second generation” CAR modified T cells was demonstrated in clinical trials targeting the CD19 molecule in patients with chronic lymphoblastic leukemia (CLL) and acute lymphoblastic leukemia (ALL). The “third generation” CARs comprise those that provide multiple costimulation (e.g., CD28 and 4-1BB) and activation (e.g., CD3ζ). Examples of CAR T therapies are described, see, e.g., U.S. Pat. No. 10,221,245 describing CAR CTL019 which has an anti-CD19 extracellular target-binding domain, a transmembrane domain from CD8, a costimulatory domain from 4-1BB, and a primary signaling domain from CD3ζ, as well as U.S. Pat. No. 9,855,298 which describes a CAR having an anti-CD19 extracellular target-binding domain, a costimulatory domain from CD28, and a primary signaling domain from CD3ζ.

Adoptive cell therapy: Adoptive cell therapy is a therapeutic approach that typically includes isolation and ex vivo expansion and/or manipulation of immune cells (e.g., NK cells or T cells) and subsequent administration of these cells to a patient, for example for the treatment of cancer. Administered cells may be autologous or allogeneic. Cells may be manipulated to express engineered receptors (including CAR) in any one of the known ways, including, for example, by using RNA and DNA transfection, viral transduction, electroporation, all of which are technologies known in the art.

The term “adoptive cell therapeutic composition” refers to any composition comprising cells suitable for adoptive cell transfer. In exemplary embodiments, the adoptive cell therapeutic composition comprises a cell type selected from a group consisting of a tumor infiltrating lymphocyte (TIL) and CAR (i.e., chimeric antigen receptor) modified lymphocytes (e.g., CAR T cells). In another embodiment, the adoptive cell therapeutic composition comprises a cell type selected from a group consisting of T-cells, CD8⁺ cells, CD4⁺ cells, NK-cells, delta-gamma T-cells, regulatory T-cells, and peripheral blood mononuclear cells. In another embodiment, TILs, T-cells, CD8⁺ cells, CD4⁺ cells, NK-cells, delta-gamma T-cells, regulatory T-cells, or peripheral blood mononuclear cells form the adoptive cell therapeutic composition. In one embodiment, the adoptive cell therapeutic composition comprises T cells.

In some embodiments, the CAR comprising a CD30 costimulatory domain expressed in the cell is a first generation, second generation, or third generation CAR, as described above. In accordance with the presently disclosed subject matter, the CARs of the engineered immune cells provided herein comprise an extracellular antigen-binding domain, a transmembrane domain, and an intracellular domain. WO 2019032699 describes T cells co-expressing a CAR and an inducible bispecific antibody.

Comparable: As used herein, refers to two or more agents, entities, situations, sets of conditions, etc. that may not be identical to one another but that are sufficiently similar to permit comparison there between so that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Control: As used herein, refers to the art-understood meaning of a “control” being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. As used herein, a “control” may refer to a “control antibody”. A “control antibody” may be a human, chimeric, humanized, CDR-grafted, multispecific, or bispecific antibody as described herein, an antibody that is different as described herein, or a parental antibody. In one experiment, the “test” (i.e., the variable being tested) is applied. In the second experiment, the “control,” the variable being tested is not applied. In some embodiments, a control is a historical control (i.e., of a test or assay performed previously, or an amount or result that is previously known). In some embodiments, a control is or comprises a printed or otherwise saved record. A control may be a positive control or a negative control.

Corresponding to: As used herein designates the position/identity of an amino acid residue in a polypeptide of interest. Those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190th amino acid in a particular amino acid chain but rather corresponds to the residue found at 190 in the reference polypeptide; those of ordinary skill in the art readily appreciate how to identify “corresponding” amino acids.

Detection entity/agent: As used herein, refers to any element, molecule, functional group, compound, fragment or moiety that is detectable. In some embodiments, a detection entity is provided or utilized alone. In some embodiments, a detection entity is provided and/or utilized in association with (e.g., joined to) another agent. Examples of detection entities include, but are not limited to: various ligands, radionuclides (e.g., 3H, 14C, 18F, 19F, 32P, 35S, 135I, 125I, 123I, 64Cu, 187Re, 111In, 90Y, 99mTc, 177Lu, 89Zr etc.), fluorescent dyes (for specific exemplary fluorescent dyes, see below), chemiluminescent agents (such as, for example, acridinum esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes (for specific examples of enzymes, see below), colorimetric labels (such as, for example, dyes, colloidal gold, and the like), biotin, dioxigenin, haptens, and proteins for which antisera or monoclonal antibodies are available.

Effector function: As used herein refers a biochemical event that results from the interaction of an antibody Fc region with an Fc receptor or ligand. Effector functions include but are not limited to antibody-dependent cell-mediated cytotoxicity (ADCC), antibody-dependent cell-mediated phagocytosis (ADCP), and complement-mediated cytotoxicity (CMC). In some embodiments, an effector function is one that operates after the binding of an antigen, one that operates independent of antigen binding, or both.

Effector cell: As used herein refers to a cell of the immune system that mediates one or more effector functions. In some embodiments, effector cells may include, but may not be limited to, one or more of monocytes, macrophages, neutrophils, dendritic cells, eosinophils, mast cells, platelets, large granular lymphocytes, Langerhans' cells, natural killer (NK) cells, T-lymphocytes, B-lymphocytes and may be from any organism including but not limited to humans, mice, rats, rabbits, and monkeys.

Engineered: As used herein refers, in general, to the aspect of having been manipulated by the hand of man. For example, in some embodiments, a polynucleotide may be considered to be “engineered” when two or more sequences that are not linked together in that order in nature are manipulated by the hand of man to be directly linked to one another in the engineered polynucleotide. In some particular such embodiments, an engineered polynucleotide may comprise a regulatory sequence that is found in nature in operative association with a first coding sequence but not in operative association with a second coding sequence, is linked by the hand of man so that it is operatively associated with the second coding sequence. Alternatively or additionally, in some embodiments, first and second nucleic acid sequences that each encode polypeptide elements or domains that in nature are not linked to one another may be linked to one another in a single engineered polynucleotide. Comparably, in some embodiments, a cell or organism may be considered to be “engineered” if it has been manipulated so that its genetic information is altered (e.g., new genetic material not previously present has been introduced, or previously present genetic material has been altered or removed). As is common practice and is understood by those in the art, progeny of an engineered polynucleotide or cell are typically still referred to as “engineered” even though the actual manipulation was performed on a prior entity. Furthermore, as will be appreciated by those skilled in the art, a variety of methodologies are available through which “engineering” as described herein may be achieved. For example, in some embodiments, “engineering” may involve selection or design (e.g., of nucleic acid sequences, polypeptide sequences, cells, tissues, and/or organisms) through use of computer systems programmed to perform analysis or comparison, or otherwise to analyze, recommend, and/or select sequences, alterations, etc.). Alternatively or additionally, in some embodiments, “engineering” may involve use of in vitro chemical synthesis methodologies and/or recombinant nucleic acid technologies such as, for example, nucleic acid amplification (e.g., via the polymerase chain reaction) hybridization, mutation, transformation, transfection, etc., and/or any of a variety of controlled mating methodologies. As will be appreciated by those skilled in the art, a variety of established such techniques (e.g., for recombinant DNA, oligonucleotide synthesis, and tissue culture and transformation (e.g., electroporation, lipofection, etc.) are well known in the art and described in various general and more specific references that are cited and/or discussed throughout the present specification. See e.g., Sambrook et al., Molecular Cloning: A Laboratory Manual (2d ed., Cold Spring Harbor Laboratory Press, Cold Spring Harbor, N.Y., 1989).

Epitope: As used herein, includes any moiety that is specifically recognized by an immunoglobulin (e.g., antibody or receptor) binding component. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized). An antibody moiety described herein may bind to an epitope comprising between 7 and 50 amino acids (e.g., between 7 and 50 contigous amino acids), e.g., between 7 and 45, between 7 and between 7 and 40, between 7 and 35, between 7 and 30, between 7 and 25, between 7 and 20, between 7 and 15, between 7 and 10, between 10 and 50, between 15 and 50, between 20 and 50, between 25 and 50, between 30 and 50, between 35 and 50, between 40 and 50, between 45 and 50, between 10 and 45, between 15 and 40, between 20 and 35, or between 25 and 30 amino acids.

Excipient: As used herein, refers to a non-therapeutic agent that may be included in a pharmaceutical composition, for example to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Expression cassette: As used herein, refers to a nucleic acid construct that, when introduced into a host cell, results in transcription and/or translation of an RNA or polypeptide, respectively.

Heterologous: As used herein, refers to a polynucleotide or polypeptide that does not naturally occur in a host cell or a host organism. A heterologous polynucleotide or polypeptide may be introduced into the host cell or host organism using well-known recombinant methods, e.g., using an expression cassette comprising the heterologous polynucleotide optionally linked to a promoter.

Framework or framework region: As used herein, refers to the sequences of a variable region minus the CDRs. Because a CDR sequence can be determined by different systems, likewise a framework sequence is subject to correspondingly different interpretations. The six CDRs divide the framework regions on the heavy and light chains into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, FR1, for example, represents the first framework region closest to the amino terminal end of the variable region and 5′ with respect to CDR1, and FRs represents two or more of the sub-regions constituting a framework region.

Host cell: As used herein, refers to a cell into which exogenous DNA (recombinant or otherwise) has been introduced. Persons of skill upon reading this disclosure will understand that such terms refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. In some embodiments, host cells include prokaryotic and eukaryotic cells selected from any of the Kingdoms of life that are suitable for expressing an exogenous DNA (e.g., a recombinant nucleic acid sequence). Exemplary cells include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g., S. cerevisiae, S. pombe, P. pastoris, P. methanolica, etc.), plant cells, insect cells (e.g., SF-9, SF-21, baculovirus-infected insect cells, Trichoplusia ni, etc.), non-human animal cells, human cells, or cell fusions such as, for example, hybridomas or quadromas. In some embodiments, a host cell is a human, monkey, ape, hamster, rat, or mouse cell. In some embodiments, a host cell is eukaryotic and is selected from the following cells: CHO (e.g., CHO K1, DXB-1 1 CHO, Veggie-CHO), COS (e.g., COS-7), retinal cell, Vero, CV1, kidney (e.g., HEK293, 293 EBNA, MSR 293, MDCK, HaK, BHK), HeLa, HepG2, WI38, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3 A cell, HT1080 cell, myeloma cell, tumor cell, and a cell line derived from an aforementioned cell. In some embodiments, a host cell comprises one or more viral genes, e.g., a retinal cell that expresses a viral gene (e.g., a PER.C6™ cell).

Human antibody: As used herein, is intended to include antibodies having variable and constant regions generated (or assembled) from human immunoglobulin sequences. In some embodiments, antibodies (or antibody moieties) may be considered to be “human” even though their amino acid sequences include residues or elements not encoded by human germline immunoglobulin sequences (e.g., include sequence variations, for example, that may (originally) have been introduced by random or site-specific mutagenesis in vitro or by somatic mutation in vivo), for example in one or more CDRs and in particular CDR3. Human antibodies, human antibody moieties, and their fragments can be isolated from human immune cells or generated recombinantly or synthetically, including semi-synthetically.

Humanized: As is known in the art, the term “humanized” is commonly used to refer to antibodies (or moieties) whose amino acid sequence includes V_{H} and V_{L} region sequences from a reference antibody raised in a non-human species (e.g., a mouse), but also includes modifications in those sequences relative to the reference antibody intended to render them more “human-like”, i.e., more similar to human germline variable sequences. In some embodiments, a “humanized” antibody (or antibody moiety) is one that immunospecifically binds to an antigen of interest and that has a framework (FR) region having substantially the amino acid sequence as that of a human antibody, and a complementary determining region (CDR) having substantially the amino acid sequence as that of a non-human antibody. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor immunoglobulin) and all or substantially all of the framework regions are those of a human immunoglobulin consensus sequence. In some embodiments, a humanized antibody also comprises at least a portion of an immunoglobulin constant region (Fc), typically that of a human immunoglobulin constant region. In some embodiments, a humanized antibody contains both the light chain as well as at least the variable domain of a heavy chain. The antibody also may include a C_{H}1, hinge, C_{H}2, C_{H}3, and, optionally, a C_{H}4 region of a heavy chain constant region. In some embodiments, a humanized antibody only contains a humanized V_{L} region. In some embodiments, a humanized antibody only contains a humanized V_{H} region. In some certain embodiments, a humanized antibody contains humanized V_{H} and V_{L} regions.

Hydrophilic: As used herein, the term “hydrophilic” and/or “polar” refers to a tendency to mix with, or dissolve easily in, water.

Hydrophobic: As used herein, the term “hydrophobic” and/or “non-polar”, refers to a tendency to repel, not combine with, or an inability to dissolve easily in, water.

Improve, increase, or reduce: As used herein, or grammatical equivalents thereof, indicate values that are relative to a baseline measurement, such as a measurement in the same individual prior to initiation of a treatment described herein, or a measurement in a control individual (or multiple control individuals) in the absence of the treatment described herein. A “control individual” is an individual afflicted with the same form of disease or injury as the individual being treated. In some embodiments, the methods for treating a cancer (e.g., a hematological cancer or a solid tumor cancer) using a CAR described herein may increase cell apoptosis (e.g., increase tumor cell apoptosis) in an individual by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% compared to the individual prior to receiving treatment or to a control individual. In some embodiments, the methods for treating a cancer (e.g., a hematological cancer or a solid tumor cancer) using a CAR described herein may reduce tumor size (e.g., reduce tumor size) in an individual by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% compared to the individual prior to receiving treatment or to a control individual.

In vitro: As used herein refers to events that occur in an artificial environment, e.g., in a test tube or reaction vessel, in cell culture, etc., rather than within a multi-cellular organism.

In vivo: As used herein refers to events that occur within a multi-cellular organism, such as a human and a non-human animal. In the context of cell-based systems, the term may be used to refer to events that occur within a living cell (as opposed to, for example, in vitro systems).

Isolated: As used herein, refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

K_{D}: As used herein, refers to the dissociation constant of a binding agent (e.g., an antibody agent or binding component thereof) from a complex with its partner (e.g., the epitope to which the antibody agent or binding component thereof binds).

k_{off}: As used herein, refers to the off rate constant for dissociation of a binding agent (e.g., an antibody agent or binding component thereof) from a complex with its partner (e.g., the epitope to which the antibody agent or binding component thereof binds).

kₒₙ: As used herein, refers to the on rate constant for association of a binding agent (e.g., an antibody agent or binding component thereof) with its partner (e.g., the epitope to which the antibody agent or binding component thereof binds).

Linker: As used herein, is used to refer to that portion of a multi-element polypeptide that connects different elements to one another. For example, those of ordinary skill in the art appreciate that a polypeptide whose structure includes two or more functional or organizational domains often includes a stretch of amino acids between such domains that links them to one another. In some embodiments, a polypeptide comprising a linker element has an overall structure of the general form S1-L-S2, wherein S1 and S2 may be the same or different and represent two domains associated with one another by the linker. In some embodiments, a linker is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acids in length. In some embodiments, a linker has between 3 and 7 amino acids, between 7 and 15 amino acids, or between 20 and 30 (e.g., between 20 and 25 or between 25 and 30) amino acids. In some embodiments, a linker is characterized in that it tends not to adopt a rigid three-dimensional structure, but rather provides flexibility to the polypeptide. A variety of different linker elements that can appropriately be used when engineering polypeptides (e.g., fusion polypeptides) known in the art (see e.g., Holliger, P., et al., 1993, Proc. Natl. Acad. Sci. U.S.A. 90:6444-6448; Poljak, R. J. et al., 1994, Structure 2:1121-1123).

Multivalent binding antibody (or multispecific antibody): As used herein, refers an antibody capable of binding to two or more antigens, which can be on the same molecule or on different molecules. Multivalent binding antibodies as described herein are, in some embodiments, engineered to have the two or more antigen binding sites, and are typically not naturally occurring proteins. Multivalent binding antibodies as described herein refer to antibodies capable of binding two or more related or unrelated targets. Multivalent binding antibodies may be composed of multiple copies of a single antibody moiety or multiple copies of different antibody moieties. Such antibodies are capable of binding to two or more antigens and may be tetravalent or multivalent. Multivalent binding antibodies may additionally comprise a therapeutic agent, such as, for example, an immunomodulator, toxin or an RNase. Multivalent binding antibodies as described herein are, in some embodiments, capable of binding simultaneously to at least two targets that are of different structure, e.g., two different antigens, two different epitopes on the same antigen, or a hapten and/or an antigen or epitope. Multivalent binding antibodies of the present invention may be monospecific (capable of binding one antigen) or multispecific (capable of binding two or more antigens), and may be composed of two heavy chain polypeptides and two light chain polypeptides. Each binding site, in some embodiments, is composed of a heavy chain variable domain and a light chain variable domain with a total of six CDRs involved in antigen binding per antigen binding site.

Nucleic acid: As used herein, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to individual nucleic acid residues (e.g., nucleotides and/or nucleosides); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds.

In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is single stranded; in some embodiments, a nucleic acid is double stranded. In some embodiments a nucleic acid has a nucleotide sequence comprising at least one element that encodes, or is the complement of a sequence that encodes, a polypeptide. In some embodiments, a nucleic acid has enzymatic activity.

Operably linked: As used herein, refers to a juxtaposition wherein the components described are in a relationship permitting them to function in their intended manner. A control sequence “operably linked” to a coding sequence is ligated in such a way that expression of the coding sequence is achieved under conditions compatible with the control sequences. “Operably linked” sequences include both expression control sequences that are contiguous with a gene of interest and expression control sequences that act in trans or at a distance to control said gene of interest. The term “expression control sequence” as used herein refers to polynucleotide sequences that are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism. For example, in prokaryotes, such control sequences generally include promoter, ribosomal binding site, and transcription termination sequence, while in eukaryotes, typically, such control sequences include promoters and transcription termination sequence. The term “control sequences” is intended to include components whose presence is essential for expression and processing, and can also include additional components whose presence is advantageous, for example, leader sequences and fusion partner sequences.

Physiological conditions: As used herein, has its art-understood meaning referencing conditions under which cells or organisms live and/or reproduce. In some embodiments, the term refers to conditions of the external or internal milieu that may occur in nature for an organism or cell system. In some embodiments, physiological conditions are those conditions present within the body of a human or non-human animal, especially those conditions present at and/or within a surgical site. Physiological conditions typically include, e.g., a temperature range of 20-40° C., atmospheric pressure of 1, pH of 6-8, glucose concentration of 1-20 mM, oxygen concentration at atmospheric levels, and gravity as it is encountered on earth. In some embodiments, conditions in a laboratory are manipulated and/or maintained at physiologic conditions. In some embodiments, physiological conditions are encountered in an organism.

Polypeptide: As used herein, refers to any polymeric chain of amino acids. In some embodiments, the amino acids are joined to each other by peptide bonds or modified peptide bonds. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is synthetically designed and/or produced. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids.

In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class.

In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30 to 40%, and is often greater than about 50%, 60%, 70%, 80%, 90%, 91%, 92%, 93%, 94%, 95%, 96%, 97%, 98%, 99% or more and/or includes at least one region (i.e., a conserved region that may in some embodiments may be or comprise a characteristic sequence element) that shows very high sequence identity, often greater than 90% or even 95%, 96%, 97%, 98%, or 99%. Such a conserved region usually encompasses at least three to four and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice-versa, and/or fragments may be present in a different order in the polypeptide of interest than in the parent), so that the polypeptide of interest is a derivative of its parent polypeptide.

Prevent or prevention: As used herein when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Recombinant: As used herein, is intended to refer to polypeptides (e.g., antibodies or antibody moieties) that are designed, engineered, prepared, expressed, created or isolated by recombinant means, such as polypeptides expressed using a recombinant expression vector transfected into a host cell, polypeptides isolated from a recombinant, combinatorial human polypeptide library (Hoogenboom H. R., 1997, TIB Tech. 15:62-70; Azzazy H., and Highsmith W. E., 2002, Clin. Biochem. 35:425-45; Gavilondo J. V., and Larrick J. W., 2002, BioTechniques 29:128-45; Hoogenboom H., and Chames P., 2000, Immunol. Today 21:371-8), antibodies isolated from an animal (e.g., a mouse) that is transgenic for human immunoglobulin genes (see e.g., Taylor, L. D., et al., 1992, Nucl. Acids Res. 20:6287-95; Kellermann S-A., and Green L. L., 2002, Curr. Opin. Biotech. 13:593-7; Little, M. et al., 2000, Immunol. Today 21:364-70; Murphy, A. J. et al., 2014, Proc. Natl. Acad. Sci. U.S.A. 111(14):5153-8) or polypeptides prepared, expressed, created or isolated by any other means that involves splicing selected sequence elements to one another. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source. For example, in some embodiments, a recombinant antibody is comprised of sequences found in the germline of a source organism of interest (e.g., human, mouse, etc.). In some embodiments, a recombinant antibody has an amino acid sequence that resulted from mutagenesis (e.g., in vitro or in vivo, for example in a transgenic animal), so that the amino acid sequences of the V_{H} and V_{L} regions of the recombinant antibodies are sequences that, while originating from and related to germline V_{H} and V_{L} sequences, may not naturally exist within the germline antibody repertoire in vivo.

Reference: As used herein describes a standard, control, or other appropriate reference against which a comparison is made as described herein. For example, in some embodiments, a reference is a standard or control agent, animal, individual, population, sample, sequence, series of steps, set of conditions, or value against which an agent, animal, individual, population, sample, sequence, series of steps, set of conditions, or value of interest is compared. In some embodiments, a reference is tested and/or determined substantially simultaneously with the testing or determination of interest. In some embodiments, a reference is a historical reference, optionally embodied in a tangible medium. Typically, as would be understood by those skilled in the art, a reference is determined or characterized under conditions comparable to those utilized in the assessment of interest.

Specific binding: As used herein, refers to a binding agent's ability to discriminate between possible partners in the environment in which binding is to occur. A binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex; in some embodiments, specific binding is assessed by detecting or determining ability of the binding agent to compete an alternative interaction between its partner and another entity. In some embodiments, specific binding is assessed by performing such detections or determinations across a range of concentrations. In some embodiments, specific binding is assessed by determining the difference in binding affinity between cognate and non-cognate targets. For example, a binding agent may have a binding affinity for a cognate target that is about 3-fold, 4-fold, 5-fold, 6-fold, 7-fold, 8-fold, 9-fold, 10-fold or more than binding affinity for a non-cognate target.

Specificity: As is known in the art, “specificity” is a measure of the ability of a particular ligand to distinguish its binding partner from other potential binding partners.

Subject: As used herein, means any mammal, including humans. In certain embodiments of the present invention the subject is an adult, an adolescent or an infant. In some embodiments, terms “individual” or “patient” are used and are intended to be interchangeable with “subject.” Also contemplated by the present invention are the administration of the pharmaceutical compositions and/or performance of the methods of treatment in utero.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantial sequence homology: As used herein, the phrase “substantial homology” to refers to a comparison between amino acid or nucleic acid sequences. As will be appreciated by those of ordinary skill in the art, two sequences are generally considered to be “substantially homologous” if they contain homologous residues in corresponding positions. Homologous residues may be identical residues. Alternatively, homologous residues may be non-identical residues with appropriately similar structural and/or functional characteristics. For example, as is well known by those of ordinary skill in the art, certain amino acids are typically classified as “hydrophobic” or “hydrophilic” amino acids, and/or as having “polar” or “non-polar” side chains. Substitution of one amino acid for another of the same type may often be considered a “homologous” substitution. Typical amino acid categorizations are summarized as follows:

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown amino acid | Xaa | X |

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul et al., 1990, J. Mol. Biol., 215(3):403-410; Altschul et al., 1996, Meth. Enzymology 266:460-480; Altschul et al., 1997, Nucleic Acids Res. 25:3389-3402; Baxevanis et al, Bioinformatics: A Practical Guide to the Analysis of Genes and Proteins, Wiley, 1998; and Misener, et al, (eds.), Bioinformatics Methods and Protocols (Methods in Molecular Biology, Vol. 132), Humana Press, 1999. In addition to identifying homologous sequences, the programs mentioned above typically provide an indication of the degree of homology. In some embodiments, two sequences are considered to be substantially homologous if at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more of their corresponding residues are homologous over a relevant stretch of residues. In some embodiments, the relevant stretch is a complete sequence. In some embodiments, the relevant stretch is at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500 or more residues.

Surface plasmon resonance: As used herein, refers to an optical phenomenon that allows for the analysis of specific binding interactions in real-time, for example through detection of alterations in protein concentrations within a biosensor matrix, such as by using a BIAcore system (Pharmacia Biosensor AB, Uppsala, Sweden and Piscataway, N.J.). For further descriptions, see Jonsson, U. et al., 1993, Ann. Biol. Clin. 51:19-26; Jonsson, U. et al., 1991, Biotechniques 11:620-627; Johnsson, B. et al., 1995, J. Mol. Recognit. 8:125-131; and Johnsson, B. et al., 1991, Anal. Biochem. 198:268-277.

Therapeutic agent: As used herein, generally refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Therapeutically effective amount: As used herein, is meant an amount that produces the desired effect for which it is administered. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

Treatment: As used herein, the term “treatment” (also “treat” or “treating”), in its broadest sense, refers to any administration of a substance (e.g., provided compositions) that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be administered to a subject who does not exhibit signs of the relevant disease, disorder and/or condition and/or of a subject who exhibits only early signs of the disease, disorder, and/or condition. Alternatively or additionally, in some embodiments, treatment may be administered to a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Variant: As used herein, the term “variant” refers to an entity that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. As will be appreciated by those skilled in the art, any biological or chemical reference entity has certain characteristic structural elements. A variant, by definition, is a distinct chemical entity that shares one or more such characteristic structural elements. To give but a few examples, a polypeptide may have a characteristic sequence element comprised of a plurality of amino acids having designated positions relative to one another in linear or three-dimensional space and/or contributing to a particular biological function, a nucleic acid may have a characteristic sequence element comprised of a plurality of nucleotide residues having designated positions relative to on another in linear or three-dimensional space. For example, a variant polypeptide may differ from a reference polypeptide as a result of one or more differences in amino acid sequence and/or one or more differences in chemical moieties (e.g., carbohydrates, lipids, etc.) covalently attached to the polypeptide backbone. In some embodiments, a variant polypeptide shows an overall sequence identity with a reference polypeptide that is at least 85%, 86%, 87%, 88%, 89%, 90%, 91%, 92%, 93%, 94%, 95%, 96%, 97%, or 99%. Alternatively or additionally, in some embodiments, a variant polypeptide does not share at least one characteristic sequence element with a reference polypeptide.

In some embodiments, the reference polypeptide has one or more biological activities. In some embodiments, a variant polypeptide shares one or more of the biological activities of the reference polypeptide. In some embodiments, a variant polypeptide lacks one or more of the biological activities of the reference polypeptide. In some embodiments, a variant polypeptide shows a reduced level of one or more biological activities as compared with the reference polypeptide. In many embodiments, a polypeptide of interest is considered to be a “variant” of a parent or reference polypeptide if the polypeptide of interest has an amino acid sequence that is identical to that of the parent but for a small number of sequence alterations at particular positions. Typically, fewer than 20%, 15%, 10%, 9%, 8%, 7%, 6%, 5%, 4%, 3%, 2% of the residues in the variant are substituted as compared with the parent. In some embodiments, a variant has 10, 9, 8, 7, 6, 5, 4, 3, 2, or 1 substituted residue as compared with a parent. Often, a variant has a very small number (e.g., fewer than 5, 4, 3, 2, or 1) number of substituted functional residues (i.e., residues that participate in a particular biological activity). Furthermore, a variant typically has not more than 5, 4, 3, 2, or 1 insertions or deletions, and often has no insertions or deletions, as compared with the parent. Moreover, any additions or deletions are typically fewer than about 25, about 20, about 19, about 18, about 17, about 16, about 15, about 14, about 13, about 10, about 9, about 8, about 7, about 6, and commonly are fewer than about 5, about 4, about 3, or about 2 residues. In some embodiments, the parent or reference polypeptide is one found in nature. As will be understood by those of ordinary skill in the art, a plurality of variants of a particular polypeptide of interest may commonly be found in nature, particularly when the polypeptide of interest is an infectious agent polypeptide.

Vector: As used herein, refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.”

Wild type: As used herein, the term “wild type” has its art-understood meaning that refers to an entity having a structure and/or activity as found in nature in a “normal” (as contrasted with mutant, variant, diseased, altered, etc.) state or context. Those of ordinary skill in the art will appreciate that wild type genes and polypeptides often exist in multiple different forms (e.g., alleles).

### BRIEF DESCRIPTION OF THE DRAWINGS

FIG. 1: Flow cytometric analysis of CD8⁺ Receptor+CAR T-cells. FIG. 1 shows the expression of T cell differentiation markers CCR7 and CD45RA in CAR-T cells prior to target engagement. Values in Q1 (low CCR7 and high CD45RA expression levels), Q2 (high CCR7 and high CD45RA), Q3 (high CCR7 and low CD45RA), and Q4 (low CCR7 and low CD45RA) represent the percentage of the T cells in this population belonging to the Effector, the Naïve, the Central Memory, and the Effector Memory T cell subsets, respectively.

FIG. 2: T cell-mediated short-term target cell killing (16 hours) by a panel of αCD19 CARs tested on the CD19⁺ Nalm6 leukemia cell line.

FIG. 3A: A CFSE dilution/proliferation assay of αCD19 CAR T-cells following Nalm6 target cell engagement. The proliferation of αCD19-CAR T-cells at Day 3 and Day 7 post-first and second engagements, respectively are shown.

FIG. 3B: A CFSE dilution profile of the CAR T cells following engagement with Raji cells.

FIGS. 4A and 4B: The killing of target Nalm6 cells mediated by T cells expressing a panel of αCD19 CARs. FIG. 4A shows the number of CD3⁺ αCD19 CAR-T cells and FIG. 4B shows the number of CD19⁺ target cells remaining after several engagements.

FIGS. 5A and 5B: Measurement of exhaustion marker PD-1 expression in αCD19-CD28z and -CD30z CAR T cells at 3 days post-second engagement (E2D3) with αCD19⁺ Nalm6 or Raji target cells.

FIGS. 6A and 6B: Exhaustion marker PD-1 expression in αCD19-CD8T-41BBz and -CD8T-CD30z CAR T cells at 3 days post-second engagement (E2D3) with Nalm6 or Raji cells.

FIGS. 7A and 7B: MFI of PD-1 expression over time (E1D3-E2D7) in αCD19 CAR T cells with Nalm6 engagement.

FIGS. 7C and 7D: MFI of PD-1 expression over time (E1D3-E2D7) in αCD19 CAR T cells with Raji cell engagement.

FIG. 8: Short-term killing (16 hours) assay of a panel of αAFP CAR T cells were tested on the AFP⁺ HepG2 cell line.

FIG. 9: A CFSE proliferation assay of αAFP CAR-T cells following engagement with the AFP⁺ target cell line HepG2 at Day 3 and Day 7 post-first engagement and post-second engagement, respectively.

FIGS. 10A and 10B: MFI of PD-1 expression in αAFP CAR T cells at day 3 following the second engagement with HepG2 target cells.

FIGS. 11A and 11B: Comparison of the median fluorescence intensity (MFI) in αAFP CAR T cells after HepG2 engagement across several timepoints.

FIGS. 12A and 12B: Raji Target cell killing by αCD19-CARs. FIG. 12A shows Raji cell killing during a period of days after engagement with T cells expressing αCD19 CARs. FIG. 12B shows the number of αCD19 CD3⁺ CAR T cells over the same time period post-engagement.

FIG. 13A: Raji target cell killing several days post-engagement by αCD19-CD30z and αCD19-CD8T-41BBz CAR T cells.

FIG. 13B: Remaining αCD19-CD30z and αCD19-CD8T-41BBz CAR T cell count during several days post-engagement with Raji target cells.

### DETAILED DESCRIPTION OF THE EMBODIMENTS

The present invention relates to the discovery of CARs that use a costimulatory domain from CD30 (also referred to herein as a CD30 costimulatory domain) and T cells expressing these CARs having far less expression of PD-1, an inhibitor of T cell activation, than T cells with CARs containing a costimulatory domain from, e.g., CD28 or 4-1BB. The T cells with CARs comprising a CD30 costimulatory domain provide superior persistance of tumor cell killing. The invention also provides the use of such CARs and T cells expressing such CARs to treat cancer (e.g., a hematological cancer or a solid tumor cancer).

### I. Chimeric Antigen Receptors (CARs)

The disclosure provides a chimeric antigen receptor (CAR) comprising: (a) an extracellular target-binding domain comprising an antibody moiety; (b) a transmembrane domain; (c) a CD30 costimulatory domain; and (d) a primary signaling domain. The costimulatory domain and primary signaling domain are parts of the the intracellular signaling domain of the CAR. As described and demonstrated herein, T cells with CARs containing a costimulatory domain from CD30 express far less PD-1, an inhibitor of T cell activation, than T cells with CARs containing a costimulatory domain from, e.g., CD28 or 4-1BB. T cells with CARs containing a costimulatory domain from CD30 also demonstrate persistence in cytotoxic potential. The costimulatory domain from CD30 may ameliorate the functional unresponsiveness that leads to T cell exhaustion, i.e., anergy. The ability of a CD30 costimulatory domain to provide T cells with superior persistence of tumor cell killing is unexpected since CD30 lacks a p56lck-binding site that is thought to be crucial for CAR costimulation.

In some embodiments, a spacer domain may be present between (a) and (b), between (b) and (c), and/or between (c) and (d). The spacer domain can be any oligo- or polypeptide that functions to link two parts of the CAR. A spacer domain may comprise up to about 300 amino acids, including for example about 10 to about 100, or about 25 to about 50 amino acids. Examplary sequences of CARs described herein can be found in the Informal Sequence Listing table. In some embodiments, the CARs with myc-tags are used in in vitro and pre-clinical assays. For in vivo use, i.e., in vivo use in humans, the corresponding CAR constructs without myc-tags are used.

Target Antigen

In some embodiments, a CAR described herein comprises an antigen-binding module that specifically binds to a cell surface antigen, wherein the cell surface antigen is CD19, CD20, CD22, CD47, CD158e, GPC3, ROR1, ROR2, BCMA, GPRC5D, FcRL5, MUC16, MCT4, or PSMA, including variants or mutants thereof. Specific binding to a full antigen, e.g., a cell surface antigen, is sometimes referred to as “non-MHC-restricted binding”. In some embodiments, a CAR described herein comprises an antigen-binding module that specifically binds to a complex comprising a peptide and an MHC protein, wherein the peptide is derived from a protein selected from the group consisting of WT-1, AFP, HPV16-E7, NY-ESO-1, PRAME, EBV-LMP2A, HIV-1, KRAS, FoxP3, Histone H3.3, and PSA, including variants or mutants thereof. Specific binding to a complex comprising a peptide and an MHC protein is sometimes referred to as “MHC-restricted binding”.

In some embodiments, according to any of the CARs described herein comprising an antibody moiety that specifically binds to a target antigen, the antibody moiety comprises the CDRs or variable domains (V_{H} and/or V_{L} domains) of an antibody moiety specific for the target antigen. In some embodiments, the antibody moiety comprises the CDRs or variable domains (V_{H} and/or V_{L} domains) of an antibody moiety specific for CD19 (see, e.g., WO2017/066136A2). In some embodiments, the antibody moiety comprises the CDRs or variable domains (V_{H} and/or V_{L} domains) of an antibody moiety specific for CD19 (e.g., V_{H} domain comprising, consisting essentially of, or consisting of the amino acid sequence of SEQ ID NO:62 and/or V_{L} domain comprising, consisting essentially of, or consisting of the amino acid sequence of SEQ ID NO:63, or CDRs contained therein). In some embodiments, the antibody moiety comprises the CDRs or variable domains (V_{H} and/or V_{L} domains) of an antibody moiety specific for CD20 (e.g., V_{H} domain comprising, consisting essentially of, or consisting of the amino acid sequence of SEQ ID NO:64 and/or V_{L} domain comprising, consisting essentially of, or consisting of the amino acid sequence of SEQ ID NO:65, or CDRs contained therein). In some embodiments, the antibody moiety comprises the CDRs or variable domains (V_{H} and/or V_{L} domains) of an antibody moiety specific for CD22 (see, e.g., U.S. Ser. No. 62/650,955 filed Mar. 30, 2018 and PCT/US2019/025032, filed Mar. 29, 2019), the contents of which are incorporated herein by reference in their entirety). In some embodiments, the antibody moiety comprises the CDRs or variable domains (V_{H} and/or V_{L} domains) of an antibody moiety specific for CD22 (e.g., V_{H} domain comprising, consisting essentially of, or consisting of the amino acid sequence of SEQ ID NO:58 and/or V_{L} domain comprising, consisting essentially of, or consisting of the amino acid sequence of SEQ ID NO:59, or CDRs contained therein). In some embodiments, the antibody moiety comprises the CDRs or variable domains (V_{H} and/or V_{L} domains) of an antibody moiety specific for CD22 (e.g., V_{H} domain comprising, consisting essentially of, or consisting of the amino acid sequence of SEQ ID NO:60 and/or V_{L} domain comprising, consisting essentially of, or consisting of the amino acid sequence of SEQ ID NO:61, or CDRs contained therein). In some embodiments, the antibody moiety comprises the CDRs or variable domains (V_{H} and/or V_{L} domains) of an antibody moiety specific for CD47 (see, e.g., WO2018/200585A1). In some embodiments, the antibody moiety comprises the CDRs or variable domains (V_{H} and/or V_{L} domains) of an antibody moiety specific for CD47 (e.g., V_{H} domain comprising, consisting essentially of, or consisting of the amino acid sequence of SEQ ID NO:66 and/or V_{L} domain comprising, consisting essentially of, or consisting of the amino acid sequence of SEQ ID NO:67, or CDRs contained therein).

In some embodiments, the antibody moiety comprises the CDRs or variable domains (V_{H} and/or V_{L} domains) of an antibody moiety specific for GPC3 (see, e.g., WO2018/200586A1, the contents of which are incorporated herein by reference in their entirety). In some embodiments, the antibody moiety comprises the CDRs or variable domains (V_{H} and/or V_{L} domains) of an antibody moiety specific for GPC3 (e.g., V_{H} domain comprising, consisting essentially of, or consisting of the amino acid sequence of SEQ ID NO:68 and/or V_{L} domain comprising, consisting essentially of, or consisting of the amino acid sequence of SEQ ID NO:69, or CDRs contained therein). In some embodiments, the antibody moiety comprises the CDRs or variable domains (V_{H} and/or V_{L} domains) of an antibody moiety specific for GPC3 (e.g., V_{H} domain comprising, consisting essentially of, or consisting of the amino acid sequence of SEQ ID NO:70 and/or V_{L} domain comprising, consisting essentially of, or consisting of the amino acid sequence of SEQ ID NO:71, or CDRs contained therein). In some embodiments, the antibody moiety comprises the CDRs or variable domains (V_{H} and/or V_{L} domains) of an antibody moiety specific for ROR1 (see, e.g., WO2016/187220 and WO2016/187216). In some embodiments, the antibody moiety comprises the CDRs or variable domains (V_{H} and/or V_{L} domains) of an antibody moiety specific for ROR2 (see, e.g., WO2016/142768). In some embodiments, the antibody moiety comprises the CDRs or variable domains (V_{H} and/or V_{L} domains) of an antibody moiety specific for BCMA (see, e.g., WO2016/090327 and WO2016/090320). In some embodiments, the antibody moiety comprises the CDRs or variable domains (V_{H} and/or V_{L} domains) of an antibody moiety specific for GPRC5D (see, e.g., WO2016/090329 and WO2016/090312). In some embodiments, the antibody moiety comprises the CDRs or variable domains (V_{H} and/or V_{L} domains) of an antibody moiety specific for FCRL5 (see, e.g., WO2016/090337). In some embodiments, the antibody moiety comprises the CDRs or variable domains (V_{H} and/or V_{L} domains) of an antibody moiety specific for MUC16 (see, e.g., U.S. Ser. No. 62/845,065, filed May 8, 2019 and U.S. Ser. No. 62/768,730, filed Nov. 16, 2018 the contents of which are incorporated herein by reference in their entirety). In some embodiments, the antibody moiety comprises the CDRs or variable domains (V_{H} and/or V_{L} domains) of an antibody moiety specific for MCT4 (see, e.g., PCT/US2019/023402, filed Mar. 21, 2019, the contents of which are incorporated herein by reference in their entirety). In some embodiments, the antibody moiety comprises the CDRs or variable domains (V_{H} and/or V_{L} domains) of an antibody moiety specific for PSMA (see, e.g., PCT/US2019/037534, filed Jun. 17, 2019, the contents of which are incorporated herein by reference in their entirety). In some embodiments, the antibody moiety comprises the CDRs or variable domains (V_{H} and/or V_{L} domains) of an antibody moiety specific for a WT-1 peptide/MHC complex (see, e.g., WO2012/135854, WO2015/070078, and WO2015/070061). In some embodiments, the antibody moiety comprises the CDRs or variable domains (V_{H} and/or V_{L} domains) of an antibody moiety specific for an AFP peptide/MHC complex (see, e.g., WO2016/161390). In some embodiments, the antibody moiety comprises the CDRs or variable domains (V_{H} and/or V_{L} domains) of an antibody moiety specific for a HPV16-E7 peptide/MHC complex (see, e.g., WO2016/182957). In some embodiments, the antibody moiety comprises the CDRs or variable domains (V_{H} and/or V_{L} domains) of an antibody moiety specific for a NY-ESO-1 peptide/MHC complex (see, e.g., WO2016/210365). In some embodiments, the antibody moiety comprises the CDRs or variable domains (V_{H} and/or V_{L} domains) of an antibody moiety specific for a PRAME peptide/MHC complex (see, e.g., WO2016/191246). In some embodiments, the antibody moiety comprises the CDRs or variable domains (V_{H} and/or V_{L} domains) of an antibody moiety specific for a EBV-LMP2A peptide/MHC complex (see, e.g., WO2016/201124). In some embodiments, the antibody moiety comprises the CDRs or variable domains (V_{H} and/or V_{L} domains) of an antibody moiety specific for a KRAS peptide/MHC complex (see, e.g., WO2016/154047). In some embodiments, the antibody moiety comprises the CDRs or variable domains (V_{H} and/or V_{L} domains) of an antibody moiety specific for a PSA peptide/MHC complex (see, e.g., WO2017/015634). In some embodiments, the antibody moiety comprises the CDRs or variable domains (V_{H} and/or V_{L} domains) of an antibody moiety specific for a FoxP3 peptide/MHC complex (see, e.g., PCT/US2019/018112 filed Feb. 14, 2018, the contents of which are incorporated herein by reference in their entirety). In some embodiments, the antibody moiety comprises the CDRs or variable domains (V_{H} and/or V_{L} domains) of an antibody moiety specific for a Histone H3.3 peptide/MHC complex (see, e.g., WO2018/132597). In some embodiments, the antibody moiety comprises the CDRs or variable domains (V_{H} and/or V_{L} domains) of an antibody moiety specific for a HIV-1 peptide/MHC complex (see, e.g., WO2018057967). In some embodiments, the antibody moiety is a scFv (single chain variable fragment) comprising a V_{H} domain and a V_{L} domain. In some embodiments, the scFv comprises an antigen-binding module that specifically binds to a complex comprising a peptide and an MHC protein, known as a peptide/MHC complex.

Table A lists exemplary proteins whose fragments or peptides can be targeted by the CAR. Also listed are possible diseases, specifically possible cancers that such T cells can treat.

TABLE A

| CAR Target | |
|---|---|
| (Peptide-MHC Complex, including | Exemplary Cancers |
| mutant peptide, other than GPC3) | to be Treated |
| AFP | Liver Cancer |
| GPC3 | Liver Cancer |
| KRAS | Pancreatic Cancer |
| PSA | Prostate Cancer |
| NY-ESO-1 and PRAME | Melanoma |
| KRAS and PRAME | Gastrointestinal |
| | Cancers |
| PRAME, PSA | Breast Cancers |
| | (including Metastatic |
| | Breast Cancer) |
| WT1, NY-ESO-1 | Ovarian Cancer |
| KRAS | Colorectal Cancers |
| | (including Metastatic |
| | Colorectal Cancer) |
| Histone H3.3 | Glioblastoma |
| KRAS, NY-ESO-1 | Lung Cancer |
| PRAME | Renal Cell |
| | Carcinoma |

Extracellular Target-Binding Domain

An extracellular target-binding domain of a CAR described herein may comprise an antibody moieity or an antigen-binding fragment thereof. In certain embodiments, the extracellular target-binding domain can be a single-chain variable fragment derived from an antibody (scFv), a tandem scFv, a single-domain antibody fragment (VHHs or sdAbs), a single domain bispecific antibody (BsAbs), an intrabody, a nanobody, an immunokine in a single chain format, and Fab, Fab′, or (Fab′)₂ in a single chain format. In other embodiments, the extracellular target-binding domain can be an antibody moiety that comprises covalently bound multiple chains of variable fragments. The extracellular target-binding domain can be joined to the TM domain via a flexible hinge/spacer region.

scFv and Tandem scFv

A CAR described herein may comprise an antibody moiety that is a single chain Fv (scFv) antibody. An scFv antibody may comprise a light chain variable region and a heavy chain variable region, in which the light chain variable region and the heavy chain variable region may be joined using recombinant methods by a synthetic linker to make a single polypeptide chain. In some embodiments, the scFv may have the structure “(N-terminus) light chain variable region-linker-heavy chain variable region (C-terminus),” in which the heavy chain variable region is joined to the C-terminus of the light chain variable region by way of a linker. In other embodiments, the scFv may have the structure “(N-terminus) heavy chain variable region-linker-light chain variable region (C-terminus),” in which the light chain variable region is joined to the C-terminus of the heavy chain variable region by way of a linker. A linker may be a polypeptide including 2 to 200 (e.g., 2, 3, 5, 10, 15, 20, 25, 30, 35, 40, 45, 50, 55, 60, 65, 70, 75, 80, 85, 90, 95, 100, 110, 120, 130, 140, 150, 160, 170, 180, 190, or 200) amino acids. Suitable linkers may contain flexible amino acid residues such as glycine and serine.

A CAR described herein may comprise an extracellular target-binding domain comprising an antibody moiety that is a tandem scFv comprising a first scFv and a second scFv (also referred to herein as a “tandem scFv multispecific antibody”). In some embodiments, the tandem scFv multispecific antibody further comprises at least one (such as at least about any of 2, 3, 4, 5, or more) additional scFv.

In some embodiments, there is provided a tandem scFv multispecific (e.g., bispecific) antibody comprising a) a first scFv that specifically binds to an extracellular region of a target ligand, and b) a second scFv. In some embodiments, the target ligand is CD22 and the first scFv specifically binds to an extracellular region of CD22. In some embodiments, the target ligand is CD19 and the first scFv specifically binds to an extracellular region of CD19. In some embodiments, the target ligand is a complex comprising an alpha-fetoprotein (AFP) peptide and a MHC class I protein, and the first scFv specifically binds to the complex but not to AFP or the AFP peptide alone or MHC alone.

In some embodiments, the second scFv specifically binds to another antigen. In some embodiments, the second scFv specifically binds to an antigen on the surface of a cancer cell. In some embodiments, the second scFv specifically binds to an antigen on the surface of a cell that does not express CD22. In some embodiments, the second scFv specifically binds to an antigen on the surface of a cell that does not express CD19. In some embodiments, the second scFv specifically binds to an antigen on the surface of a cell that does not express AFP peptide. In some embodiments, the second scFv specifically binds to an antigen on the surface of a cytotoxic cell. In some embodiments, the second scFv specifically binds to an antigen on the surface of a lymphocyte, such as a T cell, an NK cell, a neutrophil, a monocyte, a macrophage, or a dendritic cell. In some embodiments, the second scFv specifically binds to an antigen on the surface of an effector T cell, such as a cytotoxic T cell. In some embodiments, the second scFv specifically binds to an antigen on the surface of an effector cell, including for example CD3γ, CD3δ, CD3ε, CD3ζ, CD28, CD16a, CD56, CD68, GDS2D, OX40, GITR, CD137, CD27, CD40L and HVEM.

In some embodiments, the first scFv is human, humanized, or semi-synthetic. In some embodiments, the second scFv is human, humanized, or semi-synthetic. In some embodiments, both the first scFv and the second scFv are human, humanized, or semi-synthetic. In some embodiments, the tandem scFv multispecific antibody further comprises at least one (such as at least about any of 2, 3, 4, 5, or more) additional scFv.

In some embodiments, there is provided a tandem scFv multispecific (e.g., bispecific) antibody comprising a) a first scFv that specifically binds to an extracellular region of a target antigen, and b) a second scFv, wherein the tandem scFv multispecific antibody is a tandem di-scFv or a tandem tri-scFv. In some embodiments, the tandem scFv multispecific antibody is a tandem di-scFv. In some embodiments, the tandem scFv multispecific antibody is a bispecific T-cell engager.

In some embodiments, the tandem di-scFv bispecific antibody binds to an extracellular region of a target antigen or a portion thereof with a Kd between about 0.1 pM to about 500 nM (such as about any of 0.1 pM, 1.0 pM, 10 pM, 50 pM, 100 pM, 500 pM, 1 nM, 10 nM, 50 nM, 100 nM, or 500 nM, including any ranges between these values). In some embodiments, the tandem di-scFv bispecific antibody binds to an extracellular region of a target antigen or a portion thereof with a Kd between about 1 nM to about 500 nM (such as about any of 1, 10, 25, 50, 75, 100, 150, 200, 250, 300, 350, 400, 450, or 500 nM, including any ranges between these values).

A variety of technologies are known in the art for designing, constructing, and/or producing multispecific antibodies. Multispecific antibodies may be constructed that either utilize the full immunoglobulin framework (e.g., IgG), single chain variable fragment (scFv), or combinations thereof. Bispecific antibodies may be composed of two scFv units in tandem as described above. In the case of anti-tumor immunotherapy, bispecific antibodies that comprise two single chain variable fragments (scFvs) in tandem may be designed such that an scFv that binds a tumor antigen is linked with an scFv that engages T cells, i.e., by binding CD3 on the T cells. Thus, T cells are recruited to a tumor site to mediate killing of the tumor cells. Bispecific antibodies can be made, for example, by combining heavy chains and/or light chains that recognize different epitopes of the same or different antigen. In some embodiments, by molecular function, a bispecific binding agent binds one antigen (or epitope) on one of its two binding arms (one V_{H}/V_{L} pair), and binds a different antigen (or epitope) on its second arm (a different V_{H}/V_{L} pair). By this definition, a bispecific binding agent has two distinct antigen binding arms (in both specificity and CDR sequences), and is monovalent for each antigen to which it binds. In certain embodiments, a bispecific binding agent according to the present invention comprises a first and a second scFv. In some certain embodiments, a first scFv is linked to the C-terminal end of a second scFv. In some certain embodiments, a second scFv is linked to the C-terminal end of a first scFv. In some certain embodiments, scFvs are linked to each other via a linker (e.g., SRGGGGSGGGGSGGGGSLEMA (SEQ ID NO:38)). In some certain embodiments, scFvs are linked to each other without a linker.

A linker may be a polypeptide including 2 to 200 (e.g., 2, 3, 5, 10, 15, 20, 25, 30, 35, 40, 45, 50, 55, 60, 65, 70, 75, 80, 85, 90, 95, 100, 110, 120, 130, 140, 150, 160, 170, 180, 190, or 200) amino acids. Suitable linkers may contain flexible amino acid residues such as glycine and serine. In certain embodiments, a linker may contain motifs, e.g., multiple or repeating motifs, of GS, GGS, GGGGS (SEQ ID NO:39), GGSG (SEQ ID NO:40), or SGGG (SEQ ID NO:41). In some embodiments, a linker may have the sequence GSGS (SEQ ID NO:42), GSGSGS (SEQ ID NO:43), GSGSGSGS (SEQ ID NO:44), GSGSGSGSGS (SEQ ID NO:45), GGSGGS (SEQ ID NO:46), GGSGGSGGS (SEQ ID NO:47), GGSGGSGGSGGS (SEQ ID NO:48). GGSG (SEQ ID NO:49), GGSGGGSG (SEQ ID NO:50), or GGSGGGSGGGSG (SEQ ID NO:51). In other embodiments, a linker may also contain amino acids other than glycine and serine, e.g., SRGGGGSGGGGSGGGGSLEMA (SEQ ID NO:38).

Transmembrane Domain (TM)

The transmembrane domain may be derived either from a natural or from a synthetic source. Where the source is natural, the domain may be derived from any membrane-bound or transmembrane protein. Transmembrane regions of particular use in this invention may be derived from (i.e., comprise at least the transmembrane region(s) of) the α, β, δ, γ, or ζ chain of the T-cell receptor, CD28, CD3ε, CD3ζ, CD45, CD4, CD5, CD8, CD9, CD16, CD22, CD30, CD33, CD37, CD64, CD80, CD86, CD134, CD137, or CD154. In some embodiments, a transmembrane domain can be chosen based on, for example, the nature of the various other proteins or trans-elements that bind the transmembrane domain or the cytokines induced by the transmembrane domain. For example, the transmembrane domain derived from CD30 lacks a binding site for the p56lck kinase, a common motif in the TNF receptor family. In some embodiments, a transmembrane region of particular use in this invention may be derived from (i.e., comprise at least the transmembrane region(s) of) CD8, e.g., a transmembrane region comprising a sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the sequence of SEQ ID NO:26. In some embodiments, a transmembrane region of particular use in this invention may be derived from (i.e., comprise at least the transmembrane region(s) of) CD30, e.g., a transmembrane region comprising a sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the sequence of SEQ ID NO:30.

In certain embodiments, the transmembrane domain may be chosen based on the target antigen. For example, a CAR containing an antibody moiety specific for an AFP peptide/MHC complex and a transmembrane domain derived from CD8 appeared to have better in vitro killing properties than a corresponding CAR containing a transmembrane domain derived from CD30. This result was not observed in a CAR containing an antibody moiety specific for CD19.

In some embodiments, the transmembrane domain may be synthetic, in which case it may comprise predominantly hydrophobic residues such as leucine and valine. In some embodiments, a triplet of phenylalanine, tryptophan, and valine may be found at each end of a synthetic transmembrane domain. In some embodiments, a short oligo- or polypeptide linker, having a length of, for example, between about 2 and about 10 (such as about any of 2, 3, 4, 5, 6, 7, 8, 9, or 10) amino acids in length may form the linkage between the transmembrane domain and the intracellular signaling domain of a CAR described herein. In some embodiments, the linker is a glycine-serine doublet. In some embodiments, a transmembrane domain comprises a partial extracellular domain (ECD). For example, a transmembrane domain derived from CD8 or CD30 comprises an ECD. In some embodiments, an ECD links the transmembrane domain to the extracellular target-binding domain of a CAR.

In some embodiments, the transmembrane domain that naturally is associated with one of the sequences in the intracellular signaling domain of the CAR is used (e.g., if an anti-CD22 CAR (or an anti-CD19 CAR, or an anti-AFP CAR) intracellular signaling domain comprises a CD30 costimulatory sequence, the transmembrane domain of the CAR is derived from the CD30 transmembrane domain). In some embodiments, the transmembrane domain can be selected or modified by amino acid substitution to avoid binding of such domains to the transmembrane domains of the same or different surface membrane proteins to minimize interactions with other members of the receptor complex.

Intracellular Signaling Domain

The intracellular signaling domain of the CAR is responsible for activation of at least one of the normal effector functions of the immune cell in which the CAR has been placed in. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term “intracellular signaling sequence” is thus meant to include any truncated portion of the intracellular signaling domain sufficient to transduce the effector function signal.

Examples of intracellular signaling domains for use in the CAR of the invention include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone are insufficient for full activation of the T cell and that a secondary or costimulatory signal is also required. Thus, T cell activation can be said to be mediated by two distinct classes of intracellular signaling sequence: those that initiate antigen-dependent primary activation through the TCR (primary signaling sequences) and those that act in an antigen-independent manner to provide a secondary or costimulatory signal (costimulatory signaling sequences).

Primary signaling sequences regulate primary activation of the TCR complex either in a stimulatory way, or in an inhibitory way. Primary signaling sequences that act in a stimulatory manner may contain signaling motifs which are known as immunoreceptor tyrosine-based activation motifs or ITAMs. In some embodiments, the CARs described herein comprise one or more ITAMs.

Examples of ITAM containing primary signaling sequences that are of particular use in the invention include those derived from TCRζ, FcRγ, FcRβ, CD3γ, CD3δ, CD3ε, CD3ζ, CD5, CD22, CD79a, CD79b, and CD66d. In some embodiments, an ITAM containing primary signaling sequence is derived from CD3ζ.

In some embodiments, the CAR comprises a primary signaling sequence derived from CD3ζ. For example, the intracellular signaling domain of the CAR can comprise the CD3ζ intracellular signaling sequence by itself or combined with any other desired intracellular signaling sequence(s) useful in the context of the CAR of the invention. For example, the intracellular signaling domain of the CAR can comprise a CD3ζ primary intracellular signaling sequence and a CD30 costimulatory signaling sequence. As described herein, T cells with CARs containing a costimulatory domain from CD30 express far less PD-1, an inhibitor of T cell activation, than T cells with CARs containing a costimulatory domain from, e.g., CD28 or 4-1BB. T cells with CARs containing a costimulatory domain from CD30 also demonstrate persistence in cytotoxic potential. The costimulatory domain from CD30 may ameliorate the functional unresponsiveness that leads to T cell exhaustion, i.e., anergy. The ability of a CD30 costimulatory domain to provide T cells with superior persistence of tumor cell killing is unexpected since CD30 lacks a p56lck-binding site that is thought to be crucial for CAR costimulation.

Thus, for example, in some embodiments, there is provided a CAR comprising a) an extracellular target-binding domain comprising an antibody moiety that specifically binds to an extracellular region of a target ligand or a portion thereof, b) a transmembrane domain, and c) an intracellular signaling domain comprising a CD30 costimulartory domain and a primary signaling domain. In some embodiments, the intracellular signaling domain is capable of activating an immune cell. In some embodiments, the intracellular signaling domain comprises a primary signaling sequence and a costimulatory signaling sequence. In some embodiments, the primary signaling sequence comprises a CD3ζ intracellular signaling sequence. In some embodiments, the costimulatory signaling sequence comprises a CD30 intracellular signaling sequence. In some embodiments, the intracellular signaling domain comprises a CD3ζ primary intracellular signaling sequence and a CD30 intracellular signaling sequence.

### II. Multispecific Antibodies

A CAR described herein may comprise an antibody moiety that is a multispecific antibody. A multispecific antibody may comprise a first binding moiety and a second binding moiety (such as a second antigen-binding moiety). Multispecific antibodies are antibodies that have binding specificities for at least two different antigens or epitopes (e.g., bispecific antibodies have binding specificities for two antigens or epitopes). Multispecific antibodies with more than two specificities are also contemplated. For example, trispecific antibodies can be prepared (see, e.g., Tutt et al., J. Immunol. 147: 60 (1991)). It is to be appreciated that one of skill in the art could select appropriate features of individual multispecific antibodies described herein to combine with one another to form a multispecific antibodies of the invention.

Thus, for example, in some embodiments, there is provided a multispecific (e.g., bispecific) antibody comprising a) a first binding moiety that specifically binds to an extracellular region of a first target antigen, and b) a second binding moiety (such as an antigen-binding moiety). In some embodiments, the second binding moiety specifically binds to a different target antigen. In some embodiments, the second binding moiety specifically binds to an antigen on the surface of a cell, such as a cytotoxic cell. In some embodiments, the second binding moiety specifically binds to an antigen on the surface of a lymphocyte, such as a T cell, an NK cell, a neutrophil, a monocyte, a macrophage, or a dendritic cell. In some embodiments, the second binding moiety specifically binds to an effector T cell, such as a cytotoxic T cell (also known as cytotoxic T lymphocyte (CTL) or T killer cell).

In some embodiments, the second binding moiety specifically binds to a tumor antigen. Examples of tumor antigens include, but are not limited to, alpha fetoprotein (AFP), CA15-3, CA27-29, CA19-9, CA-125, calretinin, carcinoembryonic antigen, CD34, CD99, CD117, chromogranin, cytokeratin, desmin, epithelial membrane protein (EMA), Factor VIII, CD31 FL1, glial fibrillary acidic protein (GFAP), gross cystic disease fluid protein (GCDFP-15), HMB-45, human chorionic gonadotropin (hCG), inhibin, keratin, CD45, a lymphocyte marker, MART-1 (Melan-A), Myo Dl, muscle-specific actin (MSA), neurofilament, neuron-specific enolase (NSE), placental alkaline phosphatase (PLAP), prostate-specific antigen, S100 protein, smooth muscle actin (SMA), synaptophysin, thyroglobulin, thyroid transcription factor-1, tumor M2-PK, and vimentin.

In some embodiments, the second antigen-binding moiety in a bispecific antibody binds to CD3. In some embodiments, the second antigen-binding moiety specifically binds to CD3ε. In some embodiments, the second antigen-binding moiety specifically binds to an agonistic epitope of CD3ε. The term “agonistic epitope”, as used herein, means (a) an epitope that, upon binding of the multispecific antibody, optionally upon binding of several multispecific antibodies on the same cell, allows said multispecific antibodies to activate T cell receptor (TCR) signaling and induce T cell activation, and/or (b) an epitope that is solely composed of amino acid residues of the epsilon chain of CD3 and is accessible for binding by the multispecific antibody, when presented in its natural context on T cells (i.e., surrounded by the TCR, the CD37 chain, etc.), and/or (c) an epitope that, upon binding of the multispecific antibody, does not lead to stabilization of the spatial position of CD3ε relative to CD3γ.

In some embodiments, the second antigen-binding moiety binds specifically to an antigen on the surface of an effector cell, including for example CD3γ, CD3δ, CD3ε, CD3ζ, CD28, CD16a, CD56, CD68, GDS2D, OX40, GITR, CD137, CD27, CD40L and HVEM. In other embodiments, the second antigen-binding moiety binds to a component of the complement system, such as C1q. C1q is a subunit of the C1 enzyme complex that activates the serum complement system. In other embodiments, the second antigen-binding moiety specifically binds to an Fc receptor. In some embodiments, the second antigen-binding moiety specifically binds to an Fcγ receptor (FcγR). The FcγR may be an FcγRIII present on the surface of natural killer (NK) cells or one of FcγRI, FcγRIIA, FcγRIIBI, FcγRIIB2, and FcγRIIIB present on the surface of macrophages, monocytes, neutrophils and/or dendritic cells. In some embodiments, the second antigen-binding moiety is an Fc region or functional fragment thereof. A “functional fragment” as used in this context refers to a fragment of an antibody Fc region that is still capable of binding to an FcR, in particular to an FcγR, with sufficient specificity and affinity to allow an FcγR bearing effector cell, in particular a macrophage, a monocyte, a neutrophil and/or a dendritic cell, to kill the target cell by cytotoxic lysis or phagocytosis. A functional Fc fragment is capable of competitively inhibiting the binding of the original, full-length Fc portion to an FcR such as the activating FcγRI. In some embodiments, a functional Fc fragment retains at least 30%, 40%, 50%, 60%, 70%, 80%, 90% or 95% of its affinity to an activating FcγR. In some embodiments, the Fc region or functional fragment thereof is an enhanced Fc region or functional fragment thereof. The term “enhanced Fc region”, as used herein, refers to an Fc region that is modified to enhance Fc receptor-mediated effector-functions, in particular antibody-dependent cell-mediated cytotoxicity (ADCC), complement-dependent cytotoxicity (CDC), and antibody-mediated phagocytosis. This can be achieved as known in the art, for example by altering the Fc region in a way that leads to an increased affinity for an activating receptor (e.g. FcγRIIIA (CD16A) expressed on natural killer (NK) cells) and/or a decreased binding to an inhibitory receptor (e.g., FcγRIIB1/B2 (CD32B)).

In some embodiments, the multispecific antibodies allow killing of antigen-presenting target cells and/or can effectively redirect CTLs to lyse target-presenting target cells. In some embodiments, the multispecific (e.g., bispecific) antibodies of the present invention show an in vitro EC50 ranging from 10 to 500 ng/ml, and is able to induce redirected lysis of about 50% of the target cells through CTLs at a ratio of CTLs to target cells of from about 1:1 to about 50:1 (such as from about 1:1 to about 15:1, or from about 2:1 to about 10:1).

In some embodiments, the multispecific (e.g., bispecific) antibody is capable of cross-linking a stimulated or unstimulated CTL and the target cell in such a way that the target cell is lysed. This offers the advantage that no generation of target-specific T cell clones or common antigen presentation by dendritic cells is required for the multispecific antibody to exert its desired activity. In some embodiments, the multispecific antibody of the present invention is capable of redirecting CTLs to lyse the target cells in the absence of other activating signals. In some embodiments, the second antigen-binding moiety specifically binds to CD3 (e.g., specifically binds to CD3ε), and signaling through CD28 and/or TL-2 is not required for redirecting CTLs to lyse the target cells.

Methods for measuring the preference of the multispecific antibody to simultaneously bind to two antigens (e.g., antigens on two different cells) are within the normal capabilities of a person skilled in the art. For example, when the second binding moiety specifically binds to the second antigen, the multispecific antibody may be contacted with a mixture of first antigen⁺/second antigen⁻ cells and first antigen/second antigen⁺ cells. The number of multispecific antibody-positive single cells and the number of cells cross-linked by multispecific antibodies may then be assessed by microscopy or fluorescence-activated cell sorting (FACS) as known in the art.

In some embodiments, the multispecific antibody is, for example, a diabody (db), a single-chain diabody (scDb), a tandem scDb (Tandab), a linear dimeric scDb (LD-scDb), a circular dimeric scDb (CD-scDb), a di-diabody, a tandem scFv, a tandem di-scFv (e.g., a bispecific T cell engager), a tandem tri-scFv, a tri(a)body, a bispecific Fab₂, a di-miniantibody, a tetrabody, an scFv-Fc-scFv fusion, a dual-affinity retargeting (DART) antibody, a dual variable domain (DVD) antibody, an IgG-scFab, an scFab-ds-scFv, an Fv2-Fe, an IgG-scFv fusion, a dock and lock (DNL) antibody, a knob-into-hole (KiH) antibody (bispecific IgG prepared by the KiH technology), a DuoBody (bispecific IgG prepared by the Duobody technology), a single-domain antibody fragment (VHHs or sdAbs), a single domain bispecific antibody (BsAbs), an intrabody, a nanobody, an immunokine in a single chain format, a heteromultimeric antibody, or a heteroconjugate antibody. In some embodiments, the multispecific antibody is a single chain antibody fragment. In some embodiments, the multispecific antibody is a tandem scFv (e.g., a tandem di-scFv, such as a bispecific T cell engager).

### III. Antibody-Drug Conjugates

In some embodiments, there is provided an immunoconjugate comprising an antibody moiety and a therapeutic agent (also referred to herein as an “antibody-drug conjugate”, or “ADC”). In some embodiments, the therapeutic agent is a toxin that is either cytotoxic, cytostatic, or otherwise prevents or reduces the ability of the target cells to divide. The use of ADCs for the local delivery of cytotoxic or cytostatic agents, i.e., drugs to kill or inhibit tumor cells in the treatment of cancer (Syrigos and Epenetos, Anticancer Research 19:605-614 (1999); Niculescu-Duvaz and Springer, Adv. Drg. Del. Rev. 26:151-172 (1997); U.S. Pat. No. 4,975,278) allows targeted delivery of the drug moiety to target cells, and intracellular accumulation therein, where systemic administration of these unconjugated therapeutic agents may result in unacceptable levels of toxicity to normal cells as well as the target cells sought to be eliminated (Baldwin et al., Lancet (Mar. 15, 1986):603-605 (1986); Thorpe, (1985) “Antibody Carriers Of Cytotoxic Agents In Cancer Therapy: A Review,” in Monoclonal Antibodies '84: Biological And Clinical Applications, A. Pinchera et al. (eds.), pp. 475-506). Maximal efficacy with minimal toxicity is sought thereby.

Therapeutic agents used in immunoconjugates (e.g., an ADC) include, for example, daunomycin, doxorubicin, methotrexate, and vindesine (Rowland et al., Cancer Immunol. Immunother. 21:183-187 (1986)). Toxins used in immunoconjugates include bacterial toxins such as diphtheria toxin, plant toxins such as ricin, small molecule toxins such as geldanamycin (Mandler et al., J. Nat. Cancer Inst. 92(19):1573-1581 (2000); Mandler et al., Bioorganic & Med. Chem. Letters 10:1025-1028 (2000); Mandler et al., Bioconjugate Chem. 13:786-791 (2002)), maytansinoids (EP 1391213; Liu et al., Proc. Natl. Acad. Sci. USA 93:8618-8623 (1996)), and calicheamicin (Lode et al., Cancer Res. 58:2928 (1998); Hinman et al., Cancer Res. 53:3336-3342 (1993)). The toxins may exert their cytotoxic and cytostatic effects by mechanisms including tubulin binding, DNA binding, or topoisomerase inhibition. Some cytotoxic drugs tend to be inactive or less active when conjugated to large antibodies or protein receptor ligands.

Enzymatically active toxins and fragments thereof that can be used include, for example, diphtheria A chain, nonbinding active fragments of diphtheria toxin, exotoxin A chain (from Pseudomonas aeruginosa), ricin A chain, abrin A chain, modeccin A chain, α-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. See, e.g., WO 93/21232 published Oct. 28, 1993.

Immunoconjugates (e.g., an ADC) of an antibody moiety and one or more small molecule toxins, such as a calicheamicin, maytansinoids, dolastatins, aurostatins, a trichothecene, and CC1065, and the derivatives of these toxins that have toxin activity, are also contemplated herein.

In some embodiments, there is provided an immunoconjugate (e.g., an ADC) comprising a therapeutic agent that has an intracellular activity. In some embodiments, the immunoconjugate is internalized and the therapeutic agent is a cytotoxin that blocks the protein synthesis of the cell, therein leading to cell death. In some embodiments, the therapeutic agent is a cytotoxin comprising a polypeptide having ribosome-inactivating activity including, for example, gelonin, bouganin, saporin, ricin, ricin A chain, bryodin, diphtheria toxin, restrictocin, Pseudomonas exotoxin A and variants thereof. In some embodiments, where the therapeutic agent is a cytotoxin comprising a polypeptide having a ribosome-inactivating activity, the immunoconjugate must be internalized upon binding to the target cell in order for the protein to be cytotoxic to the cells.

In some embodiments, there is provided an immunoconjugate (e.g., an ADC) comprising a therapeutic agent that acts to disrupt DNA. In some embodiments, the therapeutic agent that acts to disrupt DNA is, for example, selected from the group consisting of enediyne (e.g., calicheamicin and esperamicin) and non-enediyne small molecule agents (e.g., bleomycin, methidiumpropyl-EDTA-Fe(II)).

The present invention further contemplates an immunoconjugate (e.g., an ADC) formed between the antibody moiety and a compound with nucleolytic activity (e.g., a ribonuclease or a DNA endonuclease such as a deoxyribonuclease; DNase).

In some embodiments, the immunoconjugate comprises an agent that acts to disrupt tubulin. Such agents may include, for example, rhizoxin/maytansine, paclitaxel, vincristine and vinblastine, colchicine, auristatin dolastatin 10 MMAE, and peloruside A.

In some embodiments, the immunoconjugate (e.g., an ADC) comprises an alkylating agent including, for example, Asaley NSC 167780, AZQ NSC 182986, BCNU NSC 409962, Busulfan NSC 750, carboxyphthalatoplatinum NSC 271674, CBDCA NSC 241240, CCNU NSC 79037, CHIP NSC 256927, chlorambucil NSC 3088, chlorozotocin NSC 178248, cis-platinum NSC 119875, clomesone NSC 338947, cyanomorpholinodoxorubicin NSC 357704, cyclodisone NSC 348948, dianhydrogalactitol NSC 132313, fluorodopan NSC 73754, hepsulfam NSC 329680, hycanthone NSC 142982, melphalan NSC 8806, methyl CCNU NSC 95441, mitomycin C NSC 26980, mitozolamide NSC 353451, nitrogen mustard NSC 762, PCNU NSC 95466, piperazine NSC 344007, piperazinedione NSC 135758, pipobroman NSC 25154, porfiromycin NSC 56410, spirohydantoin mustard NSC 172112, teroxirone NSC 296934, tetraplatin NSC 363812, thio-tepa NSC 6396, triethylenemelamine NSC 9706, uracil nitrogen mustard NSC 34462, and Yoshi-864 NSC 102627.

In some embodiments, the immunoconjugate (e.g., an ADC) comprises a highly radioactive atom. A variety of radioactive isotopes are available for the production of radioconjugated antibodies. Examples include 211At, 131I, 125I, 90Y, 186Re, 188Re, 153Sm, 212Bi, 32P, 212Pb and radioactive isotopes of Lu.

In some embodiments, the antibody moiety can be conjugated to a “receptor” (such as streptavidin) for utilization in tumor pre-targeting wherein the antibody-receptor conjugate is administered to the patient, followed by removal of unbound conjugate from the circulation using a clearing agent and then administration of a “ligand” (e.g., avidin) that is conjugated to a cytotoxic agent (e.g., a radionucleotide).

In some embodiments, an immunoconjugate (e.g., an ADC) may comprise an antibody moiety conjugated to a prodrug-activating enzyme. In some such embodiments, a prodrug-activating enzyme converts a prodrug to an active drug, such as an anti-viral drug. Such immunoconjugates are useful, in some embodiments, in antibody-dependent enzyme-mediated prodrug therapy (“ADEPT”). Enzymes that may be conjugated to an antibody include, but are not limited to, alkaline phosphatases, which are useful for converting phosphate-containing prodrugs into free drugs; arylsulfatases, which are useful for converting sulfate-containing prodrugs into free drugs; proteases, such as serratia protease, thermolysin, subtilisin, carboxypeptidases and cathepsins (such as cathepsins B and L), which are useful for converting peptide-containing prodrugs into free drugs; D-alanylcarboxypeptidases, which are useful for converting prodrugs that contain D-amino acid substituents; carbohydrate-cleaving enzymes such as β-galactosidase and neuraminidase, which are useful for converting glycosylated prodrugs into free drugs; β-lactamase, which is useful for converting drugs derivatized with β-lactams into free drugs; and penicillin amidases, such as penicillin V amidase and penicillin G amidase, which are useful for converting drugs derivatized at their amine nitrogens with phenoxyacetyl or phenylacetyl groups, respectively, into free drugs. In some embodiments, enzymes may be covalently bound to antibody moieties by recombinant DNA techniques well known in the art. See, e.g., Neuberger et al., Nature 312:604-608 (1984).

In some embodiments, the therapeutic portion of the immunoconjugates (e.g., an ADC) may be a nucleic acid. Nucleic acids that may be used include, but are not limited to, anti-sense RNA, genes or other polynucleotides, including nucleic acid analogs such as thioguanine and thiopurine.

The present application further provides immunoconjugates (e.g., an ADC) comprising an antibody moiety attached to an effector molecule, wherein the effector molecule is a label, which can generate a detectable signal, indirectly or directly. These immunoconjugates can be used for research or diagnostic applications, such as for the in vivo detection of cancer. The label is preferably capable of producing, either directly or indirectly, a detectable signal. For example, the label may be radio-opaque or a radioisotope, such as 3H, 14C, 32P, 35S, 123I, 125I, 131I; a fluorescent (fluorophore) or chemiluminescent (chromophore) compound, such as fluorescein isothiocyanate, rhodamine or luciferin; an enzyme, such as alkaline phosphatase, β-galactosidase or horseradish peroxidase; an imaging agent; or a metal ion. In some embodiments, the label is a radioactive atom for scintigraphic studies, for example 99Tc or 123I, or a spin label for nuclear magnetic resonance (NMR) imaging (also known as magnetic resonance imaging, MRI), such as zirconium-89, iodine-123, iodine-131, indium-111, fluorine-19, carbon-13, nitrogen-15, oxygen-17, gadolinium, manganese or iron. Zirconium-89 may be complexed to various metal chelating agents and conjugated to antibodies, e.g., for PET imaging (WO 2011/056983).

In some embodiments, the immunoconjugate is detectable indirectly. For example, a secondary antibody that is specific for the immunoconjugate and contains a detectable label can be used to detect the immunoconjugate.

### IV. CAR Immune Cells

The present invention provides an immune cell (such as a T cell) presenting on its surface a CAR according to any of the CAR described herein (such an immune cell is also referred to herein as a “CAR immune cell”). In some embodiments, the immune cell comprises nucleic acid encoding the CAR, wherein the CAR is expressed from the nucleic acid and localized to the immune cell surface. In some embodiments, the immune cell is a T cell. In some embodiments, the immune cell is selected from the group consisting of a cytotoxic T cell, a helper T cell, a natural killer T cell, and a suppressor T cell. In some embodiments, the immune cell is modified to block or decrease the expression of one or more of the endogenous TCR subunits of the immune cell. For example, in some embodiments, the immune cell is an β T cell modified to block or decrease the expression of the TCR α and/or β chains or the immune cell is a γδ T cell modified to block or decrease the expression of the TCR γ and/or δ chains. Modifications of cells to disrupt gene expression include any such techniques known in the art, including for example RNA interference (e.g., siRNA, shRNA, miRNA), gene editing (e.g., CRISPR- or TALEN-based gene knockout), and the like.

In exemplary embodiments, the cell of the present disclosure is an immune cell or a cell of the immune system. Accordingly, the cell may be a B-lymphocyte, T-lymphocyte, thymocyte, dendritic cell, natural killer (NK) cell, monocyte, macrophage, granulocyte, eosinophil, basophil, neutrophil, myelomonocytic cell, megakaryocyte, peripheral blood mononuclear cell, myeloid progenitor cell, or a hematopoietic stem cell. In exemplary aspects, the cell is a T lymphocyte. In exemplary aspects, the T lymphocyte is CD8⁺, CD4⁺, CD8⁺/CD4⁺, or a T-regulatory (T-reg) cell. In exemplary embodiments, the T lymphocyte is genetically engineered to silence the expression of an endogenous TCR. In exemplary aspects, the cell is a natural killer (NK) cell.

For example, in some embodiments, there is provided an immune cell (such as a T cell) comprising nucleic acid encoding a CAR according to any of the CAR described herein, wherein the CAR is expressed from the nucleic acid and localized to the immune cell surface. In some embodiments, the CAR nucleic acid sequence is contained in a vector. Vectors may be selected, for example, from the group consisting of mammalian expression vectors and viral vectors (such as those derived from retroviruses, adenoviruses, adeno-associated viruses, herpes viruses, and lentiviruses). In some embodiments, one or more of the vectors is integrated into the host genome of the immune cell. In some embodiments, the CAR nucleic acid sequence is under the control of a promoter. In some embodiments, the promoter is inducible. In some embodiments, the promoter is operably linked to the 5′ end of the CAR nucleic acid sequence. In some embodiments, the immune cell is selected from the group consisting of a cytotoxic T cell, a helper T cell, a natural killer T cell, and a suppressor T cell.

Thus, in some embodiments, there is provided a CAR immune cell (such as a T cell) expressing on its surface a CAR described herein, wherein the CAR immune cell comprises: a CAR nucleic acid sequence encoding a CAR polypeptide chain of the CAR, wherein the CAR polypeptide chain is expressed from the CAR nucleic acid sequence to form the CAR, and wherein the CAR localizes to the surface of the immune cell.

### V. Fc Variants

In some embodiments, CARs described herein may comprise a variant Fc region, wherein the variant Fc region may comprise at least one amino acid modification relative to a reference Fc region (or parental Fc region or a wild-type Fc region). Amino acid modifications may be made in an Fc region to alter effector function and/or to increase serum stability of the CAR. CARs comprising variant Fc regions may demonstrate an altered affinity for an Fc receptor (e.g., an FcγR), provided that the variant Fc regions do not have a substitution at positions that make a direct contact with Fc receptor based on crystallographic and structural analysis of Fc-Fc receptor interactions such as those disclosed by Sondermann et al., 2000, Nature, 406:267-273. Examples of positions within the Fc region that make a direct contact with an Fc receptor such as an FcγR are amino acids 234-239 (hinge region), amino acids 265-269 (B/C loop), amino acids 297-299 (C′/E loop), and amino acids 327-332 (F/G) loop. In some embodiments, CARs comprising variant Fc regions may comprise a modification of at least one residue that makes a direct contact with an FcγR based on structural and crystallographic analysis.

Amino acid modifications in Fc regions to create variant Fc regions that, e.g., alter affinity for activating and/or inhibitory receptors, lead to improved effector function such as, e.g., Antibody-Dependent Cell-Mediated Cytotoxicity (ADCC) and Complement Dependent Cytotoxicity (CDC), increase binding affinity for C1q, reduce or eliminate FcR binding, increase half-life are known in the art (see, e.g., U.S. Pat. Nos. 9,051,373, 9,040,041, 8,937,158, 8,883,973, 8,883,147, 8,858,937, 8,852,586, 8,809,503, 8,802,823, 8,802,820, 8,795,661, 8,753,629, 8,753,628, 8,735,547, 8,735,545, 8,734,791, 8,697,396, 8,546,543, 8,475,792, 8,399,618, 8,394,925, 8,388,955, 8,383,109, 8,367,805, 8,362,210, 8,338,574, 8,324,351, 8,318,907, 8,188,231, 8,124,731, 8,101,720, 8,093,359, 8,093,357, 8,088,376, 8,084,582, 8,039,592, 8,012,476, 7,799,900, 7,790,858, 7,785,791, 7,741,072, 7,704,497, 7,662,925, 7,416,727, 7,371,826, 7,364,731, 7,335,742, 7,332,581, 7,317,091, 7,297,775, 7,122,637, 7,083,784, 6,737,056, 6,538,124, 6,528,624 and 6,194,551).

In some embodiments, a variant Fc region may have different glycosylation patterns as compared to a parent Fc region (e.g., aglycosylated). In some embodiments, different glycosylation patterns may arise from expression in different cell lines, e.g., an engineered cell line.

CARs described herein may comprise variant Fc regions that bind with a greater affinity to one or more FcγRs. Such CARs preferably mediate effector function more effectively as discussed infra. In some embodiments, CARs described herein may comprise variant Fc regions that bind with a weaker affinity to one or more FcγRs. Reduction or elimination of effector function may be desirable in certain cases, for example, in the case of CARs whose mechanism of action involves blocking or antagonism but not killing of the cells bearing a target antigen. In some embodiments, increased effector function may be directed to tumor cells and cells expressing foreign antigens.

### VI. CAR Production

Provided CARs or portions thereof, or nucleic acids encoding them, may be produced by any available means. Methods for production are well-known in the art. Technologies for generating antibodies (e.g., scFv antibodies, monoclonal antibodies, and/or polyclonal antibodies) are available in the art. It will be appreciated that a wide range of animal species can be used for the production of antisera, e.g., mouse, rat, rabbit, pig, cow, deer, sheep, goat, cat, dog, monkey, and chicken. The choice of animal may be decided upon the ease of manipulation, costs or the desired amount of sera, as would be known to one of skill in the art. It will be appreciated that antibodies can also be produced transgenically through the generation of a mammal or plant that is transgenic for the immunoglobulin heavy and light chain sequences of interest (e.g., a transgenic rodent transgenic for human immunoglobulin heavy and light chain genes). In connection with the transgenic production in mammals, antibodies can be produced in, and recovered from, the milk of goats, cows, or other mammals (see, e.g., U.S. Pat. Nos. 5,827,690, 5,756,687, 5,750,172, and 5,741,957; herein incorporated by reference in their entireties). Alternatively, antibodies may be made in chickens, producing IgY molecules (Schade et al., 1996, ALTEX 13(5):80-85).

Although embodiments employing CARs that contain human antibodies having, i.e., human heavy and light chain variable region sequences including human CDR sequences, are extensively discussed herein, the present invention also provides CARs that contain non-human antibodies. In some embodiments, non-human antibodies comprise human CDR sequences from an antibody as described herein and non-human framework sequences. Non-human framework sequences include, in some embodiments, any sequence that can be used for generating synthetic heavy and/or light chain variable regions using one or more human CDR sequences as described herein, including, e.g., sequences generated from mouse, rat, rabbit, pig, cow, deer, sheep, goat, cat, dog, monkey, chicken, etc. In some embodiments, a provided CAR includes an antibody generated by grafting one or more human CDR sequences as described herein onto a non-human framework sequence (e.g., a mouse or chicken framework sequence). In many embodiments, provided CAR comprise or are human antibodies (e.g., a human monoclonal antibody or fragment thereof, human antigen-binding protein or polypeptide, human multispecific antibody (e.g., a human bispecific antibody), a human polypeptide having one or more structural components of a human immunoglobulin polypeptide).

In some embodiments, antibodies suitable for the present invention are subhuman primate antibodies. For example, general techniques for raising therapeutically useful antibodies in baboons may be found, for example, in International Patent Application Publication No. 1991/11465 and in Losman et al., 1990, Int. J. Cancer 46:310. In some embodiments, antibodies (e.g., monoclonal antibodies) may be prepared using hybridoma methods (Milstein and Cuello, 1983, Nature 305(5934):537-40). In some embodiments, antibodies (e.g., monoclonal antibodies) may also be made by recombinant methods (see, e.g., U.S. Pat. No. 4,166,452).

Many of the difficulties associated with generating antibodies by B-cell immortalization can be overcome by engineering and expressing CAR components in E. coli or yeast using phage display. To ensure the recovery of high affinity antibodies a combinatorial immunoglobulin library must typically contain a large repertoire size. A typical strategy utilizes mRNA obtained from lymphocytes or spleen cells of immunized mice to synthesize cDNA using reverse transcriptase. The heavy and light chain genes are amplified separately by PCR and ligated into phage cloning vectors. Two different libraries may be produced, one containing the heavy chain genes and one containing the light chain genes. The libraries can be naïve or they can be semi-synthetic, i.e., with all amino acids (with the exception of cysteine) equally likely to be present at any given position in a CDR. Phage DNA is isolated from each library, and the heavy and light chain sequences are ligated together and packaged to form a combinatorial library. Each phage contains a random pair of heavy and light chain cDNAs and upon infection of E. coli directs the expression of the polypeptides in a CAR in infected cells. To identify a CAR that recognizes the antigen of interest, the phage library is plated, and the CAR molecules present in the plaques are transferred to filters. The filters are incubated with radioactively labeled antigen and then washed to remove excess unbound ligand. A radioactive spot on the autoradiogram identifies a plaque that contains a CAR that binds the antigen. Alternatively, identification of a CAR that recognizes the antigen of interest may be achieved by iterative binding of phage to the antigen, which is bound to a solid support, for example, beads or mammalian cells followed by removal of non-bound phage and by elution of specifically bound phage. In such embodiments, antigens are first biotinylated for immobilization to, for example, streptavidin-conjugated Dynabeads M-280. The phage library is incubated with the cells, beads or other solid support and non-binding phage is removed by washing. CAR phage clones that bind the antigen of interest are selected and tested for further characterization.

Once selected, positive clones may be tested for their binding to the antigen of interest expressed on the surface of live cells by flow cytometry. Briefly, phage clones may be incubated with cells (e.g., engineered to express the antigen of interest, or those that naturally express the antigen) that either do or do not express the antigen. The cells may be washed and then labeled with a mouse anti-M13 coat protein monoclonal antibody. Cells may be washed again and labeled with a fluorescent-conjugated secondary antibody (e.g., FITC-goat (Fab)2 anti-mouse IgG) prior to flow cytometry. Cloning and expression vectors that are useful for producing a human immunoglobulin phage library can be obtained, for example, from Stratagene Cloning Systems (La Jolla, Calif.).

A similar strategy may be employed to obtain high-affinity scFv clones. A library with a large repertoire may be constructed by isolating V-genes from non-immunized human donors using PCR primers corresponding to all known V_{H}, Vκ and Vλ gene families. Following amplification, the Vκ and Vλ pools may be combined to form one pool. These fragments may be ligated into a phagemid vector. An scFv linker (e.g., (G4S)n) may be ligated into the phagemid upstream of the V_{L} fragment (or upstream of the V_{H} fragment as so desired). The V_{H} and linker-V_{L} fragments (or V_{L} and linker-V_{H} fragments) may be amplified and assembled on the JH region. The resulting V_{H}-linker-V_{L} (or V_{L}-linker-V_{H}) fragments may be ligated into a phagemid vector. The phagemid library may be panned using filters, as described above, or using immunotubes (Nunc; Maxisorp). Similar results may be achieved by constructing a combinatorial immunoglobulin library from lymphocytes or spleen cells of immunized rabbits and by expressing the scFv in P. pastoris (see, e.g., Ridder et al., 1995, Biotechnology, 13:255-260). Additionally, following isolation of appropriate scFv antibodies, higher binding affinities and slower dissociation rates may be obtained through affinity maturation processes such as mutagenesis and chain-shuffling (see, e.g., Jackson et al., 1998, Br. J. Cancer, 78:181-188); Osbourn et al., 1996, Immunotechnology, 2:181-196).

Human antibodies may be produced using various techniques, i.e., introducing human Ig genes into transgenic animals in which the endogenous Ig genes have been partially or completely inactivated can be exploited to synthesize human antibodies. In some embodiments, human antibodies may be made by immunization of non-human animals engineered to make human antibodies in response to antigen challenge with human antigen.

Provided CARs may be also produced, for example, by utilizing a host cell system engineered to express a CAR-encoding nucleic acid. Alternatively or additionally, provided CARs may be partially or fully prepared by chemical synthesis (e.g., using an automated peptide synthesizer or gene synthesis of CAR-encoding nucleic acids). CARs described herein may be expressed using any appropriate vector or expression cassette. A variety of vectors (e.g., viral vectors) and expression cassettes are known in the art and cells into which such vectors or expression cassettes may be introduced may be cultured as known in the art (e.g., using continuous or fed-batch culture systems). In some embodiments, cells may be genetically engineered; technologies for genetically engineering cells to express engineered polypeptides are well known in the art (see, e.g., Ausabel et al., eds., 1990, Current Protocols in Molecular Biology (Wiley, New York)).

CARs described herein may be purified, i.e., using filtration, centrifugation, and/or a variety of chromatographic technologies such as HPLC or affinity chromatography. In some embodiments, fragments of provided CARs are obtained by methods that include digestion with enzymes, such as pepsin or papain, and/or by cleavage of disulfide bonds by chemical reduction.

It will be appreciated that provided CARs may be engineered, produced, and/or purified in such a way as to improve characteristics and/or activity of the CARs. For example, improved characteristics include, but are not limited to, increased stability, improved binding affinity and/or avidity, increased binding specificity, increased production, decreased aggregation, decreased nonspecific binding, among others. In some embodiments, provided CARs may comprise one or more amino acid substitutions (e.g., in a framework region in the context of an immunoglobulin or fragment thereof (e.g., an scFv antibody)) that improve protein stability, antigen binding, expression level, or provides a site or location for conjugation of a therapeutic, diagnostic or detection agent.

Purification Tag

In some embodiments, a purification tag may be joined to a CAR described herein. A purification tag refers to a peptide of any length that can be used for purification, isolation, or identification of a polypeptide. A purification tag may be joined to a polypeptide (e.g., joined to the N- or C-terminus of the polypeptide) to aid in purifying the polypeptide and/or isolating the polypeptide from, e.g., a cell lysate mixture. In some embodiments, the purification tag binds to another moiety that has a specific affinity for the purification tag. In some embodiments, such moieties which specifically bind to the purification tag are attached to a solid support, such as a matrix, a resin, or agarose beads. Examples of a purification tag that may be joined to a CAR include, but are not limited to, a hexa-histidine peptide, a hemagglutinin (HA) peptide, a FLAG peptide, and a myc peptide. In some embodiments, two or more purification tags may be joined to a CAR, e.g., a hexa-histidine peptide and a HA peptide. A hexa-histidine peptide (HHHHHH (SEQ ID NO:53)) binds to nickel-functionalized agarose affinity column with micromolar affinity. In some embodiments, an HA peptide includes the sequence YPYDVPDYA (SEQ ID NO:54) or YPYDVPDYAS (SEQ ID NO:55). In some embodiments, an HA peptide includes integer multiples of the sequence YPYDVPDYA (SEQ ID NO:54) or YPYDVPDYAS (SEQ ID NO:55) in tandem series, e.g., 3×YPYDVPDYA or 3×YPYDVPDYAS. In some embodiments, a FLAG peptide includes the sequence DYKDDDDK (SEQ ID NO:56). In some embodiments, a FLAG peptide includes integer multiples of the sequence DYKDDDDK (SEQ ID NO:56) in tandem series, e.g., 3×DYKDDDDK. In some embodiments, a myc peptide includes the sequence EQKLISEEDL (SEQ ID NO:57). In some embodiments, a myc peptide includes integer multiples of the sequence EQKLISEEDL in tandem series, e.g., 3×EQKLISEEDL.

### VII. Therapeutic and Detection Agents

A therapeutic agent or a detection agent may be attached to a CAR described herein. Therapeutic agents may be any class of chemical entity including, for example, but not limited to, proteins, carbohydrates, lipids, nucleic acids, small organic molecules, non-biological polymers, metals, ions, radioisotopes, etc. In some embodiments, therapeutic agents for use in accordance with the present invention may have a biological activity relevant to the treatment of one or more symptoms or causes of cancer. In some embodiments, therapeutic agents for use in accordance with the present invention may have a biological activity relevant to modulation of the immune system and/or enhancement of T-cell mediated cytotoxicity. In some embodiments, therapeutic agents for use in accordance with the present invention have one or more other activities.

A detection agent may comprise any moiety that may be detected using an assay, for example due to its specific functional properties and/or chemical characteristics. Non-limiting examples of such agents include enzymes, radiolabels, haptens, fluorescent labels, phosphorescent molecules, chemiluminescent molecules, chromophores, luminescent molecules, photoaffinity molecules, colored particles or ligands, such as biotin.

Many detection agents are known in the art, as are systems for their attachment to proteins and peptides (see, for e.g., U.S. Pat. Nos. 5,021,236; 4,938,948; and 4,472,509). Examples of such detection agents include paramagnetic ions, radioactive isotopes, fluorochromes, NMR-detectable substances, X-ray imaging agents, among others. For example, in some embodiments, a paramagnetic ion is one or more of chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III), erbium (III), lanthanum (III), gold (III), lead (II), and/or bismuth (III).

The radioactive isotope may be one or more of actinium-225, astatine-211, bismuth-212, carbon-14, chromium-51, chlorine-36, cobalt-57, cobalt-58, copper-67, Europium-152, gallium-67, hydrogen-3, iodine-123, iodine-124, iodine-125, iodine-131, indium-111, iron-59, lead-212, lutetium-177, phosphorus-32, radium-223, radium-224, rhenium-186, rhenium-188, selenium-75, sulphur-35, technicium-99m, thorium-227, yttrium-90, and zirconium-89. Radioactively labeled CARs may be produced according to well-known technologies in the art.

A fluorescent label may be or may comprise one or more of Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red, among others.

### VIII. Methods of Treatment

The CARs and/or compositions of the invention can be administered to individuals (e.g., mammals such as humans) to treat cancer (e.g., a hematological cancer or a solid tumor cancer).

Cancers that may be treated using any of the methods described herein include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the CARs and CAR cells of the invention include, but are not limited to, carcinoma, blastoma, sarcoma, melanoma, neuroendocrine tumors, and glioma, and certain leukemia or lymphoid malignancies, benign and malignant tumors, and malignancies e.g., sarcomas, carcinomas, melanomas, and gliomas. Adult tumors/cancers and pediatric tumors/cancers are also included.

Solid tumors contemplated for treatment by any of the methods described herein include CNS tumors, such as glioma (e.g., brainstem glioma and mixed gliomas), glioblastoma (also known as glioblastoma multiforme), astrocytoma (such as high-grade astrocytoma), pediatric glioma or glioblastoma (such as pediatric high-grade glioma (HGG) and diffuse intrinsic pontine glioma (DIPG)), CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases.

In some embodiments, the cancer is pediatric glioma. In some embodiments, the pediatric glioma is a low-grade glioma. In some embodiments, the pediatric glioma is a high-grade glioma (HGG). In some embodiments, the pediatric glioma is glioblastoma multiforme. In some embodiments, the pediatric glioma is diffuse intrinsic pontine glioma (DIPG). In some embodiments, the DIPG is grade II. In some embodiments, the DIPG is grade III. In some embodiments, the DIPG is grade IV.

Additional solid tumors contemplated for treatment include fibrosarcoma, myxosarcoma, liposarcoma, chondrosarcoma (such as clear-cell chondrosarcoma), chondroblastoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer (e.g., cervical carcinoma and pre-invasive cervical dysplasia), cancer of the anus, anal canal, or anorectum, vaginal cancer, cancer of the vulva (e.g., squamous cell carcinoma, intraepithelial carcinoma, adenocarcinoma, and fibrosarcoma), penile cancer, oropharyngeal cancer, head cancers (e.g., squamous cell carcinoma), neck cancers (e.g., squamous cell carcinoma), testicular cancer (e.g., seminoma, teratoma, embryonal carcinoma, teratocarcinoma, choriocarcinoma, sarcoma, Leydig cell tumor, fibroma, fibroadenoma, adenomatoid tumors, and lipoma), bladder carcinoma, melanoma, cancer of the uterus (e.g., endometrial carcinoma), and urothelial cancers (e.g., squamous cell carcinoma, transitional cell carcinoma, adenocarcinoma, ureter cancer, and urinary bladder cancer).

Hematologic cancers contemplated for treatment by any of the methods described herein include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Examples of other cancers include, without limitation, acute lymphoblastic leukemia (ALL), Hodgkin's lymphoma, non-Hodgkin's lymphoma, B cell chronic lymphocytic leukemia (CLL), multiple myeloma, follicular lymphoma, mantle cell lymphoma, pro-lymphocytic leukemia, hairy cell leukemia, common acute lymphocytic leukemia, and null-acute lymphoblastic leukemia.

Cancer treatments can be evaluated, for example, by tumor regression, tumor weight or size shrinkage, time to progression, duration of survival, progression free survival, overall response rate, duration of response, quality of life, protein expression and/or activity. Approaches to determining efficacy of the therapy can be employed, including for example, measurement of response through radiological imaging.

In some embodiments of any of the methods for treating cancer (e.g., a hematological cancer or a solid tumor cancer), the CAR is conjugated to a cell (such as an immune cell, e.g., a T cell) prior to being administered to the individual. Thus, for example, there is provided a method of treating cancer (e.g., a hematological cancer or a solid tumor cancer) in an individual comprising a) conjugating a CAR described herein or an antibody moiety thereof to a cell (such as an immune cell, e.g., a T cell) to form a CAR/cell conjugate, and b) administering to the individual an effective amount of a composition comprising the CAR/cell conjugate. In some embodiments, the cell is derived from the individual. In some embodiments, the cell is not derived from the individual. In some embodiments, the CAR is conjugated to the cell by covalent linkage to a molecule on the surface of the cell. In some embodiments, the CAR is conjugated to the cell by non-covalent linkage to a molecule on the surface of the cell. In some embodiments, the CAR is conjugated to the cell by insertion of a portion of the CAR into the outer membrane of the cell.

Treatments can be evaluated, for example, by tumor regression, tumor weight or size shrinkage, time to progression, duration of survival, progression free survival, overall response rate, duration of response, quality of life, protein expression and/or activity. Approaches to determining efficacy of the therapy can be employed, including for example, measurement of response through radiological imaging.

In some embodiments, the efficacy of treatment may be measured as the percentage tumor growth inhibition (% TGI), which may be calculated using the equation 100-(T/C×100), where T is the mean relative tumor volume of the treated tumor, and C is the mean relative tumor volume of a non-treated tumor. In some embodiments, the % TGI is about 2%, about 4%, about 6, about 8%, 10%, about 20%, about 30%, about 40%, about 50%, about 60%, about 70%, about 80%, about 90%, about 91%, about 92%, about 93%, about 94%, about 95%, or more than 95%.

### IX. CAR Effector Cell Therapy

The present application also provides methods of using a CAR as described herein to redirect the specificity of an effector cell (such as a primary T cell) to a cancer cell. Thus, the present invention also provides a method of stimulating an effector cell-mediated response (such as a T cell-mediated immune response) to a target cell population or tissue comprising cancer cells in a mammal, comprising the step of administering to the mammal an effector cell (such as a T cell) that expresses a CAR as described herein. In some embodiments, “stimulating” an immune cell refers to eliciting an effector cell-mediated response (such as a T cell-mediated immune response), which is different from activating an immune cell.

CAR effector cells (such as CAR T cells) expressing the CAR can be infused to a recipient in need thereof. The infused cell is able to kill cancer cells in the recipient. In some embodiments, unlike antibody therapies, CAR effector cells (such as CAR T cells) are able to replicate in vivo resulting in long-term persistence that can lead to sustained tumor control.

In some embodiments, the CAR effector cells are CAR T cells that can undergo robust in vivo T cell expansion and can persist for an extended amount of time. In some embodiments, the CAR T cells of the invention develop into specific memory T cells that can be reactivated to inhibit any additional tumor formation or growth.

The CART cells (such as CAR T cells) of the invention may also serve as a type of vaccine for ex vivo immunization and/or in vivo therapy in a mammal. In some embodiments, the mammal is a human.

With respect to ex vivo immunization, at least one of the following occurs in vitro prior to administering the cell into a mammal: i) expansion of the cells, ii) introducing a nucleic acid encoding a CAR to the cells, and/or iii) cryopreservation of the cells. Ex vivo procedures are well-known in the art. Briefly, cells are isolated from a mammal (preferably a human) and genetically modified (i.e., transduced or transfected in vitro) with a vector expressing a CAR disclosed herein. The CAR cell can be administered to a mammalian recipient to provide a therapeutic benefit. The mammalian recipient may be a human and the CAR cell can be autologous with respect to the recipient. Alternatively, the cells can be allogeneic, syngeneic or xenogeneic with respect to the recipient. The procedure for ex vivo expansion of hematopoietic stem and progenitor cells is described in U.S. Pat. No. 5,199,942, incorporated herein by reference, can be applied to the cells of the present invention. Other suitable methods are known in the art, therefore the present invention is not limited to any particular method of ex vivo expansion of the cells. Briefly, ex vivo culture and expansion of T cells comprises: (1) collecting T cells from peripheral blood mononuclear cells (PBMC); and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

In addition to using a cell-based vaccine in terms of ex vivo immunization, the present invention also provides compositions and methods for in vivo immunization to elicit an immune response directed against an antigen in a patient. The CAR effector cells (such as CAR T cells) of the present invention may be administered either alone, or as a pharmaceutical composition in combination with diluents and/or with other components such as IL-2 or other cytokines or cell populations. Briefly, pharmaceutical compositions of the present invention may comprise CAR effector cells (such as T cells), in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. In some embodiments, CAR effector cell (such as T cell) compositions are formulated for administration by intravenous, intrathecal, intracranial, intracerebral, or intracerebroventricular route.

The precise amount of the CAR effector cell (such as CAR T cell) compositions of the present invention to be administered can be determined by a physician with consideration of individual differences in age, weight, tumor size, extent of infection or metastasis, and condition of the patient (subject). In some embodiments, a pharmaceutical composition comprising the CAR effector cells (such as CAR T cells) is administered at a dosage of about 10⁴ to about 10⁹ cells/kg body weight, such any of about 10⁴ to about 10⁵, about 10⁵ to about 10⁶, about 10⁶ to about 10⁷, about 10⁷ to about 10⁸, or about 10⁸ to about 10⁹ cells/kg body weight, including all integer values within those ranges. CAR effect cell (such as CAR T cell) compositions may also be administered multiple times at these dosages. The cells can be administered by using infusion techniques that are commonly known in immunotherapy (see, e.g., Rosenberg et al., New Eng. J of Med. 319:1676, 1988). The optimal dosage and treatment regimen for a particular patient can readily be determined by one skilled in the art of medicine by monitoring the patient for signs of disease and adjusting the treatment accordingly.

In some embodiments, it may be desired to administer activated CAR effector cells (such as CAR T cells) to a subject and then subsequently redraw blood (or have an apheresis performed), activate T cells therefrom according to the present invention, and reinfuse the patient with these activated and expanded T cells. This process can be carried out multiple times every few weeks. In some embodiments, T cells can be activated from blood draws of from 10 cc to 400 cc. In some embodiments, T cells are activated from blood draws of 20 cc, 30 cc, 40 cc, 50 cc, 60 cc, 70 cc, 80 cc, 90 cc, or 100 cc.

The administration of the CAR effector cells (such as CAR T cells) may be carried out in any convenient manner, including by injection, ingestion, transfusion, implantation or transplantation. The compositions described herein may be administered to a patient subcutaneously, intradermally, intratumorally, intranodally, intramedullary, intramuscularly, intrathecally, intracranially, intracerebrally, intracerebroventricularly, by intravenous (i.v.) injection, or intraperitoneally. In some embodiments, the CAR effector cell (such as CAR T cell) compositions of the present invention are administered to a patient by intradermal or subcutaneous injection. In some embodiments, the CAR effector cell (such as CAR T cell) compositions of the present invention are administered by i.v. injection. In some embodiments, the CAR effector cell (such as CAR T cell) compositions of the present invention are administered by intrathecal injection. In some embodiments, the CAR effector cell (such as CAR T cell) compositions of the present invention are administered by intracranial injection. In some embodiments, the CAR effector cell (such as CAR T cell) compositions of the present invention are administered by intracerebral injection. In some embodiments, the CAR effector cell (such as CAR T cell) compositions of the present invention are administered by intracerebroventricular injection. The compositions of CAR effector cell (such as CAR T cell) may be injected directly into a tumor, lymph node, or site of infection.

### X. Methods of Diagnosis and Imaging Using CARs

Labeled CARs can be used for diagnostic purposes to detect, diagnose, or monitor a cancer. For example, the CARs described herein can be used in in situ, in vivo, ex vivo, and in vitro diagnostic assays or imaging assays.

Additional embodiments of the invention include methods of diagnosing a cancer (e.g., a hematological cancer or a solid tumor cancer) in an individual (e.g., a mammal such as a human). The methods comprise detecting antigen-presenting cells in the individual. In some embodiments, there is provided a method of diagnosing a cancer (e.g., a hematological cancer or a solid tumor cancer) in an individual (e.g., a mammal, such as a human) comprising (a) administering an effective amount of a labeled antibody moiety according to any of the embodiments described above to the individual; and (b) determining the level of the label in the individual, such that a level of the label above a threshold level indicates that the individual has the cancer. The threshold level can be determined by various methods, including, for example, by detecting the label according to the method of diagnosing described above in a first set of individuals that have the cancer and a second set of individuals that do not have the cancer, and setting the threshold to a level that allows for discrimination between the first and second sets. In some embodiments, the threshold level is zero, and the method comprises determining the presence or absence of the label in the individual. In some embodiments, the method further comprises waiting for a time interval following the administering of step (a) to permit the labeled antibody moiety to preferentially concentrate at sites in the individual where the antigen is expressed (and for unbound labeled antibody moiety to be cleared). In some embodiments, the method further comprises subtracting a background level of the label. Background level can be determined by various methods, including, for example, by detecting the label in the individual prior to administration of the labeled antibody moiety, or by detecting the label according to the method of diagnosing described above in an individual that does not have the cancer.

Antibody moieties of the invention can be used to assay levels of antigen-presenting cell in a biological sample using methods known to those of skill in the art. Suitable antibody labels are known in the art and include enzyme labels, such as, glucose oxidase; radioisotopes, such as iodine (131I, 125I, 123I, 121I), carbon (14C), sulfur (35S), tritium (3H), indium (115mIn, 113mIn, 112In, 111In), technetium (99Tc, 99mTc), thallium (201Ti), gallium (68Ga, 67Ga), palladium (103Pd), molybdenum (99Mo), xenon (133Xe), fluorine (18F), samarium (153Sm), lutetium (177Lu), gadolinium (159Gd), promethium (149Pm), lanthanum (140La), ytterbium (175Yb), holmium (166Ho), yttrium (90Y), scandium (47Sc), rhenium (186Re, 188Re), praseodymium (142Pr), rhodium (105Rh), and ruthenium (97Ru); luminol; fluorescent labels, such as fluorescein and rhodamine; and biotin.

Techniques known in the art may be applied to labeled antibody moieties of the invention. Such techniques include, but are not limited to, the use of bifunctional conjugating agents (see e.g., U.S. Pat. Nos. 5,756,065; 5,714,631; 5,696,239; 5,652,361; 5,505,931; 5,489,425; 5,435,990; 5,428,139; 5,342,604; 5,274,119; 4,994,560; and 5,808,003). Aside from the above assays, various in vivo and ex vivo assays are available to the skilled practitioner. For example, one can expose cells within the body of the subject to an antibody moiety which is optionally labeled with a detectable label, e.g., a radioactive isotope, and binding of the antibody moiety to the cells can be evaluated, e.g., by external scanning for radioactivity or by analyzing a sample (e.g., a biopsy or other biological sample) derived from a subject previously exposed to the antibody moiety.

### XI. Pharmaceutical Compositions

Also provided herein are compositions (such as pharmaceutical compositions, also referred to herein as formulations) comprising a CAR described herein, a nucleic acid encoding one or more polypeptides contained in a CAR described herein, an expression cassette comprising the nucleic acid, or a host cell expressing a CAR. In some embodiments, the composition further comprises a cell (such as an effector cell, e.g., a T cell) associated with the CAR. In some embodiments, there is provided a pharmaceutical composition comprising a CAR and a pharmaceutically acceptable carrier. In some embodiments, the pharmaceutical composition further comprises a cell (such as an effector cell, e.g., a T cell) associated with the CAR.

Suitable formulations of the CARs are obtained by mixing a CAR having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propylparaben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as olyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™ PLURONICS™ or polyethylene glycol (PEG). Exemplary formulations are described in WO98/56418, expressly incorporated herein by reference. Lyophilized formulations adapted for subcutaneous administration are described in WO97/04801. Such lyophilized formulations may be reconstituted with a suitable diluent to a high protein concentration and the reconstituted formulation may be administered subcutaneously to the individual to be treated herein. Lipofectins or liposomes can be used to deliver the CARs of this invention into cells.

The formulation herein may also contain one or more active compounds in addition to the CAR as necessary for the particular indication being treated, preferably those with complementary activities that do not adversely affect each other. For example, it may be desirable to further provide an anti-neoplastic agent, a growth inhibitory agent, a cytotoxic agent, or a chemotherapeutic agent in addition to the CAR. Such molecules are suitably present in combination in amounts that are effective for the purpose intended. The effective amount of such other agents depends on the amount of CAR present in the formulation, the type of disease or disorder or treatment, and other factors discussed above. These are generally used in the same dosages and with administration routes as described herein or about from 1 to 99% of the heretofore employed dosages.

The CARs may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980). Sustained-release preparations may be prepared.

Sustained-release preparations of the CARs can be prepared. Suitable examples of sustained-release preparations include semipermeable matrices of solid hydrophobic polymers containing the CAR (or fragment thereof), which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydro gels release proteins for shorter time periods. When encapsulated CARs remain in the body for a long time, they can denature or aggregate as a result of exposure to moisture at 37° C., resulting in a loss of biological activity and possible changes in immunogenicity. Rational strategies can be devised for stabilization of CARs depending on the mechanism involved. For example, if the aggregation mechanism is discovered to be intermolecular S—S bond formation through thio-disulfide interchange, stabilization can be achieved by modifying sulfhydryl residues, lyophilizing from acidic solutions, controlling moisture content, using appropriate additives, and developing specific polymer matrix compositions.

In some embodiments, the CAR is formulated in a buffer comprising a citrate, NaCl, acetate, succinate, glycine, polysorbate 80 (Tween 80), or any combination of the foregoing. In some embodiments, the CAR is formulated in a buffer comprising about 100 mM to about 150 mM glycine. In some embodiments, the CAR is formulated in a buffer comprising about 50 mM to about 100 mM NaCl. In some embodiments, the CAR is formulated in a buffer comprising about 10 mM to about 50 mM acetate. In some embodiments, the CAR is formulated in a buffer comprising about 10 mM to about 50 mM succinate. In some embodiments, the CAR is formulated in a buffer comprising about 0.005% to about 0.02% polysorbate 80. In some embodiments, the CAR is formulated in a buffer having a pH between about 5.1 and 5.6. In some embodiments, the CAR is formulated in a buffer comprising 10 mM citrate, 100 mM NaCl, 100 mM glycine, and 0.01% polysorbate 80, wherein the formulation is at pH 5.5.

The formulations to be used for in vivo administration must be sterile. This is readily accomplished by, e.g., filtration through sterile filtration membranes.

### XI. Dosage and Administration

The dose of the CAR compositions administered to an individual (such as a human) may vary with the particular composition, the mode of administration, and the type of disease being treated. In some embodiments, the amount of the CAR composition is sufficient to result in a complete response in the individual. In some embodiments, the amount of the CAR composition is sufficient to result in a partial response in the individual. In some embodiments, the amount of the CAR composition administered (for example when administered alone) is sufficient to produce an overall response rate of more than about any of 2%, 4%, 6%, 8%, 10%, 15%, 20%, 25%, 30%, 35%, 40%, 45%, 50%, 55%, 60%, 64%, 65%, 70%, 75%, 80%, 85%, or 90% among a population of individuals treated with the CAR composition. Responses of an individual to the treatment of the methods described herein can be determined, for example, based on the percentage tumor growth inhibition (% TGI).

In some embodiments, the amount of the composition is sufficient to prolong overall survival of the individual. In some embodiments, the amount of the composition (for example when administered along) is sufficient to produce clinical benefit of more than about any of 2%, 4%, 6%, 8%, 10%, 15%, 20%, 25%, 30%, 35%, 40%, 45%, 50%, 60%, 70%, or 77% among a population of individuals treated with the CAR composition.

In some embodiments, the amount of the composition is an amount sufficient to decrease the size of a tumor, decrease the number of cancer cells, or decrease the growth rate of a tumor by at least about any of 2%, 4%, 6%, 8%, 10%, 20%, 30%, 40%, 50%, 60%, 70%, 80%, 90%, 95% or 100% compared to the corresponding tumor size, number of cancer cells, or tumor growth rate in the same subject prior to treatment or compared to the corresponding activity in other subjects not receiving the treatment. Standard methods can be used to measure the magnitude of this effect, such as in vitro assays with purified enzyme, cell-based assays, animal models, or human testing.

In some embodiments, the amount of the CAR in the composition is below the level that induces a toxicological effect (i.e., an effect above a clinically acceptable level of toxicity) or is at a level where a potential side effect can be controlled or tolerated when the composition is administered to the individual. In some embodiments, the amount of the composition is close to a maximum tolerated dose (MTD) of the composition following the same dosing regimen. In some embodiments, the amount of the composition is more than about any of 80%, 90%, 95%, or 98% of the MTD. In some embodiments, the amount of a CAR in the composition is included in a range of about 0.001 pg to about 1000 pg. In some embodiments of any of the above aspects, the effective amount of a CAR in the composition is in the range of about 0.1 pg/kg to about 100 mg/kg of total body weight.

The CAR compositions can be administered to an individual (such as human) via various routes, including, for example, intravenous, intra-arterial, intraperitoneal, intrapulmonary, oral, nasal, inhalation, intravesicular, intramuscular, intra-tracheal, subcutaneous, intraocular, intrathecal, intracranial, intracerebral, intracerebroventricular, transmucosal, and transdermal. In some embodiments, sustained continuous release formulation of the composition may be used. In some embodiments, the composition is administered intravenously. In some embodiments, the composition is administered intraarterially. In some embodiments, the composition is administered intraperitoneally. In some embodiments, the composition is administered intrathecally. In some embodiments, the composition is administered intracranially. In some embodiments, the composition is administered intracerebrally. In some embodiments, the composition is administered intracerebroventricularly. In some embodiments, the composition is administered nasally.

### XIII. Methods of Manufacturing

The present disclosure further provides a method of making a cell as presently disclosed. The cell may be considered a therapeutic cell if the first and third targets are antigens of a diseased or infected cell. In exemplary aspects, the method comprises contacting a cell with a composition comprising (i) a nucleic acid comprising a first nucleotide sequence encoding a cell surface receptor comprising an extracellular domain (ECD) which binds to a first target, a transmembrane domain (TMD), and an intracellular domain (ICD) comprising at least a portion of a T-cell signaling molecule, and (ii) a nucleic acid comprising a second nucleotide sequence encoding an antigen-binding protein which binds to a second target and a third target, wherein the second nucleotide sequence is operably linked to an inducible promoter, wherein expression of the second nucleotide sequence is activated upon binding of the first target to the cell surface receptor. The composition comprising the nucleic acid may be any of those described herein. In exemplary aspects, the cell which is contacted with the composition is an immune cell. In exemplary aspects, the cell is obtained from a human. In some aspects, the method comprises obtaining immune cells from a human then contacting the cells with the expression vector system. In exemplary aspects, the method comprises culturing the cells for a time period sufficient to expand the cells to a population of at least 10⁶ cells. In exemplary aspects, the cells are expanded to a population of at least 10⁷, 10⁸, 10⁹, 10¹⁰, 10¹¹, 10¹² or more cells.

Methods of delivering nucleic acids for expression in cells are known in the art and include for example, lipid delivery using cationic lipids or other chemical methods (e.g., calcium phosphate precipitation, DEAE-dextran, polybrene), electroporation, or viral delivery. See, e.g., Sambrook and Russell, Molecular Cloning: A Laboratory Manual, 3rd ed. Cold Spring Harbor Press, Cold Spring Harbor, N.Y. (2001), Nayerossadat et al., Adv Biomed Res 1: 27 (2012); and Hesier, William (ed.), Gene Delivery to Mammalian Cells, Vol 1., Non-viral Gene Transfer Techniques, Methods in Molecular Biology, Humana Press, (2004).

### EXAMPLES

### Materials and Methods

### Cell Samples, Cell Lines, and Antibodies

The cell lines HepG2 (ATCC HB-8065; HLA-A2⁺, AFP⁺, GPC3⁺), SK-HEP-1 (ATCC HTB-52; HLA-A2⁺, AFP⁻), Raji (ATCC CCL-86; CD19⁺), Nalm6 (ATCC CRL-1567; CD19⁺), RPMI-8226 (ATCC CRM-CCL-155, ROR1⁺), LNCaP (ATCC CRL-1740; PSMA⁺), and IM9 (ATCC CCL-159; HLA-A2⁺, NY-ESO-1⁺) are obtained from the American Type Culture Collection.

HepG2 is a hepatocellular carcinoma cell line that expresses AFP and GPC3 SK-HEP-1 is a liver adenocarcinoma cell line that does not express AFP. Raji is a Burkitt lymphoma cell line that expresses CD19. Nalm6 is a leukemia cell line that also expresses CD19. RPMI-8226 cells are myeloma cells that express ROR1. The LNCaP prostate tumor cell line expresses PSMA. IM9 is a multiple myeloma cell line that expresses NY-ESO-1. All cell lines are cultured in RPMI 1640 or DMEM supplemented with 10% FBS and 2 mM glutamine at 37° C./5% CO₂.

Antibodies against human or mouse CD3, CD4, CD8, CD28, CCR7, CD45RA or myc tag, (Invitrogen) are purchased. The AFP158/HLA-A*02:01-specific antibody, the CD19-specific antibody, the ROR1-specific antibody, the GPC3-specific antibody, the PSMA-specific antibody and the NY-ESO-1 antibody are developed and produced in house at Eureka Therapeutics. Flow cytometry data are collected using BD FACSCanto II and analyzed using FlowJo software package.

All peptides are purchased and synthesized by Elim Biopharma. Peptides are >90% pure. The peptides are dissolved in DMSO or diluted in saline at 10 mg/mL and frozen at −80° C. Biotinylated single chain AFP158/HLA-A*02:01 and control peptides/HLA-A*02:01 complex monomers are generated by refolding the peptides with recombinant HLA-A*02:01 and beta-2 microglobulin (02M). The monomers are biotinylated via the BSP peptide linked to the C-terminal end of HLA-A*02:01 extracellular domain (ECD) by the BirA enzyme. Fluorescence-labelled streptavidin is mixed with biotinylated peptide/HLA-A*02:01 complex monomer to form fluorescence-labelled peptide/HLA-A*02:01 tetramer.

Lentiviruses containing CARs are produced, for example, by transfection of 293T cells with vectors encoding the chimeric CARs. Primary human T-cells are used for transduction after one-day stimulation with CD3/CD28 beads (Dynabeads®, Invitrogen) in the presence of interleukin-2 (IL-2) at 100 U/ml. Concentrated lentiviruses are applied to T-cells in Retronectin- (Takara) coated 6-well plates for 96 hours. Transduction efficiencies of the anti-AFP and anti-AFP chimeric CARs are assessed by flow cytometry. For anti-CD19 CARs, the assay was performed using a PE-conjugated anti-CD19 anti-idiotype antibody. For anti-AFP CARs, a biotinylated AFP158/HLA-A*02:01 tetramer (“AFP158 tetramer”) with PE-conjugated streptavidin or anti-myc antibody respectively was used. Repeat flow cytometry analyses are done on day 5 and every 3-4 days thereafter.

Cell lines are transduced with a vector that encodes the CAR. Five days post-transduction, cell lysates are generated for western blot using an anti-myc antibody.

Tumor cytotoxicities are assayed by Cytox 96 Non-radioactive LDH Cytotoxicity Assay (Promega). CD3⁺ T cells are prepared from PBMC-enriched whole blood using EasySep Human T Cell Isolation Kit (StemCell Technologies) which negatively depletes CD14, CD16, CD19, CD20, CD36, CD56, CD66b, CD123, glycophorin A expressing cells. Human T cells are activated and expanded with, for example, CD3/CD28 Dynabeads (Invitrogen) according to manufacturer's protocol. Activated T cells (ATC) are cultured and maintained in RPMI 1640 medium with 10% FBS plus 100 U/ml IL-2, and used at day 7-14. Activated T cells (immune cells) and target cells are co-cultured at various effector-to-target ratios (e.g., 2.5:1 or 5:1) for 16 hours and assayed for cytotoxicities.

### Example 1—CAR Designs

Various CAR designs were contemplated and made as described in Table 2 below.

TABLE 2

| CAR | Description |
|---|---|
| Anti-CD19-CD28z-CAR | a 2nd generation CAR comprising anti-CD19 extracellular target- |
| (SEQ ID NO: 1) | binding domain, CD28 TM, CD28 IC costimulatory domain, and |
| | CD3zeta IC primary signaling domain |
| Anti-CD19-CD30z-CAR | a 2nd generation CAR comprising anti-CD19 extracellular target- |
| (SEQ ID NO: 2) | binding domain, CD30 TM, CD30 IC costimulatory domain, and |
| | CD3zeta IC primary signaling domain |
| Anti-CD19-CD8T- | a 2nd generation CAR comprising anti-CD19 extracellular target- |
| CD30z-CAR (SEQ ID | binding domain, CD8 TM, CD30 IC costimulatory domain, and |
| NO: 3) | CD3zeta IC primary signaling domain |
| Anti-CD19-CD8T- | a 2nd generation CAR comprising anti-CD19 extracellular target- |
| 41BBz-CAR (SEQ ID | binding domain, CD8 TM, 4-1BB IC costimulatory domain, and |
| NO: 4) | CD3zeta IC primary signaling domain |
| Anti-AFP-CD28z-CAR | a 2^{nd} generation CAR comprising anti-AFP/MHC EC extracellular |
| (SEQ ID NO: 5) | target-binding domain, CD28 TM, CD28 IC costimulatory |
| | domain, and CD3zeta IC primary signaling domain |
| Anti-AFP-CD30z-CAR | a 2^{nd} generation CAR comprising anti-AFP/MHC EC extracellular |
| (SEQ ID NO: 6) | target-binding domain, CD30 TM, CD30 IC costimulatory |
| | domain, and CD3zeta IC primary signaling domain |
| Anti-AFP-CD8T- | a 2^{nd} generation CAR comprising anti-AFP/MHC EC extracellular |
| CD30z-CAR (SEQ ID | target-binding domain, CD8 TM, CD30 IC costimulatory domain, |
| NO: 7) | and CD3zeta IC primary signaling domain |
| Anti-AFP-CD8T- | a 2^{nd} generation CAR comprising anti-AFP/MHC EC extracellular |
| 41BBz-CAR (SEQ ID | target-binding domain, CD8 TM, 4-1BB IC costimulatory domain, |
| NO: 8) | and CD3zeta IC primary signaling domain |
| Anti-CD19-CD8T- | a 2nd generation CAR comprising anti-CD19 EC extracellular |
| CD28z-CAR (SEQ ID | target-binding domain, CD8 TM, CD28 IC costimularoty domain, |
| NO: 9) | and CD3zeta IC primary signaling domain |
| Anti-AFP-CD8T- | a 2nd generation CAR comprising anti-AFP/MHC EC extracellular |
| CD28z-CAR (SEQ ID | target-binding domain, CD8 TM, CD28 IC costimularoty domain, |
| NO: 10) | and CD3zeta IC primary signaling domain |

Other CAR designs are contemplated and made as described in Table 3 below.

TABLE 3

| CAR | Description |
|---|---|
| Anti-CD19-CD28T- | a 2nd generation CAR comprising anti-CD19 EC extracellular |
| CD30z-CAR | target-binding domain, CD28 TM, CD30 IC costimularoty domain, |
| | and CD3zeta IC primary signaling domain |
| Anti-CD19-CD30- | a 3rd generation CAR comprising anti-CD19 EC extracellular |
| CD30z-CAR | target-binding domain, CD30 TM, two copies of CD30 IC |
| | costimularoty domain, and CD3zeta IC primary signaling domain |
| Anti-CD19-CD8T- | a 3rd generation CAR comprising anti-CD19 EC extracellular |
| CD30-CD30z-CAR | target-binding domain, CD8 TM, two copies of CD30 IC |
| | costimularoty domain, and CD3zeta IC primary signaling domain |
| Anti-CD19-CD30T- | a 3rd generation CAR comprising anti-CD19 EC extracellular |
| 41BB-CD30z-CAR | target-binding domain, CD30 TM, 4-1BB IC costimularoty |
| | domain, CD30 IC costimularoty domain, and CD3zeta IC primary |
| | signaling domain |
| Anti-CD19-CD30T- | a 3rd generation CAR comprising anti-CD19 EC extracellular |
| CD30-41BBz-CAR | target-binding domain, CD30 TM, CD30 IC costimularoty domain, |
| | 4-1BB IC costimularoty domain, and CD3zeta IC primary |
| | signaling domain |
| Anti-CD19-CD8T- | a 3rd generation CAR comprising anti-CD19 EC extracellular |
| 41BB-CD30z-CAR | target-binding domain, CD8 TM, 4-1BB IC costimularoty domain, |
| | CD30 IC costimularoty domain, and CD3zeta IC primary signaling |
| | domain |
| Anti-CD19-CD8T- | a 3rd generation CAR comprising anti-CD19 EC extracellular |
| CD30-41BBz-CAR | target-binding domain, CD8 TM, CD30 IC costimularoty domain, |
| | 4-1BB IC, and CD3zeta IC primary signaling domain |
| Anti-CD19-anti-CD22- | a 2nd generation CAR comprising anti-CD19 EC extracellular |
| CD30z-CAR | target-binding domain, anti-CD22 EC extracellular target-binding |
| | domain, CD30 TM, CD30 IC costimularoty domain, and CD3zeta |
| | IC primary signaling domain |
| Anti-CD19-anti-CD22- | a 2nd generation CAR comprising anti-CD19 EC extracellular |
| CD8T-CD30z-CAR | target-binding domain, anti-CD22 EC extracellular target-binding |
| | domain, CD8 TM, CD30 IC costimularoty domain, and CD3zeta |
| | IC primary signaling domain |
| Anti-CD19-anti-CD22- | a 2nd generation CAR comprising anti-CD19 EC extracellular |
| anti-CD20-CD30z-CAR | target-binding domain, anti-CD22 EC extracellular target-binding |
| | domain, anti-CD20 EC extracellular target-binding domain, CD30 |
| | TM, CD30 IC costimularoty domain, and CD3zeta IC primary |
| | signaling domain |
| Anti-CD19-anti-CD22- | a 2nd generation CAR comprising anti-CD19 EC extracellular |
| anti-CD20-CD8T- | target-binding domain, anti-CD22 EC extracellular target-binding |
| CD30z-CAR | domain, anti-CD20 EC extracellular target-binding domain, CD8 |
| | TM, CD30 IC costimularoty domain, and CD3zeta IC primary |
| | signaling domain |
| Anti-AFP-CD28T- | a 2nd generation CAR comprising anti-AFP/MHC EC extracellular |
| CD30z-CAR | target-binding domain, CD28 TM, CD30 IC costimularoty domain, |
| | and CD3zeta IC primary signaling domain |
| Anti-AFP-CD30- | a 3rd generation CAR comprising anti-AFP/MHC EC extracellular |
| CD30z-CAR | target-binding domain, CD30 TM, two copies of CD30 IC |
| | costimularoty domain, and CD3zeta IC primary signaling domain |
| Anti-AFP-CD8T-CD30- | a 3rd generation CAR comprising anti-AFP/MHC EC extracellular |
| CD30z-CAR | target-binding domain, CD8 TM, two copies of CD30 IC |
| | costimularoty domain, and CD3zeta IC primary signaling domain |
| Anti-AFP-CD30T- | a 3rd generation CAR comprising anti-AFP/MHC EC extracellular |
| 41BB-CD30z-CAR | target-binding domain, CD30 TM, 4-1BB IC costimularoty |
| | domain, CD30 IC costimularoty domain, and CD3zeta IC primary |
| | signaling domain |
| Anti-AFP-CD30T- | a 3rd generation CAR comprising anti-AFP/MHC EC extracellular |
| CD30-41BBz-CAR | target-binding domain, CD30 TM, CD30 IC costimularoty domain, |
| | 4-1BB IC costimularoty domain, and CD3zeta IC primary |
| | signaling domain |
| Anti-AFP-CD8T-41BB- | a 3rd generation CAR comprising anti-AFP/MHC EC extracellular |
| CD30z-CAR | target-binding domain, CD8 TM, 4-1BB IC costimularoty domain, |
| | CD30 IC costimularoty domain, and CD3zeta IC primary signaling |
| | domain |
| Anti-AFP-CD8T-CD30- | a 3rd generation CAR comprising anti-AFP/MHC EC extracellular |
| 41BBz-CAR | target-binding domain, CD8 TM, CD30 IC costimularoty domain, |
| | 4-1BB IC costimularoty domain, and CD3zeta IC primary |
| | signaling domain |

### Example 2—CAR T Cells Prior to Engagement

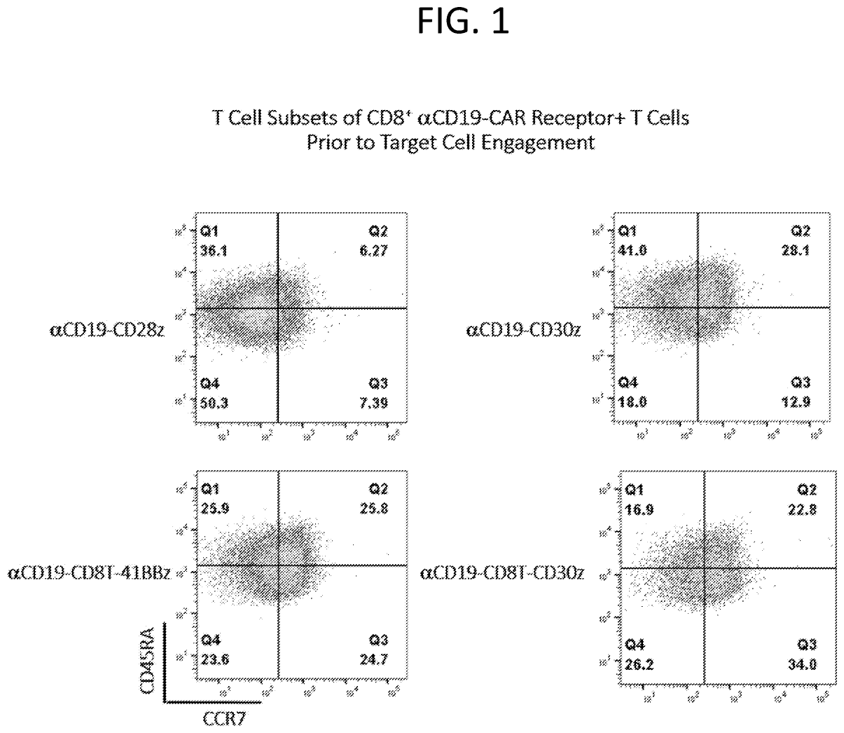

In assays to determine the composition of the T cell subsets represented in the population of CAR-expressing T cells prior to target cell engagement, flow cytometric analyses were performed on the CD8+ Receptor+ CAR T-cells using markers CCR7 (memory T cells) and CD45RA (naïve T cells). FIG. 1 and Table 4 below show the distribution of the differentiation markers CCR7 and CD45RA on αCD19 CAR T cells. These data indicated that T cells expressing the αCD19-CD30z CAR comprise a less differentiated (more naïve) population as well as more central memory T cells than do the -CD28z CAR prior to engagement with target cells. Comparing the differentiation status of anti-CD19-CD8T-CD30z to -CD8T-41BBz CAR T cells in FIG. 1 and Table 4, the population of central memory T cells is also greater in the -CD8T-CD30z population compared to -CD8-41BBz transduced T cells.

TABLE 4
CCR7+ CD8+ T cells prior to target engagement

| | aCD19-CD28z | aCD19-CD30z |
|---|---|---|
| Q2 Naive | 6.27% | 28.1% |
| Q3 Central Memory | 7.39% | 12.9% |

| | aCD19-CD8T-41BBz | aCD19-CD30z |
|---|---|---|
| Q2 Naive | 25.8% | 22.8% |
| Q3 Central Memory | 24.7% | 34.0% |

The differentiation state prior to target engagement may affect the survival and ability of the CAR-expressing T cells to persist in vitro or in vivo, and to contribute to the CAR T cell's anti-tumor efficacy. When in response to antigen encounter, naïve T cells proliferate and differentiate into effector cells, most of which carry out the job of destroying targets and then die, while a small pool of T cells ultimately develops into long-lived memory T cells which can store the T cell immunity against the specific target. Among the memory T cells, the central memory T cells were found to have longer lives than effector memory T cells and be capable of generating effector memory T cells, but not vice versa. Therefore, the ability to develop into and maintain memory T cells, especially central memory T cells, is an important and desired feature for potentially successful T cell therapies.

### Example 3—Short-Term Target Cell Killing by Anti-CD19 CAR T Cells

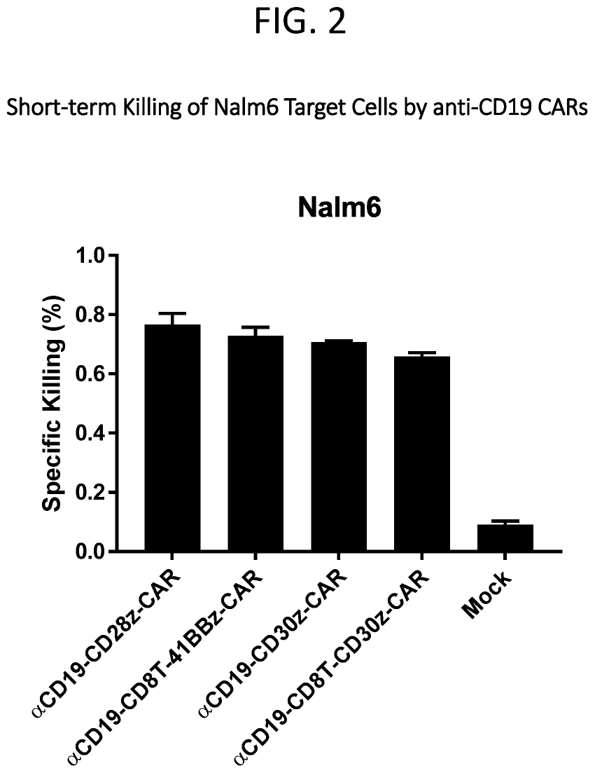

A FACS-based assay comparing the short-term killing ability of the various CAR-T cells was performed. FIG. 2 demonstrates that the anti-CD19-CD30z CAR T cells showed comparable target cell killing (Nalm6) as did the -CD28z CAR T cells. The anti-CD19-CD8T-CD30z CAR T cells likewise showed comparable target cell killing as did the -CD8T-41BBz CAR T cells.

Activated T cells and target cells were co-cultured at a 5:1 ratio with αCD19 or αcAFP antibodies for 16 hours. Specific killing was determined by measuring LDH activity in culture supernatants. Tumor cytotoxicity was assayed by LDH Cytotoxicity Assay (Promega). Human T cells purchased from AllCells were activated and expanded with CD3/CD28 Dynabeads (Invitrogen) according to manufacturer's protocol. Activated T cells (ATC) were cultured and maintained in RPMI 1640 medium with 10% FBS plus 100 U/ml IL-2, and used at day 7-14. The T cells were >99% CD3⁺ by FACS analysis. Activated T cells (Effector cells) and the target cells, Nalm6 or HepG2 cells were co-cultured at a 5:1 ratio with different concentrations of αCD19 or αAFP antibodies, respectively for 16 hours. Cytotoxicities were then determined by measuring LDH activities in culture supernatants.

### Example 4—Anti-CD19 CAR T Cell Proliferation

The proliferation and persistence of genetically modified T-cells is crucial for the success of adoptive T-cell transfer therapies when treating cancers. To assay the effect of the CAR on T-cell proliferation and persistence we labeled T-cells with the intracellular dye CFSE and observed the dilution of the dye as the T-cells divided when stimulated with tumor cells. We were also able to measure persistence of the T-cells by counting the number of CFSE-positive cells remaining at the indicated day.

Respective T-cells were serum starved overnight and labeled with CFSE using CellTrace CFSE (Thermo Fisher C34554). 100,000 T-cells were incubated at an E:T ratio of 2:1 and flow cytometry was used to observe serial dilution of the CFSE dye as the T-cells divide at the indicated day. The total number of T-cells were counted with FACs.

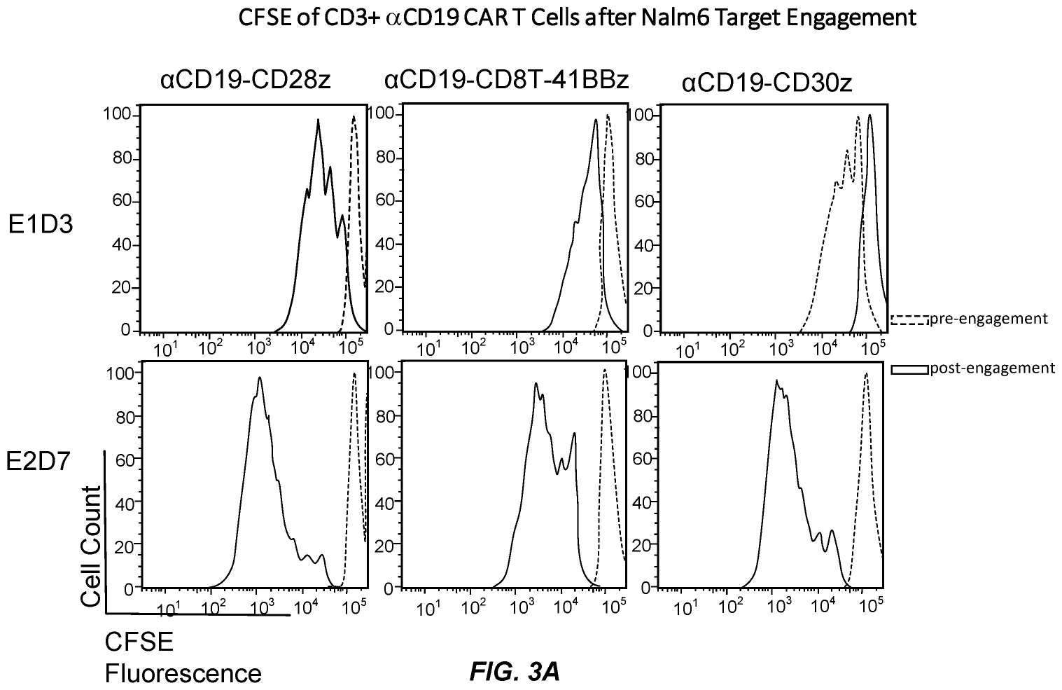

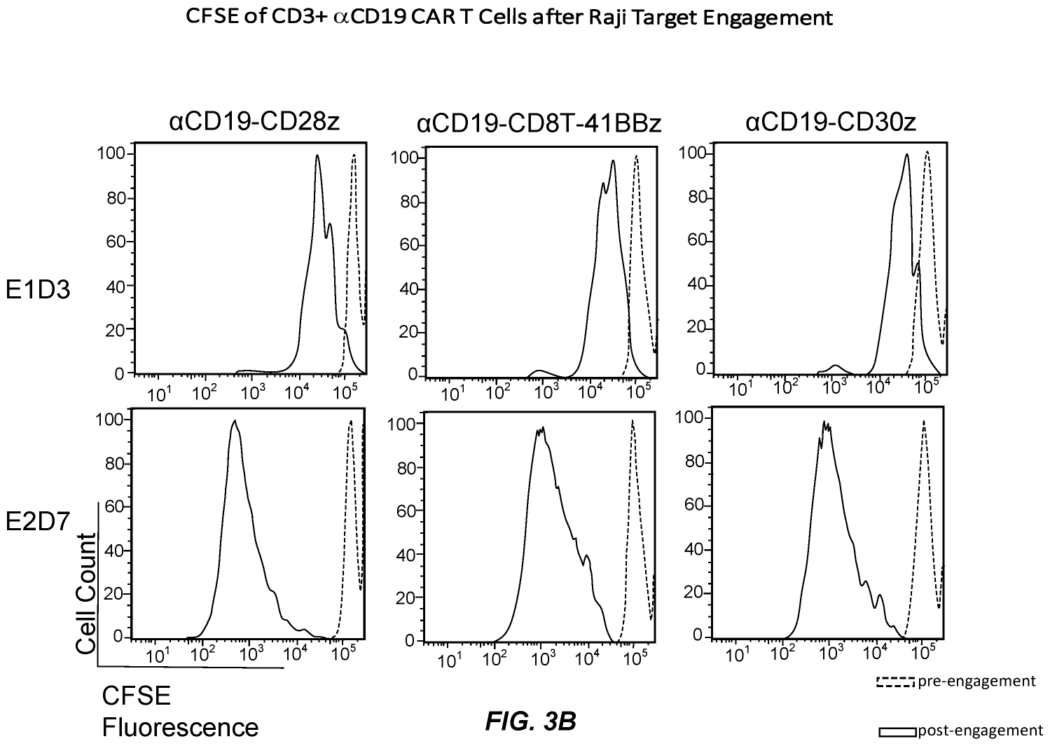

As shown in FIGS. 3A and 3B, anti-CD19 CAR T cell proliferation following target cell engagement among the anti-CD19 CAR-T cells tested, the -CD28z and -CD30z or -CD8T-CD30z and -CD8T-41BBz CAR T cells show robust levels of cell division in response to both over time with Nalm6 (A) and Raji (B) cell engagement as measured by CFSE dilution.

### Example 5—Long Term Killing by Anti-CD19 CAR T Cells Following Multiple Cancer Cell Engagements

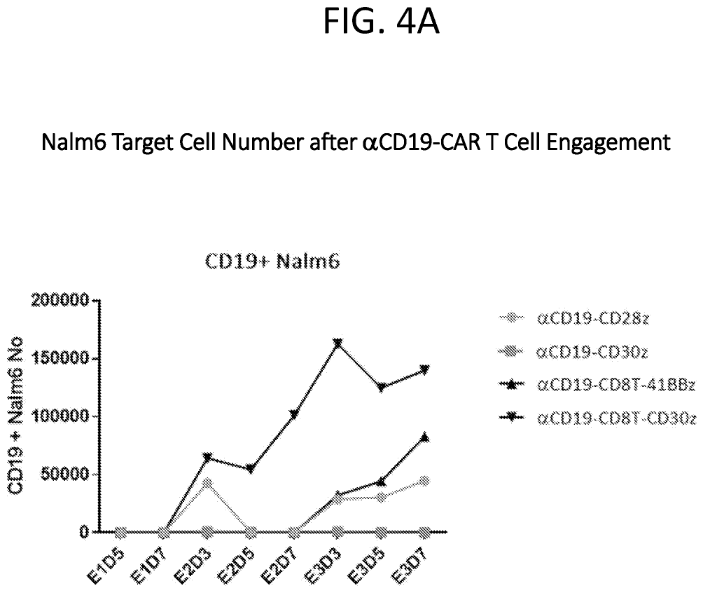

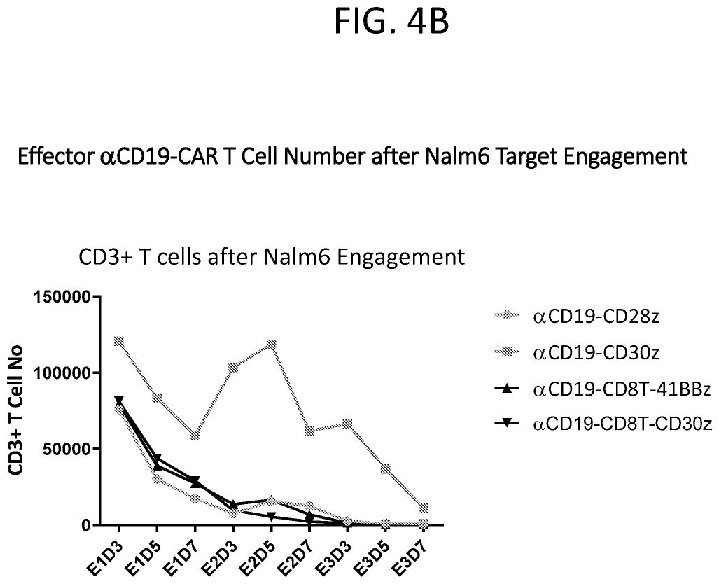

A FACS based assay for counting target cells was used to compare the long-term killing potential of CAR T cells. As shown in FIGS. 4A and 4B, anti-CD19 CARs effectively mediated the killing of cancer cell lines in a human CD19-specific manner, measured over several days post-engagement. FIG. 4A shows the Nalm6 target cell counts across several days (E1D3 through E3D7) following engagement. FIG. 4B shows the T cell number measured over the same period. The effector to target ratio in this experiment was initially 5:1. The result in FIG. 4A shows that anti-CD19-CAR T cells with CD30 TM and CD30z IC regions (αCD19-CD30z) killed more Nalm6 tumor cells than corresponding CAR T cells with CD28 TM and CD28z IC (αCD19-CD28z) and corresponding CAR T cells with CD8 TM and 4-1BB IC (αCD19-CD8T-41BBz). The result in FIG. 4B shows that anti-CD19-CAR T cells with CD30 TM and CD30z IC survived at higher rates and for longer time than corresponding CAR T cells with CD28 TM and CD28z IC and corresponding CAR T cells with CD8 TM and 4-1BB IC.

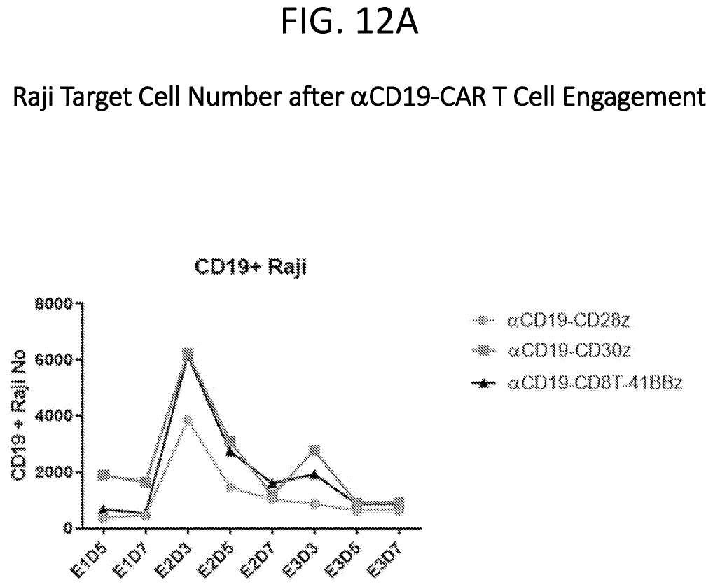

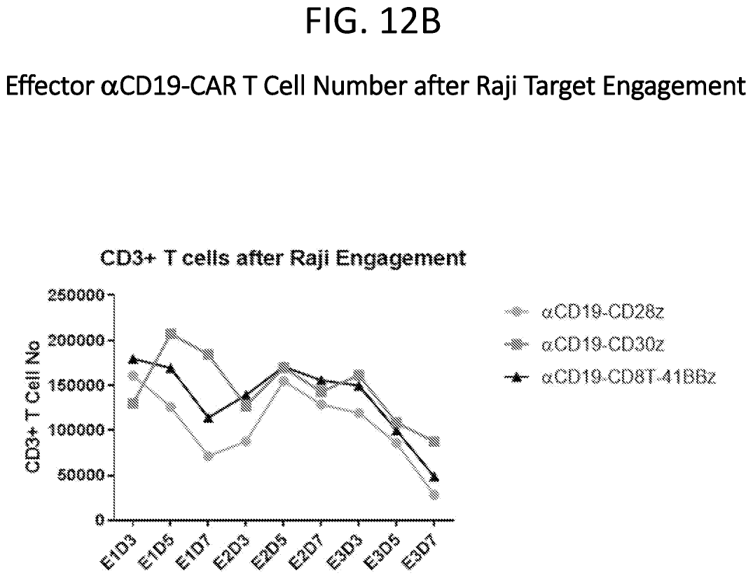

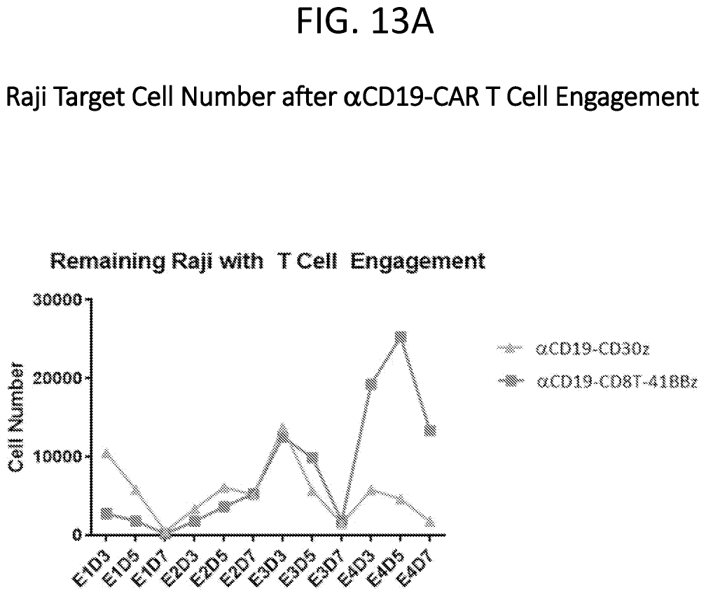

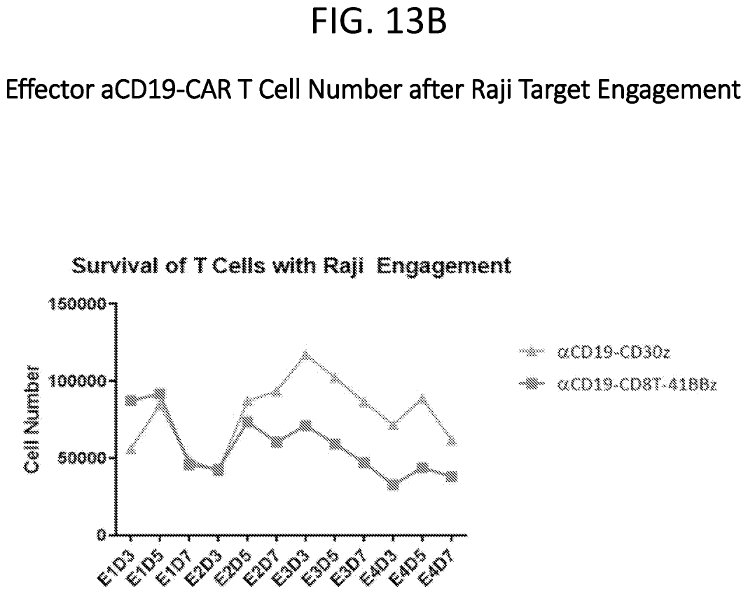

Long-term killing by anti-CD19 CAR T cells was also measured by co-culture with Raji cells as shown in FIGS. 12A, 12B, 13A, and 13B. In FIG. 12A, anti-CD19 CAR T cells with -CD30z and -CD8T-41BBz show comparable efficacy in target cell killing measured over several days. FIG. 12B shows that anti-CD19-CAR T cells with CD30 TM and CD30z IC survived at high rates and for longer time than corresponding CAR T cells with CD28 TM and CD28z IC and corresponding CAR T cells with CD8 TM and 4-1BB IC.

In FIGS. 13A and 13B, anti CD19-CD30z CAR T cells were compared to the corresponding -CD8T-41BBz CAR T cells in a long-term killing assay, using Raji cells as the target. The result shows that CAR T cells with CD30 TM and CD30z IC killed more target cells and survived better than corresponding CD8T-41BB CAR T cells.

Our experience with various antibody moieties paired with diverse TM and costimulatory domains revealed that for the anti-CD19-CAR, both the CD30TM-CD30z CAR and the CD8TM-CD30z CAR configurations were able to kill Nalm6 and Raji cells, although the former seemed to work somewhat better. On the contrary, for the anti-AFP-CAR, the CD30TM-CD30z CAR only showed low levels of HepG2 cell killing, while the CD8T-CD30z CAR configuration is significantly more able to kill HepG2 cells.

### Example 6—Expression of T Cell Exhaustion Markers in Anti-CD19 CAR T Cells after Co-Culture with Target Cells

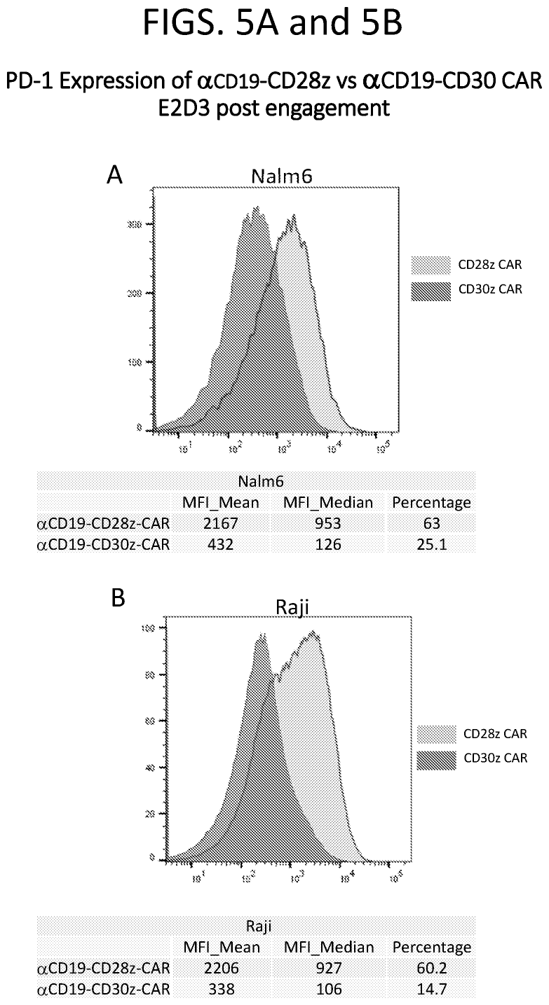

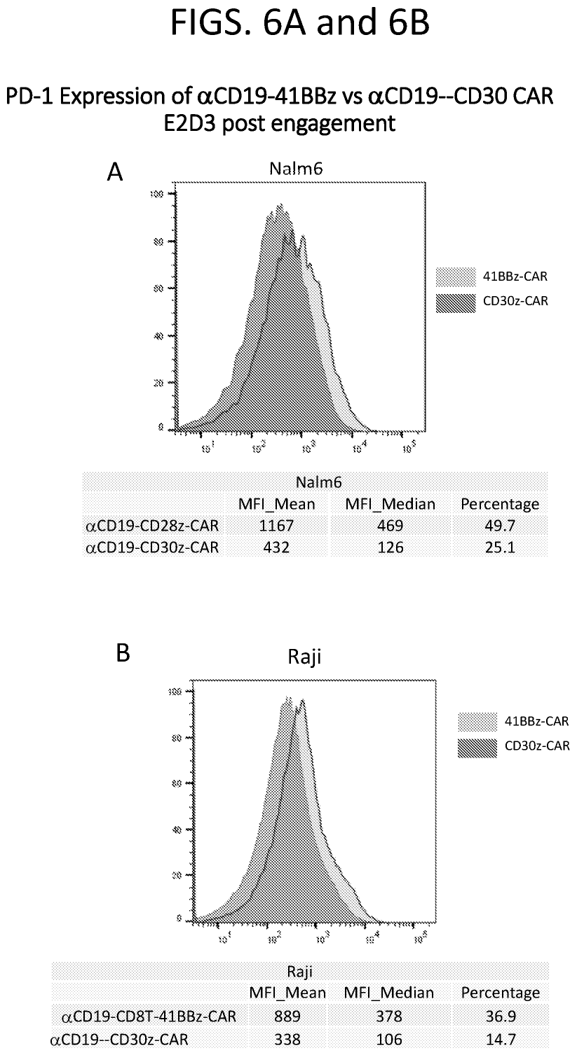

To examine the level of exhaustion markers expressed on CAR-transduced cells upon antigen stimulation, CD3⁺ T cells were prepared from PBMC-enriched whole blood using EasySep Human T Cell Isolation Kit (StemCell Technologies) and activated with CD3/CD28 Dynabeads as above. The activated and expanded cell population was >99% CD3⁺ by flow cytometry. These cells were then transduced with lentiviral vectors encoding a CAR containing an αCD19-CD28z, αCD19-CD30z or an αCD19-CD8T-41BBz or αCD19-CD8T-CD30z CAR construct (SEQ ID NOS:1, 2, 4, and 3, respectively) for 7-9 days. The transduced cells were co-cultured with target cells for 16 hours at an effector-to-target ratio of 2.5:1, using anti-CD19 antibody and anti-CD4 antibody, along with antibodies to exhaustion marker PD-1, LAG3 or TIM3. The level of exhaustion markers on the transduced T cells were analyzed by flow cytometry. FIGS. 5A and 5B show the MFI of PD-1 expression in -CD30z and -CD28z CAR T cells at day 3 following the second engagement with target Nalm6 cells. FIGS. 6A and 6B show the MFI values on the same day for -CD8T-CD30z and -CD8T-41BBz CAR T cells.

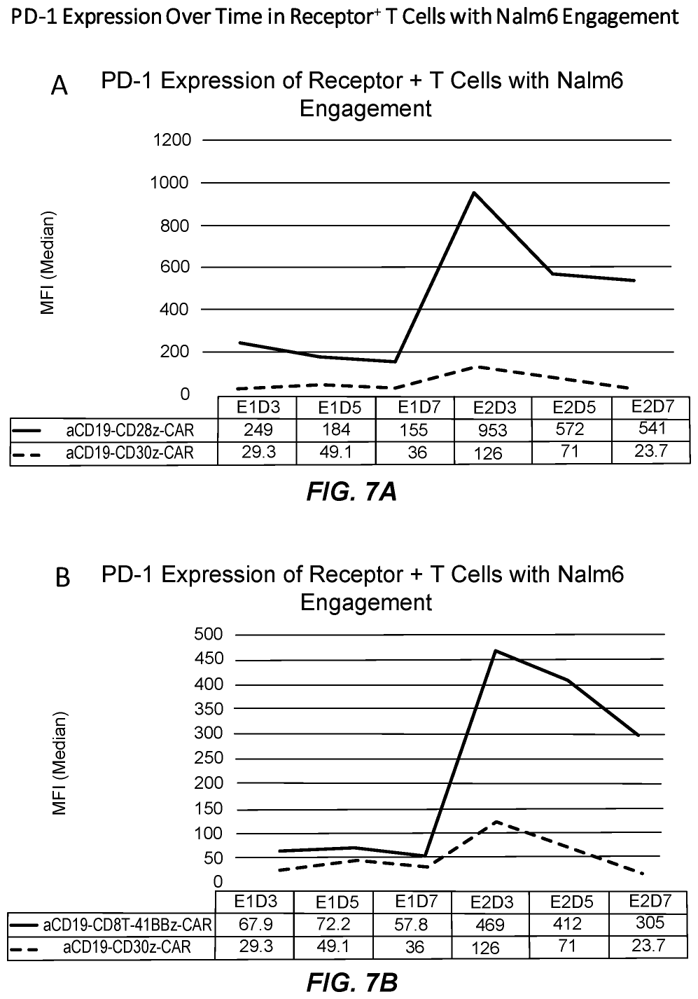

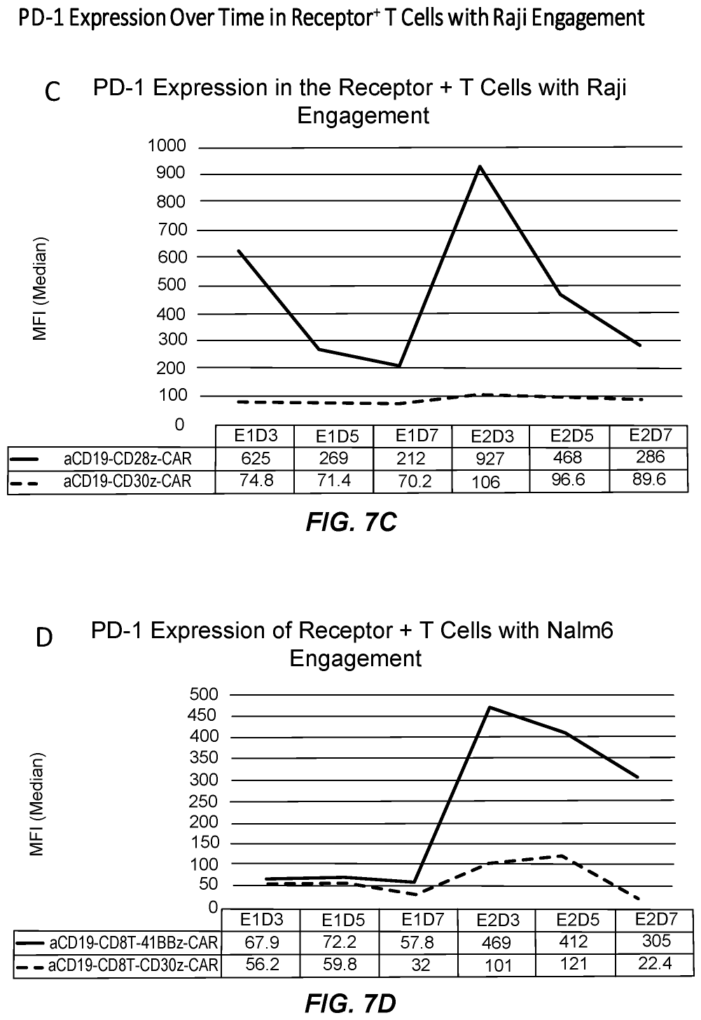

FIGS. 7A and 7B show the effect of anti-CD19 CARs on the expression of three exhaustion markers, PD-1, TIM3 and LAG3 on the surface of the CAR T cells from Day 3 to Day 5 post-engagement. Tables 5A and 5B compare the MFI value ratios of the anti-CD19-CD30z and -CD8T-CD30z CAR T cells with the anti-CD19-CD28z and -CD8T-41BBz CAR T cells over a longer period. These experiments show that the -CD30z CAR is superior to the -CD28z or -CD8T-41BBz CARs in repressing the expression of PD-1, LAG3 or TIM3 in CAR T cells. The implication is that the anti-CD30z or anti-CD8T-CD30z CAR T cells may be able to persist longer, delaying T cell exhaustion (anergy) following engagement than do -CD28 or -CD8T-41BB CAR T cells.

TABLE 5A
Comparison of the MFI of exhaustion markers PD-1,
TIM3 and LAG3 over time in a panel of αCD19
CAR T cells following Nalm6 cell engagement

| | | | Ratio PD-1 | Ratio PD-1 |
|---|---|---|---|---|
| | Ratio PD-1 | Ratio PD-1 | Median | Median CD8T- |
| | Median | Median CD8T- | CD30z/CD8T- | CD30z/CD8T- |
| | CD30z/CD28z | CD30z/CD28z | 41BBz | 41BBz |
| E1D3 | 0.12 | 0.27 | 0.19 | 0.83 |
| E1D5 | 0.27 | 0.12 | 0.68 | 0.83 |
| E1D7 | 0.23 | 0.04 | 0.62 | 0.55 |
| E2D3 | 0.13 | 0.12 | 0.27 | 0.22 |
| E2D5 | 0.12 | 0.12 | 0.17 | 0.29 |
| E2D7 | 0.04 | 0.04 | 0.08 | 0.07 |
| | | | Ratio TIM3 | Ratio TIM3 |
| | Ratio TIM3 | Ratio TIM3 | Median | Median CD8T- |
| | Median | Median CD8T- | CD30z/CD8T- | CD30z/CD8T- |
| | CD30z/CD28z | CD30z/CD28z | 41BBz | 41BBz |
| E1D3 | 0.45 | 0.36 | 1.53 | 1.22 |
| E1D5 | 0.37 | 0.56 | 0.95 | 1.43 |
| E1D7 | 0.29 | 0.43 | 0.73 | 1.07 |
| E2D3 | 0.61 | 0.82 | 0.58 | 0.78 |
| E2D5 | 0.41 | 0.57 | 0.64 | 0.88 |
| E2D7 | 0.26 | 0.26 | 0.36 | 0.68 |
| | | | Ratio LAG3 | Ratio LAG3 |
| | Ratio LAG3 | Ratio LAG3 | Median | Median CD8T- |
| | Median | Median CD8T- | CD30z/CD8T- | CD30z/CD8T- |
| | CD30z/CD28z | CD30z/CD28z | 41BBz | 41BBz |
| E1D3 | 0.33 | 0.33 | 1.00 | 1.00 |
| E1D5 | 0.48 | 0.48 | 1.00 | 1.00 |
| E1D7 | 0.15 | 0.15 | 1.00 | 1.00 |
| E2D3 | 0.05 | 0.02 | 0.10 | 0.05 |
| E2D5 | 0.03 | 0.05 | 0.05 | 0.08 |
| E2D7 | 0.03 | 0.13 | 0.03 | 0.14 |

TABLE 5B
Exhaustion markers PD-1 and LAG3 expressed on αCD19 CAR
T cells several days post engagement with Raji target cells

| | | | Ratio PD-1 |
|---|---|---|---|
| | Ratio PD-1 | Ratio PD-1 | Median |
| | Median | Median CD8T- | CD30z/CD8T- |
| | CD30z/CD28z | CD30z/CD28z | 41BBz |
| E1D3 | 0.12 | 0.21 | 0.91 |
| E1D5 | 0.26 | 0.85 | 0.46 |
| E1D7 | 0.33 | 0.34 | 0.50 |
| E2D3 | 0.11 | 0.35 | 0.28 |
| E2D5 | 0.20 | 0.62 | 0.35 |
| E2D7 | 0.31 | 1.08 | 0.27 |

| | | Ratio LAG3 |
|---|---|---|
| | Ratio LAG3 | Median |
| | Median | CD30z/CD8T- |
| | CD30z/CD28z | 41BBz |
| E1D3 | 0.27 | 1.00 |
| E1D5 | 1.00 | 1.00 |
| E1D7 | 1.00 | 0.56 |
| E2D3 | 0.01 | 0.03 |
| E2D5 | 0.08 | 0.05 |
| E2D7 | 0.28 | 0.03 |

Following the same protocol as described herein, in addition to measuring exhaustion marker expression, the expression of stem cell markers can also be measured to further address the notion of induced anergy. Examples of stem cell markers include, but are not limited to, stem cell antigen-1 (Sca-1), Bcl-2, and IL,-2 and IL-15 receptor β chain (CD122). Stem cell marker levels would measure the remaining T cell memory subset of the starting population of transduced T cells, which is important to maintain in a solid tumor microenvironment.

### Example 7—Short Term Target Cell Killing by Anti-AFP CAR T Cells

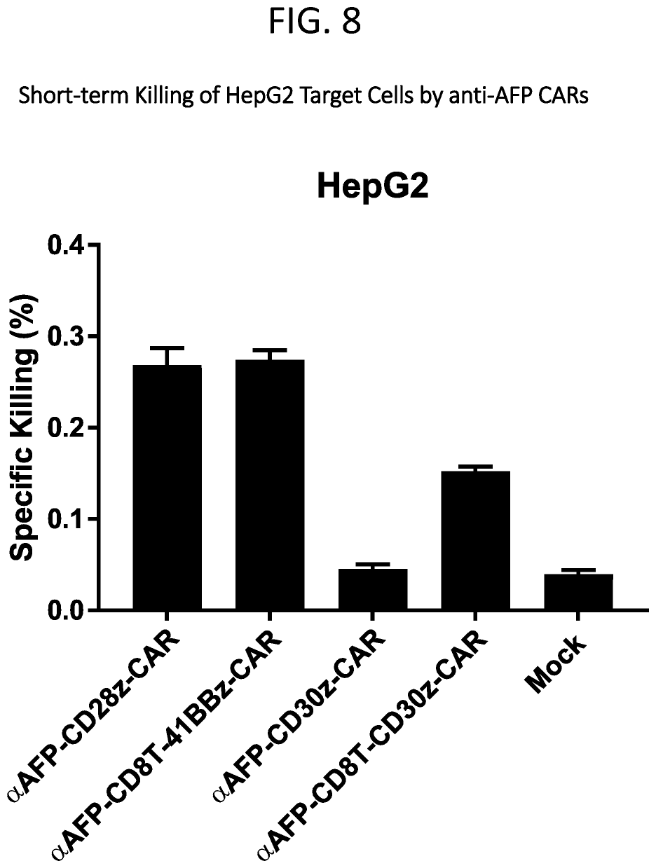

Using methods described for short-term killing in Nalm6 target cells, above, FIG. 8 shows that HepG2 killing mediated by anti-AFP-CD30z CAR T cells was comparable to that of CD28z CAR T cells and CD8T-CD30z CAR T cell killing comparable to that of CD8T-41BBz CAR T cells. The number of surviving CD3+ anti-AFP CAR T cells across several days was also comparable.

### Example 8—Anti-AFP CAR T Cell Proliferation

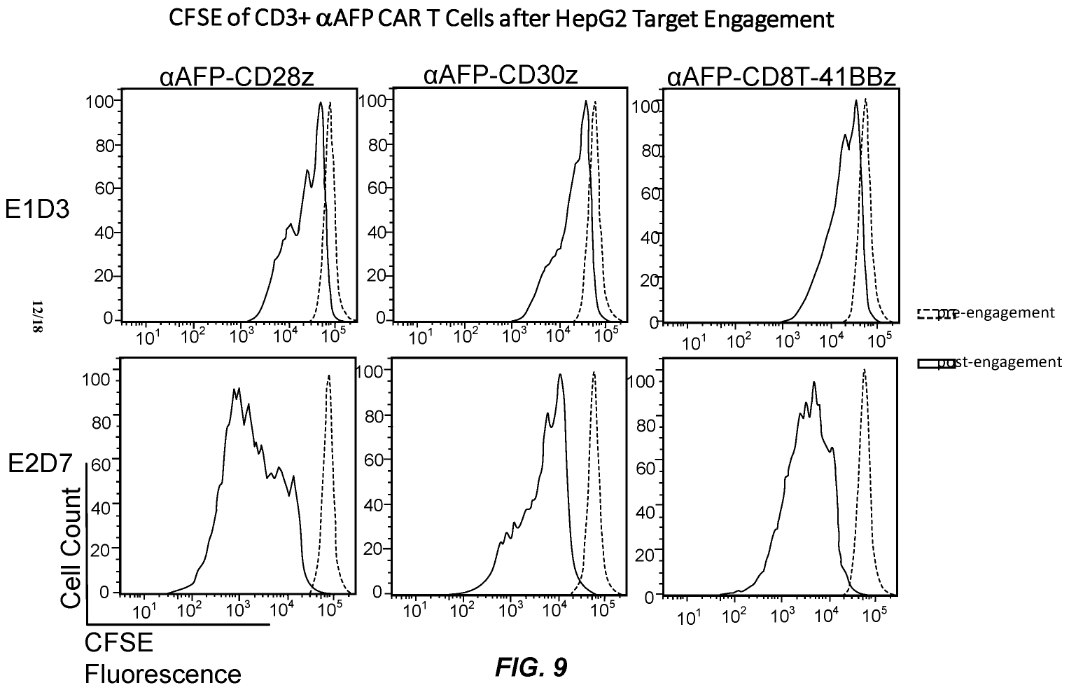

As shown in FIG. 9, using methods described above for the CFSE dilution assay, anti-AFP CAR T cell proliferation following target cell engagement among the anti-AFP CAR-T cells tested (-CD28z and -CD30z or -CD8T-CD30z and -CD8T-41BBz CAR T cells) show vigorous levels of cell division over time following HepG2 engagement.

### Example 9—Long Term Killing by Anti-AFP CAR T Cells

The long-term effect of the αAFP CAR T cells on liver cancer target cells over multiple engagements was measured using the same method for long term killing as described for anti-CD19 CAR T cells except that the HepG2 AFP+ hepatocyte cell line was used. The results show that the CD30 CAR T cell number was maintained at a comparable and significant level among the anti-AFP CARs analyzed (-CD28z, -CD8T-41BBz and -CD8T-CD30z) across the multiple day assay period. In addition, the long-term killing of the HepG2 target cells was observed similarly among the CAR T cells tested.

### Example 10—PD-1 Exhaustion Marker in Anti-AFP CAR T Cells

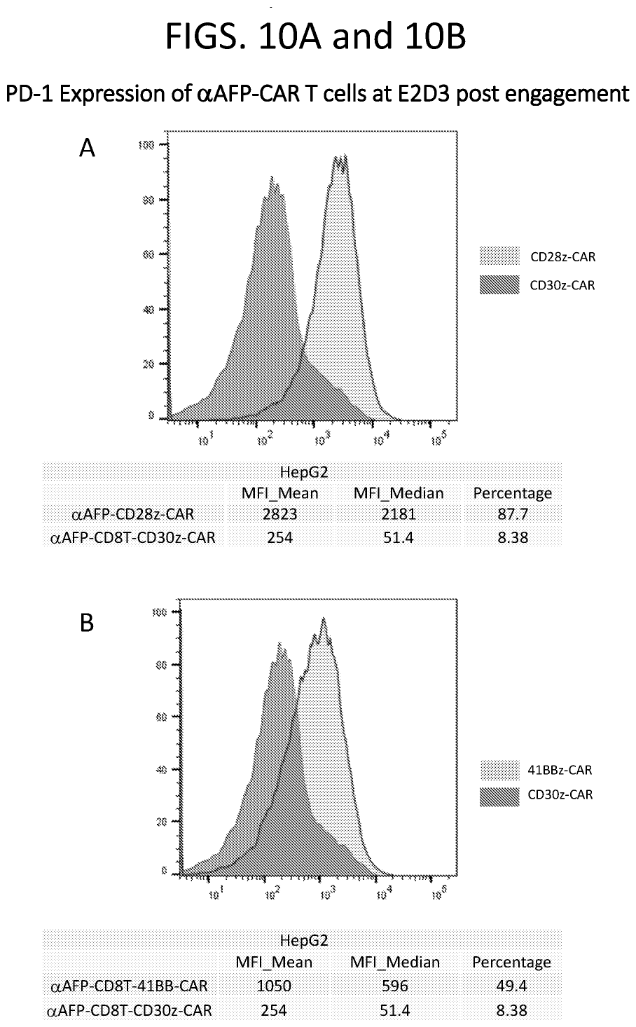

In FIGS. 10A and 10B, exhaustion marker PD-1 expression in anti-AFP CAR T cells, measured by MFI as described above for the anti-CD19 CAR T cells except that HepG2 cell line was used, showed significantly lower levels of PD-1 expression in CD8T-CD30z CAR T cells than in CD28z CAR T cells. Likewise, the CD8T-CD30z expressing CAR T cells showed a repression of PD-1 expression compared to that of the CD8T-41BB CAR T cells.

### Example 11—In Vitro Killing, T Cell Proliferation, and Expression of Exhaustion Markers PD-1, TIM3, and LAG3 Expressed on αAFP CAR T Cells Several Days Post Engagement with Target HepG2 Cells

CD3⁺ T cells were prepared from PBMC-enriched whole blood as above, and also transduced with lentiviral vectors encoding anti-AFP-CAR constructs -CD28z, and -CD30z or -CD8T-41BBz and -CD8T-CD30z (SEQ ID NOS:5, 6, 8, and 7, respectively) for 7-9 days. The transduced cells were then co-cultured with target cells for 16 hours at an effector-to-target ratio of 2.5:1 and co-stained with AFP158 tetramer and anti-CD4 antibody, along with antibodies to exhaustion marker PD-1, LAG3 or TIM3. The level of exhaustion markers on the transduced T cells were analyzed by flow cytometry by gating on the tetramer+(i.e., transduced) T cells.

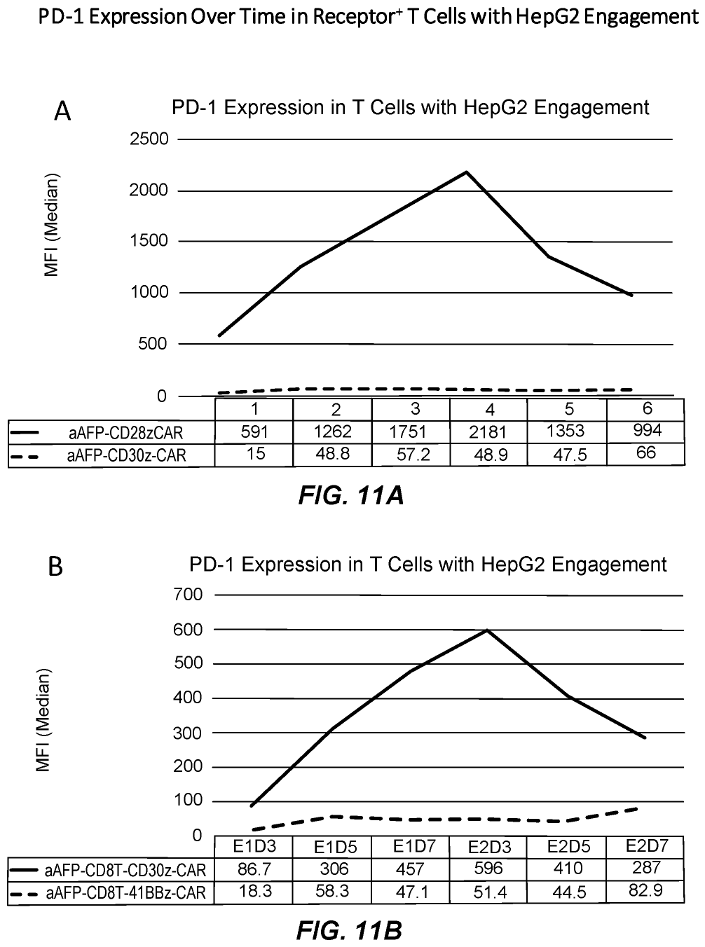

A comparison of the median MFI values representing PD-1, TIM3, and LAG3 expression in anti-AFP CAR T cells over time are shown in FIGS. 11A and 11n. Engagement through a -CD30z CAR receptor results in T cells that accumulate less exhaustion markers than T cells activated through -CD28z CAR receptor. Table 6 compares the ratio of MFI values in the anti-AFP-CD30z and -CD8-CD30z CARs compared to the -CD28z and -CD8T-41BB CARs following HepG2 engagement.

TABLE 6
Exhaustion markers PD-1, TIM3, and LAG3 expressed on αAFP
CAR T cells several days post engagement with HepG2 target cells

| | | | Ratio PD-1 | Ratio PD-1 |
|---|---|---|---|---|
| | Ratio PD-1 | Ratio PD-1 | Median | Median CD8T- |
| | Median | Median CD8T- | CD30z/CD8T- | CD30z/CD8T- |
| | CD30z/CD28z | CD30z/CD28z | 41BBz | 41BBz |
| E1D3 | 0.03 | 0.03 | 0.17 | 0.21 |
| E1D5 | 0.04 | 0.05 | 0.16 | 0.19 |
| E1D7 | 0.03 | 0.03 | 0.12 | 0.10 |
| E2D3 | 0.02 | 0.02 | 0.08 | 0.09 |
| E2D5 | 0.04 | 0.03 | 0.12 | 0.11 |
| E2D7 | 0.07 | 0.08 | 0.23 | 0.29 |
| | | | Ratio TIM3 | Ratio TIM3 |
| | Ratio TIM3 | Ratio TIM3 | Median | Median CD8T- |
| | Median | Median CD8T- | CD30z/CD8T- | CD30z/CD8T- |
| | CD30z/CD28z | CD30z/CD28z | 41BBz | 41BBz |
| E1D3 | 0.42 | 0.58 | 0.66 | 0.92 |
| E1D5 | 0.18 | 0.37 | 0.37 | 0.71 |
| E1D7 | 0.13 | 0.34 | 0.27 | 0.71 |
| E2D3 | 0.08 | 0.19 | 0.28 | 0.56 |
| E2D5 | 0.08 | 0.37 | 0.08 | 0.37 |
| E2D7 | 0.13 | 0.41 | 0.13 | 0.41 |
| | | | Ratio LAG3 | Ratio LAG3 |
| | Ratio LAG3 | Ratio LAG3 | Median | Median CD8T- |
| | Median | Median CD8T- | CD30z/CD8T- | CD30z/CD8T- |
| | CD30z/CD28z | CD30z/CD28z | 41BBz | 41BBz |
| E1D3 | 1.00 | 1.00 | 1.00 | 1.00 |
| E1D5 | 0.10 | 0.10 | 1.00 | 1.00 |
| E1D7 | 0.13 | 0.13 | 1.00 | 1.00 |
| E2D3 | 0.08 | 0.08 | 1.00 | 1.00 |
| E2D5 | 0.07 | 0.07 | 0.71 | 0.71 |
| E2D7 | 0.17 | 0.17 | 1.00 | 1.00 |

### Example 12—Comparison of Exhaustion Markers in αROR1 CAR T Cells

We have previously demonstrated (WO2016187220, WO2016187216) that antibodies or CARs targeting ROR1 are effective in antigen-dependent killing assays both in vitro in in vivo tumor models. To further address the effectiveness of ROR1 as a target for B-lymphocytic cancers, we express ROR1 in 2^{nd} generation-CD8T-CD30z CAR T cells using CD30, CD28 and 4-1BB costimulatory regions. In overnight killing assays and in long-term killing assays, the αROR1-CD30z CARs are anticipated to perform better than their -CD28z and -4-1BBz counter parts as measured by LDH release using the ROR1⁺ RPMI-8226 myeloma cell line.

Proliferation and survival of ROR1V T cells is measured before and after target cell engagement in two independent flow cytometric assays. FACS analysis of CD8⁺ receptor⁺ CAR T-cells is anticipated to show the enhanced expression of T cell differentiation markers CCR7 and CD45RA in CD4⁺ receptor⁺ CAR-T cells prior to target engagement. The values Q2 and Q3 are anticipated to show that αROR1-CD30z T cells comprise a more naïve population that do T cells expressing αROR1-CD28z or -41BBz expressing T cells.

A CFSE dilution/proliferation (FACS) assay of αROR1-CAR T cells is performed following RPMI-8226 ROR1⁺ target cell engagement. The proliferation of αROR1-CAR T-cells at day 3 (E1D3) and day 7 (E2D7) post-engagement is measured by the reduction of CFSE fluorescence as cells divide. The proliferation is expected to be comparable in all T cell populations tested.

Exhaustion marker expression (PD-1, TIM3, and LAG3) in αROR1 CAR T cells is also analyzed following engagement over time with RPMI-8226 ROR1⁺ target cells. We expect the αROR1-CD30z T cells to express substantially less of all markers than do the αROR1-CD28z and -41BBz CAR T cells.

### Example 13—Phenotypes of αGPC3, αNY-ESO-1, and αPSMA CAR T Cells

A comparison of solid tumor immunotherapy targets GPC3, NY-ESO-1 and PSMA is analyzed by the expression of αGPC3, αNY-ESO-1 and αPSMA CARs with various costimulatory domains in T cells for (a) proliferative capacity, (b) cytotoxicity on target cells and (c) expression of exhaustion markers using the assays described for αCD19 CAR T cells (see Methods).

GPC3 (glypican 3) is a surface-expressed cancer target antigen present on many solid tumors. It is a member of the heparin sulfate proteoglycan family, and the mature form is tethered to the cell surface by a glycosylphosphatidylinositol anchor (GPI). We have previously described an antibody moiety directed to the surface-bound variant of GPC3 (WO2018200586). Targets for GPC3 immunotherapies would be solid tumors such as HCC, melanoma, lung squamous cell carcinoma, ovarian carcinoma, yolk sac tumor, choriocarcinoma, neuroblastoma, hepatoblastoma, Wilms' tumor, testicular nonseminomatous germ cell tumor, gastric carcinoma, and liposarcoma.

The NY-ESO-1 tumor antigen is an 18 kDa intracellular protein belonging to the cancer-testis antigen family. We have described the properties of an NY-ESO-1 antibody previously (WO2016210365); it harbors a binding domain derived from an antibody directed to a peptide-MHC complex, called a TCR mimic. The NY-ESO-1 CAR described herein is derived from the antibody disclosed in that publication; potential immunotherapies will be directed to solid tumor NY-ESO-1 positive targets such as bladder cancer, breast cancer, esophageal cancer, hepatocellular carcinoma, head and neck cancer, melanoma, multiple myeloma, plasmacytoma, neuroblastoma, non-small cell lung cancer (NSCLC), ovarian cancer, prostate cancer, sarcoma, or thyroid cancer.

PSMA (prostate-specific membrane antigen) is a type II transmembrane glycoprotein highly expressed in prostate cancers (adenocarcinomas) and is encoded by the folate hydrolase 1 gene. Our previous work describes an anti-PMSA/anti-CD3 bispecific antibody (WO2019032699). The anti-PSMA CAR described herein is derived from the PSMA portion of the bispecific antibody.

The methods and examples in the patent applications cited above for GPC3, NY-ESO-1 and PSMA provide the guidance and materials necessary for testing the CARs of the present invention in terms of target cell killing, CAR T cell proliferation and exhaustion marker expression. We anticipate that expression of these CARS will further substantiate the hypothesis that the CD30 costimulatory domain confers an exhaustion-resistant phenotype to CAR expressing T cells.

### Example 14—In Vivo Efficacy Studies

### In Vivo Antitumor Activity in a Human Hepatocellular Carcinoma Xenograft Model

The in vivo antitumor activity of T cells transduced with CARs described herein is tested using a subcutaneous (s.c.) model of SK-HEP-1-AFP-MG in SCID-beige mice. The SK-HEP-1-AFP-MG cells are s.c. implanted over the right flank of the SCID-beige mice at 5×10⁶ cells per mouse. When the average tumor volume reaches 100 mm³, animals are randomized based on tumor volume to two groups (with 8 mice per group) receiving: (i) mock-transduced T cells and (ii) CAR-transduced T cells. The animals are treated immediately after randomization by injecting 10⁷ mock or CAR-transduced per mouse, intravenously (i.v.) once every two weeks, for three doses. The mice are closely monitored for general health condition, possible adverse response, if any, and changes in tumor volume. Both mock and the CAR-transduced T cells are well-tolerated at the current dose and schedule. While SK-HEP-1-AFP-MG tumors continue to grow after i.v. administration of mock or abTCR-transduced T cells, the growth rate of CAR-transduced T cell treated tumors is slower compared to mock T-treated tumors.

The antitumor activity of CAR-transduced T cells is further evaluated in larger SK-HEP-1-AFP-MG s.c. tumors. In a study with SK-HEP-1-AFP-MG tumor-bearing mice, animals are randomized into two groups when average tumor volume reached 300 mm³ (n=4 mice per group). Animals received either no treatment or a single intratumoral (i.t.) injection of 107 abTCR-transduced T cells per mouse. The i.t. delivery of CAR-transduced T cells slow down the growth of large SK-HEP-1-AFP-MG tumors as measured by change in tumor volume over time. Both i.v. and i.t. administration of abTCR-transduced T cells significantly inhibit the growth of established s.c. xenografts of SK-HEP-1-AFP-MG.

### In Vivo Antitumor Activity in a Lymphoma Xenograft Model

The in vivo antitumor activity of T cells transduced with CAR is tested in a human lymphoma xenograft model in NOD SCID gamma (NSG) mice. Raji-luc-GFP cells are purchased from Comparative Biosciences, Inc. (Sunnyville, Calif. 94085) and are cultured in RPMI Medium+10% FBS and 1% L-Glutamine at 37° C. in a humidified atmosphere with 5% C02. The Raji-luc-GFP cells are derived from the CD19-positive Burkitt lymphoma cell line, Raji, after stable transfection with dual reporter genes encoding both firefly luciferase (luc) and green fluorescent protein, resulting in cells that can be traced in vivo using bioluminescent imaging. NSG mice are purchased from Jackson Laboratories (Bar Harbor, Me. USA 04609) and are acclimated for at least 7 days prior to the experiment. Raji-luc-GFP cells are re-suspended in PBS and implanted intravenously (i.v.) into NSG mice through tail vein at 1×10⁶ cells/100 μl/mouse. Five days post tumor implantation, animals are imaged using Xenogen IVIS imaging system for assessment of tumor burden. Mice are randomized based on the photon emission into the following four groups at average photon emission of 6.7×10⁵ photons (n=6 mice per group): (i) no treatment, (ii) mock-transduced human T cells, and (iii) CAR-T treated. The animals are treated i.v. with mock or CAR-T cells immediately after randomization at a dose of 10⁷ cells per mouse, once every two weeks for 3 doses.

Animals are closely monitored after dosing. Bioluminescent imaging using Xenogen IVIS system is taken once a week for up to 8 weeks.

Animal studies are carried out as described above to evaluate in vivo anti-tumor capabilities of T cells transduced with CAR.

6-8 weeks old female NSG mice are used in this study. The Raji-luc-GFP cell line is cultured in RPMI Medium+10% FBS and 1% L-Glutamine at 37° C. in a humidified atmosphere with 5% CO₂. Raji-luc-GFP cells are re-suspended in PBS and implanted i.v. into 40 NSG mice at 1×10⁶ cells/100p1/mouse.

At four days post tumor implantation, the mice are imaged using the Ivis Spectrum to confirm tumor growth. The mice are then randomized, based on photon emission, into six groups for the following treatments (n=6 mice/group): 1) Vehicle (PBS); 2) Mock (8×10⁶ mock-transduced T cells); 3) CAR (8×10⁶ T cells transduced with CAR).

Animals are closely monitored after tumor implantation and dosing with 8 million receptor-positive T cells. Animals are weighed and Xenogen imaging is conducted twice a week for the duration of the study. Animals showing the following conditions are euthanized and recorded as “conditional death”: a) acute adverse response: labored breathing, tremor, passive behavior (loss of appetite and lethargy); b) body weight loss more than 25% initial body weight; and c) limb paralysis that affect mouse movement.

All of the CAR T cells targeted and lysed the Raji tumors in vivo, demonstrating efficacy of in the CAR platform to inhibit tumor growth.

### In Vivo Antitumor Activity in a Leukemia Xenograft Model

The in vivo antitumor activity of T cells transduced with a CAR was tested in a human leukemia xenograft model in NSG mice. Nalm6-luc-GFP cells are cultured in RPMI Medium+10% FBS at 37° C. in a humidified atmosphere with 5% CO₂. Nalm6-luc-GFP cells are derived from the acute lymphoblastic leukemia cell line, Nalm6, after stable transfection with dual reporter genes encoding both firefly luciferase (luc) and green fluorescent protein, resulting in cells that can be traced in vivo using bioluminescent imaging. NSG mice are purchased from Jackson Laboratories (Bar Harbor, Me. USA 04609) and acclimated for at least 3 days prior to the experiment. Nalm6-luc-GFP cells are re-suspended in PBS and implanted intravenously (i.v.) into thirty 6-8 week-old female NSG mice through tail vein at 5×10⁵ cells/100 μl/mouse. Four days post tumor implantation, animals are imaged using Xenogen IVIS imaging system for assessment of tumor burden. Mice are randomized based on the photon emission into the following four groups: (i) Vehicle, PBS only (n=6 mice); (ii) 10×10⁶ mock-transduced human T cells (n=6 mice), and (iii) 5×10⁶ CAR T cells.

Animals are closely monitored after tumor implantation and dosing with receptor-positive T cells. Animals are weighed and Xenogen imaging is conducted twice a week for the duration of the study. Animals showing the following conditions are euthanized and recorded as “conditional death”: a) acute adverse response: labored breathing, tremor, passive behavior (loss of appetite and lethargy); b) body weight loss more than 25% initial body weight; and c) limb paralysis that affect mouse movement.

At 24-hours post treatment, blood is collected from 3 mice per group. At 7 days and 13 days post treatment, blood is collected from representative mice from each group and analyzed by flow cytometry using the “123count eBeads” kit from Affymetrix eBioscience, Inc. to determine the numbers of CD3⁺ T cells, CAR-expressing T cells, and tumor cells per μl of blood, and the level of PD-1 expression on T cells. At 13 days post treatment, 2 mice per group are euthanized and bone marrow extracts are analyzed by flow cytometry for CD3⁺/CAR T cells, the presence of tumor cells, and PD-1 expression levels on T cells.

Mice treated with CAR T cells show a reduction in tumor cells (indicated by FITC staining) in both peripheral blood and bone marrow compared to vehicle- and mock-treated control animals at 13 days post treatment. The expression level of PD-1, a T cell exhaustion marker, on the surface of T cells from both peripheral blood and bone marrow is lower in mice treated with CAR T cells, for both CD4⁺ and CD8⁺ T cells. These results suggest that T cell exhaustion is repressed in CD30 CAR-expressing T cells.

### Example 15—Development and Maintenance of Memory Cells from Anti-CD19 CAR T Cells

This example shows that anti-CD19 CAR T cells develop into and maintain a high memory T cell population including central memory and effector memory T cells following target stimulation. To determine the effect of expressing anti-CD19 CAR on T cells' ability to develop into and maintain memory T cells, we measured the cell surface expression of memory T cell markers CCR7 and CD45RA. As is known in the field, T cells with high CCR7 expression levels and low CD45RA expression levels are considered central memory T cells, T cells with low CCR7 and low CD45RA expression levels are effector memory T cells; T cells with low CCR7 and high CD45RA expression levels are effector T cells, while T cells with high CCR7 and high CD45RA are naïve T cells which are released from the thymus, but are as of yet incapble of illiciting an immune response. NaïveT cells require activation, target/antigen challenge/recognition, to differentiate into distinct T subpopulations (Eur J Immunol. 2013 November; 43(11):2797-809. doi: 10.1002/eji.201343751. Epub 2013 Oct. 30. The who's who of T-cell differentiation: human memory T-cell subsets. Mahnke YD1, Brodie T M, Sallusto F, Roederer M, Lugli E.). When in response to antigen encounter, naïve T cells proliferate and differentiate into effector cells, most of which carry out the job of destroying targets and die, while a small pool of T cells ultimately develops into long-lived memory T cells which can “store” T cell immune function against a tumor or viral target upon re-exposure to their cognate antigen. Among the memory T cells, the central memory T cells were found to have longer lives than effector memory T cells and be capable of generating effector memory T cells, but not vice versa. Therefore, the ability to develop into and maintain memory T cells, especially central memory T cells, is an important and desired feature for potentially successful T cell therapies.

Primary T cells were mock transduced or transduced with vectors encoding various CAR constructs. Effector cells were incubated with 100,000 Nalm6 target cells and 100,000 T cells with an effector to target ratio of 1.2:1 into 96-well plates and incubated for 7 days (all wells having the same number of total T cells). The cells were then rechallenged with 100,000 Nalm6 cells per well every 7 days.

Each different T cell and target cell mixture sample was made in replicates to ensure at least one mixture to be available for quantification on each selected day. The effector and target cell mixtures were diluted 1:6 before the fourth and fifth target cell engagement (E4 and E5) to avoid the overcrowdedness of T cells due to the significant T cell expansion, so that only one sixth of the previously remaining cells were rechallenged with 100,000 Nalm6 cells.

On selected days after each target cell engagement, the entire cell mixture in a well from each sample was stained with antibodies against CCR7 and CD45RA and analyzed by flow cytometry. Receptor⁺ T cell numbers were counted, and cells were grouped into various T cell types based on their CCR7 and CD45RA expression levels: central memory T cells (CD45RA⁻ CCR7⁺), effector memory T cells (CD45RA⁻ CCR7⁻), effector T cells (CD45RA⁺ CCR7⁻), and naïve T cells (CD45RA⁺ CCR7⁺). Percentages of various types of T cells among the total number of receptor⁺ T cells were calculated. In some experiments, the cells were also stained with antibodies against CD8 or CD4 to determine the CD8-CD4 characteristics of the counted T cells.

Central memory or effector memory T cells were counted after the various anti-CD19 CAR T cell groups as shown below were engaged with Nalm6 target cells multiple times. The results, including the memory cell counts and calculated ratio of memory cell counts from CD30-CAR T to those from CD28-CAR T or 4-1BB-CAR T cell groups are shown in Tables 7A-7E.

TABLE 7A
Central memory T cell (Tcm) count and ratio
of total CD8⁺ CD30-CAR vs. CD28-CAR T.

| | | | Tcm | | |
|---|---|---|---|---|---|
| | Tcm | Tcm | count | | |
| | count | count | αCD19- | Ratio of Tcm | Ratio of Tcm |
| | αCD19- | αCD19- | CD8T- | cell count | cell count |
| Date | CD28z | CD30z | CD30z | CD30z/CD28z | CD30z/CD28z |
| E1D3 | 1779 | 2426 | 2959 | 1.36 | 1.66 |
| E1D5 | 2108 | 2826 | 2724 | 1.34 | 1.29 |
| E2D3 | 714 | 1758 | 1576 | 2.46 | 2.21 |
| E2D5 | 1703 | 3283 | 2844 | 1.93 | 1.67 |
| E3D5 | 586 | 1284 | 1788 | 2.19 | 3.05 |
| E3D7 | 468 | 918 | 840 | 1.96 | 1.79 |

TABLE 7B
Central memory T cell (Tcm) count and ratio of total
CD8⁺ Receptor⁺ CD30-CAR vs. CD28-CAR T.

| | | | Tcm | | |
|---|---|---|---|---|---|
| | Tcm | Tcm | count | | |
| | count | count | αCD19- | Ratio of Tcm | Ratio of Tcm |
| | αCD19- | αCD19- | CD8T- | cell count | cell count |
| Date | CD28z | CD30z | CD30z | CD30z/CD28z | CD30z/CD28z |
| E1D3 | 1292 | 2004 | 2074 | 1.55 | 1.61 |
| E1D5 | 1264 | 2073 | 1902 | 1.64 | 1.5 |
| E2D3 | 491 | 1673 | 1269 | 3.41 | 2.58 |
| E2D5 | 1477 | 3211 | 2603 | 2.17 | 1.76 |
| E3D5 | 496 | 1243 | 1249 | 2.51 | 2.52 |
| E3D7 | 408 | 889 | 567 | 2.18 | 1.39 |

TABLE 7C
Central memory T cell (Tcm) count and ratio
of total CD8⁺ CD30-CAR vs. CD8T-41BB-CAR T.

| | Tcm | | Tcm | | Ratio of Tcm |
|---|---|---|---|---|---|
| | count | Tcm | count | Ratio of Tcm | cell count |
| | αCD19- | count | αCD19- | cell count | CD8T- |
| | CD8T- | αCD19- | CD8T- | CD30z/CD8T- | CD30z/CD8T- |
| Date | 41BBz | CD30z | CD30z | 41BBz | 41BBz |
| E1D3 | 2296 | 2426 | 2959 | 1.06 | 1.29 |
| E1D5 | 2507 | 2826 | 2724 | 1.13 | 1.09 |
| E2D3 | 390 | 1758 | 1576 | 4.51 | 4.04 |
| E2D5 | 980 | 3283 | 2844 | 3.35 | 2.9 |
| E3D5 | 169 | 1284 | 1788 | 7.6 | 10.58 |
| E3D7 | 536 | 1161 | 860 | 2.17 | 1.6 |

TABLE 7D
Central memory T cell (Tcm) count and ratio of CD8⁺
Receptor⁺ CD30-CAR vs. CD8T-41BB-CAR T.

| | Tcm | | Tcm | | Ratio of Tcm |
|---|---|---|---|---|---|
| | count | Tcm | count | Ratio of Tcm | cell count |
| | αCD19- | count | αCD19- | cell count | CD8T- |
| | CD8T- | αCD19- | CD8T- | CD30z/CD8T- | CD30z/CD8T- |
| Date | 41BBz | CD30z | CD30z | 41BBz | 41BBz |
| E1D3 | 1292 | 1374 | 2004 | 1.46 | 1.51 |
| E1D5 | 1264 | 1056 | 2073 | 1.96 | 1.8 |
| E2D3 | 491 | 245 | 1673 | 6.83 | 5.18 |
| E2D5 | 1477 | 804 | 3211 | 3.99 | 3.24 |
| E3D5 | 496 | 50 | 1243 | 24.86 | 24.98 |
| E3D7 | 408 | 57 | 889 | 1 | 9.95 |

TABLE 7E
Effector memory T cell (Tem) count and ratio
of total CD8⁺ CD30-CAR vs. CD8T-41BB-CAR.

| | Tem | | Tem | | Ratio of Tem |
|---|---|---|---|---|---|
| | count | Tem | count | Ratio of Tem | cell count |
| | αCD19- | count | αCD19- | cell count | CD8T- |
| | CD8T- | αCD19- | CD8T- | CD30z/CD8T- | CD30z/CD8T- |
| Date | 41BBz | CD30z | CD30z | 41BBz | 41BBz |
| E1D3 | 11322 | 12157 | 7062 | 1.07 | 0.62 |
| E1D5 | 5061 | 5796 | 4287 | 1.15 | 0.85 |
| E2D3 | 3837 | 6072 | 5188 | 1.58 | 1.35 |
| E2D5 | 2915 | 5281 | 4601 | 1.81 | 1.58 |
| E3D5 | 1050 | 3451 | 3018 | 3.29 | 2.87 |
| E3D7 | 536 | 1161 | 860 | 2.17 | 1.60 |

TABLE 7F
Effector memory T cell (Tem) count and ratio of
CD8⁺ Receptor⁺ CD30-CAR vs. CD8T-41BB-CAR.

| | Tem | | Tem | | Ratio of Tem |
|---|---|---|---|---|---|
| | count | Tem | count | Ratio of Tem | cell count |
| | αCD19- | count | αCD19- | cell count | CD8T- |
| | CD8T- | αCD19- | CD8T- | CD30z/CD8T- | CD30z/CD8T- |
| Date | 41BBz | CD30z | CD30z | 41BBz | 41BBz |
| E1D3 | 20498 | 7158 | 4267 | 1.32 | 0.6 |
| E1D5 | 13203 | 4270 | 2666 | 1.74 | 1.09 |
| E2D3 | 11669 | 5200 | 3782 | 1.77 | 1.28 |
| E2D5 | 10569 | 4723 | 3951 | 1.91 | 1.59 |
| E3D5 | 8355 | 3027 | 1540 | 17.2 | 8.75 |
| E3D7 | 2983 | 1005 | 380 | 6.32 | 2.39 |

These surprising results showed that CD8⁺ cytotoxic T cells expressing CAR+CD30 were able to develop into and maintain high numbers and percentages of central memory and effector memory T cells higher than those by CD8⁺ T cells expressing CAR+CD28 or CAR+41BB. These results suggest that the CAR+CD30 T cell platform is an excellent T cell therapy platform for treating cancer patients, including patients suffering from B cell malignancies.

### EXEMPLARY EMBODIMENTS

Exemplary embodiments provided in accordance with the presently disclosed subject matter include, but are not limited to, the claims and the following embodiments:

1. A chimeric antigen receptor (CAR) comprising:
(a) an extracellular target-binding domain comprising an antibody moiety;
(b) a transmembrane domain;
(c) a CD30 costimulatory domain; and
(d) a primary signaling domain.
2. The CAR of embodiment 1, wherein the CD30 costimulatory domain comprises a sequence that can bind to an intracellular TRAF signaling protein.
3. The CAR of embodiment 2, wherein the sequence that can bind to an intracellular TRAF signaling protein corresponds to residues 561-573 or 578-586 of a full-length CD30 having the sequence of SEQ ID NO:11.
4. The CAR of any one of embodiments 1 to 3, wherein the CD30 costimulatory domain comprises a sequence that is at least 80%, 85%, 90%, 95%, or 100% identical to residues 561-573 or 578-586 of SEQ ID NO:11.
5. The CAR of any one of embodiments 1 to 4, wherein the CD30 costimulatory domain comprises a sequence that is at least 70%, 75%, 80%, 85%, 90%, 95%, or 100% identical to the sequence of SEQ ID NO:35.
6. The CAR of any one of embodiments 1 to 5, wherein the CAR comprises more than one CD30 costimulatory domain.
7. The CAR of any one of embodiments 1 to 6, wherein the CAR further comprises at least one costimulatory domain which comprises the intracellular sequence of a costimulatory molecule that is different from CD30.
8. The CAR of embodiment 7, wherein the costimulatory molecule that is different from CD30 is selected from the group consisting of CD27, CD28, 4-1BB (CD137), OX40, CD40, PD-1, ICOS, lymphocyte function-associated antigen-1 (LFA-1), CD2, CD7, LIGHT, NKG2C, B7-H3, and a ligand that specifically binds with CD83.
9. The CAR of any one of embodiments 1 to 8, wherein the antibody moiety is a single chain antibody fragment.
10. The CAR of any one of embodiments 1 to 9, wherein the antibody moiety is a single chain Fv (scFv), a single chain Fab, a single chain Fab′, a single domain antibody fragment, a single domain multispecific antibody, an intrabody, a nanobody, or a single chain immunokine.
11. The CAR of embodiment 10, wherein the antibody moiety is a single domain multispecific antibody.
12. The CAR of embodiment 11, wherein the single domain multispecific antibody is a single domain bispecific antibody.
13. The CAR of any one of embodiments 10 to 12, wherein the antibody moiety is a single chain Fv (scFv).
14. The CAR of embodiment 13, wherein the scFv is a tandem scFv.
15. The CAR of any one of embodiments 1 to 14, wherein the transmembrane domain of the CAR is derived from the transmembrane domain of a TCR co-receptor or a T cell costimulatory molecule.
16. The CAR of embodiment 15, wherein the TCR co-receptor or T cell costimulatory molecule is selected from the group consisting of CD8, 4-1BB, CD27, CD28, CD30, OX40, CD3ε, CD3ζ, CD45, CD4, CD5, CD9, CD16, CD22, CD33, CD37, CD64, CD80, CD86, CD134, CD137, and CD154.
17. The CAR of embodiment 15 or 16, wherein the TCR co-receptor or T cell costimulatory molecule is CD30 or CD8.
18. The CAR of embodiment 17, wherein the T cell costimulatory molecule is CD30.
19. The CAR of embodiment 17, wherein the TCR co-receptor is CD8.
20. The CAR of any one of embodiments 1 to 14, wherein the transmembrane domain of the CAR is the transmembrane domain of CD8, 4-1BB, CD27, CD28, CD30, OX40, CD3ε, CD3ζ, CD45, CD4, CD5, CD9, CD16, CD22, CD33, CD37, CD64, CD80, CD86, CD134, CD137, or CD154.
21. The CAR of embodiment 20, wherein the transmembrane domain of the CAR is the transmembrane domain of CD30 or CD8.
22. The CAR of embodiment 21, wherein the transmembrane domain of the CAR is the transmembrane domain of CD30.
23. The CAR of embodiment 21, wherein the transmembrane domain of the CAR is the transmembrane domain of CD8.
24. The CAR of any one of embodiments 1 to 23, wherein the transmembrane domain of the CAR comprises an amino acid sequence selected from the group consisting of SEQ ID NOS:26-31.
25. The CAR of any one of embodiments 1 to 24, wherein the primary signaling domain comprises a sequence derived from the intracellular signaling sequence of a molecule selected from the group consisting of CD3ζ, TCRζ, FcRγ, FcRβ, CD3γ, CD3δ, CD3ε, CD5, CD22, CD79a, CD79b, and CD66d.
26. The CAR of any one of embodiments 1 to 25, wherein the primary signaling domain comprises a sequence derived from the intracellular signaling sequence of CD3ζ.
27. The CAR of embodiment 26, wherein the primary signaling domain comprises the intracellular signaling sequence of CD3ζ.
28. The CAR of any one of embodiments 1 to 27, wherein the primary signaling domain comprises a sequence that is at least 80%, 85%, 90%, 95%, or 100% identical to the sequence of SEQ ID NO:37.
29. The CAR of any one of embodiments 1 to 28, further comprises a peptide linker between the extracellular target-binding domain and the transmembrane domain.
30. The CAR of any one of embodiments 1 to 29, further comprises a peptide linker between the transmembrane domain and the CD30 costimulatory domain.
31. The CAR of any one of embodiments 1 to 30, further comprises a peptide linker between the CD30 costimulatory domain and the primary signaling domain.
32. The CAR of any one of embodiments 1 to 31, wherein the antibody moiety specifically binds to a disease-related antigen.
33. The CAR of embodiment 32, wherein the disease-related antigen is a cancer-related antigen.
34. The CAR of embodiment 32, wherein the disease-related antigen is a virus-related antigen.
35. The CAR of any one of embodiments 1 to 34, wherein the antibody moiety specifically binds to a cell surface antigen.
36. The CAR of embodiment 35, wherein the cell surface antigen is selected from the group consisting of protein, carbohydrate, and lipid.
37. The CAR of embodiment 35 or 36, wherein the cell surface antigen is CD19, CD20, CD22, CD47, CD158e, GPC3, ROR1, ROR2, BCMA, GPRC5D, FcRL5, MUC16, MCT4, PSMA, or a variant or mutant thereof.
38. The CAR of any one of embodiments 1 to 37, wherein the antibody moiety specifically binds to human CD19.
39. The CAR of any one of embodiments 1 to 37, wherein the antibody moiety specifically binds to human CD22.
40. The CAR of any one of embodiments 1 to 37, wherein the antibody moiety specifically binds to human CD20.
41. The CAR of any one of embodiments 1 to 37, wherein the antibody moiety specifically binds to both human CD19 and human CD22.
42. The CAR of any one of embodiments 1 to 37, wherein the antibody moiety specifically binds to both human CD19 and human CD20.
43. The CAR of any one of embodiments 1 to 37, wherein the antibody moiety specifically binds to both human CD20 and human CD22.
44. The CAR of any one of embodiments 1 to 37, wherein the antibody moiety specifically binds to human CD19, human CD20, and human CD22.
45. The CAR of any one of embodiments 38 to 44, wherein the transmembrane domain of the CAR is the transmembrane domain of CD30.
46. The CAR of any one of embodiments 1 to 34, wherein the antibody moiety specifically binds to a MHC-restricted antigen.
47. The CAR of embodiment 46, wherein the antibody moiety specifically binds to a complex comprising an alpha-fetoprotein (AFP) peptide and a MHC class I protein.
48. The CAR of embodiment 47, wherein the AFP peptide comprises a sequence of any one of SEQ ID NOS:72-82.
49. The CAR of embodiment 47 or 48, wherein antibody moiety comprises sequences of HCDR1, HCDR2, and HCDR3 of SEQ ID NOS:83-85, respectively, and optionally a heavy chain variable region having the sequence of SEQ ID NO:86.
50. The CAR of any one of embodiments 47 to 49, wherein the antibody moiety comprises sequences of LCDR1, LCDR2, and LCDR3 of SEQ ID NOS:87-89, respectively, and optionally a light chain variable region having the sequence of SEQ ID NO:90.
51. The CAR of embodiment 47 or 48, wherein the antibody moiety comprises sequences of HCDR1, HCDR2, and HCDR3 of SEQ ID NOS:91-93, respectively, and optionally a heavy chain variable region having the sequence of SEQ ID NO:94.
52. The CAR of any one of embodiments 47, 48, and 51, wherein the antibody moiety comprises sequences of LCDR1, LCDR2, and LCDR3 of SEQ ID NOS:95-97, respectively, and optionally a light chain variable region having the sequence of SEQ ID NO:98.
53. The CAR of embodiment 47 or 48, wherein the antibody moiety comprises sequences of HCDR1, HCDR2, and HCDR3 of SEQ ID NOS:99-101, respectively, and optionally a heavy chain variable region having the sequence of SEQ ID NO:102.
54. The CAR of any one of embodiments 47, 48, and 53, wherein the antibody moiety comprises sequences of LCDR1, LCDR2, and LCDR3 of SEQ ID NOS:103-105, respectively, and optionally a light chain variable region having the sequence of SEQ ID NO:106.
55. The CAR of embodiment 47 or 48, wherein the antibody moiety comprises sequences of HCDR1, HCDR2, and HCDR3 of SEQ ID NOS:107-109, respectively, and optionally a heavy chain variable region having the sequence of SEQ ID NO:110.
56. The CAR of any one of embodiments 47, 48, and 55, wherein the antibody moiety comprises sequences of LCDR1, LCDR2, and LCDR3 of SEQ ID NOS:111-113, respectively, and optionally a light chain variable region having the sequence of SEQ ID NO:114.
57. The CAR of embodiment 47 or 48, wherein the antibody moiety comprises sequences of HCDR1, HCDR2, and HCDR3 of SEQ ID NOS:115-117, respectively, and optionally a heavy chain variable region having the sequence of SEQ ID NO:118.
58. The CAR of any one of embodiments 47, 48, and 57, wherein the antibody moiety comprises sequences of LCDR1, LCDR2, and LCDR3 of SEQ ID NOS:119-121, respectively, and optionally a light chain variable region having the sequence of SEQ ID NO:122.
59. The CAR of any one of embodiments 1 to 33 and 35 to 37, wherein the antibody moiety specifically binds to a glypican 3 (GPC3) peptide.
60. The CAR of embodiment 59, wherein the antibody moiety comprises sequences of HCDR1, HCDR2, and HCDR3 of SEQ ID NOS:123-125, respectively, and optionally a heavy chain variable region having the sequence of SEQ ID NO:126.
61. The CAR of embodiment 59 or 60, wherein the antibody moiety comprises sequences of LCDR1, LCDR2, and LCDR3 of SEQ ID NOS:127-129, respectively, and optionally a light chain variable region having the sequence of SEQ ID NO:130.
62. The CAR of embodiment 59, wherein the antibody moiety comprises sequences of HCDR1, HCDR2, and HCDR3 of SEQ ID NOS:131-133, respectively, and optionally a heavy chain variable region having the sequence of SEQ ID NO:134.
63. The CAR of embodiment 59 or 62, wherein the antibody moiety comprises sequences of LCDR1, LCDR2, and LCDR3 of SEQ ID NOS:135-137, respectively, and optionally a light chain variable region having the sequence of SEQ ID NO:138.
64. The CAR of embodiment 59, wherein the antibody moiety comprises sequences of HCDR1, HCDR2, and HCDR3 of SEQ ID NOS:139-141, respectively, and optionally a heavy chain variable region having the sequence of SEQ ID NO:142.
65. The CAR of embodiment 59 or 64, wherein the antibody moiety comprises sequences of LCDR1, LCDR2, and LCDR3 of SEQ ID NOS:143-145, respectively, and optionally a light chain variable region having the sequence of SEQ ID NO:146.
66. The CAR of embodiment 59, wherein the antibody moiety comprises sequences of HCDR1, HCDR2, and HCDR3 of SEQ ID NOS:147-149, respectively, and optionally a heavy chain variable region having the sequence of SEQ ID NO:150.
67. The CAR of embodiment 59 or 66, wherein the antibody moiety comprises sequences of LCDR1, LCDR2, and LCDR3 of SEQ ID NOS:151-153, respectively, and optionally a light chain variable region having the sequence of SEQ ID NO:154.
68. The CAR of embodiment 59, wherein the antibody moiety comprises sequences of HCDR1, HCDR2, and HCDR3 of SEQ ID NOS:155-157, respectively, and optionally a heavy chain variable region having the sequence of SEQ ID NO:158.
69. The CAR of embodiment 59 or 68, wherein the antibody moiety comprises sequences of LCDR1, LCDR2, and LCDR3 of SEQ ID NOS:159-161, respectively, and optionally a light chain variable region having the sequence of SEQ ID NO:162.
70. The CAR of embodiment 59, wherein the antibody moiety comprises sequences of HCDR1, HCDR2, and HCDR3 of SEQ ID NOS:163-165, respectively, and optionally a heavy chain variable region having the sequence of SEQ ID NO:68.
71. The CAR of embodiment 59 or 70, wherein the antibody moiety comprises sequences of LCDR1, LCDR2, and LCDR3 of SEQ ID NOS:166-168, respectively, and optionally a light chain variable region having the sequence of SEQ ID NO:69.
72. The CAR of embodiment 59, wherein the antibody moiety comprises sequences of HCDR1, HCDR2, and HCDR3 of SEQ ID NOS:169-171, respectively, and optionally a heavy chain variable region having the sequence of SEQ ID NO:70.
73. The CAR of embodiment 59 or 72, wherein the antibody moiety comprises sequences of LCDR1, LCDR2, and LCDR3 of SEQ ID NOS:172-174, respectively, and optionally a light chain variable region having the sequence of SEQ ID NO:71.
74. The CAR of any one of embodiments 70 to 73, wherein the antibody moiety comprises a sequence of SEQ ID NO:12 or 13.
75. The CAR of embodiment 46, wherein the antibody moiety specifically binds to a complex comprising a KRAS peptide and a MHC class I protein.
76. The CAR of embodiment 75, wherein the KRAS peptide comprises a sequence of any one of SEQ ID NOS:175-183.
77. The CAR of embodiment 75 or 76, wherein antibody moiety comprises sequences of HCDR1, HCDR2, and HCDR3 of SEQ ID NOS:184-186, respectively, and optionally a heavy chain variable region having the sequence of SEQ ID NO:187.
78. The CAR of any one of embodiments 75 to 77, wherein the antibody moiety comprises sequences of LCDR1, LCDR2, and LCDR3 of SEQ ID NOS:188-190, respectively, and optionally a light chain variable region having the sequence of SEQ ID NO:191.
79. The CAR of any one of embodiments 75 to 78, wherein the antibody moiety comprises a sequence of SEQ ID NO:192.
80. The CAR of embodiment 75 or 76, wherein the antibody moiety comprises sequences of HCDR1, HCDR2, and HCDR3 of SEQ ID NOS:193-195, respectively, and optionally a heavy chain variable region having the sequence of SEQ ID NO:196.
81. The CAR of any one of embodiments 75, 76, and 80, wherein the antibody moiety comprises sequences of LCDR1, LCDR2, and LCDR3 of SEQ ID NOS:197-199, respectively, and optionally a light chain variable region having the sequence of SEQ ID NO:200.
82. The CAR of any one of embodiments 75, 76, 80, and 81, wherein the antibody moiety comprises a sequence of SEQ ID NO:201.
83. The CAR of embodiment 75 or 76, wherein the antibody moiety comprises sequences of HCDR1, HCDR2, and HCDR3 of SEQ ID NOS:202-204, respectively, and optionally a heavy chain variable region having the sequence of SEQ ID NO:205.
84. The CAR of any one of embodiments 75, 76, and 83, wherein the antibody moiety comprises sequences of LCDR1, LCDR2, and LCDR3 of SEQ ID NOS:206-208, respectively, and optionally a light chain variable region having the sequence of SEQ ID NO:209.
85. The CAR of any one of embodiments 75, 76, 83, and 84, wherein the antibody moiety comprises a sequence of SEQ ID NO:210.
86. The CAR of embodiment 75 or 76, wherein the antibody moiety comprises sequences of HCDR1, HCDR2, and HCDR3 of SEQ ID NOS:211-213, respectively, and optionally a heavy chain variable region having the sequence of SEQ ID NO:214.
87. The CAR of any one of embodiments 75, 76, and 86, wherein the antibody moiety comprises sequences of LCDR1, LCDR2, and LCDR3 of SEQ ID NOS:215-217, respectively, and optionally a light chain variable region having the sequence of SEQ ID NO:218.
88. The CAR of any one of embodiments 75, 76, 86, and 87, wherein the antibody moiety comprises a sequence of SEQ ID NO:219.
89. The CAR of embodiment 75 or 76, wherein the antibody moiety comprises sequences of HCDR1, HCDR2, and HCDR3 of SEQ ID NOS:220-222, respectively, and optionally a heavy chain variable region having the sequence of SEQ ID NO:223.
90. The CAR of any one of embodiments 75, 76, and 89, wherein the antibody moiety comprises sequences of LCDR1, LCDR2, and LCDR3 of SEQ ID NOS:224-226, respectively, and optionally a light chain variable region having the sequence of SEQ ID NO:227.
91. The CAR of any one of embodiments 75, 76, 89, and 90, wherein the antibody moiety comprises a sequence of SEQ ID NO:228.
92. The CAR of embodiment 75 or 76, wherein the antibody moiety comprises sequences of HCDR1, HCDR2, and HCDR3 of SEQ ID NOS:229-231, respectively, and optionally a heavy chain variable region having the sequence of SEQ ID NO:232.
93. The CAR of any one of embodiments 75, 76, and 92, wherein the antibody moiety comprises sequences of LCDR1, LCDR2, and LCDR3 of SEQ ID NOS:233-235, respectively, and optionally a light chain variable region having the sequence of SEQ ID NO:236.
94. The CAR of any one of embodiments 75, 76, 92, and 93, wherein the antibody moiety comprises a sequence of SEQ ID NO:237.
95. The CAR of embodiment 75 or 76, wherein the antibody moiety comprises sequences of HCDR1, HCDR2, and HCDR3 of SEQ ID NOS:238-240, respectively, and optionally a heavy chain variable region having the sequence of SEQ ID NO:241.
96. The CAR of any one of embodiments 75, 76, and 95, wherein the antibody moiety comprises sequences of LCDR1, LCDR2, and LCDR3 of SEQ ID NOS:242-244, respectively, and optionally a light chain variable region having the sequence of SEQ ID NO:245.
97. The CAR of any one of embodiments 75, 76, 95, and 96, wherein the antibody moiety comprises a sequence of SEQ ID NO:246.
98. The CAR of embodiment 75 or 76, wherein the antibody moiety comprises sequences of HCDR1, HCDR2, and HCDR3 of SEQ ID NOS:247-249, respectively, and optionally a heavy chain variable region having the sequence of SEQ ID NO:250.
99. The CAR of any one of embodiments 75, 76, and 98, wherein the antibody moiety comprises sequences of LCDR1, LCDR2, and LCDR3 of SEQ ID NOS:251-253, respectively, and optionally a light chain variable region having the sequence of SEQ ID NO:254.
100. The CAR of any one of embodiments 75, 76, 98, and 99, wherein the antibody moiety comprises a sequence of SEQ ID NO:255.
101. The CAR of embodiment 46, wherein the antibody moiety specifically binds to a complex comprising a NY-ESO-1 peptide and a MHC class I protein.
102. The CAR of embodiment 101, wherein the NY-ESO-1 peptide comprises a sequence of any one of SEQ ID NOS:256-266.
103. The CAR of embodiment 101 or 102, wherein antibody moiety comprises sequences of HCDR1, HCDR2, and HCDR3 of SEQ ID NOS:267-269, respectively, and optionally a heavy chain variable region having the sequence of SEQ ID NO:270.
104. The CAR of any one of embodiments 101 to 103, wherein the antibody moiety comprises sequences of LCDR1, LCDR2, and LCDR3 of SEQ ID NOS:271-273, respectively, and optionally a light chain variable region having the sequence of SEQ ID NO:274.
105. The CAR of embodiment 101 or 102, wherein the antibody moiety comprises sequences of HCDR1, HCDR2, and HCDR3 of SEQ ID NOS:275-277, respectively, and optionally a heavy chain variable region having the sequence of SEQ ID NO:278.
106. The CAR of any one of embodiments 101, 102, and 105, wherein the antibody moiety comprises sequences of LCDR1, LCDR2, and LCDR3 of SEQ ID NOS:279-281, respectively, and optionally a light chain variable region having the sequence of SEQ ID NO:282.
107. The CAR of embodiment 101 or 102, wherein the antibody moiety comprises sequences of HCDR1, HCDR2, and HCDR3 of SEQ ID NOS:283-285, respectively, and optionally a heavy chain variable region having the sequence of SEQ ID NO:286.
108. The CAR of any one of embodiments 101, 102, and 107, wherein the antibody moiety comprises sequences of LCDR1, LCDR2, and LCDR3 of SEQ ID NOS:287-289, respectively, and optionally a light chain variable region having the sequence of SEQ ID NO:290.
109. The CAR of embodiment 101 or 102, wherein the antibody moiety comprises sequences of HCDR1, HCDR2, and HCDR3 of SEQ ID NOS:291-293, respectively, and optionally a heavy chain variable region having the sequence of SEQ ID NO:294.
110. The CAR of any one of embodiments 101, 102, and 109, wherein the antibody moiety comprises sequences of LCDR1, LCDR2, and LCDR3 of SEQ ID NOS:295-297, respectively, and optionally a light chain variable region having the sequence of SEQ ID NO:298.
111. The CAR of embodiment 101 or 102, wherein the antibody moiety comprises sequences of HCDR1, HCDR2, and HCDR3 of SEQ ID NOS:299-301, respectively, and optionally a heavy chain variable region having the sequence of SEQ ID NO:302.
112. The CAR of any one of embodiments 101, 102, and 111, wherein the antibody moiety comprises sequences of LCDR1, LCDR2, and LCDR3 of SEQ ID NOS:303-305, respectively, and optionally a light chain variable region having the sequence of SEQ ID NO:306.
113. The CAR of embodiment 101 or 102, wherein the antibody moiety comprises sequences of HCDR1, HCDR2, and HCDR3 of SEQ ID NOS:307-309, respectively, and optionally a heavy chain variable region having the sequence of SEQ ID NO:310.
114. The CAR of any one of embodiments 101, 102, and 113, wherein the antibody moiety comprises sequences of LCDR1, LCDR2, and LCDR3 of SEQ ID NOS:311-313, respectively, and optionally a light chain variable region having the sequence of SEQ ID NO:314.
115. The CAR of embodiment 101 or 102, wherein the antibody moiety comprises sequences of HCDR1, HCDR2, and HCDR3 of SEQ ID NOS:315-317, respectively, and optionally a heavy chain variable region having the sequence of SEQ ID NO:318.
116. The CAR of any one of embodiments 101, 102, and 115, wherein the antibody moiety comprises sequences of LCDR1, LCDR2, and LCDR3 of SEQ ID NOS:319-321, respectively, and optionally a light chain variable region having the sequence of SEQ ID NO:322.
117. The CAR of embodiment 46, wherein the antibody moiety specifically binds to a complex comprising a PRAME peptide and a MHC class I protein.
118. The CAR of embodiment 117, wherein the PRAME peptide comprises a sequence of any one of SEQ ID NOS:323-327.
119. The CAR of embodiment 117 or 118, wherein antibody moiety comprises sequences of HCDR1, HCDR2, and HCDR3 of SEQ ID NOS:328-330, respectively, and optionally a heavy chain variable region having the sequence of SEQ ID NO:331.
120. The CAR of any one of embodiments 117 to 119, wherein the antibody moiety comprises sequences of LCDR1, LCDR2, and LCDR3 of SEQ ID NOS:332-334, respectively, and optionally a light chain variable region having the sequence of SEQ ID NO:335.
121. The CAR of embodiment 117 or 118, wherein the antibody moiety comprises sequences of HCDR1, HCDR2, and HCDR3 of SEQ ID NOS:336-338, respectively, and optionally a heavy chain variable region having the sequence of SEQ ID NO:339.
122. The CAR of any one of embodiments 117, 118, and 121, wherein the antibody moiety comprises sequences of LCDR1, LCDR2, and LCDR3 of SEQ ID NOS:340-342, respectively, and optionally a light chain variable region having the sequence of SEQ ID NO:343.
123. The CAR of embodiment 117 or 118, wherein the antibody moiety comprises sequences of HCDR1, HCDR2, and HCDR3 of SEQ ID NOS:344-346, respectively, and optionally a heavy chain variable region having the sequence of SEQ ID NO:347.
124. The CAR of any one of embodiments 117, 118, and 123, wherein the antibody moiety comprises sequences of LCDR1, LCDR2, and LCDR3 of SEQ ID NOS:348-350, respectively, and optionally a light chain variable region having the sequence of SEQ ID NO:351.
125. The CAR of embodiment 117 or 118, wherein the antibody moiety comprises sequences of HCDR1, HCDR2, and HCDR3 of SEQ ID NOS:352-354, respectively, and optionally a heavy chain variable region having the sequence of SEQ ID NO:355.
126. The CAR of any one of embodiments 117, 118, and 125, wherein the antibody moiety comprises sequences of LCDR1, LCDR2, and LCDR3 of SEQ ID NOS:356-358, respectively, and optionally a light chain variable region having the sequence of SEQ ID NO:359.
127. The CAR of embodiment 117 or 118, wherein the antibody moiety comprises sequences of HCDR1, HCDR2, and HCDR3 of SEQ ID NOS:360-362, respectively, and optionally a heavy chain variable region having the sequence of SEQ ID NO:363.
128. The CAR of any one of embodiments 117, 118, and 127, wherein the antibody moiety comprises sequences of LCDR1, LCDR2, and LCDR3 of SEQ ID NOS:364-366, respectively, and optionally a light chain variable region having the sequence of SEQ ID NO:367.
129. The CAR of embodiment 117 or 118, wherein the antibody moiety comprises sequences of HCDR1, HCDR2, and HCDR3 of SEQ ID NOS:368-370, respectively, and optionally a heavy chain variable region having the sequence of SEQ ID NO:371.
130. The CAR of any one of embodiments 117, 118, and 129, wherein the antibody moiety comprises sequences of LCDR1, LCDR2, and LCDR3 of SEQ ID NOS:372-374, respectively, and optionally a light chain variable region having the sequence of SEQ ID NO:375.
131. The CAR of embodiment 117 or 118, wherein the antibody moiety comprises sequences of HCDR1, HCDR2, and HCDR3 of SEQ ID NOS:376-378, respectively, and optionally a heavy chain variable region having the sequence of SEQ ID NO:379.
132. The CAR of any one of embodiments 117, 118, and 131, wherein the antibody moiety comprises sequences of LCDR1, LCDR2, and LCDR3 of SEQ ID NOS:380-382, respectively, and optionally a light chain variable region having the sequence of SEQ ID NO:383.
133. The CAR of embodiment 46, wherein the antibody moiety specifically binds to a complex comprising a histone H3.3 peptide and a MHC class I protein.
134. The CAR of embodiment 133, wherein the histone H3.3 peptide comprises a sequence of any one of SEQ ID NOS:384-403.
135. The CAR of embodiment 133 or 134, wherein antibody moiety comprises sequences of HCDR1, HCDR2, and HCDR3 of SEQ ID NOS:404-406, respectively, and optionally a heavy chain variable region having the sequence of SEQ ID NO:407.
136. The CAR of any one of embodiments 133 to 135, wherein the antibody moiety comprises sequences of LCDR1, LCDR2, and LCDR3 of SEQ ID NOS:408-410, respectively, and optionally a light chain variable region having the sequence of SEQ ID NO:411.
137. The CAR of embodiment 133 or 134, wherein the antibody moiety comprises sequences of HCDR1, HCDR2, and HCDR3 of SEQ ID NOS:412-414, respectively, and optionally a heavy chain variable region having the sequence of SEQ ID NO:415.
138. The CAR of any one of embodiments 133, 134, and 137, wherein the antibody moiety comprises sequences of LCDR1, LCDR2, and LCDR3 of SEQ ID NOS:416-418, respectively, and optionally a light chain variable region having the sequence of SEQ ID NO:419.
139. The CAR of embodiment 133 or 134, wherein the antibody moiety comprises sequences of HCDR1, HCDR2, and HCDR3 of SEQ ID NOS:420-422, respectively, and optionally a heavy chain variable region having the sequence of SEQ ID NO:423.
140. The CAR of any one of embodiments 133, 134, and 139, wherein the antibody moiety comprises sequences of LCDR1, LCDR2, and LCDR3 of SEQ ID NOS:424-426, respectively, and optionally a light chain variable region having the sequence of SEQ ID NO:427.
141. The CAR of embodiment 133 or 134, wherein the antibody moiety comprises sequences of HCDR1, HCDR2, and HCDR3 of SEQ ID NOS:428-430, respectively, and optionally a heavy chain variable region having the sequence of SEQ ID NO:431.
142. The CAR of any one of embodiments 133, 134, and 141, wherein the antibody moiety comprises sequences of LCDR1, LCDR2, and LCDR3 of SEQ ID NOS:432-434, respectively, and optionally a light chain variable region having the sequence of SEQ ID NO:435.
143. The CAR of embodiment 133 or 134, wherein the antibody moiety comprises sequences of HCDR1, HCDR2, and HCDR3 of SEQ ID NOS:436-438, respectively, and optionally a heavy chain variable region having the sequence of SEQ ID NO:439.
144. The CAR of any one of embodiments 133, 134, and 143, wherein the antibody moiety comprises sequences of LCDR1, LCDR2, and LCDR3 of SEQ ID NOS:440-442, respectively, and optionally a light chain variable region having the sequence of SEQ ID NO:443.
145. The CAR of embodiment 133 or 134, wherein the antibody moiety comprises sequences of HCDR1, HCDR2, and HCDR3 of SEQ ID NOS:444-446, respectively, and optionally a heavy chain variable region having the sequence of SEQ ID NO:447.
146. The CAR of any one of embodiments 133, 134, and 145, wherein the antibody moiety comprises sequences of LCDR1, LCDR2, and LCDR3 of SEQ ID NOS:448-450, respectively, and optionally a light chain variable region having the sequence of SEQ ID NO:451.
147. The CAR of embodiment 133 or 134, wherein the antibody moiety comprises sequences of HCDR1, HCDR2, and HCDR3 of SEQ ID NOS:452-454, respectively, and optionally a heavy chain variable region having the sequence of SEQ ID NO:455.
148. The CAR of any one of embodiments 133, 134, and 147, wherein the antibody moiety comprises sequences of LCDR1, LCDR2, and LCDR3 of SEQ ID NOS:456-458, respectively, and optionally a light chain variable region having the sequence of SEQ ID NO:459.
149. The CAR of embodiment 133 or 134, wherein the antibody moiety comprises sequences of HCDR1, HCDR2, and HCDR3 of SEQ ID NOS:460-462, respectively, and optionally a heavy chain variable region having the sequence of SEQ ID NO:463.
150. The CAR of any one of embodiments 133, 134, and 149, wherein the antibody moiety comprises sequences of LCDR1, LCDR2, and LCDR3 of SEQ ID NOS:464-466, respectively, and optionally a light chain variable region having the sequence of SEQ ID NO:467.
151. The CAR of embodiment 133 or 134, wherein the antibody moiety comprises sequences of HCDR1, HCDR2, and HCDR3 of SEQ ID NOS:468-470, respectively, and optionally a heavy chain variable region having the sequence of SEQ ID NO:471.
152. The CAR of any one of embodiments 133, 134, and 151, wherein the antibody moiety comprises sequences of LCDR1, LCDR2, and LCDR3 of SEQ ID NOS:472-474, respectively, and optionally a light chain variable region having the sequence of SEQ ID NO:475.
153. The CAR of embodiment 133 or 134, wherein the antibody moiety comprises sequences of HCDR1, HCDR2, and HCDR3 of SEQ ID NOS:476-478, respectively, and optionally a heavy chain variable region having the sequence of SEQ ID NO:479.
154. The CAR of any one of embodiments 133, 134, and 153, wherein the antibody moiety comprises sequences of LCDR1, LCDR2, and LCDR3 of SEQ ID NOS:480-482, respectively, and optionally a light chain variable region having the sequence of SEQ ID NO:483.
155. The CAR of embodiment 133 or 134, wherein the antibody moiety comprises sequences of HCDR1, HCDR2, and HCDR3 of SEQ ID NOS:484-486, respectively, and optionally a heavy chain variable region having the sequence of SEQ ID NO:487.
156. The CAR of any one of embodiments 133, 134, and 155, wherein the antibody moiety comprises sequences of LCDR1, LCDR2, and LCDR3 of SEQ ID NOS:488-490, respectively, and optionally a light chain variable region having the sequence of SEQ ID NO:491.
157. The CAR of embodiment 133 or 134, wherein the antibody moiety comprises sequences of HCDR1, HCDR2, and HCDR3 of SEQ ID NOS:492-494, respectively, and optionally a heavy chain variable region having the sequence of SEQ ID NO:495.
158. The CAR of any one of embodiments 133, 134, and 157, wherein the antibody moiety comprises sequences of LCDR1, LCDR2, and LCDR3 of SEQ ID NOS:496-498, respectively, and optionally a light chain variable region having the sequence of SEQ ID NO:499.
159. The CAR of embodiment 46, wherein the antibody moiety specifically binds to a complex comprising a WT1 peptide and a MHC class I protein.
160. The CAR of embodiment 159, wherein the WT1 peptide comprises a sequence of SEQ ID NO:500.
161. The CAR of embodiment 159 or 160, wherein antibody moiety comprises sequences of HCDR1, HCDR2, and HCDR3 of SEQ ID NOS:501-503, respectively, and optionally a heavy chain variable region having the sequence of SEQ ID NO:504.
162. The CAR of any one of embodiments 159 to 161, wherein the antibody moiety comprises sequences of LCDR1, LCDR2, and LCDR3 of SEQ ID NOS:505-507, respectively, and optionally a light chain variable region having the sequence of SEQ ID NO:508.
163. The CAR of any one of embodiments 159 to 162, wherein the antibody moiety comprises a sequence of SEQ ID NO:509.
164. The CAR of embodiment 159 or 160, wherein the antibody moiety comprises sequences of HCDR1, HCDR2, and HCDR3 of SEQ ID NOS:510-512, respectively, and optionally a heavy chain variable region having the sequence of SEQ ID NO:513.
165. The CAR of any one of embodiments 159, 160, and 164, wherein the antibody moiety comprises sequences of LCDR1, LCDR2, and LCDR3 of SEQ ID NOS:514-516, respectively, and optionally a light chain variable region having the sequence of SEQ ID NO:517.
166. The CAR of any one of embodiments 159, 160, 164, and 165, wherein the antibody moiety comprises a sequence of SEQ ID NO:518.
167. The CAR of embodiment 159 or 160, wherein the antibody moiety comprises sequences of HCDR1, HCDR2, and HCDR3 of SEQ ID NOS:519-521, respectively, and optionally a heavy chain variable region having the sequence of SEQ ID NO:522.
168. The CAR of any one of embodiments 159, 160, and 167, wherein the antibody moiety comprises sequences of LCDR1, LCDR2, and LCDR3 of SEQ ID NOS:523-525, respectively, and optionally a light chain variable region having the sequence of SEQ ID NO:526.
169. The CAR of any one of embodiments 159, 160, 167, and 168, wherein the antibody moiety comprises a sequence of SEQ ID NO:527.
170. The CAR of embodiment 159 or 160, wherein the antibody moiety comprises sequences of HCDR1, HCDR2, and HCDR3 of SEQ ID NOS:528-530, respectively, and optionally a heavy chain variable region having the sequence of SEQ ID NO:531.
171. The CAR of any one of embodiments 159, 160, and 170, wherein the antibody moiety comprises sequences of LCDR1, LCDR2, and LCDR3 of SEQ ID NOS:532-534, respectively, and optionally a light chain variable region having the sequence of SEQ ID NO:535.
172. The CAR of any one of embodiments 159, 160, 170, and 171, wherein the antibody moiety comprises a sequence of SEQ ID NO:536.
173. The CAR of embodiment 159 or 160, wherein the antibody moiety comprises sequences of HCDR1, HCDR2, and HCDR3 of SEQ ID NOS:537-539, respectively, and optionally a heavy chain variable region having the sequence of SEQ ID NO:540.
174. The CAR of any one of embodiments 159, 160, and 173, wherein the antibody moiety comprises sequences of LCDR1, LCDR2, and LCDR3 of SEQ ID NOS:541-543, respectively, and optionally a light chain variable region having the sequence of SEQ ID NO:544.
175. The CAR of any one of embodiments 159, 160, 173, and 174, wherein the antibody moiety comprises a sequence of SEQ ID NO:545.
176. The CAR of embodiment 159 or 160, wherein the antibody moiety comprises sequences of HCDR1, HCDR2, and HCDR3 of SEQ ID NOS:546-548, respectively, and optionally a heavy chain variable region having the sequence of SEQ ID NO:549.
177. The CAR of any one of embodiments 159, 160, and 176, wherein the antibody moiety comprises sequences of LCDR1, LCDR2, and LCDR3 of SEQ ID NOS:550-552, respectively, and optionally a light chain variable region having the sequence of SEQ ID NO:553.
178. The CAR of any one of embodiments 159, 160, 176, and 177, wherein the antibody moiety comprises a sequence of SEQ ID NO:554.
179. The CAR of embodiment 46, wherein the antibody moiety specifically binds to a complex comprising a PSA peptide and a MHC class I protein.
180. The CAR of embodiment 179, wherein the PSA peptide comprises a sequence of any one of SEQ ID NOS:555-565.
181. The CAR of embodiment 179 or 180, wherein antibody moiety comprises an HCDR1 sequence of any one of SEQ ID NOS:566-580, an HCDR2 sequence of any one of SEQ ID NOS:581-594, and an HCDR3 sequence of any one of SEQ ID NOS:595-612, and optionally a heavy chain variable region having a sequence of any one of SEQ ID NOS:613-630.
182. The CAR of embodiment 179 to 181, wherein antibody moiety comprises a LCDR1 sequence of any one of SEQ ID NOS:631-647, a LCDR2 sequence of any one of SEQ ID NOS:648-660, and a LCDR3 sequence of any one of SEQ ID NOS:661-678, and optionally a light chain variable region having a sequence of any one of SEQ ID NOS:679-696.
183. The CAR of embodiment 46, wherein the antibody moiety specifically binds to a complex comprising a ROR1 peptide and a MHC class I protein.
184. The CAR of embodiment 183, wherein the ROR1 peptide comprises a sequence of any one of SEQ ID NOS:697-700.
185. The CAR of embodiment 183 or 184, wherein the antibody moiety comprises sequences of HCDR1, HCDR2, and HCDR3 of SEQ ID NOS:701-703, respectively, and optionally a heavy chain variable region having the sequence of SEQ ID NO:704.
186. The CAR of any one of embodiments 183 to 185, wherein the antibody moiety comprises sequences of LCDR1, LCDR2, and LCDR3 of SEQ ID NOS:705-707, respectively, and optionally a light chain variable region having the sequence of SEQ ID NO:708.
187. The CAR of embodiment 183 or 184, wherein the antibody moiety comprises sequences of HCDR1, HCDR2, and HCDR3 of SEQ ID NOS:709-711, respectively, and optionally a heavy chain variable region having the sequence of SEQ ID NO:712.
188. The CAR of any one of embodiments 183, 184, and 187, wherein the antibody moiety comprises sequences of LCDR1, LCDR2, and LCDR3 of SEQ ID NOS:713-715, respectively, and optionally a light chain variable region having the sequence of SEQ ID NO:716.
189. A nucleic acid molecule encoding, in whole or in part, the CAR of any one of embodiments 1 to 188.
190. A vector comprising the nucleic acid molecule of embodiment 189
191. A CD30-CAR effector cell: (a) expressing the CAR of any one of embodiments 1 to 188, or (b) comprising the nucleic acid molecule of embodiment 189 or the vector of embodiment 190.
192. The CD30-CAR effector cell of embodiment 191, wherein the effector cell is a T cell.
193. A pharmaceutical composition comprising the CAR of any one of embodiments 1 to 188, the nucleic acid molecule of embodiment 189, the vector of embodiment 190, or the CD30-CAR effector cell of embodiment 191 or 192, and a pharmaceutically acceptable carrier or diluent.
194. A method of killing target cells, comprising:
contacting one or more target cells with one or more CD30-CAR effector cells of embodiment 191 or 192 under conditions and for a time sufficient so that the CD30-CAR effector cells mediate killing of the target cells,
   wherein the target cells express an antigen specific to the CD30-CAR effector cells, and
   wherein the CD30-CAR effector cells express a low cell exhaustion level upon contacting the target cells.
   195. The method of embodiment 194, wherein the CD30-CAR effector T cells express a low level of an exhaustion marker selected from the group consisting of PD-1, TIM-3, and LAG-3.
   196. The method of embodiment 194 or 195, wherein the CD30-CAR effector cells are T cells.
   197. The method of any one of embodiments 194 to 196, wherein the CD30-CAR effector T cells express a low level of PD-1.
   198. The method of any one of embodiments 194 to 197, wherein the CD30-CAR effector T cells express a low level of TIM-3.
   199. The method of any one of embodiments 194 to 198, wherein the CD30-CAR effector T cells express a low level of LAG-3.
   200. The method of any one of embodiments 194 to 199, wherein the CD30-CAR effector cells express a lower level of PD-1, TIM-3, or LAG-3 than corresponding effector cells expressing a CAR comprising a CD28 costimulatory domain.
   201. The method of any one of embodiments 194 to 200 wherein the CD30-CAR effector cells express a lower level of PD-1 than the corresponding CD28 CAR effector cells, and wherein the ratio of PD-1 expression level of the CD30-CAR effector cells to the corresponding CD28 CAR effector cells is 0.9, 0.8, 0.7, 0.6, 0.5, 0.4, 0.3, 0.2, 0.1 or lower.
   202. The method of any one of embodiments 194 to 201, wherein the CD30-CAR effector cells express a lower level of TIM-3 than the corresponding CD28 CAR effector cells, and wherein the ratio of TIM-3 expression level of the CD30-CAR effector cells to the corresponding CD28 CAR effector cells is 0.9, 0.8, 0.7, 0.6, 0.5, 0.4, 0.3, 0.2, 0.1 or lower.
   203. The method of any one of embodiments 194 to 202, wherein the CD30-CAR effector cells express a lower level of LAG-3 than the corresponding CD28 CAR effector cells, and wherein the ratio of LAG-3 expression level of the CD30-CAR effector cells to the corresponding CD28 CAR effector cells is 0.9, 0.8, 0.7, 0.6, 0.5, 0.4, 0.3, 0.2, 0.1 or lower.
   204. The method of any one of embodiments 194 to 203, wherein the CD30-CAR effector T cells express a lower level of PD-1, TIM-3, or LAG-3 than corresponding effector T cells expressing a CAR comprising a 4-1BB costimulatory domain.
   205. The method of any one of embodiments 194 to 204, wherein the CD30-CAR effector T cells express a lower cell exhaustion level of PD-1 than the corresponding 4-1BB CAR effector cells, and wherein the ratio of PD-1 expression level of the CD30-CAR effector cells to the corresponding 4-1BB CAR effector cells is 0.9, 0.8, 0.7, 0.6, 0.5, 0.4, 0.3, 0.2, 0.1 or lower.
   206. The method of any one of embodiments 194 to 205, wherein the CD30-CAR effector cells express a lower level of TIM-3 than the corresponding 4-1BB CAR effector cells, and wherein the ratio of TIM-3 expression level of the CD30-CAR effector cells to the corresponding 4-1BB CAR effector cells is 0.9, 0.8, 0.7, 0.6, 0.5, 0.4, 0.3, 0.2, 0.1 or lower.
   207. The method of any one of embodiments 194 to 206, wherein the CD30-CAR effector cells express a lower level of LAG-3 than the corresponding 4-1BB CAR effector cells, and wherein the ratio of LAG-3 expression level of the CD30-CAR effector cells to the corresponding 4-1BB CAR effector cells is 0.9, 0.8, 0.7, 0.6, 0.5, 0.4, 0.3, 0.2, 0.1 or lower.
   208. The method of any one of embodiments 194 to 207, wherein the target cells are cancer cells.
   209. The method of embodiment 208, wherein the cancer cells are from a cancer selected from the group consisting of adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, leukemia, lymphoma, lung cancer, melanoma, mesothelioma, multiple myeloma, pancreatic cancer, pheochromocytoma, plasmacytoma, neuroblastoma, ovarian cancer, prostate cancer, sarcoma, stomach cancer, uterine cancer, and thyroid cancer.
   210. The method of embodiment 208 or 209, wherein the cancer cells are hematological cancer cells.
   211. The method of embodiment 208 or 209, wherein the cancer cells are solid tumor cells.
   212. The method of any one of embodiments 194 to 207, wherein the target cells are virus-infected cells.
   213. The method of embodiment 212, wherein the virus-infected cells are from a viral infection caused by a virus selected from the group consisting of Cytomegalovirus (CMV), Epstein-Barr Virus (EBV), Hepatitis B Virus (HBV), Kaposi's Sarcoma associated herpesvirus (KSHV), Human papillomavirus (HPV), Molluscum contagiosum virus (MCV), Human T cell leukemia virus 1 (HTLV-1), HIV (Human immunodeficiency virus), and Hepatitis C Virus (HCV).
   214. A method of treating a disease, the method comprising a step of administering to a subject the CAR of any one of embodiments 1 to 188, the nucleic acid molecule of embodiment 189, the vector of embodiment 190, the CD30-CAR effector cell of embodiment 191 or 192, or the pharmaceutical composition of embodiment 193 to the subject.
   215. The method of embodiment 214, wherein the disease is cancer.
   216. The method of embodiment 215, wherein the cancer is selected from the group consisting of adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, leukemia, lymphoma, lung cancer, melanoma, mesothelioma, multiple myeloma, pancreatic cancer, pheochromocytoma, plasmacytoma, neuroblastoma, ovarian cancer, prostate cancer, sarcoma, stomach cancer, uterine cancer, and thyroid cancer.
   217. The method of embodiment 215 or 216, wherein the cancer is a hematological cancer.
   218. The method of embodiment 215 or 216, wherein the cancer is a solid tumor cancer.
   219. The method of embodiment 214, wherein the disease is a viral infection.
   220. A method for preventing and/or reversing T cell exhaustion in a subject, comprising administering to the subject the CAR of any one of embodiments 1 to 188, the nucleic acid molecule of embodiment 189, the vector of embodiment 190, the CD30-CAR effector cell of embodiment 191 or 192, or the pharmaceutical composition of embodiment 193 comprising the nucleic acid molecule or the vector to the subject.
   221. The method of embodiment 220, wherein the method decreases the expression of an exhaustion marker in a T cell.
   222. The method of embodiment 220 or 221, wherein the exhaustion marker is selected from the group consisting of PD-1, TIM-3, and LAG-3.
   223. A method for generating central memory T cells and/or effector memory T cells in a subject, comprising administering to the subject the CAR of any one of embodiments 1 to 188, the nucleic acid molecule of embodiment 189, the vector of embodiment 190, the CD30-CAR effector cell of embodiment 191 or 192, or the pharmaceutical composition of embodiment 193 comprising the nucleic acid molecule or the vector to the subject.
   224. The method of embodiment 223, wherein the method:
   (a) increases the number of central memory T cells and/or the percentage of central memory T cells among all T cells in the subject; and/or
   (b) increases the number of effector memory T cells and/or the percentage of effector memory T cells among all T cells in the subject
   225. A method for generating central memory T cells and/or effector memory T cells in vitro comprising:
   contacting one or more target cells with the CD30-CAR effector cell of embodiment 191 or 192 under conditions and for a time sufficient so that the effector cell develops into central memory T cells and/or effector memory T cells, wherein the target cells express an antigen specific to the effector cell.
   226. The method of embodiment 225, wherein the method:
   (a) increases the number of central memory T cells and/or the percentage of central memory T cells among all T cells descended from the effector cell; and/or
   (b) increases the number of effector memory T cells and/or the percentage of effector memory T cells among all T cells descended from the effector cell.
   227. The method of embodiment 225 or 226, wherein the method:
   (a) generates higher number of central memory T cells and/or higher percentage of central memory T cells than corresponding effector cell expressing a CAR comprising a CD28 or 4-1BB costimulatory domain; and/or
   (b) generates higher number of effector memory T cells and/or higher percentage of effector memory T cells than corresponding effector cell expressing a CAR comprising a CD28 or 4-1BB costimulatory domain.
   228. The method of embodiment 227, wherein the method generates at least 10%, 20%, 30%, 40%, 50%, 60%, 70%, 80%, 90%, 100%, 200%, 300%, 400%, or 500% higher number of:
   (a) central memory T cells and/or percentage of central memory T cells than corresponding effector cell expressing a CAR comprising a CD28 or 4-1BB costimulatory domain; and/or
   (b) effector memory T cells and/or percentage of effector memory T cells than corresponding effector cell expressing a CAR comprising a CD28 or 4-1BB costimulatory domain.
   229. The method of any one of embodiments 225 to 228, wherein the central memory T cells express high levels of CCR7 and low levels of CD45RA.
   230. The method of any one of embodiments 225 to 229, wherein the central memory T cells are CD8⁺ T cells.

INFORMAL SEQUENCE LISTING

| SEQ | | |
|---|---|---|
| ID NO. | Sequence | Notes |
| 1 | METDTLLLWVLLLWVPGSTGLPVLTQPPSVSVAPGKTARITCGGNNIGSKSVHWYQQ | Anti-CD19-CD28z- |
| | KPGQAPVLVVYDDSDRPSGIPERFSGSNSGNTATLTISRVEAGDEADYYCQVWDSSSD | CAR |
| | YVVFGGGTKLTVLGSRGGGGSGGGGSGGGGSLEMAEVQLVQSGAEVKKPGESLKIS | |
| | CKGSGYSFTSYWIGWVRQMPGKGLEWMGIIYPGDSDTRYSPSFQGQVTISADKSISTA | |
| | YLQWSSLKASDTAMYYCARQVWGWQGGMYPRSNWWYNLDSWGQGTLVTVSSEQ | |
| | KLISEEDLAAAIEVMYPPPYLDNEKSNGTIIHVKGKHLCPSPLFPGPSKPFWVLVVVGG | |
| | VLACYSLLVTVAFIIFWVRSKRSRLLHSDYMNMTPRRPGPTRKHYQPYAPPRDFAAY | |
| | RSRVKFSRSADAPAYQQGQNQLYNELNLGRREEYDVLDKRRGRDPEMGGKPRRKNP | |
| | QEGLYNELQKDKMAEAYSEIGMKGERRRGKGHDGLYQGLSTATKDTYDALHMQAL | |
| | PPR | |
| 2 | METDTLLLWVLLLWVPGSTGLPVLTQPPSVSVAPGKTARITCGGNNIGSKSVHWYQQ | Anti-CD19-CD30z- |
| | KPGQAPVLVVYDDSDRPSGIPERFSGSNSGNTATLTISRVEAGDEADYYCQVWDSSSD | CAR |
| | YVVFGGGTKLTVLGSRGGGGSGGGGSGGGGSLEMAEVQLVQSGAEVKKPGESLKIS | |
| | CKGSGYSFTSYWIGWVRQMPGKGLEWMGIIYPGDSDTRYSPSFQGQVTISADKSISTA | |
| | YLQWSSLKASDTAMYYCARQVWGWQGGMYPRSNWWYNLDSWGQGTLVTVSSEQ | |
| | KLISEEDLAAATGAPPLGTQPDCNPTPENGEAPASTSPTQSLLVDSQASKTLPIPTSAPV | |
| | ALSSTGKPVLDAGPVLFWVILVLVVVVGSSAFLLCHRRACRKRIRQKLHLCYPVQTSQ | |
| | PKLELVDSRPRRSSTQLRSGASVTEPVAEERGLMSQPLMETCHSVGAAYLESLPLQDA | |
| | SPAGGPSSPRDLPEPRVSTEHTNNKIEKIYEVIKADTVIVGTVKAELPEGRGLAGPAEPE | |
| | LEEELEADHTPHYPEQETEPPLGSCSDVMLSVEEEGKEDPLPTAASGKRVKFSRSADA | |
| | PAYQQGQNQLYNELNLGRREEYDVLDKRRGRDPEMGGKPRRKNPQEGLYNELQKD | |
| | KMAEAYSEIGMKGERRRGKGHDGLYQGLSTATKDTYDALHMQALPPR | |
| 3 | METDTLLLWVLLLWVPGSTGLPVLTQPPSVSVAPGKTARITCGGNNIGSKSVHWYQQ | Anti-CD19-CD8T- |
| | KPGQAPVLVVYDDSDRPSGIPERFSGSNSGNTATLTISRVEAGDEADYYCQVWDSSSD | CD30z-CAR |
| | YVVFGGGTKLTVLGSRGGGGSGGGGSGGGGSLEMAEVQLVQSGAEVKKPGESLKIS | |
| | CKGSGYSFTSYWIGWVRQMPGKGLEWMGIIYPGDSDTRYSPSFQGQVTISADKSISTA | |
| | YLQWSSLKASDTAMYYCARQVWGWQGGMYPRSNWWYNLDSWGQGTLVTVSSEQ | |
| | KLISEEDLAAATGTTTPAPRPPTPAPTIASQPLSLRPEACRPAAGGAVHTRGLDFACDIY | |
| | IWAPLAGTCGVLLLSLVITLYCHRRACRKRIRQKLHLCYPVQTSQPKLELVDSRPRRSS | |
| | TQLRSGASVTEPVAEERGLMSQPLMETCHSVGAAYLESLPLQDASPAGGPSSPRDLPE | |
| | PRVSTEHTNNKIEKIYEVIKADTVIVGTVKAELPEGRGLAGPAEPELEEELEADHTPHYP | |
| | EQETEPPLGSCSDVMLSVEEEGKEDPLPTAASGKRVKFSRSADAPAYQQGQNQLYNE | |
| | LNLGRREEYDVLDKRRGRDPEMGGKPRRKNPQEGLYNELQKDKMAEAYSEIGMKG | |
| | ERRRGKGHDGLYQGLSTATKDTYDALHMQALPPR | |
| 4 | METDTLLLWVLLLWVPGSTGLPVLTQPPSVSVAPGKTARITCGGNNIGSKSVHWYQQ | Anti-CD19-CD8T- |
| | KPGQAPVLVVYDDSDRPSGIPERFSGSNSGNTATLTISRVEAGDEADYYCQVWDSSSD | 41BBz-CAR |
| | YVVFGGGTKLTVLGSRGGGGSGGGGSGGGGSLEMAEVQLVQSGAEVKKPGESLKIS | |
| | CKGSGYSFTSYWIGWVRQMPGKGLEWMGIIYPGDSDTRYSPSFQGQVTISADKSISTA | |
| | YLQWSSLKASDTAMYYCARQVWGWQGGMYPRSNWWYNLDSWGQGTLVTVSSEQ | |
| | KLISEEDLTGTTTPAPRPPTPAPTIASQPLSLRPEACRPAAGGAVHTRGLDFACDIYIWA | |
| | PLAGTCGVLLLSLVITLYCKRGRKKLLYIFKQPFMRPVQTTQEEDGCSCRFPEEEEGGC | |
| | ELRVKFSRSADAPAYQQGQNQLYNELNLGRREEYDVLDKRRGRDPEMGGKPRRKNP | |
| | QEGLYNELQKDKMAEAYSEIGMKGERRRGKGHDGLYQGLSTATKDTYDALHMQAL | |
| | PPR | |
| 5 | METDTLLLWVLLLWVPGSTGQSVLTQPASVSGSPGQSITISCTGTSSDVGGYNYVSW | Anti-AFP-CD28z- |
| | YQQHPGKAPKLMIYDVNNRPSEVSNRFSGSKSGNTASLTISGLQAEDEADYYCSSYTT | CAR |
| | GSRAVFGGGTKLTVLGSRGGGGSGGGGSGGGGSLEMAEVQLVQSGAEVKKPGESLTI | |
| | SCKASGYSFPNYWITWVRQMSGGGLEWMGRIDPGDSYTTYNPSFQGHVTISIDKSTN | |
| | TAYLHWNSLKASDTAMYYCARYYVSLVDIWGQGTLVTVSSEQKLISEEDLAAAIEV | |
| | MYPPPYLDNEKSNGTIIHVKGKHLCPSPLFPGPSKPFWVLVVVGGVLACYSLLVTVAF | |
| | IIFWVRSKRSRLLHSDYMNMTPRRPGPTRKHYQPYAPPRDFAAYRSRVKFSRSADAPA | |
| | YQQGQNQLYNELNLGRREEYDVLDKRRGRDPEMGGKPRRKNPQEGLYNELQKDKM | |
| | AEAYSEIGMKGERRRGKGHDGLYQGLSTATKDTYDALHMQALPPR | |
| 6 | METDTLLLWVLLLWVPGSTGQSVLTQPASVSGSPGQSITISCTGTSSDVGGYNYVSW | Anti-AFP-CD30z- |
| | YQQHPGKAPKLMIYDVNNRPSEVSNRFSGSKSGNTASLTISGLQAEDEADYYCSSYTT | CAR |
| | GSRAVFGGGTKLTVLGSRGGGGSGGGGSGGGGSLEMAEVQLVQSGAEVKKPGESLTI | |
| | SCKASGYSFPNYWITWVRQMSGGGLEWMGRIDPGDSYTTYNPSFQGHVTISIDKSTN | |
| | TAYLHWNSLKASDTAMYYCARYYVSLVDIWGQGTLVTVSSEQKLISEEDLAAATGA | |
| | PPLGTQPDCNPTPENGEAPASTSPTQSLLVDSQASKTLPIPTSAPVALSSTGKPVLDAGP | |
| | VLFWVILVLVVVVGSSAFLLCHRRACRKRIRQKLHLCYPVQTSQPKLELVDSRPRRSS | |
| | TQLRSGASVTEPVAEERGLMSQPLMETCHSVGAAYLESLPLQDASPAGGPSSPRDLPE | |
| | PRVSTEHTNNKIEKIYEVIKADTVIVGTVKAELPEGRGLAGPAEPELEEELEADHTPHYP | |
| | EQETEPPLGSCSDVMLSVEEEGKEDPLPTAASGKRVKFSRSADAPAYQQGQNQLYNE | |
| | LNLGRREEYDVLDKRRGRDPEMGGKPRRKNPQEGLYNELQKDKMAEAYSEIGMKG | |
| | ERRRGKGHDGLYQGLSTATKDTYDALHMQALPPR | |
| 7 | METDTLLLWVLLLWVPGSTGQSVLTQPASVSGSPGQSITISCTGTSSDVGGYNYVSW | Anti-AFP-CD8T- |
| | YQQHPGKAPKLMIYDVNNRPSEVSNRFSGSKSGNTASLTISGLQAEDEADYYCSSYTT | CD30z-CAR |
| | GSRAVFGGGTKLTVLGSRGGGGSGGGGSGGGGSLEMAEVQLVQSGAEVKKPGESLTI | |
| | SCKASGYSFPNYWITWVRQMSGGGLEWMGRIDPGDSYTTYNPSFQGHVTISIDKSTN | |
| | TAYLHWNSLKASDTAMYYCARYYVSLVDIWGQGTLVTVSSEQKLISEEDLAAATGT | |
| | TTPAPRPPTPAPTIASQPLSLRPEACRPAAGGAVHTRGLDFACDIYIWAPLAGTCGVLL | |
| | LSLVITLYCHRRACRKRIRQKLHLCYPVQTSQPKLELVDSRPRRSSTQLRSGASVTEPV | |
| | AEERGLMSQPLMETCHSVGAAYLESLPLQDASPAGGPSSPRDLPEPRVSTEHTNNKIE | |
| | KIYEVIKADTVIVGTVKAELPEGRGLAGPAEPELEEELEADHTPHYPEQETEPPLGSCSD | |
| | VMLSVEEEGKEDPLPTAASGKRVKFSRSADAPAYQQGQNQLYNELNLGRREEYDVL | |
| | DKRRGRDPEMGGKPRRKNPQEGLYNELQKDKMAEAYSEIGMKGERRRGKGHDGLY | |
| | QGLSTATKDTYDALHMQALPPR | |
| 8 | METDTLLLWVLLLWVPGSTGQSVLTQPASVSGSPGQSITISCTGTSSDVGGYNYVSW | Anti-AFP-CD8T- |
| | YQQHPGKAPKLMIYDVNNRPSEVSNRFSGSKSGNTASLTISGLQAEDEADYYCSSYTT | 41BBz-CAR |
| | GSRAVFGGGTKLTVLGSRGGGGSGGGGSGGGGSLEMAEVQLVQSGAEVKKPGESLTI | |
| | SCKASGYSFPNYWITWVRQMSGGGLEWMGRIDPGDSYTTYNPSFQGHVTISIDKSTN | |
| | TAYLHWNSLKASDTAMYYCARYYVSLVDIWGQGTLVTVSSEQKLISEEDLTGTTTPA | |
| | PRPPTPAPTIASQPLSLRPEACRPAAGGAVHTRGLDFACDIYIWAPLAGTCGVLLLSLVI | |
| | TLYCKRGRKKLLYIFKQPFMRPVQTTQEEDGCSCRFPEEEEGGCELRVKFSRSADAPA | |
| | YQQGQNQLYNELNLGRREEYDVLDKRRGRDPEMGGKPRRKNPQEGLYNELQKDKM | |
| | AEAYSEIGMKGERRRGKGHDGLYQGLSTATKDTYDALHMQALPPR | |
| 9 | METDTLLLWVLLLWVPGSTGLPVLTQPPSVSVAPGKTARITCGGNNIGSKSVHWYQQ | Anti-CD19-CD8T- |
| | KPGQAPVLVVYDDSDRPSGIPERFSGSNSGNTATLTISRVEAGDEADYYCQVWDSSSD | CD28z-CAR |
| | YVVFGGGTKLTVLGSRGGGGSGGGGSGGGGSLEMAEVQLVQSGAEVKKPGESLKIS | |
| | CKGSGYSFTSYWIGWVRQMPGKGLEWMGIIYPGDSDTRYSPSFQGQVTISADKSISTA | |
| | YLQWSSLKASDTAMYYCARQVWGWQGGMYPRSNWWYNLDSWGQGTLVTVSSEQ | |
| | KLISEEDLTGTTTPAPRPPTPAPTIASQPLSLRPEACRPAAGGAVHTRGLDFACDIYIWA | |
| | PLAGTCGVLLLSLVITLYCRSKRSRLLHSDYMNMTPRRPGPTRKHYQPYAPPRDFAAY | |
| | RSRVKFSRSADAPAYQQGQNQLYNELNLGRREEYDVLDKRRGRDPEMGGKPRRKNP | |
| | QEGLYNELQKDKMAEAYSEIGMKGERRRGKGHDGLYQGLSTATKDTYDALHMQAL | |
| | PPR | |
| 10 | METDTLLLWVLLLWVPGSTGQSVLTQPASVSGSPGQSITISCTGTSSDVGGYNYVSW | Anti-AFP-CD8T- |
| | YQQHPGKAPKLMIYDVNNRPSEVSNRFSGSKSGNTASLTISGLQAEDEADYYCSSYTT | CD28z-CAR |
| | GSRAVFGGGTKLTVLGSRGGGGSGGGGSGGGGSLEMAEVQLVQSGAEVKKPGESLTI | |
| | SCKASGYSFPNYWITWVRQMSGGGLEWMGRIDPGDSYTTYNPSFQGHVTISIDKSTN | |
| | TAYLHWNSLKASDTAMYYCARYYVSLVDIWGQGTLVTVSSEQKLISEEDLAAATGT | |
| | TTPAPRPPTPAPTIASQPLSLRPEACRPAAGGAVHTRGLDFACDIYIWAPLAGTCGVLL | |
| | LSLVITLYCAAARSKRSRLLHSDYMNMTPRRPGPTRKHYQPYAPPRDFAAYRSRVKF | |
| | SRSADAPAYQQGQNQLYNELNLGRREEYDVLDKRRGRDPEMGGKPRRKNPQEGLY | |
| | NELQKDKMAEAYSEIGMKGERRRGKGHDGLYQGLSTATKDTYDALHMQALPPR | |
| | MRVLLAALGLLFLGALRAFPQDRPFEDTCHGNPSHYYDKAVRRCCYRCPMGLFPTQ | |
| | QCPQRPTDCRKQCEPDYYLDEADRCTACVTCSRDDLVEKTPCAWNSSRVCECRPGM | |
| 11 | FCSTSAVNSCARCFFHSVCPAGMIVKFPGTAQKNTVCEPASPGVSPACASPENCKEPSS | Full length CD30 |
| | GTIPQAKPTPVSPATSSASTMPVRGGTRLAQEAASKLTRAPDSPSSVGRPSSDPGLSPT | (NP_001234.3) |
| | QPCPEGSGDCRKQCEPDYYLDEAGRCTACVSCSRDDLVEKTPCAWNSSRTCECRPGM | |
| | ICATSATNSCARCVPYPICAAETVTKPQDMAEKDTTFEAPPLGTQPDCNPTPENGEAP | |
| | ASTSPTQSLLVDSQASKTLPIPTSAPVALSSTGKPVLDAGPVLFWVILVLVVVVGSSAF | |
| | LLCHRRACRKRIRQKLHLCYPVQTSQPKLELVDSRPRRSSTQLRSGASVTEPVAEERG | |
| | LMSQPLMETCHSVGAAYLESLPLQDASPAGGPSSPRDLPEPRVSTEHTNNKIEKIYIMK | |
| | ADTVIVGTVKAELPEGRGLAGPAEPELEEELEADHTPHYPEQETEPPLGSCSDVMLSV | |
| | EEEGKEDPLPTAASGK | |
| 12 | QPVLTQPPSVSVAPGKTARITCGGNNIGSKSVHWYQQKPGQAPVLVIYYDSDRPSGIP | GPC3-55 scFv |
| | ERFSGSNSGNTATLTISRVEAGDEADYYCQVWDSSSDHYVFGTGTKVTVLGSRGGGG | |
| | SGGGGSGGGGSLEMAQVQLVQSGAEVKKPGASVKVSCKASGYTFTSYYMHWVRQA | |
| | PGQGLEWMGIINPSGGSTSYAQKFQGRVTMTRDTSTSTVYMELSSLRSEDTAVYYCA | |
| | RWHGGPYDYWGQGTLVTVSS | |
| 13 | QSVLTQPPSVSVAPGKTARITCGGNNIGSKSVHWYQQKPGQAPVLVVYDDSDRPSGIP | GPC3-58 scFv |
| | ERFSGSNSGNTATLTISRVEAGDEADYYCQVWDSSSDHVFGTGTKVTVLGSRGGGGS | |
| | GGGGSGGGGSLEMAQVQLVQSGADVRKPGASVKVSCKASGYTFASHGISWVRQAPG | |
| | QGLEWLGWISPYTGNTNYAQKFQGRVTMATDTSTSTAYMELRSLRSDDTAIYYCAR | |
| | GKRTLASCFDYWGQGTLVTVSS | |
| 14 | LPVLTQPPSVSVAPGKTARITCGGNNIGSKSVHWYQQKPGQAPVLVVYDDSDRPSGIP | CD19 scFv |
| | ERFSGSNSGNTATLTISRVEAGDEADYYCQVWDSSSDYVVFGGGTKLTVLGSRGGGG | |
| | SGGGGSGGGGSLEMAEVQLVQSGAEVKKPGESLKISCKGSGYSFTSYWIGWVRQMP | |
| | GKGLEWMGIIYPGDSDTRYSPSFQGQVTISADKSISTAYLQWSSLKASDTAMYYCAR | |
| | QVWGWQGGMYPRSNWWYNLDSWGQGTLVTVSS | |
| 15 | QIVLSQSPAILSASPGEKVTMTCRASSSVSYHEWFQQKPGSSPKPWIYATSNLASGVPV | CD20 scFv |
| | RFSGSGSGTSYSLTISRVEAEDAATYYCQQWTSNPPTEGGGTKLEIKRSRGGGGSGGG | |
| | GSGGGGSLEQVQLQQPGAELVKPGASVKMSCKASGYTFTSYNMHWVKQTPGRGLE | |
| | WIGAIYPGNGDTSYNQKFKGKATLTADKSSSTAYMQLSSLTSEDSAVYYCARSTYYG | |
| | GDWYFNVWGAGTTVTVSS | |
| 16 | DIQLTQSPSSLSTSVGDRVTITCQASHDIRNYLNWYQQKPGKAPNLLIYAASNLQTGV | CD22 scFv |
| | PSRFSGRGSGTDFTLTISSLQPEDIATYYCQQYDGLPLTEGQGTRLEEKRSRGGGGSGG | |
| | GGSGGGGSLEMAQVQLVESGGGLVQPGGSLRLSCAASGFTFSNYAMSWVRQAPGKG | |
| | LEWVSSISGSGGSTYYADSVKGRETISRDTSKNTLYLQMNSLRAEDTAVYYCARYGS | |
| | AAWMDSWGQGTLVTVSS | |
| 17 | EIVLTQSPGTLSLSPGERATLSCRASQSVSSSYLAWYQQKPGQAPRLLIYGASSRATGIP | ROR1-18 scFv |
| | DRFSGSGSGTDFTLTISRLEPEDFAVYYCQQYGSSFGPGTKVDIKRSRGGGGSGGGGS | |
| | GGGGSLEMAQVQLVQSGTEVKKPGSSVKVSCQASGGSLSSHGVSWLRQAPGQGLEW | |
| | VGRIIPMFGVTDYAQKFQDRVTITADKSTSTVYMELISLGSDDTAVYFCARESRGATF | |
| | EYWGQGTLVTVSS | |
| 18 | QSVLTQPASVSGSPGQSITISCTGTSSDEGDYDYVSWYQQHPGKAPKLMIYDVSDRPS | ROR1-56 scFv |
| | GVSNRFSGSKSGNTASLTISGLQAEDEADYFCSSLTTSSTLVEGGGTKLTVLGSRGGG | |
| | GSGGGGSGGGGSLEMAQLQLQESGPGLVKPSETLSLTCTVSGGSISSSSYYWGWIRQP | |
| | PGKGLEWIGSIYYSGSTYYNPSLKSRVTISVDTSKNQFSLKLGSVTAADTAVYYCARH | |
| | DGTDAFDIWGQGTTVTVSS | |
| 19 | QSVLTQPPSVSGAPGQRVTISCTGSSSNIGAGYDVHWYQQLPGTAPKLLIYGNSNRPS | PSMA-A scFv |
| | GVPDRFSGSKSGTSASLAITGLQAEDEADYYCQSYDSSLSGYVEGTGTKVTVLGSRGG | |
| | GGSGGGGSGGGGSLEMAEVQLVQSGAEVKKPGESLKISCKGSGYSFTSYWIGWVRQ | |
| | MPGKGLEWMGHYPGDSDTRYSPSFQGQVTISADKSISTAYLQWSSLKASDTAMYYC | |
| | ARSMGSSLYASSDVWGQGTLVTVSS | |
| 20 | QAVLTQPPSASGTPGQRVTISCSGSSSNIGSNTVNWYQQLPGTAPKLLMYSNNQRPSG | PSMA-B scFv |
| | VPDRFSGSKSGTSASLAISGLQSEDEADYYCAAWDDSLNGYVEGTGTKVTVLGSRGG | |
| | GGSGGGGSGGGGSLEMAEVQLVQSGAEMKKPGESLKISCKGSGYNFASYWVGWVR | |
| | QMPGKGLEWMGTIYPDDSDTRYGPAFQGQVTISADKSISTAYLQWSSLKASDTAMY | |
| | YCARDSYYGEDVWGQGTLVTVSS | |
| 21 | QSVVTQPPSVSAAPGQKVTISCSGSSSNIGNNYVSWYQQLPGTAPKLLIYDNNKRPSGI | NYESO1-35 scFv |
| | PDRESASKSGTSATLGITGLQTRDEADYYCGTWDSSLSAWVFGGGTKLTVLGSRGGG | |
| | GSGGGGSGGGGSLEMAQVQLVQSGAEVKKPGSSVKVSCKASGDTESSYYISWVRQA | |
| | PGQGLEWMGRIIPILGIANYAQKYQGRVTLSADKSTSTSYMELNSLSSEDTAVYYCAL | |
| | DWSYSIDYWGQGTLVTVSS | |
| 22 | QSVVTQPPSLSGAPGQRVTISCNGSGSNIGAGYDVHWYQQLPGTAPKLLIYGNSNRPS | NYESO1-76 scFv |
| | GVPDRFSGSKSGTSASLAITGLQAEDEADYYCQSYDSSLSGWGIFGGGTKLTVLGSRG | |
| | GGGSGGGGSGGGGSLEMAEVQLVQSGAEVKKPGSSVKVSCKASGGTESSYAISWVR | |
| | QAPGQGLEWMGGIIPIEGTANYAQKFQGRVTITADESTSTAYMELSSLRSEDTAVYYC | |
| | ARYDSYVYDEWGQGTLVTVSS | |
| 23 | DILLTQSPVILSVSPGERVSFSCRASQSIGTNIHWYQQRTNGSPRLLIKYASESISGIPSRF | EGFR scFv |
| | SGSGSGTDFTLSINSVESEDIADYYCQQNNNVVPTTFGAGTKLELKRSRGGGGSGGGGS | |
| | GGGGSLEMAQVQLKQSGPGLVQPSQSLSITCTVSGESLTNYGVHWVRQSPGKGLEWL | |
| | GVIWSGGNTDYNTPFTSRLSINKDNSKSQVFFKMNSLQSNDTAIYYCARALTYYDYEF | |
| | AYWGQGTLVTVSS | |
| 24 | DIQLTQSPSSLSTSVGDRVTITCQASHDIRNYLNWYQQKPGKAPNLLIYAASNLQTGV | αCD22-αCD19 |
| | PSRFSGRGSGTDFTLTISSLQPEDIATYYCQQYDGLPLTEGQGTRLEIKRSRGGGGSGG | anti-CD22-clone 8 |
| | GGSGGGGSLEMAQVQLVESGGGLVQPGGSLRLSCAASGFTFSNYAMSWVRQAPGKG | scFv and anti-CD19 |
| | LEWVSSISGSGGSTYYADSVKGRFTISRDTSKNTLYLQMNSLRAEDTAVYYCARYGS | scFv-linker-anti- |
| | AAWMDSWGQGTLVTVSSGGGGSGGGGSLPVLTQPPSVSVAPGKTARITCGGNNIGSK | CD19 scFv |
| | SVHWYQQKPGQAPVLVVYDDSDRPSGIPERFSGSNSGNTATLTISRVEAGDEADYYC | |
| | QVWDSSSDYVVFGGGTKLTVLGSRGGGGSGGGGSGGGGSLEMAEVQLVQSGAEVK | |
| | KPGESLKISCKGSGYSFTSYWIGWVRQMPGKGLEWMGIIYPGDSDTRYSPSFQGQVTI | |
| | SADKSISTAYLQWSSLKASDTAMYYCARQVWGWQGGMYPRSNWWYNLDSWGQGT | |
| | LVTVSS | |
| 25 | LPVLTQPPSVSVAPGKTARITCGGNNIGSKSVHWYQQKPGQAPVLVVYDDSDRPSGIP | αCD19-αCD22-αCD20 |
| | ERFSGSNSGNTATLTISRVEAGDEADYYCQVWDSSSDYVVFGGGTKLTVLGSRGGGG | CD19 scFv-linker- |
| | SGGGGSGGGGSLEMAEVQLVQSGAEVKKPGESLKISCKGSGYSFTSYWIGWVRQMP | CD22 scFv-linker- |
| | GKGLEWMGIIYPGDSDTRYSPSFQGQVTISADKSISTAYLQWSSLKASDTAMYYCAR | CD20 scFv |
| | QVWGWQGGMYPRSNWWYNLDSWGQGTLVTVSSGGGGSGGGGSDIQLTQSPSSLST | |
| | SVGDRVTITCQASHDIRNYLNWYQQKPGKAPNLLIYAASNLQTGVPSRFSGRGSGTDF | |
| | TLTISSLQPEDIATYYCQQYDGLPLTFGQGTRLEEKRSRGGGGSGGGGSGGGGSLEMA | |
| | QVQLVESGGGLVQPGGSLRLSCAASGFTFSNYAMSWVRQAPGKGLEWVSSISGSGGS | |
| | TYYADSVKGRFTISRDTSKNTLYLQMNSLRAEDTAVYYCARYGSAAWMDSWGQGT | |
| | LVTVSSGGGGSGGGGSQIVLSQSPAILSASPGEKVTMTCRASSSVSYHEWFQQKPGSSP | |
| | KPWIYATSNLASGVPVRFSGSGSGTSYSLTISRVEAEDAATYYCQQWTSNPPTFGGGT | |
| | KLEIKRSRGGGGSGGGGSGGGGSLEQVQLQQPGAELVKPGASVKMSCKASGYTFTSY | |
| | NMHWVKQTPGRGLEWIGAIYPGNGDTSYNQKFKGKATLTADKSSSTAYMQLSSLTS | |
| | EDSAVYYCARSTYYGGDWYFNVWGAGTTVTVSS | |
| 26 | IYIWAPLAGTCGVLLLSLVIT | CD8 transmembrane |
| | | (TM) sequence |
| 27 | IISFFLALTSTALLFLLFFLTLRFSVV | 4-1BB TM sequence |
| 28 | ILVIFSGMFLVFTLAGALFLH | CD27 TM sequence |
| 29 | FWVLVVVGGVLACYSLLVTVAFIIFWV | CD28 TM sequence |
| 30 | PVLDAGPVLFWVILVLVVVVGSSAFLLC | CD30 TM sequence |
| 31 | VAAILGLGLVLGLLGPLAILL | OX40 TM sequence |
| 32 | KRGRKKLLYIFKQPFMRPVQTTQEEDGCSCRFPEEEEGGCEL | 4-1BB IC signaling |
| | | sequence |
| 33 | QRRKYRSNKGESPVEPAEPCRYSCPREEEGSTIPIQEDYRKPEPACSP | CD27 IC signaling |
| | | sequence |
| 34 | RSKRSRLLHSDYMNMTPRRPGPTRKHYQPYAPPRDFAAYRS | CD28 IC signaling |
| | | sequence |
| 35 | HRRACRKRIRQKLHLCYPVQTSQPKLELVDSRPRRSSTQLRSGASVTEPVAEERGLMS | CD30 IC signaling |
| | QPLMETCHSVGAAYLESLPLQDASPAGGPSSPRDLPEPRVSTEHTNNKIEKIYEVIKADT | sequence |
| | VIVGTVKAELPEGRGLAGPAEPELEEELEADHTPHYPEQETEPPLGSCSDVMLSVEEE | |
| | GKEDPLPTAASGK | |
| 36 | ALYLLRRDQRLPPDAHKPPGGGSFRTPIQEEQADAHSTLAKI | OX40 IC signaling |
| | | sequence |
| 37 | RVKFSRSADAPAYQQGQNQLYNELNLGRREEYDVLDKRRGRDPEMGGKPRRKNPQE | CD3 IC signaling |
| | GLYNELQKDKMAEAYSEIGMKGERRRGKGHDGLYQGLSTATKDTYDALHMQALPP | sequence |
| | R | |
| 38 | SRGGGGSGGGGSGGGGSLEMA | Peptide linker |
| 39 | GGGGS | Peptide linker |
| 40 | GGSG | Peptide linker |
| 41 | SGGG | Peptide linker |
| 42 | GSGS | Peptide linker |
| 43 | GSGSGS | Peptide linker |
| 44 | GSGSGSGS | Peptide linker |
| 45 | GSGSGSGSGS | Peptide linker |
| 46 | GGSGGS | Peptide linker |
| 47 | GGSGGSGGS | Peptide linker |
| 48 | GGSGGSGGSGGS | Peptide linker |
| 49 | GGSG | Peptide linker |
| 50 | GGSGGGSG | Peptide linker |
| 51 | GGSGGGSGGGSG | Peptide linker |
| 52 | SRGGGGSGGGGSGGGGSLEMA | Peptide linker |
| 53 | HHHHHH | 6xHis Tag |
| 54 | YPYDVPDYA | HA peptide |
| 55 | YPYDVPDYAS | HA peptide |
| 56 | DYKDDDDK | FLAG peptide |
| 57 | EQKLISEEDL | Myc peptide |
| 58 | EVQLVESGGGLVQPGGSLRLSCAASGFTFSNYAMSWVRQAPGKGLEWVSAISGSGGS | Anti-CD22 V_{H} region |
| | TYYADSVKGRFTISRDNSKNTLYLQMNSLRAEDTAVYYCARPYYDDWGQGTLVTVS | |
| | S | |
| 59 | QSVVTQPPSVSAAPGQKVTISCSGSSSNIGNNYVSWYQQLPGTAPKLLIYENNKRPSGI | Anti-CD22 V_{L} region |
| | PDRFSGSKSGTSATLGITGLQTGDEADYYCGTWDSSLSAGAVFGGGTKLTVLG | |
| 60 | QVQLVESGGGLVQPGGSLRLSCAASGFTFSNYAMSWVRQAPGKGLEWVSSISGSGGS | Anti-CD22 V_{H} region |
| | TYYADSVKGRFTISRDTSKNTLYLQMNSLRAEDTAVYYCARYGSAAWMDSWGQGT | |
| | LVTVSS | |
| 61 | DIQLTQSPSSLSTSVGDRVTITCQASHDIRNYLNWYQQKPGKAPNLLIYAASNLQTGV | Anti-CD22 V_{L} region |
| | PSRFSGRGSGTDFTLTISSLQPEDIATYYCQQYDGLPLTFGQGTRLEIKR | |
| 62 | EVQLVQSGAEVKKPGESLKISCKGSGYSFTSYWIGWVRQMPGKGLEWMGHYPGDSD | Anti-CD19 V_{H} region |
| | TRYSPSFQGQVTISADKSISTAYLQWSSLKASDTAMYYCARQVWGWQGGMYPRSNW | |
| | WYNLDSWGQGTLVTVSS | |
| 63 | LPVLTQPPSVSVAPGKTARITCGGNNIGSKSVHWYQQKPGQAPVLVVYDDSDRPSGIP | Anti-CD19 V_{L} region |
| | ERFSGSNSGNTATLTISRVEAGDEADYYCQVWDSSSDYVVFGGGTKLTVLG | |
| 64 | QVQLQQPGAELVKPGASVKMSCKASGYTFTSYNMHWVKQTPGRGLEWIGAIYPGNG | Anti-CD20 V_{H} region |
| | DTSYNQKFKGKATLTADKSSSTAYMQLSSLTSEDSAVYYCARSTYYGGDWYFNVWG | |
| | AGTTVTVSS | |
| 65 | QIVLSQSPAILSASPGEKVTMTCRASSSVSYIHWFQQKPGSSPKPWIYATSNLASGVPV | Anti-CD20 V_{L} region |
| | RFSGSGSGTSYSLTISRVEAEDAATYYCQQWTSNPPTFGGGTKLEIKR | |
| 66 | QVQLQESGPGLVKPSQTLSLTCTVSGYTFTNYYVFWVRQARGQRLEWIGDINPVNGD | Anti-CD47 V_{H} region |
| | TNFNEKFKNRVTISADKSISTAYLQWSSLKASDTAMYYCARGGYTMDYWGQGTLVT | |
| | VS | |
| 67 | DIVMTQTPLSLPVTPGEPASISCRSSQSLVHSNGNTYLHWYQQKPGKAPKLLIYKVSY | Anti-CD47 V_{L} region |
| | RFSGVPDRFSGSGSGTDFTLKISRVEAEDVGVYYCSQNTHVPRTFGQGTKVEEKR | |
| 68 | QVQLVQSGAEVKKPGASVKVSCKASGYTFTSYYMHWVRQAPGQGLEWMGIINPSGG | Anti-GPC3 V_{H} region |
| | STSYAQKFQGRVTMTRDTSTSTVYMELSSLRSEDTAVYYCARWHGGPYDYWGQGTL | |
| | VTVSS | |
| 69 | QPVLTQPPSVSVAPGKTARITCGGNNIGSKSVHWYQQKPGQAPVLVIYYDSDRPSGIP | Anti-GPC3 V_{L} region |
| | ERFSGSNSGNTATLTISRVEAGDEADYYCQVWDSSSDHYVFGTGTKVTVLG | |
| 70 | QVQLVQSGADVRKPGASVKVSCKASGYTFASHGISWVRQAPGQGLEWLGWISPYTG | Anti-GPC3 V_{H} region |
| | NTNYAQKFQGRVTMATDTSTSTAYMELRSLRSDDTAIYYCARGKRTLASCFDYWGQ | |
| | GTLVTVSS | |
| 71 | QSVLTQPPSVSVAPGKTARITCGGNNIGSKSVHWYQQKPGQAPVLVVYDDSDRPSGIP | Anti-GPC3 V_{L} region |
| | ERFSGSNSGNTATLTISRVEAGDEADYYCQVWDSSSDHVFGTGTKVTVLG | |
| 72 | PLFQVPEPV | AFP peptide can be |
| | | targeted by CAR; |
| | | hAFP137-145 |
| 73 | FMNKFIYEI | AFP peptide can be |
| | | targeted by CAR; |
| | | hAFP 158-166 |
| 74 | GLSPNLNRFL | AFP peptide can be |
| | | targeted by CAR; |
| | | hAFP325-334 |
| 75 | GVALQTMKQ | AFP peptide can be |
| | | targeted by CAR; |
| | | hAFP542-550 |
| 76 | AMNKFIYEI | AFP peptide can be |
| | | targeted by CAR; |
| | | hAFP158 A1 |
| 77 | FMAKFIYEI | AFP peptide can be |
| | | targeted by CAR; |
| | | hAFP158 A3 |
| 78 | FMNAFIYEI | AFP peptide can be |
| | | targeted by CAR; |
| | | hAFP158 A4 |
| 79 | FMNKAIYEI | AFP peptide can be |
| | | targeted by CAR; |
| | | hAFP158 A5 |
| 80 | FMNKFAYEI | AFP peptide can be |
| | | targeted by CAR; |
| | | hAFP158 A6 |
| 81 | FMNKFIAEI | hAFP158 A7 |
| 82 | FMNKFIYAI | AFP peptide can be |
| | | targeted by CAR; |
| | | hAFP158 A8 |
| 83 | GYTFTSYG | Anti-AFP_HCDR1 |
| 84 | ISAYNGNT | Anti-AFP_HCDR2 |
| 85 | ARDSYYYYYGMDV | Anti-AFP_HCDR3 |
| 86 | EVQLVQSGAEVKKPGASVKVSCKASGYTFTSYGISWVRQAPGQGLEWMGWISAYNG | anti-AFP VH |
| | NTNYAQKLQGRVTMTTDTSTSTAYMELRSLRSDDTAVYYCARDSYYYYYGMDVWG | |
| | QGTTVTVSS | |
| 87 | TGAVTSGHY | Anti-AFP_LCDR1 |
| 88 | DAS | Anti-AFP_LCDR2 |
| 89 | LLSYSDALV | Anti-AFP_LCDR3 |
| 90 | QAVVTQEPSLTVSPGGTVTLTCGSSTGAVTSGHYPYWFQQKPGQAPRTLIYDASDKH | anti-AFP VL |
| | SWTPARFSGSLLGGKAALTLSGAQPEDEAEYYCLLSYSDALVFGGGTKLTVLG | |
| 91 | GYSFPNYW | Anti-AFP_HCDR1 |
| 92 | IDPGDSYT | Anti-AFP_HCDR2 |
| 93 | ARYYVSLVDI | Anti-AFP_HCDR3 |
| 94 | EVQLVQSGAEVKKPGESLTISCKASGYSFPNYWITWVRQMSGGGLEWMGRIDPGDSY | anti-AFP VH |
| | TTYNPSFQGHVTISIDKSTNTAYLHWNSLKASDTAMYYCARYYVSLVDIWGQGTLVT | |
| | VSS | |
| 95 | SSDVGGYNY | Anti-AFP_LCDR1 |
| 96 | DVN | Anti-AFP_LCDR2 |
| 97 | SSYTTGSRAV | Anti-AFP_LCDR3 |
| 98 | QSVLTQPASVSGSPGQSITISCTGTSSDVGGYNYVSWYQQHPGKAPKLMIYDVNNRPS | anti-AFP VL |
| | EVSNRFSGSKSGNTASLTISGLQAEDEADYYCSSYTTGSRAVFGGGTKLTVLG | |
| 99 | GFTFSNAW | Anti-AFP_HCDR1 |
| 100 | IRSKAYGGTT | Anti-AFP_HCDR2 |
| 101 | ARDGLYSSSWYDSDY | Anti-AFP_HCDR3 |
| 102 | EVQLVESGGGLVKPGGSLRLSCAASGFTFSNAWMSWVRQAPGKGLEWVGFIRSKAY | |
| | GGTTEYAASVKGRFTISRDDSKSIAYLQMNNLKTEDTAVYYCARDGLYSSSWYDSDY | anti-AFP VH |
| | WGQGTLVTVSS | |
| 103 | SSNIGNNY | Anti-AFP_LCDR1 |
| 104 | DNN | Anti-AFP_LCDR2 |
| 105 | GTWDGSLYTML | Anti-AFP_LCDR3 |
| 106 | QSVVTQPPSVSAAPGQKVTISCSGSSSNIGNNYVSWYQQLPGTAPKLLIYDNNKRPSGI | anti-AFP VL |
| | PDRFSGSKSGTSATLGITGLQTGDEADYYCGTWDGSLYTMLFGGGTKLTVLG | |
| 107 | GFTFDDYA | Anti-AFP_HCDR1 |
| 108 | ISWNSGSI | Anti-AFP_HCDR2 |
| 109 | AKDIHSGSYYGLLYYAMDV | Anti-AFP_HCDR3 |
| 110 | QMQLVQSGGGLVQPGRSLRLSCAASGFTFDDYAMHWVRQAPGKGLEWVSGISWNS | anti-AFP VH |
| | GSIGYADSVKGRFTISRDNAKNSLYLQMNSLRAEDTALYYCAKDIHSGSYYGLLYYA | |
| | MDVWGQGTTVTVSS | |
| 111 | SSNIGAGYD | Anti-AFP_LCDR1 |
| 112 | GNS | Anti-AFP_LCDR2 |
| 113 | QSYDSSLSGSGV | Anti-AFP_LCDR3 |
| 114 | QSVLTQPPSVSGAPGQRVTISCTGSSSNIGAGYDVHWYQQLPGTAPKLLIFGNSNRPSG | anti-AFP VL |
| | VPDRFSGFKSGTSASLAITGLQAEDEADYFCQSYDSSLSGSGVFGTGTKVTVLG | |
| 115 | GYTFTSYG | Anti-AFP_HCDR1 |
| 116 | ISAYNGNT | Anti-AFP_HCDR2 |
| 117 | ARFQDWWYLGQFDQ | Anti-AFP_HCDR3 |
| 118 | EVQLVQSGAEVKKPGASVKVSCKASGYTFTSYGISWVRQAPGQGLEWMGWISAYNG | anti-AFP VH |
| | NTNYAQKLQGRVTMTTDTSTSTAYMELRSLRSDDTAVYYCARFQDWWYLGQFDQW | |
| | GQGTLVTVSS | |
| 119 | GSDVGVYYY | Anti-AFP_LCDR1 |
| 120 | DVD | Anti-AFP_LCDR2 |
| 121 | ASYTNRNSLGYV | Anti-AFP_LCDR3 |
| 122 | QSALTQPASVSGSPGQSITISCTATGSDVGVYYYVSWYQQHPGKAPKVMIYDVDNRP | anti-VL AFP |
| | PGVSNRFSGSKSGNTASLTISGLQAEDEADYYCASYTNRNSLGYVFGTGTKVTVLG | |
| 123 | GGSFSGYY | Anti-GPC3_HCDR1 |
| 124 | INHSGST | Anti-GPC3_HCDR2 |
| 125 | ARGYGGRFDY | Anti-GPC3_HCDR3 |
| 126 | QVQLQQWGAGLLKPSETLSLTCAVYGGSFSGYYWSWIRQPPGKGLEWIGEINHSGST | anti-GPC3 V_{H}_Clone |
| | NYNPSLKSRVTISVDTSKNQFSLELSSVTAADTAVYYCARGYGGRFDYWGQGTLVTV | 34 |
| | SS | |
| 127 | SSNIGSNN | Anti-GPC3_LCDR1 |
| 128 | SNH | Anti-GPC3_LCDR2 |
| 129 | AAWDDSLDGYL | Anti-GPC3_LCDR3 |
| 130 | QPVLTQPPSASGTPGQRVTISCSGSSSNIGSNNVIWYQQLPGAAPKLLIYSNHRRPSGVP | anti-GPC3 V_{L}_clone |
| | DRFSGSRSGTSASLAISGLQSEDEADYYCAAWDDSLDGYLFGTGTKVTVLG | 34 |
| 131 | GFTFSSYA | Anti-GPC3_HCDR1 |
| 132 | IYSGGSST | Anti-GPC3_HCDR2 |
| 133 | ARTSYLNHGDY | Anti-GPC3_HCDR3 |
| 134 | QVQLVESGGGLVQPGGSLRLSCAASGFTFSSYAMSWVRQAPGKGLEWVSVIYSGGSS | anti-GPC3 V_{H}_clone |
| | TYYADSVKGRFTISRDNSKNTLYLQMNSLRAEDTAVYYCARTSYLNHGDYWGQGTL | 37 |
| | VTVSS | |
| 135 | RSNIGSDY | Anti-GPC3_LCDR1 |
| 136 | GDN | Anti-GPC3_LCDR2 |
| 137 | GTWDYTLNGVV | Anti-GPC3_LCDR3 |
| 138 | QSVLTQPPSVSAAPGQRVTISCSGTRSNIGSDYVSWYQHLPGTAPKLLVYGDNLRPSGI | anti-GPC3 V_{L}_clone |
| | PDRFSASKSGTSATLGITGLQTGDEADYYCGTWDYTLNGVVFGGGTKLTVLG | 37 |
| 139 | GFTFSDYY | Anti-GPC3_HCDR1 |
| 140 | ISSSGSTI | Anti-GPC3_HCDR2 |
| 141 | ARASDLYGD | Anti-GPC3_HCDR3 |
| 142 | QMQLVQSGGGLVKPGGSLRLSCAASGFTFSDYYMSWIRQAPGKGLEWVSYISSSGSTI | anti-GPC3 V_{H}_clone |
| | YYADSVKGRFTISRDNAKNSLYLQMNSLRAEDTAVYYCARASDLYGDWGQGTLVT | 45 |
| | VSS | |
| 143 | TSNIGTNT | Anti-GPC3_LCDR1 |
| 144 | SNN | Anti-GPC3_LCDR2 |
| 145 | AAWDDSLNGVV | Anti-GPC3_LCDR3 |
| 146 | QSVLTQPPSVSGTPGQRVIISCPGSTSNIGTNTVNWYQQFPGTAPKLLIYSNNQRPSGVP | anti-GPC3 V_{L}_clone |
| | DRFSGSKSGTSASLAISGLQSEDEADYYCAAWDDSLNGVVFGGGTKLTVLG | 45 |
| 147 | GYRFSNYG | Anti-GPC3_HCDR1 |
| 148 | ISGSNGNT | Anti-GPC3_HCDR2 |
| 149 | ARGNRRYYSPIIDP | Anti-GPC3_HCDR3 |
| 150 | QVQLVQSGAEVKKPGASVTVSCKASGYRFSNYGVSWVRQAPGQGLEWMGWISGSN | anti-GPC3 V_{H}_clone |
| | GNTNYAQKFLGRVTMTTDTSTTTAYMELSSLRSDDTAVYYCARGNRRYYSPIIDPWG | 46 |
| | QGTLVTVSS | |
| 151 | SSNFGSNT | Anti-GPC3_LCDR1 |
| 152 | SNT | Anti-GPC3_LCDR2 |
| 153 | AAWDDSLTGVV | Anti-GPC3_LCDR3 |
| 154 | QAVLTQPPSVSGTPGQRVTISCSGSSSNFGSNTVHWYQQVPGTAPKLLIFSNTQRPSEIP | anti-GPC3 V_{L}_clone |
| | DRFSGSKSGTSASLAISGLQSEDEADYYCAAWDDSLTGVVFGGGTKLTVLG | 46 |
| 155 | GGTFGSYA | Anti-GPC3_HCDR1 |
| 156 | IIPVLGRT | Anti-GPC3_HCDR2 |
| 157 | ARTNDS | Anti-GPC3_HCDR3 |
| 158 | EVQLVQSGAEVRKPGSSVKVSCQASGGTFGSYAISWVRQAPGQGLEWMGRIIPVLGR | anti-GPC3 V_{H}_clone |
| | TKYAQKFQGRVTVTADTSTSTVYMELTSLTSEDTAVYYCARTNDSWGQGTLVTVSS | 87 |
| 159 | QSLLHSNGYNY | Anti-GPC3_LCDR1 |
| 160 | LGS | Anti-GPC3_LCDR2 |
| 161 | MQALQTPWT | Anti-GPC3_LCDR3 |
| 162 | DVVMTQSPLSLPVTPGEPASVSCRSSQSLLHSNGYNYLDWYLQKPGQSPQLLIYLGSN | anti-GPC3 V_{L}_clone |
| | RASGVPDRFSGSGSGTDFTLKISRVEAEDVGVYYCMQALQTPWTFGQGTKVEIKR | 87 |
| 163 | GYTFTSYY | Anti- |
| | | GPC3_HCDR1_in |
| | | SEQ 68 |
| 164 | INPSGGST | Anti- |
| | | GPC3_HCDR2_in |
| | | SEQ 68 |
| 165 | ARWHGGPYDY | Anti- |
| | | GPC3_HCDR3_in |
| | | SEQ 68 |
| 166 | NIGSKS | Anti- |
| | | GPC3_LCDR1_in |
| | | SEQ 69 |
| 167 | YDS | Anti- |
| | | GPC3_LCDR2_in |
| | | SEQ 69 |
| 168 | QVWDSSSDHYV | Anti- |
| | | GPC3_LCDR3_in |
| | | SEQ 69 |
| 169 | GYTFASHG | Anti- |
| | | GPC3_HCDR1_in |
| | | SEQ 70 |
| 170 | ISPYTGNT | Anti- |
| | | GPC3_HCDR2_in |
| | | SEQ 70 |
| 171 | ARGKRTLASCFDY | Anti- |
| | | GPC3_HCDR3_in |
| | | SEQ 70 |
| 172 | NIGSKS | Anti- |
| | | GPC3_LCDR1_in |
| | | SEQ 71 |
| 173 | DDS | Anti- |
| | | GPC3_LCDR2_in |
| | | SEQ 71 |
| 174 | QVWDSSSDHV | Anti- |
| | | GPC3_LCDR3_in |
| | | SEQ 71 |
| 175 | KLVVVGAGGV | KRAS peptide can be |
| | | targeted by CAR |
| 176 | KLVVVGAVGV | KRAS peptide can be |
| | | targeted by CAR |
| 177 | KLVVVGACGV | KRAS peptide can be |
| | | targeted by CAR |
| 178 | KLVVVGADGV | KRAS peptide can be |
| | | targeted by CAR |
| 179 | KLVVVGASGV | KRAS peptide can be |
| | | targeted by CAR |
| 180 | LVVVGAGGV | KRAS peptide can be |
| | | targeted by CAR |
| 181 | LVVVGAVGV | KRAS peptide can be |
| | | targeted by CAR |
| 182 | LVVVGACGV | KRAS peptide can be |
| | | targeted by CAR |
| 183 | LVVVGADGV | KRAS peptide can be |
| | | targeted by CAR |
| 184 | GGTFSSYA | Anti-KRAS_HCDR1 |
| 185 | IIPIFGKG | Anti-KRAS_HCDR2 |
| 186 | ARHIPTFSFDY | Anti-KRAS_HCDR3 |
| 187 | QVQLVQSGAEVKKPGSSVKVSCKASGGTFSSYAISWVRQAPGQGLEWMGGIIPIFGK | Anti-KRAS_V_{H} |
| | GNYPQKFQGRVTITADESTGTAYMELSSLRSEDTAVYYCARHIPTFSFDYWGQGTLV | |
| | TVSS | |
| 188 | SSNIGAGYD | Anti-KRAS_LCDR1 |
| 189 | GNS | Anti-KRAS_LCDR2 |
| 190 | QSYDSSLSGYV | Anti-KRAS_LCDR3 |
| 191 | QSVLTQPPSVSGAPGQRVTISCTGSSSNIGAGYDVHWYQQLPGTAPKWYGNSNRPS | Anti-KRAS_V_{L} |
| | GVPDRFSGSKSGTSASLAITGLQAEDEADYYCQSYDSSLSGYVFGTGTKVTVLG | |
| 192 | QSVLTQPPSVSGAPGQRVTISCTGSSSNIGAGYDVHWYQQLPGTAPKWYGNSNRPS | Anti-KRAS_scFv |
| | GVPDRFSGSKSGTSASLAITGLQAEDEADYYCQSYDSSLSGYVFGTGTKVTVLGSRGG | |
| | GGSGGGGSGGGGSLEMAQVQLVQSGAEVKKPGSSVKVSCKASGGTFSSYAISWVRQ | |
| | APGQGLEWMGGIIPIFGKGNYPQKFQGRVTITADESTGTAYMELSSLRSEDTAVYYCA | |
| | RHIPTFSFDYWGQGTLVTVSS | |
| 193 | GGTFSSYT | Anti-KRAS_HCDR1 |
| 194 | FIPISGTV | Anti-KRAS_HCDR2 |
| 195 | ARPLDWTEDI | Anti-KRAS_HCDR3 |
| 196 | QVQLVQSGAEVKKPGSSVKVSCKASGGTFSSYTINWVRQAPGQGLEWMGGFIPISGT | Anti-KRAS_V_{H} |
| | VNYAQKFQGRVTITADESTSTAYMELSSLRSEDTAVYYCARPLDWTEDIWGQGTLVT | |
| | VSS | |
| 197 | SSNIGAGYD | Anti-KRAS_LCDR1 |
| 198 | GNS | Anti-KRAS_LCDR2 |
| 199 | QSYDSSLSGSV | Anti-KRAS_LCDR3 |
| 200 | QSVLTQPPSVSGAPGQRVTISCTGSSSNIGAGYDVHWYQQLPGTAPKWYGNSNRPS | Anti-KRAS_V_{L} |
| | GVPDRFSGSKSGTSASLAITGLQAEDEADYYCQSYDSSLSGSVFGTGTKVTVLG | |
| 201 | QSVLTQPPSVSGAPGQRVTISCTGSSSNIGAGYDVHWYQQLPGTAPKWYGNSNRPS | Anti-KRAS_scFv |
| | GVPDRFSGSKSGTSASLAITGLQAEDEADYYCQSYDSSLSGSVFGTGTKVTVLGSRGG | |
| | GGSGGGGSGGGGSLEMAVQLVQSGAEVKKPGSSVKVSCKASGGTFSSYTINWVRQA | |
| | PGQGLEWMGGFIPISGTVNYAQKFQGRVTITADESTSTAYMELSSLRSEDTAVYYCAR | |
| | PLDWTEDIWGQGTLVTVSS | |
| 202 | GYTFTAYY | Anti-KRAS_HCDR1 |
| 203 | MNTNNGAT | Anti-KRAS_HCDR2 |
| 204 | ARGDISQDFADV | Anti-KRAS_HCDR3 |
| 205 | EVQLVQSGAEVKKPGASVKVSCKASGYTFTAYYLHWLRQAPGQGLEWMGWMNTN | Anti-KRAS_V_{H} |
| | NGATRYAQKFQDRVTMTRDTSINTAYMEMSGLSSDDTAMYYCARGDISQDFADVW | |
| | GQGTLVTVSS | |
| 206 | SGSIASNY | Anti-KRAS_LCDR1 |
| 207 | EDN | Anti-KRAS_LCDR2 |
| 208 | QSYDDINHWV | Anti-KRAS_LCDR3 |
| 209 | NFMLTQPHSVSESPGKTVTISCTGSSGSIASNYVQWYQQRPGSAPTILIYEDNKRPSGV | Anti-KRAS_V_{L} |
| | PDRFSGSIDSSSNSASLTISGLKTGDEADYYCQSYDDINHWVFGGGTKLTVLG | |
| 210 | NFMLTQPHSVSESPGKTVTISCTGSSGSIASNYVQWYQQRPGSAPTILIYEDNKRPSGV | Anti-KRAS_scFv |
| | PDRFSGSIDSSSNSASLTISGLKTGDEADYYCQSYDDINHWVFGGGTKLTVLGSRGGG | |
| | GSGGGGSGGGGSLEMAEVQLVQSGAEVKKPGASVKVSCKASGYTFTAYYLHWLRQ | |
| | APGQGLEWMGWMNTNNGATRYAQKFQDRVTMTRDTSINTAYMEMSGLSSDDTAM | |
| | YYCARGDISQDFADVWGQGTLVTVSS | |
| 211 | GYTFTAYY | Anti-KRAS_HCDR1 |
| 212 | MNTNNGAT | Anti-KRAS_HCDR2 |
| 213 | ARGDISQDFADV | Anti-KRAS_HCDR3 |
| 214 | EVQLVQSGAEVKKPGASVKVSCKASGYTFTAYYLHWLRQAPGQGLEWMGWMNTN | Anti-KRAS_V_{H} |
| | NGATRYAQKFQDRVTMTRDTSINTAYMEMSGLSSDDTAMYYCARGDISQDFADVW | |
| | GQGTLVTVSS | |
| 215 | SGSIASNY | Anti-KRAS_LCDR1 |
| 216 | EDN | Anti-KRAS_LCDR2 |
| 217 | QSYDDINHWV | Anti-KRAS_LCDR3 |
| 218 | NFMLTQPHSVSESPGKTVTISCTGSSGSIASNYVQWYQQRPGSAPTILIYEDNKRPSGV | Anti-KRAS_V_{L} |
| | PDRFSGSIDSSSNSASLTISGLKTGDEADYYCQSYDDINHWVFGGGTKLTVLG | |
| 219 | NFMLTQPHSVSESPGKTVTISCTGSSGSIASNYVQWYQQRPGSAPTILIYEDNKRPSGV | Anti-KRAS_scFv |
| | PDRFSGSIDSSSNSASLTISGLKTGDEADYYCQSYDDINHWVFGGGTKLTVLGSRGGG | |
| | GSGGGGSGGGGSLEMAEVQLVQSGAEVKKPGASVKVSCKASGYTFTAYYLHWLRQ | |
| | APGQGLEWMGWMNTNNGATRYAQKFQDRVTMTRDTSINTAYMEMSGLSSDDTAM | |
| | YYCARGDISQDFADVWGQGTLVTVSS | |
| 220 | GGSFSGYY | Anti-KRAS_HCDR1 |
| 221 | VNHSGNT | Anti-KRAS_HCDR2 |
| 222 | ARYFPPMIDV | Anti-KRAS_HCDR3 |
| 223 | QVQLQQWGAGLLKPSETLSLTCAVYGGSFSGYYWSWIRQSPGKGLEWIGEVNHSGN | Anti-KRAS_V_{H} |
| | TNYNPSLKSRVTISLDTSKNQFSLKLNSVTAADTAVYYCARYFPPMIDVWGQGTLVT | |
| | VSS | |
| 224 | SSNIENNY | Anti-KRAS_LCDR1 |
| 225 | DNN | Anti-KRAS_LCDR2 |
| 226 | GTVVDSSLSAYV | Anti-KRAS_LCDR3 |
| 227 | GQSVVTQPPSVSAAPGQKVTISCSGSSSNIENNYVSWYQQLPGTAPKLLIYDNNKRPS | Anti-KRAS_V_{L} |
| | GIPDRFSGSKSGTSATLGITGLQTGDEADYYCGTWDSSLSAYVFGTGTKVTVLG | |
| 228 | QSVVTQPPSVSAAPGQKVTISCSGSSSNIENNYVSWYQQLPGTAPKLLIYDNNKRPSGI | Anti-KRAS_scFv |
| | PDRFSGSKSGTSATLGITGLQTGDEADYYCGTWDSSLSAYVFGTGTKVTVLGSRGGG | |
| | GSGGGGSGGGGSLEMAQVQLQQWGAGLLKPSETLSLTCAVYGGSFSGYYWSWIRQS | |
| | PGKGLEWIGEVNHSGNTNYNPSLKSRVTISLDTSKNQFSLKLNSVTAADTAVYYCAR | |
| | YFPPMIDVWGQGTLVTVSS | |
| 229 | GGSISSSSYY | Anti-KRAS_HCDR1 |
| 230 | INHSGST | Anti-KRAS_HCDR2 |
| 231 | ARYSHHVDSGGYDV | Anti-KRAS_HCDR3 |
| 232 | QLQLQESGPGLVKPSETLSLSCTVSGGSISSSSYYWGWIRQPPGKGLEWIGEINHSGST | Anti-KRAS_V_{H} |
| | NYNPSLKSRVTISVDTSKNQFSLKLSSVTAADTAVYYCARYSHHVDSGGYDVWGQG | |
| | TLVTVSS | |
| 233 | SSNIGNNY | Anti-KRAS_LCDR1 |
| 234 | DNN | Anti-KRAS_LCDR2 |
| 235 | GTWDSSLSAVV | Anti-KRAS_LCDR3 |
| 236 | QSVVTQPPSVSAAPGQKVTISCSGSSSNIGNNYVSWYQQLPRTAPRLLIYDNNKRPSGI | Anti-KRAS_V_{L} |
| | PDRESASKSGTSATLGITGLQTGDEADYYCGTWDSSLSAVVEGGGTKLTVLG | |
| 237 | QSVVTQPPSVSAAPGQKVTISCSGSSSNIGNNYVSWYQQLPRTAPRLLIYDNNKRPSGI | Anti-KRAS_scFv |
| | PDRESASKSGTSATLGITGLQTGDEADYYCGTWDSSLSAVVEGGGTKLTVLGSRGGG | |
| | GSGGGGSGGGGSLEMAQLQLQESGPGLVKPSETLSLSCTVSGGSISSSSYYWGWIRQP | |
| | PGKGLEWIGEINHSGSTNYNPSLKSRVTISVDTSKNQFSLKLSSVTAADTAVYYCARY | |
| | SHHVDSGGYDVWGQGTLVTVSS | |
| 238 | GGTFSSYG | Anti-KRAS_HCDR1 |
| 239 | IIPIFGTP | Anti-KRAS_HCDR2 |
| 240 | ARSYYGYFDG | Anti-KRAS_HCDR3 |
| 241 | EVQLVESGAEVKEPGSSVKVSCKASGGTESSYGISWIRQAPGQGLEWMGEIIPIFGTPN | Anti-KRAS_V_{H} |
| | YAQKFQGRVTITADESTSTAYVELSSLRSDDTAVYYCARSYYGYFDGWGQGTLVTVS | |
| | S | |
| 242 | QDISNY | Anti-KRAS_LCDR1 |
| 243 | DAS | Anti-KRAS_LCDR2 |
| 244 | QQYKSYPLT | Anti-KRAS_LCDR3 |
| 245 | DIQMTQSPSSLSASVGDRVTITCQASQDISNYLNWYQQKPGKAPKLLIYDASNLETGV | Anti-KRAS_V_{L} |
| | PSRFSGSGSGTDFTFTISSLQPDDFATYYCQQYKSYPLTEGGGTKVEIKR | |
| 246 | DIQMTQSPSSLSASVGDRVTITCQASQDISNYLNWYQQKPGKAPKLLIYDASNLETGV | Anti-KRAS_scFv |
| | PSRFSGSGSGTDFTFTISSLQPDDFATYYCQQYKSYPLTEGGGTKVEIKRSRGGGGSGG | |
| | GGSGGGGSLEMAEVQLVESGAEVKEPGSSVKVSCKASGGTESSYGISWIRQAPGQGL | |
| | EWMGEIIPIFGTPNYAQKFQGRVTITADESTSTAYVELSSLRSDDTAVYYCARSYYGY | |
| | FDGWGQGTLVTVSS | |
| 247 | GYTFTSYY | Anti-KRAS_HCDR1 |
| 248 | INPSGGST | Anti-KRAS_HCDR2 |
| 249 | ARSMYQYFLDS | Anti-KRAS_HCDR3 |
| 250 | EVQLVESGAEVKKPGASVKISCKASGYTFTSYYMHWVRQAPGQGLEWMGIINPSGGS | Anti-KRAS_V_{H} |
| | TSYAQKFQGRVTMTRDTSTSTVYMELSSLRSEDTAVYYCARSMYQYFLDSWGQGTL | |
| | VTVSS | |
| 251 | SSNIGAGYD | Anti-KRAS_LCDR1 |
| 252 | GNI | Anti-KRAS_LCDR2 |
| 253 | QSYDSNLSG | Anti-KRAS_LCDR3 |
| 254 | QSVVTQPPSVSGAPGQRVTISCTGSSSNIGAGYDVHWYQQLPGTAPKLLIYGNINRPSG | Anti-KRAS_V_{L} |
| | VPDRFSGSKSGTSASLAITGLQAEDEADYYCQSYDSNLSGYVFATGTKVTVLG | |
| 255 | QSVVTQPPSVSGAPGQRVTISCTGSSSNIGAGYDVHWYQQLPGTAPKLLIYGNINRPSG | Anti-KRAS_scFv |
| | VPDRFSGSKSGTSASLAITGLQAEDEADYYCQSYDSNLSGYVFATGTKVTVLGSRGG | |
| | GGSGGGGSGGGGSLEMAEVQLVESGAEVKKPGASVKISCKASGYTFTSYYMHWVRQ | |
| | APGQGLEWMGIINPSGGSTSYAQKFQGRVTMTRDTSTSTVYMELSSLRSEDTAVYYC | |
| | ARSMYQYFLDSWGQGTLVTVSS | |
| 256 | QLSLLMWIT | NY-ESO-1 peptide can |
| | | be targeted by CAR |
| 257 | SLLMWITQC | NY-ESO-1 peptide can |
| | | be targeted by CAR |
| 258 | SLLMWITQCFL | NY-ESO-1 peptide can |
| | | be targeted by CAR |
| 259 | SLLMWITQV | NY-ESO-1 peptide can |
| | | be targeted by CAR |
| 260 | ALLMWITQC | NY-ESO-1 peptide can |
| | | be targeted by CAR |
| 261 | SALMWITQC | NY-ESO-1 peptide can |
| | | be targeted by CAR |
| 262 | SLAMWITQC | NY-ESO-1 peptide can |
| | | be targeted by CAR |
| 263 | SLLAWITQC | NY-ESO-1 peptide can |
| | | be targeted by CAR |
| 264 | SLLMAITQC | NY-ESO-1 peptide can |
| | | be targeted by CAR |
| 265 | SLLMWATQC | NY-ESO-1 peptide can |
| | | be targeted by CAR |
| 266 | SLLMWIAQC | NY-ESO-1 peptide can |
| | | be targeted by CAR |
| 267 | GDTFSSYS | Anti-NY-ESO- |
| | | 1_HCDR1 |
| 268 | FIPNLNKG | Anti-NY-ESO- |
| | | 1_HCDR2 |
| 269 | ARDWSYSEDY | Anti-NY-ESO- |
| | | 1_HCDR3 |
| 270 | QVQLVQSGAEVKKPGSSVKVSCKASGDTFSSYSISWVRQAPGQGLEWMGRIIPILGIA | Anti-NY-ESO-1_V_{H} |
| | NYAQKYQGRVTLSADKSTSTSYMELNSLRSEDTAVYYCARDWSYSEDYWGQGTLVT | |
| | VSS | |
| 271 | SSNIGNNY | Anti-NY-ESO- |
| | | 1_LCDR1 |
| 272 | DNN | Anti-NY-ESO- |
| | | 1_LCDR2 |
| 273 | GTWDSSLSAWV | Anti-NY-ESO- |
| | | 1_LCDR3 |
| 274 | QSVVTQPPSVSAAPGQKVTISCSGSSSNIGNNYVSWYQQLPGTAPKLLIYDNNKRPSGI | Anti-NY-ESO-1_V_{L} |
| | PDRFSGSKSGTSATLGITGLQTGDEADYYCGTWDSSLSAWVFGGGTKLTVLG | |
| 275 | GYTFTSYG | Anti-NY-ESO- |
| | | 1_HCDR1 |
| 276 | ISAYNGNT | Anti-NY-ESO- |
| | | 1_HCDR2 |
| 277 | ARYSGYYAGDS | Anti-NY-ESO- |
| | | 1_HCDR3 |
| 278 | QVQLVQSGAEVKKPGASVKVSCKASGYTFTSYGISWVRQAPGQGLEWMGWISAYN | Anti-NY-ESO-1_V_{H} |
| | GNTNYAQKLQGRVTMTTDTSTSTAYMELRSLRSDDTAVYYCARYSGYYAGDSWGQ | |
| | GTLVTVSS | |
| 279 | SSNIGAGYD | Anti-NY-ESO- |
| | | 1_LCDR1 |
| 280 | GDT | Anti-NY-ESO- |
| | | 1_LCDR2 |
| 281 | QSYDSNLYTYV | Anti-NY-ESO- |
| | | 1_LCDR3 |
| 282 | QSVLTQPPSVSGAPGQRVTISCTGSSSNIGAGYDVHWYQQLPGTAPKLLIYGDTNRPS | Anti-NY-ESO-1_V_{L} |
| | GVPDRISGSKSGTSASLAITGLQAEDEADYYCQSYDSNLYTYVFGTGTKVTVLG | |
| 283 | GYTFTSYG | Anti-NY-ESO- |
| | | 1_HCDR1 |
| 284 | FIPNLNKG | Anti-NY-ESO- |
| | | 1_HCDR2 |
| 285 | ARGDYGSDQ | Anti-NY-ESO- |
| | | 1_HCDR3 |
| 286 | EVQLVESGAEVKRPGASVKVSCKASGYTFTSYGISWVRQAPGQGLEWMGRFIPNLNK | Anti-NY-ESO-1_V_{H} |
| | GNSAHKFEGRVSFTADKFTNTAYMELRGLKSDDTAVYYCARGDYGSDQWGQGTLV | |
| | TVSS | |
| 287 | SSNIGAGY | Anti-NY-ESO- |
| | | 1_LCDR1 |
| 288 | GNS | Anti-NY-ESO- |
| | | 1_LCDR2 |
| 289 | QSYDSSLSGSWV | Anti-NY-ESO- |
| | | 1_LCDR3 |
| 290 | QSVLTQPPSVSGAPGQRVTISCTGSSSNIGAGYDVHWYQQLPGTAPKLLIYGNSNRPS | Anti-NY-ESO-1_V_{L} |
| | GVPDRFSGSKSGTSASLAITGLQAEDEADYYCQSYDSSLSGSWVFGGGTKLTVLG | |
| 291 | GGTFSSYA | Anti-NY-ESO- |
| | | 1_HCDR1 |
| 292 | IIPIFGTA | Anti-NY-ESO- |
| | | 1_HCDR2 |
| 293 | ARYDSYVYDE | Anti-NY-ESO- |
| | | 1_HCDR3 |
| 294 | EVQLVQSGAEVKKPGSSVKVSCKASGGTFSSYAISWVRQAPGQGLEWMGGIIPIFGTA | Anti-NY-ESO-1_V_{H} |
| | NYAQKFQGRVTITADESTSTAYMELSSLRSEDTAVYYCARYDSYVYDEWGQGTLVT | |
| | VSS | |
| 295 | GSNIGAGYD | Anti-NY-ESO- |
| | | 1_LCDR1 |
| 296 | GNS | Anti-NY-ESO- |
| | | 1_LCDR2 |
| 297 | QSYDSSLSGWGI | Anti-NY-ESO- |
| | | 1_LCDR3 |
| 298 | QSVVTQPPSLSGAPGQRVTISCNGSGSNIGAGYDVHWYQQLPGTAPKWYGNSNRPS | Anti-NY-ESO-1_V_{L} |
| | GVPDRFSGSKSGTSASLAITGLQAEDEADYYCQSYDSSLSGWGIFGGGTKLTVLG | |
| 299 | GYTFTKYG | Anti-NY-ESO- |
| | | 1_HCDR1 |
| 300 | ISADSGKT | Anti-NY-ESO- |
| | | 1_HCDR2 |
| 301 | ARDDDS | Anti-NY-ESO- |
| | | 1_HCDR3 |
| 302 | QVQLVQSGPEVKKPGASMKVSCKASGYTFTKYGISWVRQAPGQGLEWMGWISADS | Anti-NY-ESO-1_V_{H} |
| | GKTSYAQNLQGRVSLTEDTSTATAYMELRSLRSDDTAVYYCARDDDSWGQGTLVTV | |
| | SS | |
| 303 | SSNIGNNY | Anti-NY-ESO- |
| | | 1_LCDR1 |
| 304 | DNN | Anti-NY-ESO- |
| | | 1_LCDR2 |
| 305 | GTWDSSLSAEV | Anti-NY-ESO- |
| | | 1_LCDR3 |
| 306 | QSVLTQPPSVSAAPGQKVTISCSGSSSNIGNNYVSWYQQLPGTAPKLLIYDNNKRPSGI | Anti-NY-ESO-1_V_{L} |
| | PDRFSGSKSGTSATLGITGLQTGDEADYYCGTWDSSLSAEVFGTGTKVTVLG | |
| 307 | GYTLTDLP | Anti-NY-ESO- |
| | | 1_HCDR1 |
| 308 | FDPEDGEI | Anti-NY-ESO- |
| | | 1_HCDR2 |
| 309 | ARYVPYVSYSDS | Anti-NY-ESO- |
| | | 1_HCDR3 |
| 310 | EVQLVQSGAEVKKPGASVKVSCKVSGYTLTDLPMHWVRQAPGKGLEWMGGFDPED | Anti-NY-ESO-1_V_{H} |
| | GEHYAQKFQGRVTMTEDTFTDTAYVELSSLRSEDTAVYYCARYVPYVSYSDSWGQG | |
| | TLVTVSS | |
| 311 | QSLLHSNGYNY | Anti-NY-ESO- |
| | | 1_LCDR1 |
| 312 | LGS | Anti-NY-ESO- |
| | | 1_LCDR2 |
| 313 | MQALQTPYT | Anti-NY-ESO- |
| | | 1_LCDR3 |
| 314 | DVVMTQSPLSLPVTPGEPASISCRSSQSLLHSNGYNYLDWYLQKPGQSPQLLIYLGSN | Anti-NY-ESO-1_V_{L} |
| | RASGVPDRFSGSGSGTDFTLKISRVEAEDVGVYYCMQALQTPYTEGQGTKLEIKR | |
| 315 | GGTFSSYA | Anti-NY-ESO- |
| | | 1_HCDR1 |
| 316 | IIPIFGTA | Anti-NY-ESO- |
| | | 1_HCDR2 |
| 317 | ARSYWSWTPYDP | Anti-NY-ESO- |
| | | 1_HCDR3 |
| 318 | EVQLVQSGAEVKKPGASVKVSCKASGGTFSSYAISWVRQAPGQGLEWMGGIIPIFGT | Anti-NY-ESO-1_V_{H} |
| | ANYAQKFQGRVTITADKSTSTAYMELSSLRSEDTAVYYCARSYWSWTPYDPWGQGT | |
| | LVTVSS | |
| 319 | NIGSKS | Anti-NY-ESO- |
| | | 1_LCDR1 |
| 320 | YDS | Anti-NY-ESO- |
| | | 1_LCDR2 |
| 321 | QVWDSSSDHYV | Anti-NY-ESO- |
| | | 1_LCDR3 |
| 322 | LPVLTQPPSVSVAPGKTARITCGGNNIGSKSVHWYQQKPGQAPVLVIYYDSDRPSGIPE | Anti-NY-ESO-1_V_{L} |
| | RFSGSNSGNTATLTISRVEAGDEADYYCQVWDSSSDHYVFGTGTKVTVLG | |
| 323 | VLDGLDVLL | PRAME peptide can be |
| | | targeted by CAR |
| 324 | SLYSFPEPEA | PRAME peptide can be |
| | | targeted by CAR |
| 325 | ALYVDSLFFL | PRAME peptide can be |
| | | targeted by CAR |
| 326 | SLLQHLIGL | PRAME peptide can be |
| | | targeted by CAR |
| 327 | NLTHVLYPV | PRAME peptide can be |
| | | targeted by CAR |
| 328 | GGTFSSYA | Anti-PRAME_HCDR1 |
| 329 | IIPILGIA | Anti-PRAME_HCDR2 |
| 330 | ARHYGQWWDY | Anti-PRAME_HCDR3 |
| 331 | QVQLVQSGAEVRKPGASVKVSCKASGGTFSSYAISWVRQAPGQGLEWMGRIIPILGIA | Anti-PRAME_V_{H} |
| | NYAQKFQGRVTITADKSTSTAYMELSSLRSEDTAVYYCARHYGQWWDYWGQGTLV | |
| | TVSS | |
| 332 | SSNIGSNT | Anti-PRAME_LCDR1 |
| 333 | SNN | Anti-PRAME_LCDR2 |
| 334 | AAWDDSLNGSYV | Anti-PRAME_LCDR3 |
| 335 | QAVLTQPPSASGTPGQRVTISCSGSSSNIGSNTVNWYQQLPGTAPKLLIYSNNQRPSGV | Anti-PRAME_V_{L} |
| | PDRFSGSKSGTSASLAISGLQSEDEADYYCAAWDDSLNGSYVFGTGTKVTVLG | |
| 336 | GGTFSSHP | Anti-PRAME_HCDR1 |
| 337 | IIPMLDIP | Anti-PRAME_HCDR2 |
| 338 | ARGLYYYDY | Anti-PRAME_HCDR3 |
| 339 | QVQLVQSGAEVKKPGSSVKVSCKASGGTFSSHPISWVRQAPGQGLEWMGRIIPMLDIP | Anti-PRAME_V_{H} |
| | NNAQKFQGRVTITADKSTDTAYLELSSLTSEDTAVYYCARGLYYYDYWGQGTLVTV | |
| | SS | |
| 340 | TSNIGAGFD | Anti-PRAME_LCDR1 |
| 341 | GNT | Anti-PRAME_LCDR2 |
| 342 | QSYDRSLSTIL | Anti-PRAME_LCDR3 |
| 343 | QSVVTQPPAVSGALGQRVTISCTGTTSNIGAGFDVHWYQQRPGAAPKLLISGNTHRPS | Anti-PRAME_V_{L} |
| | GVPDRISGSKSGTLASLAITGLQAEDEADYYCQSYDRSLSTILFGGGTKLTVLG | |
| 344 | GGTFSSYA | Anti-PRAME_HCDR1 |
| 345 | IIPIFGIA | Anti-PRAME_HCDR2 |
| 346 | ARSMWYMDS | Anti-PRAME_HCDR3 |
| 347 | EVQLVESGAEVKKPGSSVKVSCKASGGTFSSYAISWVRQAPGQGLEWMGRIIPIFGIA | Anti-PRAME_V_{H} |
| | NYAQKFQGRVTITADKSTSTAYMELSSLRSEDTAVYYCARSMWYMDSWGQGTLVT | |
| | VSS | |
| 348 | SSNIGAGFD | Anti-PRAME_LCDR1 |
| 349 | GNS | Anti-PRAME_LCDR2 |
| 350 | QSYDSSLSGYV | Anti-PRAME_LCDR3 |
| 351 | QSVLTQPPSVSGAPGQRVTISCTGSSSNIGAGEDVHWYQQLPGTAPKLLIFGNSNRPSG | Anti-PRAME_V_{L} |
| | VPDRFSGSKSGTSASLAITGLQAEDEADYYCQSYDSSLSGYVFGSGTKVTVLG | |
| 352 | GYTFSSYG | Anti-PRAME_HCDR1 |
| 353 | ISPYNGNT | Anti-PRAME_HCDR2 |
| 354 | ARYSGYYYVDY | Anti-PRAME_HCDR3 |
| 355 | QVQLVQSGAEVKKPGASVKVSCKASGYTFSSYGISWVRQAPGQGLEWMGWISPYNG | Anti-PRAME_V_{H} |
| | NTNYAQNLQGRVTMTTDTSTTTAYMELRSLTSDDTAVYYCARYSGYYYVDYWGQG | |
| | TLVTVSS | |
| 356 | QSISSY | Anti-PRAME_LCDR1 |
| 357 | AAS | Anti-PRAME_LCDR2 |
| 358 | QQSYSTPRT | Anti-PRAME_LCDR3 |
| 359 | DIQLTQSPSSLSASVGDRVTITCRASQSISSYLNVVYQQKPGKAPKLLIYAASSLQSGVPS | Anti-PRAME_V_{L} |
| | RFSGSGSGTDFTLTISSLQPEDFATYYCQQSYSTPRTFGQGTKVEIKR | |
| 360 | GGTFSSYA | Anti-PRAME_HCDR1 |
| 361 | IIPILGIA | Anti-PRAME_HCDR2 |
| 362 | ARQGYVWSEMDF | Anti-PRAME_HCDR3 |
| 363 | EVQLVESGAEVKKPGSSVKVSCKASGGTFSSYAISWVRQAPGQGLEWMGRIIPILGIA | Anti-PRAME_V_{H} |
| | NYAQKFQGRVTITADKSTSTAYMELSSLRSEDTAVYYCARQGYVWSEMDFWGQGTL | |
| | VTVSS | |
| 364 | NIGSKS | Anti-PRAME_LCDR1 |
| 365 | YDS | Anti-PRAME_LCDR2 |
| 366 | QVWDSITDHYV | Anti-PRAME_LCDR3 |
| 367 | KLLPVLTQPPSVSVAPGKTARITCGGNNIGSKSVHWYQQKPGQAPVLVIYYDSDRPSG | Anti-PRAME_V_{L} |
| | IPERFSGSNSGNTATLTISRVEAGDEADYYCQVWDSITDHYVFGTGTKVTVLG | |
| 368 | GYTFTSYY | Anti-PRAME_HCDR1 |
| 369 | INPSGGST | Anti-PRAME_HCDR2 |
| 370 | AAGSYYSLDI | Anti-PRAME_HCDR3 |
| 371 | EVQLVESGAEVKKPGASVKVSCKASGYTFTSYYMHWVRQAPGQGLEWMGIINPSGG | Anti-PRAME_V_{H} |
| | STSYAQKFQGRVTMTRDTSTSTVYMELSSLRSEDTAVYYCAAGSYYSLDIWGQGTM | |
| | VTVSS | |
| 372 | SGSIASNF | Anti-PRAME_LCDR1 |
| 373 | DDN | Anti-PRAME_LCDR2 |
| 374 | QSYDGSNVI | Anti-PRAME_LCDR3 |
| 375 | KLLPVLTQPHSVSESPGKTVTISCTGSSGSIASNFVQWYQQRPGSAPTTVIYDDNQRPS | Anti-PRAME_V_{L} |
| | GVPDRFSASIDRSSNSASLTISGLKTDDEADYYCQSYDGSNVIFGGGTKLTVLG | |
| 376 | GYTFSSYY | Anti-PRAME_HCDR1 |
| 377 | INPTSGST | Anti-PRAME_HCDR2 |
| 378 | ARSGGGYGDS | Anti-PRAME_HCDR3 |
| 379 | EVQLVQSGAEVEKPGASVKVSCKASGYTFSSYYMDWVRQAPGQGLEWMGRINPTSG | Anti-PRAME_V_{H} |
| | STTYAQKFQGRVTMTRDTSTFTVYMDLSSLRSEDTAVYYCARSGGGYGDSWGQGTL | |
| | VTVSS | |
| 380 | NFGSQS | Anti-PRAME_LCDR1 |
| 381 | YDQ | Anti-PRAME_LCDR2 |
| 382 | QVWDTYTDHVV | Anti-PRAME_LCDR3 |
| 383 | QPVLTQPPSVSVAPGETASVSCGGNNFGSQSVHWYQQKSGQAPLLVIYYDQDRPSEIP | Anti-PRAME_V_{L} |
| | ARESGSKSGNTATLTISRVEAGDEADYYCQVWDTYTDHVVFGGGTKLTVLG | |
| 384 | RKSAPSTGGV | Histone H3.3 peptide |
| | | can be targeted by |
| | | CAR |
| 385 | RKSAPATGGV | Histone H3.3 peptide |
| | | can be targeted by |
| | | CAR |
| 386 | RMSAPSTGGV | Histone H3.3 peptide |
| | | can be targeted by |
| | | CAR |
| 387 | RMSAPATGGV | Histone H3.3 peptide |
| | | can be targeted by |
| | | CAR |
| 388 | AMSAPSTGGV | Histone H3.3 peptide |
| | | can be targeted by |
| | | CAR |
| 389 | RASAPSTGGV | Histone H3.3 peptide |
| | | can be targeted by |
| | | CAR |
| 390 | RMAAPSTGGV | Histone H3.3 peptide |
| | | can be targeted by |
| | | CAR |
| 391 | RMSAASTGGV | Histone H3.3 peptide |
| | | can be targeted by |
| | | CAR |
| 392 | RMSAPSAGGV | Histone H3.3 peptide |
| | | can be targeted by |
| | | CAR |
| 393 | RMSAPSTAGV | Histone H3.3 peptide |
| | | can be targeted by |
| | | CAR |
| 394 | RMSAPSTGAV | Histone H3.3 peptide |
| | | can be targeted by |
| | | CAR |
| 395 | RMSAPSTGGA | Histone H3.3 peptide |
| | | can be targeted by |
| | | CAR |
| 396 | AMSAPATGGV | Histone H3.3 peptide |
| | | can be targeted by |
| | | CAR |
| 397 | RASAPATGGV | Histone H3.3 peptide |
| | | can be targeted by |
| | | CAR |
| 398 | RMAAPATGGV | Histone H3.3 peptide |
| | | can be targeted by |
| | | CAR |
| 399 | RMSAAATGGV | Histone H3.3 peptide |
| | | can be targeted by |
| | | CAR |
| 400 | RMSAPAAGGV | Histone H3.3 peptide |
| | | can be targeted by |
| | | CAR |
| 401 | RMSAPATAGV | Histone H3.3 peptide |
| | | can be targeted by |
| | | CAR |
| 402 | RMSAPATGAV | Histone H3.3 peptide |
| | | can be targeted by |
| | | CAR |
| 403 | RMSAPATGGA | Histone H3.3 peptide |
| | | can be targeted by |
| | | CAR |
| 404 | GYSFTSYW | Anti-Histone |
| | | H3.3_HCDR1 |
| 405 | IYPGDSDT | Anti-Histone |
| | | H3.3_HCDR2 |
| 406 | ARGYDG | Anti-Histone |
| | | H3.3_HCDR3 |
| 407 | EVQLVQSGAEVKKPGESLKISCKGSGYSFTSYWIGWVRQMPGKGLEWMGIIYPGDSD | Anti-Histone H3.3_V_{H} |
| | TRYSPSFQGQVTISADKSISTAYLQWSSLKASDTAMYYCARGYDGWGQGTLVTVSS | |
| 408 | QSLVYSNGNTY | Anti-Histone |
| | | H3.3_LCDR1 |
| 409 | EVS | Anti-Histone |
| | | H3.3_LCDR2 |
| 410 | MQGTHWPPT | Anti-Histone |
| | | H3.3_LCDR3 |
| 411 | DVVMTQSPLSLPVTLGQPASISCRSSQSLVYSNGNTYLNWFHQRPGQSPRRLIYEVSN | Anti-Histone H3.3_V_{L} |
| | RDSGVPDRFSGSGSGTDFTLKISRVEAEDVGVYYCMQGTHWPPTFGGGTKLEIKR | |
| 412 | GYSFTSYW | Anti-Histone |
| | | H3.3_HCDR1 |
| 413 | IYPGDSDT | Anti-Histone |
| | | H3.3_HCDR2 |
| 414 | ARGFDN | Anti-Histone |
| | | H3.3_HCDR3 |
| 415 | EVQLVQSGAEVKKPGESLKISCKGSGYSFTSYWIGWVRQMPGKGLEWMGIIYPGDSD | Anti-Histone H3.3_V_{H} |
| | TRYSPSFQGQVTISADKSISTAYLQWSSLKASDTAMYYCARGFDNWGQGTLVTVSS | |
| 416 | QSLVYSNGNTY | Anti-Histone |
| | | H3.3_LCDR1 |
| 417 | KVS | Anti-Histone |
| | | H3.3_LCDR2 |
| 418 | MQGTYWPYT | Anti-Histone |
| | | H3.3_LCDR3 |
| 419 | EIVLTQSPLSLPVTLGQPASISCRSSQSLVYSNGNTYLSWFHQRPGQSPRRLIYKVSKRD | Anti-Histone H3.3_V_{L} |
| | SGVPDRFSGSGSGTDFTLKISRVEAEDVGVFYCMQGTYWPYTFGQGTKLEIKR | |
| 420 | GYSFTSYW | Anti-Histone |
| | | H3.3_HCDR1 |
| 421 | IYPGDSDT | Anti-Histone |
| | | H3.3_HCDR2 |
| 422 | ARGYDV | Anti-Histone |
| | | H3.3_HCDR3 |
| 423 | EVQLVQSGAEVKKPGESLKISCKGSGYSFTSYWIGWVRQMPGKGLEWMGIIYPGDSD | Anti-Histone H3.3_V_{H} |
| | TRYSPSFQGQVTISADKSISTAYLQWSSLKASDTAMYYCARGYDVWGQGTLVTVSS | |
| 424 | QSLIYSNGNTY | Anti-Histone |
| | | H3.3_LCDR1 |
| 425 | KVS | Anti-Histone |
| | | H3.3_LCDR2 |
| 426 | MQGTHWPPT | Anti-Histone |
| | | H3.3_LCDR3 |
| 427 | DVVMTQSPLSLPVTLGQPASISCRSSQSLIYSNGNTYLTWFHQRPGQPPRRLIHKVSNR | Anti-Histone H3.3_V_{L} |
| | DSGVPDRFSGSGSGSDFTLKISRVEAEDVGIYYCMQGTHWPPTFGGGTKLEIKR | |
| 428 | GYSFTSYW | Anti-Histone |
| | | H3.3_HCDR1 |
| 429 | IYPGDSDT | Anti-Histone |
| | | H3.3_HCDR2 |
| 430 | ARGYDS | Anti-Histone |
| | | H3.3_HCDR3 |
| 431 | EVQLVQSGAEVKKPGESLKISCKGSGYSFTSYWIGWVRQMPGKGLEWMGIIYPGDSD | Anti-Histone H3.3_V_{H} |
| | TRYSPSFQGQVTISADKSISTAYLQWSSLKASDTAMYYCARGYDSWGQGTLVTVSS | |
| 432 | QSLIYSNGNTY | Anti-Histone |
| | | H3.3_LCDR1 |
| 433 | KVS | Anti-Histone |
| | | H3.3_LCDR2 |
| 434 | MQGTHWPPT | Anti-Histone |
| | | H3.3_LCDR3 |
| 435 | DVVMTQSPLSLPVTLGQPASISCRSSQSLIYSNGNTYLTWFHQRPGQPPRRLIHKVSNR | Anti-Histone H3.3_V_{L} |
| | DSGVPDRFSGSGSGSDFTLKISRVEAEDVGIYYCMQGTHWPPTFGGGTKLEIKR | |
| 436 | GLTFDRYA | Anti-Histone |
| | | H3.3_HCDR1 |
| 437 | ITGDGYYT | Anti-Histone |
| | | H3.3_HCDR2 |
| 438 | ARLSGIGRSSYDG | Anti-Histone |
| | | H3.3_HCDR3 |
| 439 | EVQLVESGGGLVQPGGSLRLSCAASGLTFDRYAMSWVRQAAGKGLERFSAITGDGY | Anti-Histone H3.3_V_{H} |
| | YTYYADSVKGRFTISRDNSKNTLYLQMNSLGAEDTAVYYCARLSGIGRSSYDGWGQ | |
| | GTLVTVSS | |
| 440 | SGINVGTYR | Anti-Histone |
| | | H3.3_LCDR1 |
| 441 | YKSDSDK | Anti-Histone |
| | | H3.3_LCDR2 |
| 442 | MIWHSSA | Anti-Histone |
| | | H3.3_LCDR3 |
| 443 | QAVLTQPSSLSASPGASASLTCTLRSGINVGTYRIYWYQQKPGSPPQYLLRYKSDSDK | Anti-Histone H3.3_V_{L} |
| | QQGSGVPSRFSGSKDASANAGILLISGLQSEDEADYYCMIWHSSAWVFGGGTKLTVL | |
| | G | |
| 444 | GYTFTSYT | Anti-Histone |
| | | H3.3_HCDR1 |
| 445 | ISPYNGNT | Anti-Histone |
| | | H3.3_HCDR2 |
| 446 | ARSWEHGFPYDE | Anti-Histone |
| | | H3.3_HCDR3 |
| 447 | QVQLVESGAEVKKPGASVKVSCKASGYTFTSYTITWVRQAPGQGLEWMGWISPYNG | Anti-Histone H3.3_V_{H} |
| | NTNYAQNLQGRVTMTTDTSTTTAYMELRSLTSDDTAVYYCARSWEHGFPYDEWGQ | |
| | GTLVTVSS | |
| 448 | SSNLGAGYD | Anti-Histone |
| | | H3.3_LCDR1 |
| 449 | ISPYNGNT | Anti-Histone |
| | | H3.3_LCDR2 |
| 450 | QSYDSSLSASV | Anti-Histone |
| | | H3.3_LCDR3 |
| 451 | QSVLTQPPSVSGAPGQRVTISCTGSSSNLGAGYDVHWYQQLPGTAPKVLVYFNNNRP | Anti-Histone H3.3_V_{L} |
| | SGVPDRFSGSKSGTSASLAITGLQAEDEADYYCQSYDSSLSASVFGTGTKVTVLG | |
| 452 | AGTFNRYS | Anti-Histone |
| | | H3.3_HCDR1 |
| 453 | IIPIIGVA | Anti-Histone |
| | | H3.3_HCDR2 |
| 454 | ARQEYSYAMDY | Anti-Histone |
| | | H3.3_HCDR3 |
| 455 | EVQLVQSGAEVRKPGSSVKVSCKASAGTFNRYSLSWVRQAPGQGLEWVGRIIPIIGVA | Anti-Histone H3.3_V_{H} |
| | DYAQKFQGRVTITADKSATTAYMELHSLRSEDTAVYYCARQEYSYAMDYWGQGTL | |
| | VTVSS | |
| 456 | SSNIGAGYD | Anti-Histone |
| | | H3.3_LCDR1 |
| 457 | GNN | Anti-Histone |
| | | H3.3_LCDR2 |
| 458 | QSYDTSLTPV | Anti-Histone |
| | | H3.3_LCDR3 |
| 459 | QSVLTQPPSVSGAPGQRVTIFCTGSSSNIGAGYDVHWYQQLPGTAPKLLIYGNNRPS | Anti-Histone H3.3_V_{L} |
| | GVPDRFSGSKSGTSASLAITGLQAEDEADYYCQSYDTSLTPVFGGGTKLTVLG | |
| 460 | GGTFSSYA | Anti-Histone |
| | | H3.3_HCDR1 |
| 461 | IIPIFGTA | Anti-Histone |
| | | H3.3_HCDR2 |
| 462 | ARSYWTFEYSEDS | Anti-Histone |
| | | H3.3_HCDR3 |
| 463 | QVQLVQSGAEVKKPGSSVKVSCKASGGTFSSYAISWVRQAPGQGLEWMGGIIPIFGTA | Anti-Histone H3.3_V_{H} |
| | NYAQKFQGRVTITADESTSTAYMELSSLRSEDTAVYYCARSYWTFEYSEDSWGQGTL | |
| | VTVSS | |
| 464 | SLNLGAGYD | Anti-Histone |
| | | H3.3_LCDR1 |
| 465 | ANT | Anti-Histone |
| | | H3.3_LCDR2 |
| 466 | QSYDNSLSGYV | Anti-Histone |
| | | H3.3_LCDR3 |
| 467 | QSVLTQPPSVSGAPGQRVTISCTGSSLNLGAGYDVHWYQQFPGTAPKLLIFANTNRPS | Anti-Histone |
| | GVPDRFSASKSGTSASLAITGLQAEDEADYFCQSYDNSLSGYVFGTGTKVTVLG | H3.3_V_{L} |
| 468 | GYTFTSYG | Anti-Histone |
| | | H3.3_HCDR1 |
| 469 | ISAYNGNT | Anti-Histone |
| | | H3.3_HCDR2 |
| 470 | ARYYESGYPFDW | Anti-Histone |
| | | H3.3_HCDR3 |
| 471 | EVQLVQSGAEVKKPGASVKVSCKASGYTFTSYGISWVRQAPGQGLEWMGWISAYNG | Anti-Histone H3.3_V_{H} |
| | NTNYAQKLQGRVTMTTDTSTSTAYMELRSLRSDDTAVYYCARYYESGYPFDWWGQ | |
| | GTLVTVSS | |
| 472 | TFNIGSNT | Anti-Histone |
| | | H3.3_LCDR1 |
| 473 | SNN | Anti-Histone |
| | | H3.3_LCDR2 |
| 474 | AAWDDSLSGHVV | Anti-Histone |
| | | H3.3_LCDR3 |
| 475 | SYVLTQPPSASGTPGQRVTISCSGSTFNIGSNTVNWYQQLPATAPKLLIYSNNQRPSGV | Anti-Histone H3.3_V_{L} |
| | PDRFSGSKSGTSASLAISGLQSEDAAAYYCAAWDDSLSGHVVFGGGTKLTVLG | |
| 476 | GYTFTGYY | Anti-Histone |
| | | H3.3_HCDR1 |
| 477 | FDPEDGET | Anti-Histone |
| | | H3.3_HCDR2 |
| 478 | ARSSWWSPVTYYDI | Anti-Histone |
| | | H3.3_HCDR3 |
| 479 | EVQLVQSGAEVKKPGASVKVSCKASGYTFTGYYMIEWVRQAPGKGLEWMGGFDPED | Anti-Histone H3.3_V_{H} |
| | GETIYAQKFQGRVTMTEDTSTDTAYMELSSLRSEDTAVYYCARSSWWSPVTYYDIW | |
| | GQGTLVTVSS | |
| 480 | SLRSYY | Anti-Histone |
| | | H3.3_LCDR1 |
| 481 | AKS | Anti-Histone |
| | | H3.3_LCDR2 |
| 482 | NSRDSSGNR | Anti-Histone |
| | | H3.3_LCDR3 |
| 483 | SSELTQDPAVSVALGQTVRITCQGDSLRSYYANWYQQKPGQAPVLVIYAKSNRPSGIA | Anti-Histone H3.3_V_{L} |
| | DRFSGSSSGNTASLTITGAQAEDEADYYCNSRDSSGNRWVFGGGTKVTVLG | |
| 484 | GYTVTSYG | Anti-Histone |
| | | H3.3_HCDR1 |
| 485 | ISAYNGDT | Anti-Histone |
| | | H3.3_HCDR2 |
| 486 | ARSSLPFGVVPNAFDI | Anti-Histone |
| | | H3.3_HCDR3 |
| 487 | QMQLVQSGAEVKKPGASMKVSCKASGYTVTSYGLSWVRQAPGQGLEWVGWISAYN | Anti-Histone H3.3_V_{H} |
| | GDTIYAQKLQGRVTMTTDTSTSTAYMELRSLRSDDTAMYYCARSSLPFGVVPNAFDI | |
| | WGQGTMVTVSS | |
| 488 | SSNIGSNT | Anti-Histone |
| | | H3.3_LCDR1 |
| 489 | SNN | Anti-Histone |
| | | H3.3_LCDR2 |
| 490 | AAWDDSLNGYV | Anti-Histone |
| | | H3.3_LCDR3 |
| 491 | SYVLTQPPSASGTPGQRVTISCSGSSSNIGSNTVNWYQQLPGTAPKLLIYSNNQRPSGV | Anti-Histone H3.3_V_{L} |
| | PDRFSGSKSGTSASLAISGLQSEDEADYYCAAWDDSLNGYVFGTGTKVTVLG | |
| 492 | GYTFTDYY | Anti-Histone |
| | | H3.3_HCDR1 |
| 493 | INPHSGGT | Anti-Histone |
| | | H3.3_HCDR2 |
| 494 | AREDYSGSGSSDA | Anti-Histone |
| | | H3.3_HCDR3 |
| 495 | QMQLVQSGAEVKTTGASVRVSCKASGYTFTDYYLHWVRQAPGQGLEWMGWINPHS | Anti-Histone H3.3_V_{H} |
| | GGTNYAQKFQGRVTMTRDTSISTAYMELSSLRSDDTAVYYCAREDYSGSGSSDAWG | |
| | QGTLVTVSS | |
| 496 | NIGSKS | Anti-Histone |
| | | H3.3_LCDR1 |
| 497 | YDN | Anti-Histone |
| | | H3.3_LCDR2 |
| 498 | QVWNSSSDHYV | Anti-Histone |
| | | H3.3_LCDR3 |
| 499 | SYELTQPPSVSVAPGKTARITCGGNNIGSKSVNWYQQKPGQAPVLVIYYDNDRPSGIP | Anti-Histone H3.3_V_{L} |
| | ERFSGSNSGNTATLTISRVEAGDEADYYCQVWNSSSDHYVFGTGTKVTVLG | |
| 500 | RMFPNAPYL | WT1 peptide can be |
| | | targeted by CAR |
| 501 | GGTFSSYAIS | Anti-WT1_HCDR1 |
| 502 | GIIPIFGTANYAQKFQG | Anti-WT1_HCDR2 |
| 503 | RIPPYYGMDV | Anti-WT1_HCDR3 |
| 504 | QVQLVQSGAEVKKPGSSVKVSCKASGGTFSSYAISWVRQAPGQGLEWMGGIIPIFGTA | Anti-WT1_V_{H} |
| | NYAQKFQGRVTITADESTSTAYMELSSLRSEDTAVYYCARRIPPYYGMDVWGQGTTV | |
| | TVSS | |
| 505 | SGSSSNIGSNYVY | Anti-WT1_LCDR1 |
| 506 | RSNQRPS | Anti-WT1_LCDR2 |
| 507 | AAWDDSLNGVV | Anti-WT1_LCDR3 |
| 508 | QTVVTQPPSASGTPGQRVTISCSGSSSNIGSNYVYWYQQLPGTAPKLLIYRSNQRPSGV | Anti-WT1_V_{L} |
| | PDRFSGSKSGTSASLAISGPRSVDEADYYCAAWDDSLNGVVFGGGTKLTVLG | |
| 509 | QTVVTQPPSASGTPGQRVTISCSGSSSNIGSNYVYWYQQLPGTAPKLLIYRSNQRPSGV | Anti-WT1_scFv |
| | PDRFSGSKSGTSASLAISGPRSVDEADYYCAAWDDSLNGVVFGGGTKLTVLGSRGGG | |
| | GSGGGGSGGGSLEMAQVQLVQSGAEVKKPGSSVKVSCKASGGTFSSYAISWVRQAP | |
| | GQGLEWMGGIIPIFGTANYAQKFQGRVTITADESTSTAYMELSSLRSEDTAVYYCARR | |
| | IPPYYGMDVWGQGTTVTVSS | |
| 510 | GDSVSSNSAAWN | Anti-WT1_HCDR1 |
| 511 | RTYYGSKWYNDYAVSVKS | Anti-WT1_HCDR2 |
| 512 | GRLGDAFDI | Anti-WT1_HCDR3 |
| 513 | QVQLQQSGPGLVKPSQTLSLTCAISGDSVSSNSAAWNWIRQSPSRGLEWLGRTYYGS | Anti-WT1_V_{H} |
| | KWYNDYAVSVKSRITINPDTSKNQFSLQLNSVTPEDTAVYYCARGRLGDAFDIWGQG | |
| | TMVTVSS | |
| 514 | RASQSISSYLN | Anti-WT1_LCDR1 |
| 515 | AASSLQS | Anti-WT1_LCDR2 |
| 516 | QQSYSTPLT | Anti-WT1_LCDR3 |
| 517 | DIQMTQSPSSLSASVGDRVTITCRASQSISSYLNWYQQKPGKAPKLLIYAASSLQSGVP | Anti-WT1_V_{L} |
| | SRFSGSGSGTDFTLTISSLQPEDFATYYCQQSYSTPLTFGGGTKVDIKR | |
| 518 | DIQMTQSPSSLSASVGDRVTITCRASQSISSYLNWYQQKPGKAPKLLIYAASSLQSGVP | Anti-WT1_scFv |
| | SRFSGSGSGTDFTLTISSLQPEDFATYYCQQSYSTPLTFGGGTKVDIKRSRGGGGSGGG | |
| | GSGGGGSLEMAQVQLQQSGPGLVKPSQTLSLTCAISGDSVSSNSAAWNWIRQSPSRG | |
| | LEWLGRTYYGSKWYNDYAVSVKSRITINPDTSKNQFSLQLNSVTPEDTAVYYCARGR | |
| | LGDAFDIWGQGTMVTVSS | |
| 519 | GYSFTNFWIS | Anti-WT1_HCDR1 |
| 520 | RVDPGYSYSTYSPSFQG | Anti-WT1_HCDR2 |
| 521 | VQYSGYYDWFDP | Anti-WT1_HCDR3 |
| 522 | QMQLVQSGAEVKEPGESLRISCKGSGYSFTNFWISWVRQMPGKGLEWMGRVDPGYS | Anti-WT1_V_{H} |
| | YSTYSPSFQGHVTISADKSTSTAYLQWNSLKASDTAMYYCARVQYSGYYDWFDPWG | |
| | QGTLVTVSS | |
| 523 | SGSSSNIGSNTVN | Anti-WT1_LCDR1 |
| 524 | SNNQRPS | Anti-WT1_LCDR2 |
| 525 | AAWDDSLNGWV | Anti-WT1_LCDR3 |
| 526 | QAVVTQPPSASGTPGQRVTISCSGSSSNIGSNTVNWYQQVPGTAPKLLIYSNNQRPSG | Anti-WT1_V_{L} |
| | VPDRFSGSKSGTSASLAISGLQSEDEADYYCAAWDDSLNGWVFGGGTKLTVLG | |
| 527 | QAVVTQPPSASGTPGQRVTISCSGSSSNIGSNTVNWYQQVPGTAPKLLIYSNNQRPSG | Anti-WT1_scFv |
| | VPDRFSGSKSGTSASLAISGLQSEDEADYYCAAWDDSLNGWVFGGGTKLTVLGSRGG | |
| | GGSGGGGSGGGGSLEMAQMQLVQSGAEVKEPGESLRISCKGSGYSFTNFWISWVRQ | |
| | MPGKGLEWMGRVDPGYSYSTYSPSFQGHVTISADKSTSTAYLQWNSLKASDTAMYY | |
| | CARVQYSGYYDWFDPWGQGTLVTVSS | |
| 528 | GYNFSNKWIG | Anti-WT1_HCDR1 |
| 529 | IIYPGYSDITYSPSFQG | Anti-WT1_HCDR2 |
| 530 | HTALAGFDY | Anti-WT1_HCDR3 |
| 531 | QVQLVQSGAEVKKPGESLKISCKGSGYNFSNKWIGWVRQLPGRGLEWIAIIYPGYSDI | Anti-WT1_V_{H} |
| | TYSPSFQGRVTISADTSINTAYLHWHSLKASDTAMYYCVRHTALAGFDYWGLGTLVT | |
| | VSS | |
| 532 | RASQNINKWLA | Anti-WT1_LCDR1 |
| 533 | KASSLES | Anti-WT1_LCDR2 |
| 534 | QQYNSYAT | Anti-WT1_LCDR3 |
| 535 | DIQMTQSPSTLSASVGDRVTITCRASQNINKWLAWYQQRPGKAPQLLIYKASSLESGV | Anti-WT1_V_{L} |
| | PSRFSGSGSGTEYTLTISSLQPDDFATYYCQQYNSYATFGQGTKVEEKR | |
| 536 | DIQMTQSPSTLSASVGDRVTITCRASQNINKWLAWYQQRPGKAPQLLIYKASSLESGV | Anti-WT1_scFv |
| | PSRFSGSGSGTEYTLTISSLQPDDFATYYCQQYNSYATFGQGTKVEEKRSRGGGGSGG | |
| | GGSGGGGSLEMAQVQLVQSGAEVKKPGESLKISCKGSGYNFSNKWIGWVRQLPGRG | |
| | LEWIAHYPGYSDITYSPSFQGRVTISADTSINTAYLHWHSLKASDTAMYYCVRHTALA | |
| | GFDYWGLGTLVTVSS | |
| 537 | GFTFDDYGMS | Anti-WT1_HCDR1 |
| 538 | GINWNGGSTGYADSVRG | Anti-WT1_HCDR2 |
| 539 | ERGYGYHDPHDY | Anti-WT1_HCDR3 |
| 540 | EVQLVQSGGGVVRPGGSLRLSCAASGFTFDDYGMSWVRQAPGKGLEWVSGINWNG | Anti-WT1_V_{H} |
| | GSTGYADSVRGRFTISRDNAKNSLYLQMNSLRAEDTALYYCARERGYGYHDPHDYW | |
| | GQGTLVTVSS | |
| 541 | GRNNIGSKSVH | Anti-WT1_LCDR1 |
| 542 | DDSDRPS | Anti-WT1_LCDR2 |
| 543 | QVWDSSSDHVV | Anti-WT1_LCDR3 |
| 544 | QSVVTQPPSVSVAPGKTARITCGRNNIGSKSVHWYQQKPGQAPVLVVYDDSDRPSGIP | Anti-WT1_V_{L} |
| | ERFSGSNSGNTATLTISRVEAGDEADYYCQVWDSSSDHVVFGGGTKLTVLG | |
| 545 | QSVVTQPPSVSVAPGKTARITCGRNNIGSKSVHWYQQKPGQAPVLVVYDDSDRPSGIP | Anti-WT1_scFv |
| | ERFSGSNSGNTATLTISRVEAGDEADYYCQVWDSSSDHVVFGGGTKLTVLGSRGGGG | |
| | SGGGGSGGSLEMAEVQLVQSGGGVVRPGGSLRLSCAASGFTFDDYGMSWVRQAPGK | |
| | GLEWVSGINWNGGSTGYADSVRGRFTISRDNAKNSLYLQMNSLRAEDTALYYCARE | |
| | RGYGYHDPHDYWGQGTLVTVSS | |
| 546 | GFSVSGTYMG | Anti-WT1_HCDR1 |
| 547 | LLYSGGGTYHPASLQG | Anti-WT1_HCDR2 |
| 548 | GGAGGGHFDS | Anti-WT1_HCDR3 |
| 549 | EVQLVETGGGLLQPGGSLRLSCAASGFSVSGTYMGWVRQAPGKGLEWVALLYSGGG | Anti-WT1_V_{H} |
| | TYHPASLQGRFIVSRDSSKNMVYLQMNSLKAEDTAVYYCAKGGAGGGHFDSWGQG | |
| | TLVTVSS | |
| 550 | TGSSSNIGAGYDVH | Anti-WT1_LCDR1 |
| 551 | GNSNRPS | Anti-WT1_LCDR2 |
| 552 | AAWDDSLNGYV | Anti-WT1_LCDR3 |
| 553 | QSVLTQPPSVSGAPGQRVTISCTGSSSNIGAGYDVHWYQQLPGTAPKLLIYGNSNRPS | Anti-WT1_V_{L} |
| | GVPDRFSGSKSGTSASLAISGLQSEDEADYYCAAWDDSLNGYVFGTGTKLTVLG | |
| 554 | QSVLTQPPSVSGAPGQRVTISCTGSSSNIGAGYDVHWYQQLPGTAPKLLIYGNSNRPS | Anti-WT1_scFv |
| | GVPDRFSGSKSGTSASLAISGLQSEDEADYYCAAWDDSLNGYVFGTGTKLTVLGSRG | |
| | GGGSGGGGSGGGGSLEMAEVQLVETGGGLLQPGGSLRLSCAASGFSVSGTYMGWVR | |
| | QAPGKGLEWVALLYSGGGTYHPASLQGRFIVSRDSSKNMVYLQMNSLKAEDTAVYY | |
| | CAKGGAGGGHFDSWGQGTLVTVSS | |
| 555 | LTDAVKVMDL | PSA peptide can be |
| | | targeted by CAR |
| 556 | KLQCVDLHV | PSA peptide can be |
| | | targeted by CAR |
| 557 | VISNDVCAQV | PSA peptide can be |
| | | targeted by CAR |
| 558 | FLTPKKLQCV | PSA peptide can be |
| | | targeted by CAR |
| 559 | ALQCVDLHV | PSA peptide can be |
| | | targeted by CAR |
| 560 | KLACVDLHV | PSA peptide can be |
| | | targeted by CAR |
| 561 | KLQAVDLHV | PSA peptide can be |
| | | targeted by CAR |
| 562 | KLQCADLHV | PSA peptide can be |
| | | targeted by CAR |
| 563 | KLQCVALHV | PSA peptide can be |
| | | targeted by CAR |
| 564 | KLQCVDAHV | PSA peptide can be |
| | | targeted by CAR |
| 565 | KLQCVDLAV | PSA peptide can be |
| | | targeted by CAR |
| 566 | GGTFSSYA | Anti-PSA_HCDR1 |
| 567 | GFTFSSYA | Anti-PSA_HCDR1 |
| 568 | GYNFLNYG | Anti-PSA_HCDR1 |
| 569 | GYTFTGYY | Anti-PSA_HCDR1 |
| 570 | GGSFSDYY | Anti-PSA_HCDR1 |
| 571 | GYTFTSYG | Anti-PSA_HCDR1 |
| 572 | GGTFSSYA | Anti-PSA_HCDR1 |
| 573 | GYTFTSYG | Anti-PSA_HCDR1 |
| 574 | GYTFTSYY | Anti-PSA_HCDR1 |
| 575 | GYTFTGYF | Anti-PSA_HCDR1 |
| 576 | GGTFSSYA | Anti-PSA_HCDR1 |
| 577 | GYSFTSYW | Anti-PSA_HCDR1 |
| 578 | GYSFTSYW | Anti-PSA_HCDR1 |
| 579 | GYSFTSYR | Anti-PSA_HCDR1 |
| 580 | GYTFTNYG | Anti-PSA_HCDR1 |
| 581 | IIPIPGIT | Anti-PSA_HCDR2 |
| 582 | ISGSGGST | Anti-PSA_HCDR2 |
| 583 | ISTYTGNT | Anti-PSA_HCDR2 |
| 584 | FDPEDGET | Anti-PSA_HCDR2 |
| 585 | INHSGGT | Anti-PSA_HCDR2 |
| 586 | ISAYNGNT | Anti-PSA_HCDR2 |
| 587 | INPNSGGT | Anti-PSA_HCDR2 |
| 588 | ISAYNGNT | Anti-PSA_HCDR2 |
| 589 | FDPEDGET | Anti-PSA_HCDR2 |
| 590 | FDPEDGET | Anti-PSA_HCDR2 |
| 591 | IIPILGIA | Anti-PSA_HCDR2 |
| 592 | IYPGDSDT | Anti-PSA_HCDR2 |
| 593 | IYPGDSDT | Anti-PSA_HCDR2 |
| 594 | IDPSDSYT | Anti-PSA_HCDR2 |
| 595 | ARSYKWGSSLVDA | Anti-PSA_HCDR3 |
| 596 | ARNYYSQYWMMDL | Anti-PSA_HCDR3 |
| 597 | ARSSEYYTWDH | Anti-PSA_HCDR3 |
| 598 | ARYGFDY | Anti-PSA_HCDR3 |
| 599 | ARYNEYGSGYDK | Anti-PSA_HCDR3 |
| 600 | ARSSQYYVWDS | Anti-PSA_HCDR3 |
| 601 | ARWSYYYFQQFWSLDG | Anti-PSA_HCDR3 |
| 602 | ARTNYNKYDI | Anti-PSA_HCDR3 |
| 603 | ARYSYDY | Anti-PSA_HCDR3 |
| 604 | ARYSYDL | Anti-PSA_HCDR3 |
| 605 | ARVSQPVYGSSTYDI | Anti-PSA_HCDR3 |
| 606 | ARLVVPDAFDI | Anti-PSA_HCDR3 |
| 607 | ARWGSRGFLDAFDI | Anti-PSA_HCDR3 |
| 608 | ARWGLSWDGWGVTDY | Anti-PSA_HCDR3 |
| 609 | ARYNYDT | Anti-PSA_HCDR3 |
| 610 | ARSFGAGYDS | Anti-PSA_HCDR3 |
| 611 | ARYPWDH | Anti-PSA_HCDR3 |
| 612 | ARSSYYGYLSDG | Anti-PSA_HCDR3 |
| 613 | EVQLVQSGAEVKRPGSSVKVSCKASGGTFSSYAINWVRRAPGQGLEWMGKIIPIPGIT | Anti-PSA_V_{H} |
| | NYAQKFQDRVTFTADTSTNIAYMELSSLRSEDTAMYYCARSYKWGSSLVDAWGQGT | |
| | LVTVSS | |
| 614 | EVQLVESGGGLVQPGGSLRLSCAASGFTFSSYAMSWVRQAPGKGLEWVSAISGSGGS | Anti-PSA_V_{H} |
| | TYYADSVKGRFTISRDNSKNTLYLQMNSLRAEDTAVYYCARNYYSQYWMMDLWGQ | |
| | GTLVTVSS | |
| 615 | QVQLVQSGAEVKKPGDSVKVSCKPSGYNFLNYGINWVRQAPGQGLEWMGWISTYT | Anti-PSA_V_{H} |
| | GNTNYAQKLQGRVTFTTDTSTSTAYMEMRSLRSDDTAVYYCARSSEYYTWDHWGQ | |
| | GTLVTVSS | |
| 616 | EVQLVQSGAEVKKPGASVKVSCKASGYTFTGYYIHWVRQAPGQGLEWMGGFDPED | Anti-PSA_V_{H} |
| | GETIYAQKFQDRVTMTADTSTDTAYMELSSLRSEDTAVYYCARYGFDYWGQGTLVT | |
| | VSS | |
| 617 | QVQLQQWGAGLLKPSETLSLTCAVKGGSFSDYYWSWIRQPPGKGLEWIGEINHSGGT | Anti-PSA_V_{H} |
| | NYNPSLKSRVTISVDTSKNQFSLKLLSVTAADTAVYYCARYNEYGSGYDKWGQGTL | |
| | VTVSS | |
| 618 | QVQLVQSGAEVKKPGASVKVSCKASGYTFTSYGISWVRQAPGQGLEWMGWISAYN | Anti-PSA_V_{H} |
| | GNTNYAQKLQGRVTMTTDTSTSTAYMELRSLRSDDTAVYYCARSSQYYVWDSWGQ | |
| | GTLVTVSS | |
| 619 | QVQLVQSGAEVKKPGSSVKVSCKASGGTFSSYAISWVRQAPGQGLEWMGWINPNSG | Anti-PSA_V_{H} |
| | GTNYAQKFQGRVTMTRDTSISTAYMELSRLRSDDTAVYYCARWSYYYFQQFWSLDG | |
| | WGQGTLVTVSS | |
| 620 | QMQLVQSGSEVKKPGASVKVSCKASGYTFTSYGISWVRQAPGQGLEWMGWISAYN | Anti-PSA_V_{H} |
| | GNTDYVQKLQGRVTMTTDTSTNTAYMELGSLGSDDTAVYYCARTNYNKYDIWGQG | |
| | TLVTVSS | |
| 621 | QVQLVQSGAEVKKPGASVKVSCKASGYTFTSYYMHWVRQAPGQGLEWMGGFDPED | Anti-PSA_V_{H} |
| | GETIYAQKFQGRVTMTEDTSTDTAYMGLSSLRSEDTAVYYCARYSYDYWGQGTLVT | |
| | VSS | |
| 622 | EVQLVQSGAEVKKPGASVKVSCKASGYTFTGYFMHWVRQAPGQGLEWMGGFDPED | Anti-PSA_V_{H} |
| | GETIYAQKFQGRVTMTEDTSTDTAYMELSSLRSEDTAVYYCARYSYDLWGQGTLVT | |
| | VSS | |
| 623 | QVQLVQSGAEVKKPGSSVKVSCKASGGTFSSYAISWVRQAPGQGLEWMGRIIPILGIA | Anti-PSA_V_{H} |
| | NYAQKFQGRVTITADKSTSTAYMELSSLRSEDTAVYYCARVSQPVYGSSTYDIWGQG | |
| | TLVTVSS | |
| 624 | QMQLVQSGAEVKKPGESLKISCKGSGYSETSYWIGWVRQMPGKGLEWMGHYPGDS | Anti-PSA_V_{H} |
| | DTRYSPSFQGQVTISADKSISTAYLQWSSLKASDTAMYYCARLVVPDAFDIWGQGTM | |
| | VTVSS | |
| 625 | QVQLVQSGAEVKKPGESLKISCKGSGYSETSYWIGWVRQMPGKGLEWMGIIYPGDSD | Anti-PSA_V_{H} |
| | TRYSPSFQGQVTISADKSISTAYLQWSSLKASDTAMYYCARWGSRGFLDAFDIWGQG | |
| | TMVTVSS | |
| 626 | EVQLVQSGAEVKKPGESLKISCKGSGYSFTSYRIGWVRQMPGKGLEWMGHYPGDSD | Anti-PSA_V_{H} |
| | TRYSPSFQGQVTISADKSISTAYLQWSSLKASDTAMYYCARWGLSWDGWGVTDYW | |
| | GQGTLVTVSS | |
| 627 | QVQLVQSGAEVKKPGASVKVSCKASGYTFTSYYMHWVRQAPGKGLEWMGGFDPED | Anti-PSA_V_{H} |
| | GETIYAQKFQGRVTMTEDTSTDTAYMELSSLRSEDTAVYYCARYNYDTWGQGTLVT | |
| | VSS | |
| 628 | EVQLVQSGAEVKKPGESLKISCKGSGYSFTSYWIGWVRQMPGKGLEWMGRIDPSDSY | Anti-PSA_V_{H} |
| | TNYSPSFQGHVTISADKSISTAYLQWSSLKASDTAMYYCARSFGAGYDSWGQGTLVT | |
| | VSS | |
| 629 | QVQLVQSGAEVKKPGASVKVSCKASGYTFTSYYMHWVRQAPGKGLEWMGGFDPED | Anti-PSA_V_{H} |
| | GETIYAQKFQGRVTMTEDTSTDTAYMELSSLRSEDTAVYYCARYPWDHWGQGTLVT | |
| | VSS | |
| 630 | QVQLVQSGAEVKKPGASVKVSCKTSGYTFTNYGISWVRQAPGQGLEWMGWISAYN | Anti-PSA_V_{H} |
| | GNTNYAQNLQGRVTMTTDTSTSTAYMELRSLRSDDTAVYYCARSSYYGYLSDGWG | |
| | QGTLVTVSS | |
| 631 | NSNIGSNT | Anti-PSA_LCDR1 |
| 632 | SSNFGAGYD | Anti-PSA_LCDR1 |
| 633 | SSNIGAGYD | Anti-PSA_LCDR1 |
| 634 | TGAVTSGYY | Anti-PSA_LCDR1 |
| 635 | SYNIGNNY | Anti-PSA_LCDR1 |
| 636 | SSNFGAGYD | Anti-PSA_LCDR1 |
| 637 | SSNIGSNT | Anti-PSA_LCDR1 |
| 638 | SSNIGAGYD | Anti-PSA_LCDR1 |
| 639 | TGAVTSGYY | Anti-PSA_LCDR1 |
| 640 | TGAVTSGYY | Anti-PSA_LCDR1 |
| 641 | SSNLGSNS | Anti-PSA_LCDR1 |
| 642 | SSNIGNNY | Anti-PSA_LCDR1 |
| 643 | SSNIGNNY | Anti-PSA_LCDR1 |
| 644 | QSISSY | Anti-PSA_LCDR1 |
| 645 | TGTVTSTYY | Anti-PSA_LCDR1 |
| 646 | SSDVGGYNY | Anti-PSA_LCDR1 |
| 647 | SSNIGTNY | Anti-PSA_LCDR1 |
| 648 | SNN | Anti-PSA_LCDR2 |
| 649 | GDT | Anti-PSA_LCDR2 |
| 650 | GNS | Anti-PSA_LCDR2 |
| 651 | TTG | Anti-PSA_LCDR2 |
| 652 | DNN | Anti-PSA_LCDR2 |
| 653 | GNS | Anti-PSA_LCDR2 |
| 654 | STS | Anti-PSA_LCDR2 |
| 655 | DNH | Anti-PSA_LCDR2 |
| 656 | DNY | Anti-PSA_LCDR2 |
| 657 | DND | Anti-PSA_LCDR2 |
| 658 | AAS | Anti-PSA_LCDR2 |
| 659 | DVS | Anti-PSA_LCDR2 |
| 660 | STN | Anti-PSA_LCDR2 |
| 661 | ATWDDSLNGPV | Anti-PSA_LCDR3 |
| 662 | QSYDTSLSGSV | Anti-PSA_LCDR3 |
| 663 | QSYDSSLSGWV | Anti-PSA_LCDR3 |
| 664 | LLYSGGVWV | Anti-PSA_LCDR3 |
| 665 | GTVVESSLSAYV | Anti-PSA_LCDR3 |
| 666 | QSYDSSLSGWV | Anti-PSA_LCDR3 |
| 667 | AAWDDSLNGRWV | Anti-PSA_LCDR3 |
| 668 | QSYDSSLSEV | Anti-PSA_LCDR3 |
| 669 | LLYYGGAYV | Anti-PSA_LCDR3 |
| 670 | LLYYGGAQWV | Anti-PSA_LCDR3 |
| 671 | AAWDDSLNSVV | Anti-PSA_LCDR3 |
| 672 | GTVVDSSLSAGV | Anti-PSA_LCDR3 |
| 673 | GTVVDSSLSSGV | Anti-PSA_LCDR3 |
| 674 | QQSYSTPFT | Anti-PSA_LCDR3 |
| 675 | LVFYGGVWV | Anti-PSA_LCDR3 |
| 676 | SSYTSSSRYV | Anti-PSA_LCDR3 |
| 677 | LLYYGGQGV | Anti-PSA_LCDR3 |
| 678 | AAWDDSLSGLYV | Anti-PSA_LCDR3 |
| 679 | QSVLTQPPSASGTPGQRVTLSCSGSNSNIGSNTVNWYQQLPGTNPKLLIYSNNQRPSG | Anti-PSA_V_{L} |
| | VPDRFSGSKSGTSASLAISGLQSEDEADYYCATWDDSLNGPVFGGGTKLTVLG | |
| 680 | QSVLTQPPSVSGAPGQRVTISCTGSSSNFGAGYDVHWYQQLPGAAPKLLIYGDTNRPS | Anti-PSA_V_{L} |
| | GVPDRFSGSKSGTSASLAITGLQAEDEADYYCQSYDTSLSGSVFGGGTKLTVLG | |
| 681 | QSVLTQPPSVSGAPGQRVTISCTGSSSNIGAGYDVHWYQQLPGTAPKLLIYGNSNRPS | Anti-PSA_V_{L} |
| | GVPDRFSGSKSGTSASLAITGLQAEDEADYYCQSYDSSLSGWVFGGGTKLTVLG | |
| 682 | QAVVTQEPSLTVSPGGTVTLTCASSTGAVTSGYYPNWFQLKPGQAPRALIYTTGKKH | Anti-PSA_V_{L} |
| | SWAPARFSGSLLGGKAALTLSGVQPEDEAEYYCLLYSGGVWVFGGGTKLTVLG | |
| 683 | QSVLTQPPSVSAAPGQKVTISCSGSSYNIGNNYVSWYQQLPGTAPKLLIYDNNKRPSGI | Anti-PSA_V_{L} |
| | PDRFSGSKSGTSATLGITGLQTGDEAEYYCGTWESSLSAYVFGTGTKVTVLG | |
| 684 | QSVVTQPPSVSGAPGQRVTISCTGSSSNFGAGYDVHWYQQLPGTAPKLLIYGNSNRPS | Anti-PSA_V_{L} |
| | GVPDRFSGSKSGTSASLAITGLQAEDEADYYCQSYDSSLSGWVFGGGTKLTVLG | |
| 685 | QAVLTQPPSASGTPGQRVTISCSGSSSNIGSNTVNWYQQLPGTAPKLLIYSNNQRPSGV | Anti-PSA_V_{L} |
| | PDRFSGSKSGTSASLAISGLQSEDEADYYCAAWDDSLNGRWVFGGGTKLTVLG | |
| 686 | QSVLTQPPSVSGAPGQRVTISCTGSSSNIGAGYDVHWYQQLPGTAPKLLIYGNSNRPS | Anti-PSA_V_{L} |
| | GVPDRFSGSKSGTSASLAITGLQAEDEADYYCQSYDSSLSEVFGTGTKVTVLG | |
| 687 | QAVVTQEPSLTVSPGGTVTLTCASSTGAVTSGYYPNWFQQKPGQAPRALIYSTSNKHS | Anti-PSA_V_{L} |
| | WTPARFSGSLLGGKAALTLSGVQPEDEAEYYCLLYYGGAYVFGTGTKVTVLG | |
| 688 | QTVVTQEPSLTVSPGGTVTLTCASSTGAVTSGYYPNWFQQKPGQAPRALIYSTSNKHS | Anti-PSA_V_{L} |
| | WTPARFSGSLLGGKAALTLSGVQPEDEAEYYCLLYYGGAQWVFGGGTKLTVLG | |
| 689 | QAVLTQPPSASGTPGQRVTISCSGRSSNLGSNSVNWYQQVPGTAPKLLIFDNHQRPSG | Anti-PSA_V_{L} |
| | GSKSGTSASLAISGLRSEDETDYYCAAWDDSLNSVVFGGGTKLTVLG | |
| 690 | QSVVTQPPSVSAAPGQKVTISCSGSSSNIGNNYVSWYQQLPGTAPKLLIYDNYKRPSGI | Anti-PSA_V_{L} |
| | PDRFSGSKSGTSATLGITGLQTGDEADYYCGTWDSSLSAGVFGGGTKLTVLG | |
| 691 | QSVVTQPPSVSAAPGQRVTISCSGSSSNIGNNYVSWYQQLPGAAPRLLIYDNDKRPSGI | Anti-PSA_V_{L} |
| | PDRFSGSKSGTSATLGITGLQTGDEADYYCGTWDSSLSSGVFGGGTKLTVLG | |
| 692 | DIQLTQSPSSLSASVGDRVTITCRASQSISSYLNWYQQKPGKAPKLLIYAASSLQSGVPS | Anti-PSA_V_{L} |
| | RFSGSGSGTDFTLTISSLQPEDFATYYCQQSYSTPFTFGPGTKVDIKR | |
| 693 | QAVVTQEPSLTVSPGGTVTLTCASSTGTVTSTYYPNWFQQKPGQAPRALIYSTSNRHS | Anti-PSA_V_{L} |
| | WTPARFSGSLLGGKAALTVSGVQPDDEAEYYCLVFYGGVWVFGGGTKLTVLG | |
| 694 | QSALTQPASVSGSPGQSITISCTGTSSDVGGYNYVSWYQQHPGKAPKLMIYDVSKRPS | Anti-PSA_V_{L} |
| | GVSNRFSGSKSGNTASLTISGLQAEDEADYYCSSYTSSSRYVFGTGTKVTVLG | |
| 695 | QAVVTQEPSLTVSPGGTVTLTCASSTGAVTSGYYPNWFQQKPGQAPRPLIYSTNNKHS | Anti-PSA_V_{L} |
| | WTPARFSGSLLGGKAALTLSGVQPEDEAEYYCLLYYGGQGVFGGGTKLTVLG | |
| 696 | LPVLTQPPSASGTPGQRVTISCSGSSSNIGTNYVYWYQQLPGTAPKLLIYSNNQRPSGV | Anti-PSA_V_{L} |
| | PDRFSGSKSGTSASLAISGLRSEDEADYYCAAWDDSLSGLYVFGTGTKVTVLG | |
| 697 | KNDAPVVQEPRRLSFRSTIYGSR | ROR1 peptide can be |
| | | targeted by CAR |
| 698 | AANCIRIGIPMADPI | ROR1 peptide can be |
| | | targeted by CAR |
| 699 | SSTGVLFVKFGPPPTASPG | ROR1 peptide can be |
| | | targeted by CAR |
| 700 | SNPMILMRLKLPNCE | ROR1 peptide can be |
| | | targeted by CAR |
| 701 | GGSLSSHGVS | Anti-ROR1_HCDR1 |
| 702 | RIIPMFGVTDYAQKFQD | Anti-ROR1_HCDR2 |
| 703 | ESRGATFEY | Anti-ROR1_HCDR3 |
| 704 | QVQLVQSGTEVKKPGSSVKVSCQASGGSLSSHGVSWLRQAPGQGLEWVGRIIPMFGV | Anti-ROR1_V_{H} |
| | TDYAQKFQDRVTITADKSTSTVYMELISLGSDDTAVYFCARESRGATFEYWGQGTLV | |
| | TVSS | |
| 705 | RASQSVSSSYLA | Anti-ROR1_LCDR1 |
| 706 | GASSRAT | Anti-ROR1_LCDR2 |
| 707 | QQYGSS | Anti-ROR1_LCDR3 |
| 708 | EIVLTQSPGTLSLSPGERATLSCRASQSVSSSYLAWYQQKPGQAPRLLIYGASSRATGIP | Anti-ROR1_V_{L} |
| | DRFSGSGSGTDFTLTISRLEPEDFAVYYCQQYGSSFGPGTKVDIKR | |
| 709 | GGSISSSSYYWG | Anti-ROR1_HCDR1 |
| 710 | SIYYSGSTYYNPSLKS | Anti-ROR1_HCDR2 |
| 711 | HDGTDAFDI | Anti-ROR1_HCDR3 |
| 712 | QLQLQESGPGLVKPSETLSLTCTVSGGSISSSSYYWGWIRQPPGKGLEWIGSIYYSGST | Anti-ROR1_V_{H} |
| | YYNPSLKSRVTISVDTSKNQFSLKLGSVTAADTAVYYCARHDGTDAFDIWGQGTTVT | |
| | VSS | |
| 713 | TGTSSDFGDYDYVS | Anti-ROR1_LCDR1 |
| 714 | DVSDRPS | Anti-ROR1_LCDR2 |
| 715 | SSLTTSSTLV | Anti-ROR1_LCDR3 |
| 716 | QSVLTQPASVSGSPGQSITISCTGTSSDFGDYDYVSWYQQHPGKAPKLMIYDVSDRPS | Anti-ROR1_V_{L} |
| | GVSNRFSGSKSGNTASLTISGLQAEDEADYFCSSLTTSSTLVFGGGTKLTVLG | |

One or more features from any embodiments described herein or in the figures may be combined with one or more features of any other embodiment described herein in the figures without departing from the scope of the disclosure.

All publications, patents and patent applications cited in this specification are herein incorporated by reference as if each individual publication or patent application were specifically and individually indicated to be incorporated by reference. Although the foregoing disclosure has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

What is claimed:

1. A chimeric antigen receptor (CAR) comprising:
(a) an extracellular target-binding domain comprising an antibody moiety;
(b) a transmembrane domain;
(c) a CD30 costimulatory domain; and
(d) a primary signaling domain.

2. The CAR of claim 1, wherein the CD30 costimulatory domain comprises a sequence that can bind to an intracellular TRAF signaling protein.

3. The CAR of claim 2, wherein the sequence that can bind to an intracellular TRAF signaling protein corresponds to residues 561-573 or 578-586 of a full-length CD30 having the sequence of SEQ ID NO:11.

4. The CAR of any one of claims 1 to 3, wherein the CD30 costimulatory domain comprises a sequence that is at least 80%, 85%, 90%, 95%, or 100% identical to residues 561-573 or 578-586 of SEQ ID NO:11.

5. The CAR of any one of claims 1 to 4, wherein the CD30 costimulatory domain comprises a sequence that is at least 70%, 75%, 80%, 85%, 90%, 95%, or 100% identical to the sequence of SEQ ID NO:35.

6. The CAR of any one of claims 1 to 5, wherein the CAR comprises more than one CD30 costimulatory domain.

7. The CAR of any one of claims 1 to 6, wherein the CAR further comprises at least one costimulatory domain which comprises the intracellular sequence of a costimulatory molecule that is different from CD30, and optionally wherein the costimulatory molecule that is different from CD30 is selected from the group consisting of CD27, CD28, 4-1BB (CD137), OX40, CD40, PD-1, ICOS, lymphocyte function-associated antigen-1 (LFA-1), CD2, CD7, LIGHT, NKG2C, B7-H3, and a ligand that specifically binds with CD83.

8. The CAR of any one of claims 1 to 7, wherein the antibody moiety is a single chain antibody fragment, a single chain Fv (scFv), a single chain Fab, a single chain Fab′, a single domain antibody fragment, a single domain multispecific antibody, an intrabody, a nanobody, or a single chain immunokine.

9. The CAR of any one of claims 1 to 8, wherein:
(a) the transmembrane domain of the CAR is derived from the transmembrane domain of a TCR co-receptor or a T cell costimulatory molecule, and optionally wherein the TCR co-receptor or T cell costimulatory molecule is selected from the group consisting of CD8, 4-1BB, CD27, CD28, CD30, OX40, CD3ε, CD3ζ, CD45, CD4, CD5, CD9, CD16, CD22, CD33, CD37, CD64, CD80, CD86, CD134, CD137, and CD154; or
(b) the transmembrane domain of the CAR is the transmembrane domain of CD8, 4-1BB, CD27, CD28, CD30, OX40, CD3ε, CD3ζ, CD45, CD4, CD5, CD9, CD16, CD22, CD33, CD37, CD64, CD80, CD86, CD134, CD137, or CD154.

10. The CAR of any one of claims 1 to 9, wherein the transmembrane domain of the CAR comprises an amino acid sequence selected from the group consisting of SEQ ID NOS:26-31.

11. The CAR of any one of claims 1 to 10, wherein the primary signaling domain comprises a sequence derived from the intracellular signaling sequence of a molecule selected from the group consisting of CD3ζ, TCRζ, FcRγ, FcRβ, CD3γ, CD3δ, CD3ε, CD5, CD22, CD79a, CD79b, and CD66d.

12. The CAR of any one of claims 1 to 11, wherein the primary signaling domain comprises a sequence that is at least 80%, 85%, 90%, 95%, or 100% identical to the sequence of SEQ ID NO:37.

13. The CAR of any one of claims 1 to 12, further comprises a peptide linker between the extracellular target-binding domain and the transmembrane domain, and/or a peptide linker between the transmembrane domain and the CD30 costimulatory domain, and/or a peptide linker between the CD30 costimulatory domain and the primary signaling domain.

14. The CAR of any one of claims 1 to 13, wherein the antibody moiety specifically binds to a disease-related antigen.

15. The CAR of claim 14, wherein the disease-related antigen is a cancer-related antigen or a virus-related antigen.

16. The CAR of any one of claims 1 to 15, wherein the antibody moiety specifically binds to a cell surface antigen, optionally wherein the cell surface antigen is selected from the group consisting of protein, carbohydrate, and lipid, and further optionally wherein the cell surface antigen is CD19, CD20, CD22, CD47, CD158e, GPC3, ROR1, ROR2, BCMA, GPRC5D, FcRL5, MUC16, MCT4, PSMA, or a variant or mutant thereof.

17. The CAR of any one of claims 1 to 16, wherein the antibody moiety specifically binds to a MHC-restricted antigen.

18. The CAR of claim 17, wherein the antibody moiety specifically binds to:
(a) a complex comprising an alpha-fetoprotein (AFP) peptide and a MHC class I protein; or
(b) a complex comprising a KRAS peptide and a MHC class I protein; or
(c) a complex comprising a NY-ESO-1 peptide and a MHC class I protein; or
(d) a complex comprising a PRAME peptide and a MHC class I protein; or
(e) a complex comprising a histone H3.3 peptide and a MHC class I protein; or
(f) a complex comprising a WT1 peptide and a MHC class I protein; or
(g) a complex comprising a PSA peptide and a MHC class I protein; or
(h) a complex comprising a ROR1 peptide and a MHC class I protein.

19. The CAR of any one of claims 1 to 16, wherein the antibody moiety specifically binds to a glypican 3 (GPC3) peptide.

20. A nucleic acid molecule encoding, in whole or in part, the CAR of any one of claims 1 to 19.

21. A vector comprising the nucleic acid molecule of claim 20

22. A CD30-CAR effector cell: (a) expressing the CAR of any one of claims 1 to 19, or (b) comprising the nucleic acid molecule of claim 20 or the vector of claim 21, optionally wherein effector cell is a T cell.

23. A pharmaceutical composition comprising the CAR of any one of claims 1 to 19, the nucleic acid molecule of claim 20, the vector of claim 21, or the CD30-CAR effector cell of claim 22, and a pharmaceutically acceptable carrier or diluent.

24. A method of killing target cells, comprising:
contacting one or more target cells with one or more CD30-CAR effector cells of claim 22 under conditions and for a time sufficient so that the CD30-CAR effector cells mediate killing of the target cells,
wherein the target cells express an antigen specific to the CD30-CAR effector cells, and
wherein the CD30-CAR effector cells express a low cell exhaustion level upon contacting the target cells, and
optionally wherein the CD30-CAR effector cells are T cells.

25. The method of claim 24, wherein the CD30-CAR effector T cells express a low level of an exhaustion marker selected from the group consisting of PD-1, TIM-3, and LAG-3.

26. The method of claim 24 or 25, wherein the CD30-CAR effector cells express a lower level of PD-1, TIM-3, or LAG-3 than corresponding effector cells expressing a CAR comprising a CD28 costimulatory domain.

27. The method of any one of claims 24 to 26, wherein:
(a) the CD30-CAR effector cells express a lower level of PD-1 than the corresponding CD28 CAR effector cells, and wherein the ratio of PD-1 expression level of the CD30-CAR effector cells to the corresponding CD28 CAR effector cells is 0.9, 0.8, 0.7, 0.6, 0.5, 0.4, 0.3, 0.2, 0.1 or lower; and/or
(b) the CD30-CAR effector cells express a lower level of TIM-3 than the corresponding CD28 CAR effector cells, and wherein the ratio of TIM-3 expression level of the CD30-CAR effector cells to the corresponding CD28 CAR effector cells is 0.9, 0.8, 0.7, 0.6, 0.5, 0.4, 0.3, 0.2, 0.1 or lower; and/or
(c) the CD30-CAR effector cells express a lower level of LAG-3 than the corresponding CD28 CAR effector cells, and wherein the ratio of LAG-3 expression level of the CD30-CAR effector cells to the corresponding CD28 CAR effector cells is 0.9, 0.8, 0.7, 0.6, 0.5, 0.4, 0.3, 0.2, 0.1 or lower.

28. The method of any one of claims 24 to 27, wherein the CD30-CAR effector T cells express a lower level of PD-1, TIM-3, or LAG-3 than corresponding effector T cells expressing a CAR comprising a 4-1BB costimulatory domain.

29. The method of any one of claims 24 to 28, wherein:
(a) the CD30-CAR effector T cells express a lower cell exhaustion level of PD-1 than the corresponding 4-1BB CAR effector cells, and wherein the ratio of PD-1 expression level of the CD30-CAR effector cells to the corresponding 4-1BB CAR effector cells is 0.9, 0.8, 0.7, 0.6, 0.5, 0.4, 0.3, 0.2, 0.1 or lower; and/or
(b) the CD30-CAR effector cells express a lower level of TIM-3 than the corresponding 4-1BB CAR effector cells, and wherein the ratio of TIM-3 expression level of the CD30-CAR effector cells to the corresponding 4-1BB CAR effector cells is 0.9, 0.8, 0.7, 0.6, 0.5, 0.4, 0.3, 0.2, 0.1 or lower; and/or
(c) the CD30-CAR effector cells express a lower level of LAG-3 than the corresponding 4-1BB CAR effector cells, and wherein the ratio of LAG-3 expression level of the CD30-CAR effector cells to the corresponding 4-1BB CAR effector cells is 0.9, 0.8, 0.7, 0.6, 0.5, 0.4, 0.3, 0.2, 0.1 or lower.

30. The method of any one of claims 24 to 29, wherein the target cells are cancer cells, optionally wherein the cancer cells are from a cancer selected from the group consisting of adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, leukemia, lymphoma, lung cancer, melanoma, mesothelioma, multiple myeloma, pancreatic cancer, pheochromocytoma, plasmacytoma, neuroblastoma, ovarian cancer, prostate cancer, sarcoma, stomach cancer, uterine cancer, and thyroid cancer, and/or optionally wherein the cancer cells are hematological cancer cells, solid tumor cells, or virus-infected cells.

31. A method of treating a disease, the method comprising a step of administering to a subject the CAR of any one of claims 1 to 19, the nucleic acid molecule of claim 20, the vector of claim 21, the CD30-CAR effector cell of claim 22, or the pharmaceutical composition of claim 23 to the subject.

32. The method of claim 31, wherein the disease is cancer, optionally wherein the cancer is selected from the group consisting of adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, leukemia, lymphoma, lung cancer, melanoma, mesothelioma, multiple myeloma, pancreatic cancer, pheochromocytoma, plasmacytoma, neuroblastoma, ovarian cancer, prostate cancer, sarcoma, stomach cancer, uterine cancer, and thyroid cancer, and/or optionally wherein the cancer is a hematological cancer or a solid tumor cancer.

33. The method of claim 31, wherein the disease is a viral infection.

34. A method for preventing and/or reversing T cell exhaustion in a subject, comprising administering to the subject the CAR of any one of claims 1 to 19, the nucleic acid molecule of claim 20, the vector of claim 21, the CD30-CAR effector cell of claim 22, or the pharmaceutical composition of claim 23 comprising the nucleic acid molecule or the vector to the subject.

35. The method of claim 34, wherein the method decreases the expression of an exhaustion marker in a T cell, optionally wherein the exhaustion marker is selected from the group consisting of PD-1, TIM-3, and LAG-3.

36. A method for generating central memory T cells and/or effector memory T cells in a subject, comprising administering to the subject the CAR of any one of claims 1 to 19, the nucleic acid molecule of claim 20, the vector of claim 21, the CD30-CAR effector cell of claim 22, or the pharmaceutical composition of claim 23 comprising the nucleic acid molecule or the vector to the subject.

37. A method for generating central memory T cells and/or effector memory T cells in vitro comprising:
contacting one or more target cells with the CD30-CAR effector cell of claim 22 under conditions and for a time sufficient so that the effector cell develops into central memory T cells, wherein the target cells express an antigen specific to the effector cell.


SEQUENCE LISTING (720 sequences)

SEQ ID NO: 1 - protein, 512 aa, Artificial Sequence
  Description of Artificial Sequence Synthetic polypeptide
METDTLLLWVLLLWVPGSTGLPVLTQPPSVSVAPGKTARITCGGNNIGSKSVHWYQQKPGQAPVLVVYDDSDRPSGIPERFSGSNSGNTATLTISRVEAGDEADYYCQVWDSSSDYVVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQVWGWQGGMYPRSNWWYNLDSWGQGTLVTVSSEQKLISEEDLAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 677 aa, Artificial Sequence
  Description of Artificial Sequence Synthetic polypeptide
METDTLLLWVLLLWVPGSTGLPVLTQPPSVSVAPGKTARITCGGNNIGSKSVHWYQQKPGQAPVLVVYDDSDRPSGIPERFSGSNSGNTATLTISRVEAGDEADYYCQVWDSSSDYVVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQVWGWQGGMYPRSNWWYNLDSWGQGTLVTVSSEQKLISEEDLAAATGAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 664 aa, Artificial Sequence
  Description of Artificial Sequence Synthetic polypeptide
METDTLLLWVLLLWVPGSTGLPVLTQPPSVSVAPGKTARITCGGNNIGSKSVHWYQQKPGQAPVLVVYDDSDRPSGIPERFSGSNSGNTATLTISRVEAGDEADYYCQVWDSSSDYVVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQVWGWQGGMYPRSNWWYNLDSWGQGTLVTVSSEQKLISEEDLAAATGTTTPAPRPPTPAPTIASQPLSLRPEACRPAAGGAVHTRGLDFACDIYIWAPLAGTCGVLLLSLVITLYCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 515 aa, Artificial Sequence
  Description of Artificial Sequence Synthetic polypeptide
METDTLLLWVLLLWVPGSTGLPVLTQPPSVSVAPGKTARITCGGNNIGSKSVHWYQQKPGQAPVLVVYDDSDRPSGIPERFSGSNSGNTATLTISRVEAGDEADYYCQVWDSSSDYVVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQVWGWQGGMYPRSNWWYNLDSWGQGTLVTVSSEQKLISEEDLTGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 501 aa, Artificial Sequence
  Description of Artificial Sequence Synthetic polypeptide
METDTLLLWVLLLWVPGSTGQSVLTQPASVSGSPGQSITISCTGTSSDVGGYNYVSWYQQHPGKAPKLMIYDVNNRPSEVSNRFSGSKSGNTASLTISGLQAEDEADYYCSSYTTGSRAVFGGGTKLTVLGSRGGGGSGGGGSGGGGSLEMAEVQLVQSGAEVKKPGESLTISCKASGYSFPNYWITWVRQMSGGGLEWMGRIDPGDSYTTYNPSFQGHVTISIDKSTNTAYLHWNSLKASDTAMYYCARYYVSLVDIWGQGTLVTVSSEQKLISEEDLAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 666 aa, Artificial Sequence
  Description of Artificial Sequence Synthetic polypeptide
METDTLLLWVLLLWVPGSTGQSVLTQPASVSGSPGQSITISCTGTSSDVGGYNYVSWYQQHPGKAPKLMIYDVNNRPSEVSNRFSGSKSGNTASLTISGLQAEDEADYYCSSYTTGSRAVFGGGTKLTVLGSRGGGGSGGGGSGGGGSLEMAEVQLVQSGAEVKKPGESLTISCKASGYSFPNYWITWVRQMSGGGLEWMGRIDPGDSYTTYNPSFQGHVTISIDKSTNTAYLHWNSLKASDTAMYYCARYYVSLVDIWGQGTLVTVSSEQKLISEEDLAAATGAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 653 aa, Artificial Sequence
  Description of Artificial Sequence Synthetic polypeptide
METDTLLLWVLLLWVPGSTGQSVLTQPASVSGSPGQSITISCTGTSSDVGGYNYVSWYQQHPGKAPKLMIYDVNNRPSEVSNRFSGSKSGNTASLTISGLQAEDEADYYCSSYTTGSRAVFGGGTKLTVLGSRGGGGSGGGGSGGGGSLEMAEVQLVQSGAEVKKPGESLTISCKASGYSFPNYWITWVRQMSGGGLEWMGRIDPGDSYTTYNPSFQGHVTISIDKSTNTAYLHWNSLKASDTAMYYCARYYVSLVDIWGQGTLVTVSSEQKLISEEDLAAATGTTTPAPRPPTPAPTIASQPLSLRPEACRPAAGGAVHTRGLDFACDIYIWAPLAGTCGVLLLSLVITLYCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 504 aa, Artificial Sequence
  Description of Artificial Sequence Synthetic polypeptide
METDTLLLWVLLLWVPGSTGQSVLTQPASVSGSPGQSITISCTGTSSDVGGYNYVSWYQQHPGKAPKLMIYDVNNRPSEVSNRFSGSKSGNTASLTISGLQAEDEADYYCSSYTTGSRAVFGGGTKLTVLGSRGGGGSGGGGSGGGGSLEMAEVQLVQSGAEVKKPGESLTISCKASGYSFPNYWITWVRQMSGGGLEWMGRIDPGDSYTTYNPSFQGHVTISIDKSTNTAYLHWNSLKASDTAMYYCARYYVSLVDIWGQGTLVTVSSEQKLISEEDLTGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 514 aa, Artificial Sequence
  Description of Artificial Sequence Synthetic polypeptide
METDTLLLWVLLLWVPGSTGLPVLTQPPSVSVAPGKTARITCGGNNIGSKSVHWYQQKPGQAPVLVVYDDSDRPSGIPERFSGSNSGNTATLTISRVEAGDEADYYCQVWDSSSDYVVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQVWGWQGGMYPRSNWWYNLDSWGQGTLVTVSSEQKLISEEDLTGTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 509 aa, Artificial Sequence
  Description of Artificial Sequence Synthetic polypeptide
METDTLLLWVLLLWVPGSTGQSVLTQPASVSGSPGQSITISCTGTSSDVGGYNYVSWYQQHPGKAPKLMIYDVNNRPSEVSNRFSGSKSGNTASLTISGLQAEDEADYYCSSYTTGSRAVFGGGTKLTVLGSRGGGGSGGGGSGGGGSLEMAEVQLVQSGAEVKKPGESLTISCKASGYSFPNYWITWVRQMSGGGLEWMGRIDPGDSYTTYNPSFQGHVTISIDKSTNTAYLHWNSLKASDTAMYYCARYYVSLVDIWGQGTLVTVSSEQKLISEEDLAAATGTTTPAPRPPTPAPTIASQPLSLRPEACRPAAGGAVHTRGLDFACDIYIWAPLAGTCGVLLLSLVITLYCAAARSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 595 aa, Homo sapiens
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 12 - protein, 247 aa, Artificial Sequence
  Description of Artificial Sequence Synthetic polypeptide
QPVLTQPPSVSVAPGKTARITCGGNNIGSKSVHWYQQKPGQAPVLVIYYDSDRPSGIPERFSGSNSGNTATLTISRVEAGDEADYYCQVWDSSSDHYVFGTGTKVTVLGSRGGGGSGGGGSGGGGSLEMAQVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARWHGGPYDYWGQGTLVTVSS

SEQ ID NO: 13 - protein, 249 aa, Artificial Sequence
  Description of Artificial Sequence Synthetic polypeptide
QSVLTQPPSVSVAPGKTARITCGGNNIGSKSVHWYQQKPGQAPVLVVYDDSDRPSGIPERFSGSNSGNTATLTISRVEAGDEADYYCQVWDSSSDHVFGTGTKVTVLGSRGGGGSGGGGSGGGGSLEMAQVQLVQSGADVRKPGASVKVSCKASGYTFASHGISWVRQAPGQGLEWLGWISPYTGNTNYAQKFQGRVTMATDTSTSTAYMELRSLRSDDTAIYYCARGKRTLASCFDYWGQGTLVTVSS

SEQ ID NO: 14 - protein, 260 aa, Artificial Sequence
  Description of Artificial Sequence Synthetic polypeptide
LPVLTQPPSVSVAPGKTARITCGGNNIGSKSVHWYQQKPGQAPVLVVYDDSDRPSGIPERFSGSNSGNTATLTISRVEAGDEADYYCQVWDSSSDYVVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQVWGWQGGMYPRSNWWYNLDSWGQGTLVTVSS

SEQ ID NO: 15 - protein, 247 aa, Artificial Sequence
  Description of Artificial Sequence Synthetic polypeptide
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRSRGGGGSGGGGSGGGGSLEQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSS

SEQ ID NO: 16 - protein, 247 aa, Artificial Sequence
  Description of Artificial Sequence Synthetic polypeptide
DIQLTQSPSSLSTSVGDRVTITCQASHDIRNYLNWYQQKPGKAPNLLIYAASNLQTGVPSRFSGRGSGTDFTLTISSLQPEDIATYYCQQYDGLPLTFGQGTRLEIKRSRGGGGSGGGGSGGGGSLEMAQVQLVESGGGLVQPGGSLRLSCAASGFTFSNYAMSWVRQAPGKGLEWVSSISGSGGSTYYADSVKGRFTISRDTSKNTLYLQMNSLRAEDTAVYYCARYGSAAWMDSWGQGTLVTVSS

SEQ ID NO: 17 - protein, 245 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSFGPGTKVDIKRSRGGGGSGGGGSGGGGSLEMAQVQLVQSGTEVKKPGSSVKVSCQASGGSLSSHGVSWLRQAPGQGLEWVGRIIPMFGVTDYAQKFQDRVTITADKSTSTVYMELISLGSDDTAVYFCARESRGATFEYWGQGTLVTVSS

SEQ ID NO: 18 - protein, 251 aa, Artificial Sequence
  Description of Artificial Sequence Synthetic polypeptide
QSVLTQPASVSGSPGQSITISCTGTSSDFGDYDYVSWYQQHPGKAPKLMIYDVSDRPSGVSNRFSGSKSGNTASLTISGLQAEDEADYFCSSLTTSSTLVFGGGTKLTVLGSRGGGGSGGGGSGGGGSLEMAQLQLQESGPGLVKPSETLSLTCTVSGGSISSSSYYWGWIRQPPGKGLEWIGSIYYSGSTYYNPSLKSRVTISVDTSKNQFSLKLGSVTAADTAVYYCARHDGTDAFDIWGQGTTVTVSS

SEQ ID NO: 19 - protein, 254 aa, Artificial Sequence
  Description of Artificial Sequence Synthetic polypeptide
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGYVFGTGTKVTVLGSRGGGGSGGGGSGGGGSLEMAEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARSMGSSLYASSDVWGQGTLVTVSS

SEQ ID NO: 20 - protein, 249 aa, Artificial Sequence
  Description of Artificial Sequence Synthetic polypeptide
QAVLTQPPSASGTPGQRVTISCSGSSSNIGSNTVNWYQQLPGTAPKLLMYSNNQRPSGVPDRFSGSKSGTSASLAISGLQSEDEADYYCAAWDDSLNGYVFGTGTKVTVLGSRGGGGSGGGGSGGGGSLEMAEVQLVQSGAEMKKPGESLKISCKGSGYNFASYWVGWVRQMPGKGLEWMGTIYPDDSDTRYGPAFQGQVTISADKSISTAYLQWSSLKASDTAMYYCARDSYYGIDVWGQGTLVTVSS

SEQ ID NO: 21 - protein, 249 aa, Artificial Sequence
  Description of Artificial Sequence Synthetic polypeptide
QSVVTQPPSVSAAPGQKVTISCSGSSSNIGNNYVSWYQQLPGTAPKLLIYDNNKRPSGIPDRFSASKSGTSATLGITGLQTRDEADYYCGTWDSSLSAWVFGGGTKLTVLGSRGGGGSGGGGSGGGGSLEMAQVQLVQSGAEVKKPGSSVKVSCKASGDTFSSYYISWVRQAPGQGLEWMGRIIPILGIANYAQKYQGRVTLSADKSTSTSYMELNSLSSEDTAVYYCALDWSYSIDYWGQGTLVTVSS

SEQ ID NO: 22 - protein, 251 aa, Artificial Sequence
  Description of Artificial Sequence Synthetic polypeptide
QSVVTQPPSLSGAPGQRVTISCNGSGSNIGAGYDVHWYQQLPGTAPKLLIYGNSNRPSGVPDRFSGSKSGTSASLAITGLQAEDEADYYCQSYDSSLSGWGIFGGGTKLTVLGSRGGGGSGGGGSGGGGSLEMAEVQLVQSGAEVKKPGSSVKVSCKASGGTFSSYAISWVRQAPGQGLEWMGGIIPIFGTANYAQKFQGRVTITADESTSTAYMELSSLRSEDTAVYYCARYDSYVYDEWGQGTLVTVSS

SEQ ID NO: 23 - protein, 248 aa, Artificial Sequence
  Description of Artificial Sequence Synthetic polypeptide
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRSRGGGGSGGGGSGGGGSLEMAQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSS

SEQ ID NO: 24 - protein, 517 aa, Artificial Sequence
  Description of Artificial Sequence Synthetic polypeptide
DIQLTQSPSSLSTSVGDRVTITCQASHDIRNYLNWYQQKPGKAPNLLIYAASNLQTGVPSRFSGRGSGTDFTLTISSLQPEDIATYYCQQYDGLPLTFGQGTRLEIKRSRGGGGSGGGGSGGGGSLEMAQVQLVESGGGLVQPGGSLRLSCAASGFTFSNYAMSWVRQAPGKGLEWVSSISGSGGSTYYADSVKGRFTISRDTSKNTLYLQMNSLRAEDTAVYYCARYGSAAWMDSWGQGTLVTVSSGGGGSGGGGSLPVLTQPPSVSVAPGKTARITCGGNNIGSKSVHWYQQKPGQAPVLVVYDDSDRPSGIPERFSGSNSGNTATLTISRVEAGDEADYYCQVWDSSSDYVVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQVWGWQGGMYPRSNWWYNLDSWGQGTLVTVSS

SEQ ID NO: 25 - protein, 774 aa, Artificial Sequence
  Description of Artificial Sequence Synthetic polypeptide
LPVLTQPPSVSVAPGKTARITCGGNNIGSKSVHWYQQKPGQAPVLVVYDDSDRPSGIPERFSGSNSGNTATLTISRVEAGDEADYYCQVWDSSSDYVVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQVWGWQGGMYPRSNWWYNLDSWGQGTLVTVSSGGGGSGGGGSDIQLTQSPSSLSTSVGDRVTITCQASHDIRNYLNWYQQKPGKAPNLLIYAASNLQTGVPSRFSGRGSGTDFTLTISSLQPEDIATYYCQQYDGLPLTFGQGTRLEIKRSRGGGGSGGGGSGGGGSLEMAQVQLVESGGGLVQPGGSLRLSCAASGFTFSNYAMSWVRQAPGKGLEWVSSISGSGGSTYYADSVKGRFTISRDTSKNTLYLQMNSLRAEDTAVYYCARYGSAAWMDSWGQGTLVTVSSGGGGSGGGGSQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRSRGGGGSGGGGSGGGGSLEQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSS

SEQ ID NO: 26 - protein, 21 aa, Artificial Sequence
  Description of Artificial Sequence Synthetic peptide
IYIWAPLAGTCGVLLLSLVIT

SEQ ID NO: 27 - protein, 27 aa, Artificial Sequence
  Description of Artificial Sequence Synthetic peptide
IISFFLALTSTALLFLLFFLTLRFSVV

SEQ ID NO: 28 - protein, 21 aa, Artificial Sequence
  Description of Artificial Sequence Synthetic peptide
ILVIFSGMFLVFTLAGALFLH

SEQ ID NO: 29 - protein, 27 aa, Artificial Sequence
  Description of Artificial Sequence Synthetic peptide
FWVLVVVGGVLACYSLLVTVAFIIFWV

SEQ ID NO: 30 - protein, 28 aa, Artificial Sequence
  Description of Artificial Sequence Synthetic peptide
PVLDAGPVLFWVILVLVVVVGSSAFLLC

SEQ ID NO: 31 - protein, 21 aa, Artificial Sequence
  Description of Artificial Sequence Synthetic peptide
VAAILGLGLVLGLLGPLAILL

SEQ ID NO: 32 - protein, 42 aa, Artificial Sequence
  Description of Artificial Sequence Synthetic polypeptide
KRGRKKLLYIFKQPFMRPVQTTQEEDGCSCRFPEEEEGGCEL

SEQ ID NO: 33 - protein, 48 aa, Artificial Sequence
  Description of Artificial Sequence Synthetic polypeptide
QRRKYRSNKGESPVEPAEPCRYSCPREEEGSTIPIQEDYRKPEPACSP

SEQ ID NO: 34 - protein, 41 aa, Artificial Sequence
  Description of Artificial Sequence Synthetic polypeptide
RSKRSRLLHSDYMNMTPRRPGPTRKHYQPYAPPRDFAAYRS

SEQ ID NO: 35 - protein, 188 aa, Artificial Sequence
  Description of Artificial Sequence Synthetic polypeptide
HRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 36 - protein, 42 aa, Artificial Sequence
  Description of Artificial Sequence Synthetic polypeptide
ALYLLRRDQRLPPDAHKPPGGGSFRTPIQEEQADAHSTLAKI

SEQ ID NO: 37 - protein, 112 aa, Artificial Sequence
  Description of Artificial Sequence Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 21 aa, Artificial Sequence
  Description of Artificial Sequence Synthetic peptide
SRGGGGSGGGGSGGGGSLEMA

SEQ ID NO: 39 - protein, 5 aa, Artificial Sequence
  Description of Artificial Sequence Synthetic peptide
GGGGS

SEQ ID NO: 40 - protein, 4 aa, Artificial Sequence
  Description of Artificial Sequence Synthetic peptide
GGSG

SEQ ID NO: 41 - protein, 4 aa, Artificial Sequence
  Description of Artificial Sequence Synthetic peptide
SGGG

SEQ ID NO: 42 - protein, 4 aa, Artificial Sequence
  Description of Artificial Sequence Synthetic peptide
GSGS

SEQ ID NO: 43 - protein, 6 aa, Artificial Sequence
  Description of Artificial Sequence Synthetic peptide
GSGSGS

SEQ ID NO: 44 - protein, 8 aa, Artificial Sequence
  Description of Artificial Sequence Synthetic peptide
GSGSGSGS

SEQ ID NO: 45 - protein, 10 aa, Artificial Sequence
  Description of Artificial Sequence Synthetic peptide
GSGSGSGSGS

SEQ ID NO: 46 - protein, 6 aa, Artificial Sequence
  Description of Artificial Sequence Synthetic peptide
GGSGGS

SEQ ID NO: 47 - protein, 9 aa, Artificial Sequence
  Description of Artificial Sequence Synthetic peptide
GGSGGSGGS

SEQ ID NO: 48 - protein, 12 aa, Artificial Sequence
  Description of Artificial Sequence Synthetic peptide
GGSGGSGGSGGS

SEQ ID NO: 49 - protein, 4 aa, Artificial Sequence
  Description of Artificial Sequence Synthetic peptide
GGSG

SEQ ID NO: 50 - protein, 8 aa, Artificial Sequence
  Description of Artificial Sequence Synthetic peptide
GGSGGGSG

SEQ ID NO: 51 - protein, 12 aa, Artificial Sequence
  Description of Artificial Sequence Synthetic peptide
GGSGGGSGGGSG

SEQ ID NO: 52 - protein, 21 aa, Artificial Sequence
  Description of Artificial Sequence Synthetic peptide
SRGGGGSGGGGSGGGGSLEMA

SEQ ID NO: 53 - protein, 6 aa, Artificial Sequence
  Description of Artificial Sequence Synthetic peptide
HHHHHH

SEQ ID NO: 54 - protein, 9 aa, Artificial Sequence
  Description of Artificial Sequence Synthetic peptide
YPYDVPDYA

SEQ ID NO: 55 - protein, 10 aa, Artificial Sequence
  Description of Artificial Sequence Synthetic peptide
YPYDVPDYAS

SEQ ID NO: 56 - protein, 8 aa, Artificial Sequence
  Description of Artificial Sequence Synthetic peptide
DYKDDDDK

SEQ ID NO: 57 - protein, 10 aa, Artificial Sequence
  Description of Artificial Sequence Synthetic peptide
EQKLISEEDL

SEQ ID NO: 58 - protein, 114 aa, Artificial Sequence
  Description of Artificial Sequence Synthetic polypeptide
EVQLVESGGGLVQPGGSLRLSCAASGFTFSNYAMSWVRQAPGKGLEWVSAISGSGGSTYYADSVKGRFTISRDNSKNTLYLQMNSLRAEDTAVYYCARPYYDDWGQGTLVTVSS

SEQ ID NO: 59 - protein, 112 aa, Artificial Sequence
  Description of Artificial Sequence Synthetic polypeptide
QSVVTQPPSVSAAPGQKVTISCSGSSSNIGNNYVSWYQQLPGTAPKLLIYENNKRPSGIPDRFSGSKSGTSATLGITGLQTGDEADYYCGTWDSSLSAGAVFGGGTKLTVLG

SEQ ID NO: 60 - protein, 118 aa, Artificial Sequence
  Description of Artificial Sequence Synthetic polypeptide
QVQLVESGGGLVQPGGSLRLSCAASGFTFSNYAMSWVRQAPGKGLEWVSSISGSGGSTYYADSVKGRFTISRDTSKNTLYLQMNSLRAEDTAVYYCARYGSAAWMDSWGQGTLVTVSS

SEQ ID NO: 61 - protein, 108 aa, Artificial Sequence
  Description of Artificial Sequence Synthetic polypeptide
DIQLTQSPSSLSTSVGDRVTITCQASHDIRNYLNWYQQKPGKAPNLLIYAASNLQTGVPSRFSGRGSGTDFTLTISSLQPEDIATYYCQQYDGLPLTFGQGTRLEIKR

SEQ ID NO: 62 - protein, 130 aa, Artificial Sequence
  Description of Artificial Sequence Synthetic polypeptide
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQVWGWQGGMYPRSNWWYNLDSWGQGTLVTVSS

SEQ ID NO: 63 - protein, 109 aa, Artificial Sequence
  Description of Artificial Sequence Synthetic polypeptide
LPVLTQPPSVSVAPGKTARITCGGNNIGSKSVHWYQQKPGQAPVLVVYDDSDRPSGIPERFSGSNSGNTATLTISRVEAGDEADYYCQVWDSSSDYVVFGGGTKLTVLG

SEQ ID NO: 64 - protein, 121 aa, Artificial Sequence
  Description of Artificial Sequence Synthetic polypeptide
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSS

SEQ ID NO: 65 - protein, 107 aa, Artificial Sequence
  Description of Artificial Sequence Synthetic polypeptide
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKR

SEQ ID NO: 66 - protein, 115 aa, Artificial Sequence
  Description of Artificial Sequence Synthetic polypeptide
QVQLQESGPGLVKPSQTLSLTCTVSGYTFTNYYVFWVRQARGQRLEWIGDINPVNGDTNFNEKFKNRVTISADKSISTAYLQWSSLKASDTAMYYCARGGYTMDYWGQGTLVTVS

SEQ ID NO: 67 - protein, 113 aa, Artificial Sequence
  Description of Artificial Sequence Synthetic polypeptide
DIVMTQTPLSLPVTPGEPASISCRSSQSLVHSNGNTYLHWYQQKPGKAPKLLIYKVSYRFSGVPDRFSGSGSGTDFTLKISRVEAEDVGVYYCSQNTHVPRTFGQGTKVEIKR

SEQ ID NO: 68 - protein, 117 aa, Artificial Sequence
  Description of Artificial Sequence Synthetic polypeptide
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARWHGGPYDYWGQGTLVTVSS

SEQ ID NO: 69 - protein, 109 aa, Artificial Sequence
  Description of Artificial Sequence Synthetic polypeptide
QPVLTQPPSVSVAPGKTARITCGGNNIGSKSVHWYQQKPGQAPVLVIYYDSDRPSGIPERFSGSNSGNTATLTISRVEAGDEADYYCQVWDSSSDHYVFGTGTKVTVLG

SEQ ID NO: 70 - protein, 120 aa, Artificial Sequence
  Description of Artificial Sequence Synthetic polypeptide
QVQLVQSGADVRKPGASVKVSCKASGYTFASHGISWVRQAPGQGLEWLGWISPYTGNTNYAQKFQGRVTMATDTSTSTAYMELRSLRSDDTAIYYCARGKRTLASCFDYWGQGTLVTVSS

SEQ ID NO: 71 - protein, 108 aa, Artificial Sequence
  Description of Artificial Sequence Synthetic polypeptide
QSVLTQPPSVSVAPGKTARITCGGNNIGSKSVHWYQQKPGQAPVLVVYDDSDRPSGIPERFSGSNSGNTATLTISRVEAGDEADYYCQVWDSSSDHVFGTGTKVTVLG

SEQ ID NO: 72 - protein, 9 aa, Artificial Sequence
  Description of Artificial Sequence Synthetic peptide
PLFQVPEPV

SEQ ID NO: 73 - protein, 9 aa, Artificial Sequence
  Description of Artificial Sequence Synthetic peptide
FMNKFIYEI

SEQ ID NO: 74 - protein, 10 aa, Artificial Sequence
  Description of Artificial Sequence Synthetic peptide
GLSPNLNRFL

SEQ ID NO: 75 - protein, 9 aa, Artificial Sequence
  Description of Artificial Sequence Synthetic peptide
GVALQTMKQ

SEQ ID NO: 76 - protein, 9 aa, Artificial Sequence
  Description of Artificial Sequence Synthetic peptide
AMNKFIYEI

SEQ ID NO: 77 - protein, 9 aa, Artificial Sequence
  Description of Artificial Sequence Synthetic peptide
FMAKFIYEI

SEQ ID NO: 78 - protein, 9 aa, Artificial Sequence
  Description of Artificial Sequence Synthetic peptide
FMNAFIYEI

SEQ ID NO: 79 - protein, 9 aa, Artificial Sequence
  Description of Artificial Sequence Synthetic peptide
FMNKAIYEI

SEQ ID NO: 80 - protein, 9 aa, Artificial Sequence
  Description of Artificial Sequence Synthetic peptide
FMNKFAYEI

SEQ ID NO: 81 - protein, 9 aa, Artificial Sequence
  Description of Artificial Sequence Synthetic peptide
FMNKFIAEI

SEQ ID NO: 82 - protein, 9 aa, Artificial Sequence
  Description of Artificial Sequence Synthetic peptide
FMNKFIYAI

SEQ ID NO: 83 - protein, 8 aa, Artificial Sequence
  Description of Artificial Sequence Synthetic peptide
GYTFTSYG

SEQ ID NO: 84 - protein, 8 aa, Artificial Sequence
  Description of Artificial Sequence Synthetic peptide
ISAYNGNT

SEQ ID NO: 85 - protein, 13 aa, Artificial Sequence
  Description of Artificial Sequence Synthetic peptide
ARDSYYYYYGMDV

SEQ ID NO: 86 - protein, 120 aa, Artificial Sequence
  Description of Artificial Sequence Synthetic polypeptide
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDSYYYYYGMDVWGQGTTVTVSS

SEQ ID NO: 87 - protein, 9 aa, Artificial Sequence
  Description of Artificial Sequence Synthetic peptide
TGAVTSGHY

SEQ ID NO: 88 - protein, 3 aa, Artificial Sequence
  Description of Artificial Sequence Synthetic peptide
DAS

SEQ ID NO: 89 - protein, 9 aa, Artificial Sequence
  Description of Artificial Sequence Synthetic peptide
LLSYSDALV

SEQ ID NO: 90 - protein, 110 aa, Artificial Sequence
  Description of Artificial Sequence Synthetic polypeptide
QAVVTQEPSLTVSPGGTVTLTCGSSTGAVTSGHYPYWFQQKPGQAPRTLIYDASDKHSWTPARFSGSLLGGKAALTLSGAQPEDEAEYYCLLSYSDALVFGGGTKLTVLG

SEQ ID NO: 91 - protein, 8 aa, Artificial Sequence
  Description of Artificial Sequence Synthetic peptide
GYSFPNYW

SEQ ID NO: 92 - protein, 8 aa, Artificial Sequence
  Description of Artificial Sequence Synthetic peptide
IDPGDSYT

SEQ ID NO: 93 - protein, 10 aa, Artificial Sequence
  Description of Artificial Sequence Synthetic peptide
ARYYVSLVDI

SEQ ID NO: 94 - protein, 117 aa, Artificial Sequence
  Description of Artificial Sequence Synthetic polypeptide
EVQLVQSGAEVKKPGESLTISCKASGYSFPNYWITWVRQMSGGGLEWMGRIDPGDSYTTYNPSFQGHVTISIDKSTNTAYLHWNSLKASDTAMYYCARYYVSLVDIWGQGTLVTVSS

SEQ ID NO: 95 - protein, 9 aa, Artificial Sequence
  Description of Artificial Sequence Synthetic peptide
SSDVGGYNY

SEQ ID NO: 96 - protein, 3 aa, Artificial Sequence
  Description of Artificial Sequence Synthetic peptide
DVN

SEQ ID NO: 97 - protein, 10 aa, Artificial Sequence
  Description of Artificial Sequence Synthetic peptide
SSYTTGSRAV

SEQ ID NO: 98 - protein, 111 aa, Artificial Sequence
  Description of Artificial Sequence Synthetic polypeptide
QSVLTQPASVSGSPGQSITISCTGTSSDVGGYNYVSWYQQHPGKAPKLMIYDVNNRPSEVSNRFSGSKSGNTASLTISGLQAEDEADYYCSSYTTGSRAVFGGGTKLTVLG

SEQ ID NO: 99 - protein, 8 aa, Artificial Sequence
  Description of Artificial Sequence Synthetic peptide
GFTFSNAW

SEQ ID NO: 100 - protein, 10 aa, Artificial Sequence
  Description of Artificial Sequence Synthetic peptide
IRSKAYGGTT

SEQ ID NO: 101 - protein, 15 aa, Artificial Sequence
  Description of Artificial Sequence Synthetic peptide
ARDGLYSSSWYDSDY

SEQ ID NO: 102 - protein, 124 aa, Artificial Sequence
  Description of Artificial Sequence Synthetic polypeptide
EVQLVESGGGLVKPGGSLRLSCAASGFTFSNAWMSWVRQAPGKGLEWVGFIRSKAYGGTTEYAASVKGRFTISRDDSKSIAYLQMNNLKTEDTAVYYCARDGLYSSSWYDSDYWGQGTLVTVSS

SEQ ID NO: 103 - protein, 8 aa, Artificial Sequence
  Description of Artificial Sequence Synthetic peptide
SSNIGNNY

SEQ ID NO: 104 - protein, 3 aa, Artificial Sequence
  Description of Artificial Sequence Synthetic peptide
DNN

SEQ ID NO: 105 - protein, 11 aa, Artificial Sequence
  Description of Artificial Sequence Synthetic peptide
GTWDGSLYTML

SEQ ID NO: 106 - protein, 111 aa, Artificial Sequence
  Description of Artificial Sequence Synthetic polypeptide
QSVVTQPPSVSAAPGQKVTISCSGSSSNIGNNYVSWYQQLPGTAPKLLIYDNNKRPSGIPDRFSGSKSGTSATLGITGLQTGDEADYYCGTWDGSLYTMLFGGGTKLTVLG

SEQ ID NO: 107 - protein, 8 aa, Artificial Sequence
  Description of Artificial Sequence Synthetic peptide
GFTFDDYA

SEQ ID NO: 108 - protein, 8 aa, Artificial Sequence
  Description of Artificial Sequence Synthetic peptide
ISWNSGSI

SEQ ID NO: 109 - protein, 19 aa, Artificial Sequence
  Description of Artificial Sequence Synthetic peptide
AKDIHSGSYYGLLYYAMDV

SEQ ID NO: 110 - protein, 126 aa, Artificial Sequence
  Description of Artificial Sequence Synthetic polypeptide
QMQLVQSGGGLVQPGRSLRLSCAASGFTFDDYAMHWVRQAPGKGLEWVSGISWNSGSIGYADSVKGRFTISRDNAKNSLYLQMNSLRAEDTALYYCAKDIHSGSYYGLLYYAMDVWGQGTTVTVSS

SEQ ID NO: 111 - protein, 9 aa, Artificial Sequence
  Description of Artificial Sequence Synthetic peptide
SSNIGAGYD

SEQ ID NO: 112 - protein, 3 aa, Artificial Sequence
  Description of Artificial Sequence Synthetic peptide
GNS

SEQ ID NO: 113 - protein, 12 aa, Artificial Sequence
  Description of Artificial Sequence Synthetic peptide
QSYDSSLSGSGV

SEQ ID NO: 114 - protein, 113 aa, Artificial Sequence
  Description of Artificial Sequence Synthetic polypeptide
QSVLTQPPSVSGAPGQRVTISCTGSSSNIGAGYDVHWYQQLPGTAPKLLIFGNSNRPSGVPDRFSGFKSGTSASLAITGLQAEDEADYFCQSYDSSLSGSGVFGTGTKVTVLG

SEQ ID NO: 115 - protein, 8 aa, Artificial Sequence
  Description of Artificial Sequence Synthetic peptide
GYTFTSYG

SEQ ID NO: 116 - protein, 8 aa, Artificial Sequence
  Description of Artificial Sequence Synthetic peptide
ISAYNGNT

SEQ ID NO: 117 - protein, 14 aa, Artificial Sequence
  Description of Artificial Sequence Synthetic peptide
ARFQDWWYLGQFDQ

SEQ ID NO: 118 - protein, 121 aa, Artificial Sequence
  Description of Artificial Sequence Synthetic polypeptide
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARFQDWWYLGQFDQWGQGTLVTVSS

SEQ ID NO: 119 - protein, 9 aa, Artificial Sequence
  Description of Artificial Sequence Synthetic peptide
GSDVGVYYY

SEQ ID NO: 120 - protein, 3 aa, Artificial Sequence
  Description of Artificial Sequence Synthetic peptide
DVD

SEQ ID NO: 121 - protein, 12 aa, Artificial Sequence
  Description of Artificial Sequence Synthetic peptide
ASYTNRNSLGYV

SEQ ID NO: 122 - protein, 113 aa, Artificial Sequence
  Description of Artificial Sequence Synthetic polypeptide
QSALTQPASVSGSPGQSITISCTATGSDVGVYYYVSWYQQHPGKAPKVMIYDVDNRPPGVSNRFSGSKSGNTASLTISGLQAEDEADYYCASYTNRNSLGYVFGTGTKVTVLG

SEQ ID NO: 123 - protein, 8 aa, Artificial Sequence
  Description of Artificial Sequence Synthetic peptide
GGSFSGYY

SEQ ID NO: 124 - protein, 7 aa, Artificial Sequence
  Description of Artificial Sequence Synthetic peptide
INHSGST

SEQ ID NO: 125 - protein, 10 aa, Artificial Sequence
  Description of Artificial Sequence Synthetic peptide
ARGYGGRFDY

SEQ ID NO: 126 - protein, 116 aa, Artificial Sequence
  Description of Artificial Sequence Synthetic polypeptide
QVQLQQWGAGLLKPSETLSLTCAVYGGSFSGYYWSWIRQPPGKGLEWIGEINHSGSTNYNPSLKSRVTISVDTSKNQFSLELSSVTAADTAVYYCARGYGGRFDYWGQGTLVTVSS

SEQ ID NO: 127 - protein, 8 aa, Artificial Sequence
  Description of Artificial Sequence Synthetic peptide
SSNIGSNN

SEQ ID NO: 128 - protein, 3 aa, Artificial Sequence
  Description of Artificial Sequence Synthetic peptide
SNH

SEQ ID NO: 129 - protein, 11 aa, Artificial Sequence
  Description of Artificial Sequence Synthetic peptide
AAWDDSLDGYL

SEQ ID NO: 130 - protein, 111 aa, Artificial Sequence
  Description of Artificial Sequence Synthetic polypeptide
QPVLTQPPSASGTPGQRVTISCSGSSSNIGSNNVIWYQQLPGAAPKLLIYSNHRRPSGVPDRFSGSRSGTSASLAISGLQSEDEADYYCAAWDDSLDGYLFGTGTKVTVLG

SEQ ID NO: 131 - protein, 8 aa, Artificial Sequence
  Description of Artificial Sequence Synthetic peptide
GFTFSSYA

SEQ ID NO: 132 - protein, 8 aa, Artificial Sequence
  Description of Artificial Sequence Synthetic peptide
IYSGGSST

SEQ ID NO: 133 - protein, 11 aa, Artificial Sequence
  Description of Artificial Sequence Synthetic peptide
ARTSYLNHGDY

SEQ ID NO: 134 - protein, 118 aa, Artificial Sequence
  Description of Artificial Sequence Synthetic polypeptide
QVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSS

SEQ ID NO: 135 - protein, 8 aa, Artificial Sequence
  Description of Artificial Sequence Synthetic peptide
RSNIGSDY

SEQ ID NO: 136 - protein, 3 aa, Artificial Sequence
  Description of Artificial Sequence Synthetic peptide
GDN

SEQ ID NO: 137 - protein, 11 aa, Artificial Sequence
  Description of Artificial Sequence Synthetic peptide
GTWDYTLNGVV

SEQ ID NO: 138 - protein, 111 aa, Artificial Sequence
  Description of Artificial Sequence Synthetic polypeptide
QSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLG

SEQ ID NO: 139 - protein, 8 aa, Artificial Sequence
  Description of Artificial Sequence Synthetic peptide
GFTFSDYY

SEQ ID NO: 140 - protein, 8 aa, Artificial Sequence
  Description of Artificial Sequence Synthetic peptide
ISSSGSTI

SEQ ID NO: 141 - protein, 9 aa, Artificial Sequence
  Description of Artificial Sequence Synthetic peptide
ARASDLYGD

SEQ ID NO: 142 - protein, 116 aa, Artificial Sequence
  Description of Artificial Sequence Synthetic polypeptide
QMQLVQSGGGLVKPGGSLRLSCAASGFTFSDYYMSWIRQAPGKGLEWVSYISSSGSTIYYADSVKGRFTISRDNAKNSLYLQMNSLRAEDTAVYYCARASDLYGDWGQGTLVTVSS

SEQ ID NO: 143 - protein, 8 aa, Artificial Sequence
  Description of Artificial Sequence Synthetic peptide
TSNIGTNT

SEQ ID NO: 144 - protein, 3 aa, Artificial Sequence
  Description of Artificial Sequence Synthetic peptide
SNN

SEQ ID NO: 145 - protein, 11 aa, Artificial Sequence
  Description of Artificial Sequence Synthetic peptide
AAWDDSLNGVV

SEQ ID NO: 146 - protein, 111 aa, Artificial Sequence
  Description of Artificial Sequence Synthetic polypeptide
QSVLTQPPSVSGTPGQRVIISCPGSTSNIGTNTVNWYQQFPGTAPKLLIYSNNQRPSGVPDRFSGSKSGTSASLAISGLQSEDEADYYCAAWDDSLNGVVFGGGTKLTVLG

SEQ ID NO: 147 - protein, 8 aa, Artificial Sequence
  Description of Artificial Sequence Synthetic peptide
GYRFSNYG

SEQ ID NO: 148 - protein, 8 aa, Artificial Sequence
  Description of Artificial Sequence Synthetic peptide
ISGSNGNT

SEQ ID NO: 149 - protein, 14 aa, Artificial Sequence
  Description of Artificial Sequence Synthetic peptide
ARGNRRYYSPIIDP

SEQ ID NO: 150 - protein, 121 aa, Artificial Sequence
  Description of Artificial Sequence Synthetic polypeptide
QVQLVQSGAEVKKPGASVTVSCKASGYRFSNYGVSWVRQAPGQGLEWMGWISGSNGNTNYAQKFLGRVTMTTDTSTTTAYMELSSLRSDDTAVYYCARGNRRYYSPIIDPWGQGTLVTVSS

SEQ ID NO: 151 - protein, 8 aa, Artificial Sequence
  Description of Artificial Sequence Synthetic peptide
SSNFGSNT

SEQ ID NO: 152 - protein, 3 aa, Artificial Sequence
  Description of Artificial Sequence Synthetic peptide
SNT

SEQ ID NO: 153 - protein, 11 aa, Artificial Sequence
  Description of Artificial Sequence Synthetic peptide
AAWDDSLTGVV

SEQ ID NO: 154 - protein, 111 aa, Artificial Sequence
  Description of Artificial Sequence Synthetic polypeptide
QAVLTQPPSVSGTPGQRVTISCSGSSSNFGSNTVHWYQQVPGTAPKLLIFSNTQRPSEIPDRFSGSKSGTSASLAISGLQSEDEADYYCAAWDDSLTGVVFGGGTKLTVLG

SEQ ID NO: 155 - protein, 8 aa, Artificial Sequence
  Description of Artificial Sequence Synthetic peptide
GGTFGSYA

SEQ ID NO: 156 - protein, 8 aa, Artificial Sequence
  Description of Artificial Sequence Synthetic peptide
IIPVLGRT

SEQ ID NO: 157 - protein, 6 aa, Artificial Sequence
  Description of Artificial Sequence Synthetic peptide
ARTNDS

SEQ ID NO: 158 - protein, 113 aa, Artificial Sequence
  Description of Artificial Sequence Synthetic polypeptide
EVQLVQSGAEVRKPGSSVKVSCQASGGTFGSYAISWVRQAPGQGLEWMGRIIPVLGRTKYAQKFQGRVTVTADTSTSTVYMELTSLTSEDTAVYYCARTNDSWGQGTLVTVSS

SEQ ID NO: 159 - protein, 11 aa, Artificial Sequence
  Description of Artificial Sequence Synthetic peptide
QSLLHSNGYNY

SEQ ID NO: 160 - protein, 3 aa, Artificial Sequence
  Description of Artificial Sequence Synthetic peptide
LGS

SEQ ID NO: 161 - protein, 9 aa, Artificial Sequence
  Description of Artificial Sequence Synthetic peptide
MQALQTPWT

SEQ ID NO: 162 - protein, 113 aa, Artificial Sequence
  Description of Artificial Sequence Synthetic polypeptide
DVVMTQSPLSLPVTPGEPASVSCRSSQSLLHSNGYNYLDWYLQKPGQSPQLLIYLGSNRASGVPDRFSGSGSGTDFTLKISRVEAEDVGVYYCMQALQTPWTFGQGTKVEIKR

SEQ ID NO: 163 - protein, 8 aa, Artificial Sequence
  Description of Artificial Sequence Synthetic peptide
GYTFTSYY

SEQ ID NO: 164 - protein, 8 aa, Artificial Sequence
  Description of Artificial Sequence Synthetic peptide
INPSGGST

SEQ ID NO: 165 - protein, 10 aa, Artificial Sequence
  Description of Artificial Sequence Synthetic peptide
ARWHGGPYDY

SEQ ID NO: 166 - protein, 6 aa, Artificial Sequence
  Description of Artificial Sequence Synthetic peptide
NIGSKS

SEQ ID NO: 167 - protein, 3 aa, Artificial Sequence
  Description of Artificial Sequence Synthetic peptide
YDS

SEQ ID NO: 168 - protein, 11 aa, Artificial Sequence
  Description of Artificial Sequence Synthetic peptide
QVWDSSSDHYV

SEQ ID NO: 169 - protein, 8 aa, Artificial Sequence
  Description of Artificial Sequence Synthetic peptide
GYTFASHG

SEQ ID NO: 170 - protein, 8 aa, Artificial Sequence
  Description of Artificial Sequence Synthetic peptide
ISPYTGNT

SEQ ID NO: 171 - protein, 13 aa, Artificial Sequence
  Description of Artificial Sequence Synthetic peptide
ARGKRTLASCFDY

SEQ ID NO: 172 - protein, 6 aa, Artificial Sequence
  Description of Artificial Sequence Synthetic peptide
NIGSKS

SEQ ID NO: 173 - protein, 3 aa, Artificial Sequence
  Description of Artificial Sequence Synthetic peptide
DDS

SEQ ID NO: 174 - protein, 10 aa, Artificial Sequence
  Description of Artificial Sequence Synthetic peptide
QVWDSSSDHV

SEQ ID NO: 175 - protein, 10 aa, Artificial Sequence
  Description of Artificial Sequence Synthetic peptide
KLVVVGAGGV

SEQ ID NO: 176 - protein, 10 aa, Artificial Sequence
  Description of Artificial Sequence Synthetic peptide
KLVVVGAVGV

SEQ ID NO: 177 - protein, 10 aa, Artificial Sequence
  Description of Artificial Sequence Synthetic peptide
KLVVVGACGV

SEQ ID NO: 178 - protein, 10 aa, Artificial Sequence
  Description of Artificial Sequence Synthetic peptide
KLVVVGADGV

SEQ ID NO: 179 - protein, 10 aa, Artificial Sequence
  Description of Artificial Sequence Synthetic peptide
KLVVVGASGV

SEQ ID NO: 180 - protein, 9 aa, Artificial Sequence
  Description of Artificial Sequence Synthetic peptide
LVVVGAGGV

SEQ ID NO: 181 - protein, 9 aa, Artificial Sequence
  Description of Artificial Sequence Synthetic peptide
LVVVGAVGV

SEQ ID NO: 182 - protein, 9 aa, Artificial Sequence
  Description of Artificial Sequence Synthetic peptide
LVVVGACGV

SEQ ID NO: 183 - protein, 9 aa, Artificial Sequence
  Description of Artificial Sequence Synthetic peptide
LVVVGADGV

SEQ ID NO: 184 - protein, 8 aa, Artificial Sequence
  Description of Artificial Sequence Synthetic peptide
GGTFSSYA

SEQ ID NO: 185 - protein, 8 aa, Artificial Sequence
  Description of Artificial Sequence Synthetic peptide
IIPIFGKG

SEQ ID NO: 186 - protein, 11 aa, Artificial Sequence
  Description of Artificial Sequence Synthetic peptide
ARHIPTFSFDY

SEQ ID NO: 187 - protein, 118 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGKGNYPQKFQGRVTITADESTGTAYMELSSLRSEDTAVYYCARHIPTFSFDYWGQGTLVTVSS

SEQ ID NO: 188 - protein, 9 aa, Artificial Sequence
  Description of Artificial Sequence Synthetic peptide
SSNIGAGYD

SEQ ID NO: 189 - protein, 3 aa, Artificial Sequence
  Description of Artificial Sequence Synthetic peptide
GNS

SEQ ID NO: 190 - protein, 11 aa, Artificial Sequence
  Description of Artificial Sequence Synthetic peptide
QSYDSSLSGYV

SEQ ID NO: 191 - protein, 112 aa, Artificial Sequence
  Description of Artificial Sequence Synthetic polypeptide
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGYVFGTGTKVTVLG

SEQ ID NO: 192 - protein, 251 aa, Artificial Sequence
  Description of Artificial Sequence Synthetic polypeptide
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGYVFGTGTKVTVLGSRGGGGSGGGGSGGGGSLEMAQVQLVQSGAEVKKPGSSVKVSCKASGGTFSSYAISWVRQAPGQGLEWMGGIIPIFGKGNYPQKFQGRVTITADESTGTAYMELSSLRSEDTAVYYCARHIPTFSFDYWGQGTLVTVSS

SEQ ID NO: 193 - protein, 8 aa, Artificial Sequence
  Description of Artificial Sequence Synthetic peptide
GGTFSSYT

SEQ ID NO: 194 - protein, 8 aa, Artificial Sequence
  Description of Artificial Sequence Synthetic peptide
FIPISGTV

SEQ ID NO: 195 - protein, 10 aa, Artificial Sequence
  Description of Artificial Sequence Synthetic peptide
ARPLDWTEDI

SEQ ID NO: 196 - protein, 117 aa, Artificial Sequence
  Description of Artificial Sequence Synthetic polypeptide
QVQLVQSGAEVKKPGSSVKVSCKASGGTFSSYTINWVRQAPGQGLEWMGGFIPISGTVNYAQKFQGRVTITADESTSTAYMELSSLRSEDTAVYYCARPLDWTEDIWGQGTLVTVSS

SEQ ID NO: 197 - protein, 9 aa, Artificial Sequence
  Description of Artificial Sequence Synthetic peptide
SSNIGAGYD

SEQ ID NO: 198 - protein, 3 aa, Artificial Sequence
  Description of Artificial Sequence Synthetic peptide
GNS

SEQ ID NO: 199 - protein, 11 aa, Artificial Sequence
  Description of Artificial Sequence Synthetic peptide
QSYDSSLSGSV

SEQ ID NO: 200 - protein, 112 aa, Artificial Sequence
  Description of Artificial Sequence Synthetic polypeptide
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSVFGTGTKVTVLG

SEQ ID NO: 201 - protein, 249 aa, Artificial Sequence
  Description of Artificial Sequence Synthetic polypeptide
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSVFGTGTKVTVLGSRGGGGSGGGGSGGGGSLEMAVQLVQSGAEVKKPGSSVKVSCKASGGTFSSYTINWVRQAPGQGLEWMGGFIPISGTVNYAQKFQGRVTITADESTSTAYMELSSLRSEDTAVYYCARPLDWTEDIWGQGTLVTVSS

SEQ ID NO: 202 - protein, 8 aa, Artificial Sequence
  Description of Artificial Sequence Synthetic peptide
GYTFTAYY

SEQ ID NO: 203 - protein, 8 aa, Artificial Sequence
  Description of Artificial Sequence Synthetic peptide
MNTNNGAT

SEQ ID NO: 204 - protein, 12 aa, Artificial Sequence
  Description of Artificial Sequence Synthetic peptide
ARGDISQDFADV

SEQ ID NO: 205 - protein, 119 aa, Artificial Sequence
  Description of Artificial Sequence Synthetic polypeptide
EVQLVQSGAEVKKPGASVKVSCKASGYTFTAYYLHWLRQAPGQGLEWMGWMNTNNGATRYAQKFQDRVTMTRDTSINTAYMEMSGLSSDDTAMYYCARGDISQDFADVWGQGTLVTVSS

SEQ ID NO: 206 - protein, 8 aa, Artificial Sequence
  Description of Artificial Sequence Synthetic peptide
SGSIASNY

SEQ ID NO: 207 - protein, 3 aa, Artificial Sequence
  Description of Artificial Sequence Synthetic peptide
EDN

SEQ ID NO: 208 - protein, 10 aa, Artificial Sequence
  Description of Artificial Sequence Synthetic peptide
QSYDDINHWV

SEQ ID NO: 209 - protein, 112 aa, Artificial Sequence
  Description of Artificial Sequence Synthetic polypeptide
NFMLTQPHSVSESPGKTVTISCTGSSGSIASNYVQWYQQRPGSAPTILIYEDNKRPSGVPDRFSGSIDSSSNSASLTISGLKTGDEADYYCQSYDDINHWVFGGGTKLTVLG

SEQ ID NO: 210 - protein, 252 aa, Artificial Sequence
  Description of Artificial Sequence Synthetic polypeptide
NFMLTQPHSVSESPGKTVTISCTGSSGSIASNYVQWYQQRPGSAPTILIYEDNKRPSGVPDRFSGSIDSSSNSASLTISGLKTGDEADYYCQSYDDINHWVFGGGTKLTVLGSRGGGGSGGGGSGGGGSLEMAEVQLVQSGAEVKKPGASVKVSCKASGYTFTAYYLHWLRQAPGQGLEWMGWMNTNNGATRYAQKFQDRVTMTRDTSINTAYMEMSGLSSDDTAMYYCARGDISQDFADVWGQGTLVTVSS

SEQ ID NO: 211 - protein, 8 aa, Artificial Sequence
  Description of Artificial Sequence Synthetic peptide
GYTFTAYY

SEQ ID NO: 212 - protein, 8 aa, Artificial Sequence
  Description of Artificial Sequence Synthetic peptide
MNTNNGAT

SEQ ID NO: 213 - protein, 12 aa, Artificial Sequence
  Description of Artificial Sequence Synthetic peptide
ARGDISQDFADV

SEQ ID NO: 214 - protein, 119 aa, Artificial Sequence; identical to SEQ ID NO: 205
  Description of Artificial Sequence Synthetic polypeptide

SEQ ID NO: 215 - protein, 8 aa, Artificial Sequence
  Description of Artificial Sequence Synthetic peptide
SGSIASNY

SEQ ID NO: 216 - protein, 3 aa, Artificial Sequence
  Description of Artificial Sequence Synthetic peptide
EDN

SEQ ID NO: 217 - protein, 10 aa, Artificial Sequence
  Description of Artificial Sequence Synthetic peptide
QSYDDINHWV

SEQ ID NO: 218 - protein, 112 aa, Artificial Sequence; identical to SEQ ID NO: 209
  Description of Artificial Sequence Synthetic polypeptide

SEQ ID NO: 219 - protein, 252 aa, Artificial Sequence; identical to SEQ ID NO: 210
  Description of Artificial Sequence Synthetic polypeptide

SEQ ID NO: 220 - protein, 8 aa, Artificial Sequence
  Description of Artificial Sequence Synthetic peptide
GGSFSGYY

SEQ ID NO: 221 - protein, 7 aa, Artificial Sequence
  Description of Artificial Sequence Synthetic peptide
VNHSGNT

SEQ ID NO: 222 - protein, 10 aa, Artificial Sequence
  Description of Artificial Sequence Synthetic peptide
ARYFPPMIDV

SEQ ID NO: 223 - protein, 116 aa, Artificial Sequence
  Description of Artificial Sequence Synthetic polypeptide
QVQLQQWGAGLLKPSETLSLTCAVYGGSFSGYYWSWIRQSPGKGLEWIGEVNHSGNTNYNPSLKSRVTISLDTSKNQFSLKLNSVTAADTAVYYCARYFPPMIDVWGQGTLVTVSS

SEQ ID NO: 224 - protein, 8 aa, Artificial Sequence
  Description of Artificial Sequence Synthetic peptide
SSNIENNY

SEQ ID NO: 225 - protein, 3 aa, Artificial Sequence
  Description of Artificial Sequence Synthetic peptide
DNN

SEQ ID NO: 226 - protein, 11 aa, Artificial Sequence
  Description of Artificial Sequence Synthetic peptide
GTWDSSLSAYV

SEQ ID NO: 227 - protein, 112 aa, Artificial Sequence
  Description of Artificial Sequence Synthetic polypeptide
GQSVVTQPPSVSAAPGQKVTISCSGSSSNIENNYVSWYQQLPGTAPKLLIYDNNKRPSGIPDRFSGSKSGTSATLGITGLQTGDEADYYCGTWDSSLSAYVFGTGTKVTVLG

SEQ ID NO: 228 - protein, 248 aa, Artificial Sequence
  Description of Artificial Sequence Synthetic polypeptide
QSVVTQPPSVSAAPGQKVTISCSGSSSNIENNYVSWYQQLPGTAPKLLIYDNNKRPSGIPDRFSGSKSGTSATLGITGLQTGDEADYYCGTWDSSLSAYVFGTGTKVTVLGSRGGGGSGGGGSGGGGSLEMAQVQLQQWGAGLLKPSETLSLTCAVYGGSFSGYYWSWIRQSPGKGLEWIGEVNHSGNTNYNPSLKSRVTISLDTSKNQFSLKLNSVTAADTAVYYCARYFPPMIDVWGQGTLVTVSS

SEQ ID NO: 229 - protein, 10 aa, Artificial Sequence
  Description of Artificial Sequence Synthetic peptide
GGSISSSSYY

SEQ ID NO: 230 - protein, 7 aa, Artificial Sequence
  Description of Artificial Sequence Synthetic peptide
INHSGST

SEQ ID NO: 231 - protein, 14 aa, Artificial Sequence
  Description of Artificial Sequence Synthetic peptide
ARYSHHVDSGGYDV

SEQ ID NO: 232 - protein, 122 aa, Artificial Sequence
  Description of Artificial Sequence Synthetic polypeptide
QLQLQESGPGLVKPSETLSLSCTVSGGSISSSSYYWGWIRQPPGKGLEWIGEINHSGSTNYNPSLKSRVTISVDTSKNQFSLKLSSVTAADTAVYYCARYSHHVDSGGYDVWGQGTLVTVSS

SEQ ID NO: 233 - protein, 8 aa, Artificial Sequence
  Description of Artificial Sequence Synthetic peptide
SSNIGNNY

SEQ ID NO: 234 - protein, 3 aa, Artificial Sequence
  Description of Artificial Sequence Synthetic peptide
DNN

SEQ ID NO: 235 - protein, 11 aa, Artificial Sequence
  Description of Artificial Sequence Synthetic peptide
GTWDSSLSAVV

SEQ ID NO: 236 - protein, 111 aa, Artificial Sequence
  Description of Artificial Sequence Synthetic polypeptide
QSVVTQPPSVSAAPGQKVTISCSGSSSNIGNNYVSWYQQLPRTAPRLLIYDNNKRPSGIPDRFSASKSGTSATLGITGLQTGDEADYYCGTWDSSLSAVVFGGGTKLTVLG

SEQ ID NO: 237 - protein, 254 aa, Artificial Sequence
  Description of Artificial Sequence Synthetic polypeptide
QSVVTQPPSVSAAPGQKVTISCSGSSSNIGNNYVSWYQQLPRTAPRLLIYDNNKRPSGIPDRFSASKSGTSATLGITGLQTGDEADYYCGTWDSSLSAVVFGGGTKLTVLGSRGGGGSGGGGSGGGGSLEMAQLQLQESGPGLVKPSETLSLSCTVSGGSISSSSYYWGWIRQPPGKGLEWIGEINHSGSTNYNPSLKSRVTISVDTSKNQFSLKLSSVTAADTAVYYCARYSHHVDSGGYDVWGQGTLVTVSS

SEQ ID NO: 238 - protein, 8 aa, Artificial Sequence
  Description of Artificial Sequence Synthetic peptide
GGTFSSYG

SEQ ID NO: 239 - protein, 8 aa, Artificial Sequence
  Description of Artificial Sequence Synthetic peptide
IIPIFGTP

SEQ ID NO: 240 - protein, 10 aa, Artificial Sequence
  Description of Artificial Sequence Synthetic peptide
ARSYYGYFDG

SEQ ID NO: 241 - protein, 117 aa, Artificial Sequence
  Description of Artificial Sequence Synthetic polypeptide
EVQLVESGAEVKEPGSSVKVSCKASGGTFSSYGISWIRQAPGQGLEWMGEIIPIFGTPNYAQKFQGRVTITADESTSTAYVELSSLRSDDTAVYYCARSYYGYFDGWGQGTLVTVSS

SEQ ID NO: 242 - protein, 6 aa, Artificial Sequence
  Description of Artificial Sequence Synthetic peptide
QDISNY

SEQ ID NO: 243 - protein, 3 aa, Artificial Sequence
  Description of Artificial Sequence Synthetic peptide
DAS

SEQ ID NO: 244 - protein, 9 aa, Artificial Sequence
  Description of Artificial Sequence Synthetic peptide
QQYKSYPLT

SEQ ID NO: 245 - protein, 108 aa, Artificial Sequence
  Description of Artificial Sequence Synthetic polypeptide
DIQMTQSPSSLSASVGDRVTITCQASQDISNYLNWYQQKPGKAPKLLIYDASNLETGVPSRFSGSGSGTDFTFTISSLQPDDFATYYCQQYKSYPLTFGGGTKVEIKR

SEQ ID NO: 246 - protein, 246 aa, Artificial Sequence
  Description of Artificial Sequence Synthetic polypeptide
DIQMTQSPSSLSASVGDRVTITCQASQDISNYLNWYQQKPGKAPKLLIYDASNLETGVPSRFSGSGSGTDFTFTISSLQPDDFATYYCQQYKSYPLTFGGGTKVEIKRSRGGGGSGGGGSGGGGSLEMAEVQLVESGAEVKEPGSSVKVSCKASGGTFSSYGISWIRQAPGQGLEWMGEIIPIFGTPNYAQKFQGRVTITADESTSTAYVELSSLRSDDTAVYYCARSYYGYFDGWGQGTLVTVSS

SEQ ID NO: 247 - protein, 8 aa, Artificial Sequence
  Description of Artificial Sequence Synthetic peptide
GYTFTSYY

SEQ ID NO: 248 - protein, 8 aa, Artificial Sequence
  Description of Artificial Sequence Synthetic peptide
INPSGGST

SEQ ID NO: 249 - protein, 11 aa, Artificial Sequence
  Description of Artificial Sequence Synthetic peptide
ARSMYQYFLDS

SEQ ID NO: 250 - protein, 118 aa, Artificial Sequence
  Description of Artificial Sequence Synthetic polypeptide
EVQLVESGAEVKKPGASVKISCKASGYTFTSYYMHWVRQAPGQGLEWMGIINPSGGSTSYAQKFQGRVTMTRDTSTSTVYMELSSLRSEDTAVYYCARSMYQYFLDSWGQGTLVTVSS

SEQ ID NO: 251 - protein, 9 aa, Artificial Sequence
  Description of Artificial Sequence Synthetic peptide
SSNIGAGYD

SEQ ID NO: 252 - protein, 3 aa, Artificial Sequence
  Description of Artificial Sequence Synthetic peptide
GNI

SEQ ID NO: 253 - protein, 9 aa, Artificial Sequence
  Description of Artificial Sequence Synthetic peptide
QSYDSNLSG

SEQ ID NO: 254 - protein, 112 aa, Artificial Sequence
  Description of Artificial Sequence Synthetic polypeptide
QSVVTQPPSVSGAPGQRVTISCTGSSSNIGAGYDVHWYQQLPGTAPKLLIYGNINRPSGVPDRFSGSKSGTSASLAITGLQAEDEADYYCQSYDSNLSGYVFATGTKVTVLG

SEQ ID NO: 255 - protein, 251 aa, Artificial Sequence
  Description of Artificial Sequence Synthetic polypeptide
QSVVTQPPSVSGAPGQRVTISCTGSSSNIGAGYDVHWYQQLPGTAPKLLIYGNINRPSGVPDRFSGSKSGTSASLAITGLQAEDEADYYCQSYDSNLSGYVFATGTKVTVLGSRGGGGSGGGGSGGGGSLEMAEVQLVESGAEVKKPGASVKISCKASGYTFTSYYMHWVRQAPGQGLEWMGIINPSGGSTSYAQKFQGRVTMTRDTSTSTVYMELSSLRSEDTAVYYCARSMYQYFLDSWGQGTLVTVSS

SEQ ID NO: 256 - protein, 9 aa, Artificial Sequence
  Description of Artificial Sequence Synthetic peptide
QLSLLMWIT

SEQ ID NO: 257 - protein, 9 aa, Artificial Sequence
  Description of Artificial Sequence Synthetic peptide
SLLMWITQC

SEQ ID NO: 258 - protein, 11 aa, Artificial Sequence
  Description of Artificial Sequence Synthetic peptide
SLLMWITQCFL

SEQ ID NO: 259 - protein, 9 aa, Artificial Sequence
  Description of Artificial Sequence Synthetic peptide
SLLMWITQV

SEQ ID NO: 260 - protein, 9 aa, Artificial Sequence
  Description of Artificial Sequence Synthetic peptide
ALLMWITQC

SEQ ID NO: 261 - protein, 9 aa, Artificial Sequence
  Description of Artificial Sequence Synthetic peptide
SALMWITQC

SEQ ID NO: 262 - protein, 9 aa, Artificial Sequence
  Description of Artificial Sequence Synthetic peptide
SLAMWITQC

SEQ ID NO: 263 - protein, 9 aa, Artificial Sequence
  Description of Artificial Sequence Synthetic peptide
SLLAWITQC

SEQ ID NO: 264 - protein, 9 aa, Artificial Sequence
  Description of Artificial Sequence Synthetic peptide
SLLMAITQC

SEQ ID NO: 265 - protein, 9 aa, Artificial Sequence
  Description of Artificial Sequence Synthetic peptide
SLLMWATQC

SEQ ID NO: 266 - protein, 9 aa, Artificial Sequence
  Description of Artificial Sequence Synthetic peptide
SLLMWIAQC

SEQ ID NO: 267 - protein, 8 aa, Artificial Sequence
  Description of Artificial Sequence Synthetic peptide
GDTFSSYS

SEQ ID NO: 268 - protein, 8 aa, Artificial Sequence
  Description of Artificial Sequence Synthetic peptide
FIPNLNKG

SEQ ID NO: 269 - protein, 10 aa, Artificial Sequence
  Description of Artificial Sequence Synthetic peptide
ARDWSYSIDY

SEQ ID NO: 270 - protein, 117 aa, Artificial Sequence
  Description of Artificial Sequence Synthetic polypeptide
QVQLVQSGAEVKKPGSSVKVSCKASGDTFSSYSISWVRQAPGQGLEWMGRIIPILGIANYAQKYQGRVTLSADKSTSTSYMELNSLRSEDTAVYYCARDWSYSIDYWGQGTLVTVSS

SEQ ID NO: 271 - protein, 8 aa, Artificial Sequence
  Description of Artificial Sequence Synthetic peptide
SSNIGNNY

SEQ ID NO: 272 - protein, 3 aa, Artificial Sequence
  Description of Artificial Sequence Synthetic peptide
DNN

SEQ ID NO: 273 - protein, 11 aa, Artificial Sequence
  Description of Artificial Sequence Synthetic peptide
GTWDSSLSAWV

SEQ ID NO: 274 - protein, 111 aa, Artificial Sequence
  Description of Artificial Sequence Synthetic polypeptide
QSVVTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAWVFGGGTKLTVLG

SEQ ID NO: 275 - protein, 8 aa, Artificial Sequence
  Description of Artificial Sequence Synthetic peptide
GYTFTSYG

SEQ ID NO: 276 - protein, 8 aa, Artificial Sequence
  Description of Artificial Sequence Synthetic peptide
ISAYNGNT

SEQ ID NO: 277 - protein, 11 aa, Artificial Sequence
  Description of Artificial Sequence Synthetic peptide
ARYSGYYAGDS

SEQ ID NO: 278 - protein, 118 aa, Artificial Sequence
  Description of Artificial Sequence Synthetic polypeptide
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARYSGYYAGDSWGQGTLVTVSS

SEQ ID NO: 279 - protein, 9 aa, Artificial Sequence
  Description of Artificial Sequence Synthetic peptide
SSNIGAGYD

SEQ ID NO: 280 - protein, 3 aa, Artificial Sequence
  Description of Artificial Sequence Synthetic peptide
GDT

SEQ ID NO: 281 - protein, 11 aa, Artificial Sequence
  Description of Artificial Sequence Synthetic peptide
QSYDSNLYTYV

SEQ ID NO: 282 - protein, 112 aa, Artificial Sequence
  Description of Artificial Sequence Synthetic polypeptide
QSVLTQPPSVSGAPGQRVTISCTGSSSNIGAGYDVHWYQQLPGTAPKLLIYGDTNRPSGVPDRISGSKSGTSASLAITGLQAEDEADYYCQSYDSNLYTYVFGTGTKVTVLG

SEQ ID NO: 283 - protein, 8 aa, Artificial Sequence
  Description of Artificial Sequence Synthetic peptide
GYTFTSYG

SEQ ID NO: 284 - protein, 8 aa, Artificial Sequence
  Description of Artificial Sequence Synthetic peptide
FIPNLNKG

SEQ ID NO: 285 - protein, 9 aa, Artificial Sequence
  Description of Artificial Sequence Synthetic peptide
ARGDYGSDQ

SEQ ID NO: 286 - protein, 116 aa, Artificial Sequence
  Description of Artificial Sequence Synthetic polypeptide
EVQLVESGAEVKRPGASVKVSCKASGYTFTSYGISWVRQAPGQGLEWMGRFIPNLNKGNSAHKFEGRVSFTADKFTNTAYMELRGLKSDDTAVYYCARGDYGSDQWGQGTLVTVSS

SEQ ID NO: 287 - protein, 8 aa, Artificial Sequence
  Description of Artificial Sequence Synthetic peptide
SSNIGAGY

SEQ ID NO: 288 - protein, 3 aa, Artificial Sequence
  Description of Artificial Sequence Synthetic peptide
GNS

SEQ ID NO: 289 - protein, 12 aa, Artificial Sequence
  Description of Artificial Sequence Synthetic peptide
QSYDSSLSGSWV

SEQ ID NO: 290 - protein, 113 aa, Artificial Sequence
  Description of Artificial Sequence Synthetic polypeptide
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSWVFGGGTKLTVLG

SEQ ID NO: 291 - protein, 8 aa, Artificial Sequence
  Description of Artificial Sequence Synthetic peptide
GGTFSSYA

SEQ ID NO: 292 - protein, 8 aa, Artificial Sequence
  Description of Artificial Sequence Synthetic peptide
IIPIFGTA

SEQ ID NO: 293 - protein, 10 aa, Artificial Sequence
  Description of Artificial Sequence Synthetic peptide
ARYDSYVYDE

SEQ ID NO: 294 - protein, 117 aa, Artificial Sequence
  Description of Artificial Sequence Synthetic polypeptide
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCARYDSYVYDEWGQGTLVTVSS

SEQ ID NO: 295 - protein, 9 aa, Artificial Sequence
  Description of Artificial Sequence Synthetic peptide
GSNIGAGYD

SEQ ID NO: 296 - protein, 3 aa, Artificial Sequence
  Description of Artificial Sequence Synthetic peptide
GNS

SEQ ID NO: 297 - protein, 12 aa, Artificial Sequence
  Description of Artificial Sequence Synthetic peptide
QSYDSSLSGWGI

SEQ ID NO: 298 - protein, 113 aa, Artificial Sequence
  Description of Artificial Sequence Synthetic polypeptide
QSVVTQPPSLSGAPGQRVTISCNGSGSNIGAGYDVHWYQQLPGTAPKLLIYGNSNRPSGVPDRFSGSKSGTSASLAITGLQAEDEADYYCQSYDSSLSGWGIFGGGTKLTVLG

SEQ ID NO: 299 - protein, 8 aa, Artificial Sequence
  Description of Artificial Sequence Synthetic peptide
GYTFTKYG

SEQ ID NO: 300 - protein, 8 aa, Artificial Sequence
  Description of Artificial Sequence Synthetic peptide
ISADSGKT

SEQ ID NO: 301 - protein, 6 aa, Artificial Sequence
  Description of Artificial Sequence Synthetic peptide
ARDDDS

SEQ ID NO: 302 - protein, 113 aa, Artificial Sequence
  Description of Artificial Sequence Synthetic polypeptide
QVQLVQSGPEVKKPGASMKVSCKASGYTFTKYGISWVRQAPGQGLEWMGWISADSGKTSYAQNLQGRVSLTIDTSTATAYMELRSLRSDDTAVYYCARDDDSWGQGTLVTVSS

SEQ ID NO: 303 - protein, 8 aa, Artificial Sequence
  Description of Artificial Sequence Synthetic peptide
SSNIGNNY

SEQ ID NO: 304 - protein, 3 aa, Artificial Sequence
  Description of Artificial Sequence Synthetic peptide
DNN

SEQ ID NO: 305 - protein, 11 aa, Artificial Sequence
  Description of Artificial Sequence Synthetic peptide
GTWDSSLSAEV

SEQ ID NO: 306 - protein, 111 aa, Artificial Sequence
  Description of Artificial Sequence Synthetic polypeptide
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAEVFGTGTKVTVLG

SEQ ID NO: 307 - protein, 8 aa, Artificial Sequence
  Description of Artificial Sequence Synthetic peptide
GYTLTDLP

SEQ ID NO: 308 - protein, 8 aa, Artificial Sequence
  Description of Artificial Sequence Synthetic peptide
FDPEDGEI

SEQ ID NO: 309 - protein, 12 aa, Artificial Sequence
  Description of Artificial Sequence Synthetic peptide
ARYVPYVSYSDS

SEQ ID NO: 310 - protein, 119 aa, Artificial Sequence
  Description of Artificial Sequence Synthetic polypeptide
EVQLVQSGAEVKKPGASVKVSCKVSGYTLTDLPMHWVRQAPGKGLEWMGGFDPEDGEIIYAQKFQGRVTMTEDTFTDTAYVELSSLRSEDTAVYYCARYVPYVSYSDSWGQGTLVTVSS

SEQ ID NO: 311 - protein, 11 aa, Artificial Sequence
  Description of Artificial Sequence Synthetic peptide
QSLLHSNGYNY

SEQ ID NO: 312 - protein, 3 aa, Artificial Sequence
  Description of Artificial Sequence Synthetic peptide
LGS

SEQ ID NO: 313 - protein, 9 aa, Artificial Sequence
  Description of Artificial Sequence Synthetic peptide
MQALQTPYT

SEQ ID NO: 314 - protein, 113 aa, Artificial Sequence
  Description of Artificial Sequence Synthetic polypeptide
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPYTFGQGTKLEIKR

SEQ ID NO: 315 - protein, 8 aa, Artificial Sequence
  Description of Artificial Sequence Synthetic peptide
GGTFSSYA

SEQ ID NO: 316 - protein, 8 aa, Artificial Sequence
  Description of Artificial Sequence Synthetic peptide
IIPIFGTA

SEQ ID NO: 317 - protein, 12 aa, Artificial Sequence
  Description of Artificial Sequence Synthetic peptide
ARSYWSWTPYDP

SEQ ID NO: 318 - protein, 119 aa, Artificial Sequence
  Description of Artificial Sequence Synthetic polypeptide
EVQLVQSGAEVKKPGASVKVSCKASGGTFSSYAISWVRQAPGQGLEWMGGIIPIFGTANYAQKFQGRVTITADKSTSTAYMELSSLRSEDTAVYYCARSYWSWTPYDPWGQGTLVTVSS

SEQ ID NO: 319 - protein, 6 aa, Artificial Sequence
  Description of Artificial Sequence Synthetic peptide
NIGSKS

SEQ ID NO: 320 - protein, 3 aa, Artificial Sequence
  Description of Artificial Sequence Synthetic peptide
YDS

SEQ ID NO: 321 - protein, 11 aa, Artificial Sequence
  Description of Artificial Sequence Synthetic peptide
QVWDSSSDHYV

SEQ ID NO: 322 - protein, 109 aa, Artificial Sequence
  Description of Artificial Sequence Synthetic polypeptide
LPVLTQPPSVSVAPGKTARITCGGNNIGSKSVHWYQQKPGQAPVLVIYYDSDRPSGIPERFSGSNSGNTATLTISRVEAGDEADYYCQVWDSSSDHYVFGTGTKVTVLG

SEQ ID NO: 323 - protein, 9 aa, Artificial Sequence
  Description of Artificial Sequence Synthetic peptide
VLDGLDVLL

SEQ ID NO: 324 - protein, 10 aa, Artificial Sequence
  Description of Artificial Sequence Synthetic peptide
SLYSFPEPEA

SEQ ID NO: 325 - protein, 10 aa, Artificial Sequence
  Description of Artificial Sequence Synthetic peptide
ALYVDSLFFL

SEQ ID NO: 326 - protein, 9 aa, Artificial Sequence
  Description of Artificial Sequence Synthetic peptide
SLLQHLIGL

SEQ ID NO: 327 - protein, 9 aa, Artificial Sequence
  Description of Artificial Sequence Synthetic peptide
NLTHVLYPV

SEQ ID NO: 328 - protein, 8 aa, Artificial Sequence
  Description of Artificial Sequence Synthetic peptide
GGTFSSYA

SEQ ID NO: 329 - protein, 8 aa, Artificial Sequence
  Description of Artificial Sequence Synthetic peptide
IIPILGIA

SEQ ID NO: 330 - protein, 10 aa, Artificial Sequence
  Description of Artificial Sequence Synthetic peptide
ARHYGQWWDY

SEQ ID NO: 331 - protein, 117 aa, Artificial Sequence
  Description of Artificial Sequence Synthetic polypeptide
QVQLVQSGAEVRKPGASVKVSCKASGGTFSSYAISWVRQAPGQGLEWMGRIIPILGIANYAQKFQGRVTITADKSTSTAYMELSSLRSEDTAVYYCARHYGQWWDYWGQGTLVTVSS

SEQ ID NO: 332 - protein, 8 aa, Artificial Sequence
  Description of Artificial Sequence Synthetic peptide
SSNIGSNT

SEQ ID NO: 333 - protein, 3 aa, Artificial Sequence
  Description of Artificial Sequence Synthetic peptide
SNN

SEQ ID NO: 334 - protein, 12 aa, Artificial Sequence
  Description of Artificial Sequence Synthetic peptide
AAWDDSLNGSYV

SEQ ID NO: 335 - protein, 112 aa, Artificial Sequence
  Description of Artificial Sequence Synthetic polypeptide
QAVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGSYVFGTGTKVTVLG

SEQ ID NO: 336 - protein, 8 aa, Artificial Sequence
  Description of Artificial Sequence Synthetic peptide
GGTFSSHP

SEQ ID NO: 337 - protein, 8 aa, Artificial Sequence
  Description of Artificial Sequence Synthetic peptide
IIPMLDIP

SEQ ID NO: 338 - protein, 9 aa, Artificial Sequence
  Description of Artificial Sequence Synthetic peptide
ARGLYYYDY

SEQ ID NO: 339 - protein, 116 aa, Artificial Sequence
  Description of Artificial Sequence Synthetic polypeptide
QVQLVQSGAEVKKPGSSVKVSCKASGGTFSSHPISWVRQAPGQGLEWMGRIIPMLDIPNNAQKFQGRVTITADKSTDTAYLELSSLTSEDTAVYYCARGLYYYDYWGQGTLVTVSS

SEQ ID NO: 340 - protein, 9 aa, Artificial Sequence
  Description of Artificial Sequence Synthetic peptide
TSNIGAGFD

SEQ ID NO: 341 - protein, 3 aa, Artificial Sequence
  Description of Artificial Sequence Synthetic peptide
GNT

SEQ ID NO: 342 - protein, 11 aa, Artificial Sequence
  Description of Artificial Sequence Synthetic peptide
QSYDRSLSTIL

SEQ ID NO: 343 - protein, 112 aa, Artificial Sequence
  Description of Artificial Sequence Synthetic polypeptide
QSVVTQPPAVSGALGQRVTISCTGTTSNIGAGFDVHWYQQRPGAAPKLLISGNTHRPSGVPDRISGSKSGTLASLAITGLQAEDEADYYCQSYDRSLSTILFGGGTKLTVLG

SEQ ID NO: 344 - protein, 8 aa, Artificial Sequence
  Description of Artificial Sequence Synthetic peptide
GGTFSSYA

SEQ ID NO: 345 - protein, 8 aa, Artificial Sequence
  Description of Artificial Sequence Synthetic peptide
IIPIFGIA

SEQ ID NO: 346 - protein, 9 aa, Artificial Sequence
  Description of Artificial Sequence Synthetic peptide
ARSMWYMDS

SEQ ID NO: 347 - protein, 116 aa, Artificial Sequence
  Description of Artificial Sequence Synthetic polypeptide
EVQLVESGAEVKKPGSSVKVSCKASGGTFSSYAISWVRQAPGQGLEWMGRIIPIFGIANYAQKFQGRVTITADKSTSTAYMELSSLRSEDTAVYYCARSMWYMDSWGQGTLVTVSS

SEQ ID NO: 348 - protein, 9 aa, Artificial Sequence
  Description of Artificial Sequence Synthetic peptide
SSNIGAGFD

SEQ ID NO: 349 - protein, 3 aa, Artificial Sequence
  Description of Artificial Sequence Synthetic peptide
GNS

SEQ ID NO: 350 - protein, 11 aa, Artificial Sequence
  Description of Artificial Sequence Synthetic peptide
QSYDSSLSGYV

SEQ ID NO: 351 - protein, 112 aa, Artificial Sequence
  Description of Artificial Sequence Synthetic polypeptide
QSVLTQPPSVSGAPGQRVTISCTGSSSNIGAGFDVHWYQQLPGTAPKLLIFGNSNRPSGVPDRFSGSKSGTSASLAITGLQAEDEADYYCQSYDSSLSGYVFGSGTKVTVLG

SEQ ID NO: 352 - protein, 8 aa, Artificial Sequence
  Description of Artificial Sequence Synthetic peptide
GYTFSSYG

SEQ ID NO: 353 - protein, 8 aa, Artificial Sequence
  Description of Artificial Sequence Synthetic peptide
ISPYNGNT

SEQ ID NO: 354 - protein, 11 aa, Artificial Sequence
  Description of Artificial Sequence Synthetic peptide
ARYSGYYYVDY

SEQ ID NO: 355 - protein, 118 aa, Artificial Sequence
  Description of Artificial Sequence Synthetic polypeptide
QVQLVQSGAEVKKPGASVKVSCKASGYTFSSYGISWVRQAPGQGLEWMGWISPYNGNTNYAQNLQGRVTMTTDTSTTTAYMELRSLTSDDTAVYYCARYSGYYYVDYWGQGTLVTVSS

SEQ ID NO: 356 - protein, 6 aa, Artificial Sequence
  Description of Artificial Sequence Synthetic peptide
QSISSY

SEQ ID NO: 357 - protein, 3 aa, Artificial Sequence
  Description of Artificial Sequence Synthetic peptide
AAS

SEQ ID NO: 358 - protein, 9 aa, Artificial Sequence
  Description of Artificial Sequence Synthetic peptide
QQSYSTPRT

SEQ ID NO: 359 - protein, 108 aa, Artificial Sequence
  Description of Artificial Sequence Synthetic polypeptide
DIQLTQSPSSLSASVGDRVTITCRASQSISSYLNWYQQKPGKAPKLLIYAASSLQSGVPSRFSGSGSGTDFTLTISSLQPEDFATYYCQQSYSTPRTFGQGTKVEIKR

SEQ ID NO: 360 - protein, 8 aa, Artificial Sequence
  Description of Artificial Sequence Synthetic peptide
GGTFSSYA

SEQ ID NO: 361 - protein, 8 aa, Artificial Sequence
  Description of Artificial Sequence Synthetic peptide
IIPILGIA

SEQ ID NO: 362 - protein, 12 aa, Artificial Sequence
  Description of Artificial Sequence Synthetic peptide
ARQGYVWSEMDF

SEQ ID NO: 363 - protein, 119 aa, Artificial Sequence
  Description of Artificial Sequence Synthetic polypeptide
EVQLVESGAEVKKPGSSVKVSCKASGGTFSSYAISWVRQAPGQGLEWMGRIIPILGIANYAQKFQGRVTITADKSTSTAYMELSSLRSEDTAVYYCARQGYVWSEMDFWGQGTLVTVSS

SEQ ID NO: 364 - protein, 6 aa, Artificial Sequence
  Description of Artificial Sequence Synthetic peptide
NIGSKS

SEQ ID NO: 365 - protein, 3 aa, Artificial Sequence
  Description of Artificial Sequence Synthetic peptide
YDS

SEQ ID NO: 366 - protein, 11 aa, Artificial Sequence
  Description of Artificial Sequence Synthetic peptide
QVWDSITDHYV

SEQ ID NO: 367 - protein, 111 aa, Artificial Sequence
  Description of Artificial Sequence Synthetic polypeptide
KLLPVLTQPPSVSVAPGKTARITCGGNNIGSKSVHWYQQKPGQAPVLVIYYDSDRPSGIPERFSGSNSGNTATLTISRVEAGDEADYYCQVWDSITDHYVFGTGTKVTVLG

SEQ ID NO: 368 - protein, 8 aa, Artificial Sequence
  Description of Artificial Sequence Synthetic peptide
GYTFTSYY

SEQ ID NO: 369 - protein, 8 aa, Artificial Sequence
  Description of Artificial Sequence Synthetic peptide
INPSGGST

SEQ ID NO: 370 - protein, 10 aa, Artificial Sequence
  Description of Artificial Sequence Synthetic peptide
AAGSYYSLDI

SEQ ID NO: 371 - protein, 117 aa, Artificial Sequence
  Description of Artificial Sequence Synthetic polypeptide
EVQLVESGAEVKKPGASVKVSCKASGYTFTSYYMHWVRQAPGQGLEWMGIINPSGGSTSYAQKFQGRVTMTRDTSTSTVYMELSSLRSEDTAVYYCAAGSYYSLDIWGQGTMVTVSS

SEQ ID NO: 372 - protein, 8 aa, Artificial Sequence
  Description of Artificial Sequence Synthetic peptide
SGSIASNF

SEQ ID NO: 373 - protein, 3 aa, Artificial Sequence
  Description of Artificial Sequence Synthetic peptide
DDN

SEQ ID NO: 374 - protein, 9 aa, Artificial Sequence
  Description of Artificial Sequence Synthetic peptide
QSYDGSNVI

SEQ ID NO: 375 - protein, 113 aa, Artificial Sequence
  Description of Artificial Sequence Synthetic polypeptide
KLLPVLTQPHSVSESPGKTVTISCTGSSGSIASNFVQWYQQRPGSAPTTVIYDDNQRPSGVPDRFSASIDRSSNSASLTISGLKTDDEADYYCQSYDGSNVIFGGGTKLTVLG

SEQ ID NO: 376 - protein, 8 aa, Artificial Sequence
  Description of Artificial Sequence Synthetic peptide
GYTFSSYY

SEQ ID NO: 377 - protein, 8 aa, Artificial Sequence
  Description of Artificial Sequence Synthetic peptide
INPTSGST

SEQ ID NO: 378 - protein, 10 aa, Artificial Sequence
  Description of Artificial Sequence Synthetic peptide
ARSGGGYGDS

SEQ ID NO: 379 - protein, 117 aa, Artificial Sequence
  Description of Artificial Sequence Synthetic polypeptide
EVQLVQSGAEVEKPGASVKVSCKASGYTFSSYYMDWVRQAPGQGLEWMGRINPTSGSTTYAQKFQGRVTMTRDTSTFTVYMDLSSLRSEDTAVYYCARSGGGYGDSWGQGTLVTVSS

SEQ ID NO: 380 - protein, 6 aa, Artificial Sequence
  Description of Artificial Sequence Synthetic peptide
NFGSQS

SEQ ID NO: 381 - protein, 3 aa, Artificial Sequence
  Description of Artificial Sequence Synthetic peptide
YDQ

SEQ ID NO: 382 - protein, 11 aa, Artificial Sequence
  Description of Artificial Sequence Synthetic peptide
QVWDTYTDHVV

SEQ ID NO: 383 - protein, 109 aa, Artificial Sequence
  Description of Artificial Sequence Synthetic polypeptide
QPVLTQPPSVSVAPGETASVSCGGNNFGSQSVHWYQQKSGQAPLLVIYYDQDRPSEIPARFSGSKSGNTATLTISRVEAGDEADYYCQVWDTYTDHVVFGGGTKLTVLG

SEQ ID NO: 384 - protein, 10 aa, Artificial Sequence
  Description of Artificial Sequence Synthetic peptide
RKSAPSTGGV

SEQ ID NO: 385 - protein, 10 aa, Artificial Sequence
  Description of Artificial Sequence Synthetic peptide
RKSAPATGGV

SEQ ID NO: 386 - protein, 10 aa, Artificial Sequence
  Description of Artificial Sequence Synthetic peptide
RMSAPSTGGV

SEQ ID NO: 387 - protein, 10 aa, Artificial Sequence
  Description of Artificial Sequence Synthetic peptide
RMSAPATGGV

SEQ ID NO: 388 - protein, 10 aa, Artificial Sequence
  Description of Artificial Sequence Synthetic peptide
AMSAPSTGGV

SEQ ID NO: 389 - protein, 10 aa, Artificial Sequence
  Description of Artificial Sequence Synthetic peptide
RASAPSTGGV

SEQ ID NO: 390 - protein, 10 aa, Artificial Sequence
  Description of Artificial Sequence Synthetic peptide
RMAAPSTGGV

SEQ ID NO: 391 - protein, 10 aa, Artificial Sequence
  Description of Artificial Sequence Synthetic peptide
RMSAASTGGV

SEQ ID NO: 392 - protein, 10 aa, Artificial Sequence
  Description of Artificial Sequence Synthetic peptide
RMSAPSAGGV

SEQ ID NO: 393 - protein, 10 aa, Artificial Sequence
  Description of Artificial Sequence Synthetic peptide
RMSAPSTAGV

SEQ ID NO: 394 - protein, 10 aa, Artificial Sequence
  Description of Artificial Sequence Synthetic peptide
RMSAPSTGAV

SEQ ID NO: 395 - protein, 10 aa, Artificial Sequence
  Description of Artificial Sequence Synthetic peptide
RMSAPSTGGA

SEQ ID NO: 396 - protein, 10 aa, Artificial Sequence
  Description of Artificial Sequence Synthetic peptide
AMSAPATGGV

SEQ ID NO: 397 - protein, 10 aa, Artificial Sequence
  Description of Artificial Sequence Synthetic peptide
RASAPATGGV

SEQ ID NO: 398 - protein, 10 aa, Artificial Sequence
  Description of Artificial Sequence Synthetic peptide
RMAAPATGGV

SEQ ID NO: 399 - protein, 10 aa, Artificial Sequence
  Description of Artificial Sequence Synthetic peptide
RMSAAATGGV

SEQ ID NO: 400 - protein, 10 aa, Artificial Sequence
  Description of Artificial Sequence Synthetic peptide
RMSAPAAGGV

SEQ ID NO: 401 - protein, 10 aa, Artificial Sequence
  Description of Artificial Sequence Synthetic peptide
RMSAPATAGV

SEQ ID NO: 402 - protein, 10 aa, Artificial Sequence
  Description of Artificial Sequence Synthetic peptide
RMSAPATGAV

SEQ ID NO: 403 - protein, 10 aa, Artificial Sequence
  Description of Artificial Sequence Synthetic peptide
RMSAPATGGA

SEQ ID NO: 404 - protein, 8 aa, Artificial Sequence
  Description of Artificial Sequence Synthetic peptide
GYSFTSYW

SEQ ID NO: 405 - protein, 8 aa, Artificial Sequence
  Description of Artificial Sequence Synthetic peptide
IYPGDSDT

SEQ ID NO: 406 - protein, 6 aa, Artificial Sequence
  Description of Artificial Sequence Synthetic peptide
ARGYDG

SEQ ID NO: 407 - protein, 113 aa, Artificial Sequence
  Description of Artificial Sequence Synthetic polypeptide
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARGYDGWGQGTLVTVSS

SEQ ID NO: 408 - protein, 11 aa, Artificial Sequence
  Description of Artificial Sequence Synthetic peptide
QSLVYSNGNTY

SEQ ID NO: 409 - protein, 3 aa, Artificial Sequence
  Description of Artificial Sequence Synthetic peptide
EVS

SEQ ID NO: 410 - protein, 9 aa, Artificial Sequence
  Description of Artificial Sequence Synthetic peptide
MQGTHWPPT

SEQ ID NO: 411 - protein, 113 aa, Artificial Sequence
  Description of Artificial Sequence Synthetic polypeptide
DVVMTQSPLSLPVTLGQPASISCRSSQSLVYSNGNTYLNWFHQRPGQSPRRLIYEVSNRDSGVPDRFSGSGSGTDFTLKISRVEAEDVGVYYCMQGTHWPPTFGGGTKLEIKR

SEQ ID NO: 412 - protein, 8 aa, Artificial Sequence
  Description of Artificial Sequence Synthetic peptide
GYSFTSYW

SEQ ID NO: 413 - protein, 8 aa, Artificial Sequence
  Description of Artificial Sequence Synthetic peptide
IYPGDSDT

SEQ ID NO: 414 - protein, 6 aa, Artificial Sequence
  Description of Artificial Sequence Synthetic peptide
ARGFDN

SEQ ID NO: 415 - protein, 113 aa, Artificial Sequence
  Description of Artificial Sequence Synthetic polypeptide
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARGFDNWGQGTLVTVSS

SEQ ID NO: 416 - protein, 11 aa, Artificial Sequence
  Description of Artificial Sequence Synthetic peptide
QSLVYSNGNTY

SEQ ID NO: 417 - protein, 3 aa, Artificial Sequence
  Description of Artificial Sequence Synthetic peptide
KVS

SEQ ID NO: 418 - protein, 9 aa, Artificial Sequence
  Description of Artificial Sequence Synthetic peptide
MQGTYWPYT

SEQ ID NO: 419 - protein, 113 aa, Artificial Sequence
  Description of Artificial Sequence Synthetic polypeptide
EIVLTQSPLSLPVTLGQPASISCRSSQSLVYSNGNTYLSWFHQRPGQSPRRLIYKVSKRDSGVPDRFSGSGSGTDFTLKISRVEAEDVGVFYCMQGTYWPYTFGQGTKLEIKR

SEQ ID NO: 420 - protein, 8 aa, Artificial Sequence
  Description of Artificial Sequence Synthetic peptide
GYSFTSYW

SEQ ID NO: 421 - protein, 8 aa, Artificial Sequence
  Description of Artificial Sequence Synthetic peptide
IYPGDSDT

SEQ ID NO: 422 - protein, 6 aa, Artificial Sequence
  Description of Artificial Sequence Synthetic peptide
ARGYDV

SEQ ID NO: 423 - protein, 113 aa, Artificial Sequence
  Description of Artificial Sequence Synthetic polypeptide
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARGYDVWGQGTLVTVSS

SEQ ID NO: 424 - protein, 11 aa, Artificial Sequence
  Description of Artificial Sequence Synthetic peptide
QSLIYSNGNTY

SEQ ID NO: 425 - protein, 3 aa, Artificial Sequence
  Description of Artificial Sequence Synthetic peptide
KVS

SEQ ID NO: 426 - protein, 9 aa, Artificial Sequence
  Description of Artificial Sequence Synthetic peptide
MQGTHWPPT

SEQ ID NO: 427 - protein, 113 aa, Artificial Sequence
  Description of Artificial Sequence Synthetic polypeptide
DVVMTQSPLSLPVTLGQPASISCRSSQSLIYSNGNTYLTWFHQRPGQPPRRLIHKVSNRDSGVPDRFSGSGSGSDFTLKISRVEAEDVGIYYCMQGTHWPPTFGGGTKLEIKR

SEQ ID NO: 428 - protein, 8 aa, Artificial Sequence
  Description of Artificial Sequence Synthetic peptide
GYSFTSYW

SEQ ID NO: 429 - protein, 8 aa, Artificial Sequence
  Description of Artificial Sequence Synthetic peptide
IYPGDSDT

SEQ ID NO: 430 - protein, 6 aa, Artificial Sequence
  Description of Artificial Sequence Synthetic peptide
ARGYDS

SEQ ID NO: 431 - protein, 113 aa, Artificial Sequence
  Description of Artificial Sequence Synthetic polypeptide
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARGYDSWGQGTLVTVSS

SEQ ID NO: 432 - protein, 11 aa, Artificial Sequence
  Description of Artificial Sequence Synthetic peptide
QSLIYSNGNTY

SEQ ID NO: 433 - protein, 3 aa, Artificial Sequence
  Description of Artificial Sequence Synthetic peptide
KVS

SEQ ID NO: 434 - protein, 9 aa, Artificial Sequence
  Description of Artificial Sequence Synthetic peptide
MQGTHWPPT

SEQ ID NO: 435 - protein, 113 aa, Artificial Sequence; identical to SEQ ID NO: 427
  Description of Artificial Sequence Synthetic polypeptide

SEQ ID NO: 436 - protein, 8 aa, Artificial Sequence
  Description of Artificial Sequence Synthetic peptide
GLTFDRYA

SEQ ID NO: 437 - protein, 8 aa, Artificial Sequence
  Description of Artificial Sequence Synthetic peptide
ITGDGYYT

SEQ ID NO: 438 - protein, 13 aa, Artificial Sequence
  Description of Artificial Sequence Synthetic peptide
ARLSGIGRSSYDG

SEQ ID NO: 439 - protein, 120 aa, Artificial Sequence
  Description of Artificial Sequence Synthetic polypeptide
EVQLVESGGGLVQPGGSLRLSCAASGLTFDRYAMSWVRQAAGKGLERFSAITGDGYYTYYADSVKGRFTISRDNSKNTLYLQMNSLGAEDTAVYYCARLSGIGRSSYDGWGQGTLVTVSS

SEQ ID NO: 440 - protein, 9 aa, Artificial Sequence
  Description of Artificial Sequence Synthetic peptide
SGINVGTYR

SEQ ID NO: 441 - protein, 7 aa, Artificial Sequence
  Description of Artificial Sequence Synthetic peptide
YKSDSDK

SEQ ID NO: 442 - protein, 7 aa, Artificial Sequence
  Description of Artificial Sequence Synthetic peptide
MIWHSSA

SEQ ID NO: 443 - protein, 116 aa, Artificial Sequence
  Description of Artificial Sequence Synthetic polypeptide
QAVLTQPSSLSASPGASASLTCTLRSGINVGTYRIYWYQQKPGSPPQYLLRYKSDSDKQQGSGVPSRFSGSKDASANAGILLISGLQSEDEADYYCMIWHSSAWVFGGGTKLTVLG

SEQ ID NO: 444 - protein, 8 aa, Artificial Sequence
  Description of Artificial Sequence Synthetic peptide
GYTFTSYT

SEQ ID NO: 445 - protein, 8 aa, Artificial Sequence
  Description of Artificial Sequence Synthetic peptide
ISPYNGNT

SEQ ID NO: 446 - protein, 12 aa, Artificial Sequence
  Description of Artificial Sequence Synthetic peptide
ARSWEHGFPYDE

SEQ ID NO: 447 - protein, 119 aa, Artificial Sequence
  Description of Artificial Sequence Synthetic polypeptide
QVQLVESGAEVKKPGASVKVSCKASGYTFTSYTITWVRQAPGQGLEWMGWISPYNGNTNYAQNLQGRVTMTTDTSTTTAYMELRSLTSDDTAVYYCARSWEHGFPYDEWGQGTLVTVSS

SEQ ID NO: 448 - protein, 9 aa, Artificial Sequence
  Description of Artificial Sequence Synthetic peptide
SSNLGAGYD

SEQ ID NO: 449 - protein, 8 aa, Artificial Sequence
  Description of Artificial Sequence Synthetic peptide
ISPYNGNT

SEQ ID NO: 450 - protein, 11 aa, Artificial Sequence
  Description of Artificial Sequence Synthetic peptide
QSYDSSLSASV

SEQ ID NO: 451 - protein, 112 aa, Artificial Sequence
  Description of Artificial Sequence Synthetic polypeptide
QSVLTQPPSVSGAPGQRVTISCTGSSSNLGAGYDVHWYQQLPGTAPKVLVYFNNNRPSGVPDRFSGSKSGTSASLAITGLQAEDEADYYCQSYDSSLSASVFGTGTKVTVLG

SEQ ID NO: 452 - protein, 8 aa, Artificial Sequence
  Description of Artificial Sequence Synthetic peptide
AGTFNRYS

SEQ ID NO: 453 - protein, 8 aa, Artificial Sequence
  Description of Artificial Sequence Synthetic peptide
IIPIIGVA

SEQ ID NO: 454 - protein, 11 aa, Artificial Sequence
  Description of Artificial Sequence Synthetic peptide
ARQEYSYAMDY

SEQ ID NO: 455 - protein, 118 aa, Artificial Sequence
  Description of Artificial Sequence Synthetic polypeptide
EVQLVQSGAEVRKPGSSVKVSCKASAGTFNRYSLSWVRQAPGQGLEWVGRIIPIIGVADYAQKFQGRVTITADKSATTAYMELHSLRSEDTAVYYCARQEYSYAMDYWGQGTLVTVSS

SEQ ID NO: 456 - protein, 9 aa, Artificial Sequence
  Description of Artificial Sequence Synthetic peptide
SSNIGAGYD

SEQ ID NO: 457 - protein, 3 aa, Artificial Sequence
  Description of Artificial Sequence Synthetic peptide
GNN

SEQ ID NO: 458 - protein, 10 aa, Artificial Sequence
  Description of Artificial Sequence Synthetic peptide
QSYDTSLTPV

SEQ ID NO: 459 - protein, 111 aa, Artificial Sequence
  Description of Artificial Sequence Synthetic polypeptide
QSVLTQPPSVSGAPGQRVTIFCTGSSSNIGAGYDVHWYQQLPGTAPKLLIYGNNNRPSGVPDRFSGSKSGTSASLAITGLQAEDEADYYCQSYDTSLTPVFGGGTKLTVLG

SEQ ID NO: 460 - protein, 8 aa, Artificial Sequence
  Description of Artificial Sequence Synthetic peptide
GGTFSSYA

SEQ ID NO: 461 - protein, 8 aa, Artificial Sequence
  Description of Artificial Sequence Synthetic peptide
IIPIFGTA

SEQ ID NO: 462 - protein, 13 aa, Artificial Sequence
  Description of Artificial Sequence Synthetic peptide
ARSYWTFEYSIDS

SEQ ID NO: 463 - protein, 120 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARSYWTFEYSIDSWGQGTLVTVSS

SEQ ID NO: 464 - protein, 9 aa, Artificial Sequence
  Description of Artificial Sequence Synthetic peptide
SLNLGAGYD

SEQ ID NO: 465 - protein, 3 aa, Artificial Sequence
  Description of Artificial Sequence Synthetic peptide
ANT

SEQ ID NO: 466 - protein, 11 aa, Artificial Sequence
  Description of Artificial Sequence Synthetic peptide
QSYDNSLSGYV

SEQ ID NO: 467 - protein, 112 aa, Artificial Sequence
  Description of Artificial Sequence Synthetic polypeptide
QSVLTQPPSVSGAPGQRVTISCTGSSLNLGAGYDVHWYQQFPGTAPKLLIFANTNRPSGVPDRFSASKSGTSASLAITGLQAEDEADYFCQSYDNSLSGYVFGTGTKVTVLG

SEQ ID NO: 468 - protein, 8 aa, Artificial Sequence
  Description of Artificial Sequence Synthetic peptide
GYTFTSYG

SEQ ID NO: 469 - protein, 8 aa, Artificial Sequence
  Description of Artificial Sequence Synthetic peptide
ISAYNGNT

SEQ ID NO: 470 - protein, 12 aa, Artificial Sequence
  Description of Artificial Sequence Synthetic peptide
ARYYESGYPFDW

SEQ ID NO: 471 - protein, 119 aa, Artificial Sequence
  Description of Artificial Sequence Synthetic polypeptide
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARYYESGYPFDWWGQGTLVTVSS

SEQ ID NO: 472 - protein, 8 aa, Artificial Sequence
  Description of Artificial Sequence Synthetic peptide
TFNIGSNT

SEQ ID NO: 473 - protein, 3 aa, Artificial Sequence
  Description of Artificial Sequence Synthetic peptide
SNN

SEQ ID NO: 474 - protein, 12 aa, Artificial Sequence
  Description of Artificial Sequence Synthetic peptide
AAWDDSLSGHVV

SEQ ID NO: 475 - protein, 112 aa, Artificial Sequence
  Description of Artificial Sequence Synthetic polypeptide
SYVLTQPPSASGTPGQRVTISCSGSTFNIGSNTVNWYQQLPATAPKLLIYSNNQRPSGVPDRFSGSKSGTSASLAISGLQSEDAAAYYCAAWDDSLSGHVVFGGGTKLTVLG

SEQ ID NO: 476 - protein, 8 aa, Artificial Sequence
  Description of Artificial Sequence Synthetic peptide
GYTFTGYY

SEQ ID NO: 477 - protein, 8 aa, Artificial Sequence
  Description of Artificial Sequence Synthetic peptide
FDPEDGET

SEQ ID NO: 478 - protein, 14 aa, Artificial Sequence
  Description of Artificial Sequence Synthetic peptide
ARSSWWSPVTYYDI

SEQ ID NO: 479 - protein, 121 aa, Artificial Sequence
  Description of Artificial Sequence Synthetic polypeptide
EVQLVQSGAEVKKPGASVKVSCKASGYTFTGYYMHWVRQAPGKGLEWMGGFDPEDGETIYAQKFQGRVTMTEDTSTDTAYMELSSLRSEDTAVYYCARSSWWSPVTYYDIWGQGTLVTVSS

SEQ ID NO: 480 - protein, 6 aa, Artificial Sequence
  Description of Artificial Sequence Synthetic peptide
SLRSYY

SEQ ID NO: 481 - protein, 3 aa, Artificial Sequence
  Description of Artificial Sequence Synthetic peptide
AKS

SEQ ID NO: 482 - protein, 9 aa, Artificial Sequence
  Description of Artificial Sequence Synthetic peptide
NSRDSSGNR

SEQ ID NO: 483 - protein, 109 aa, Artificial Sequence
  Description of Artificial Sequence Synthetic polypeptide
SSELTQDPAVSVALGQTVRITCQGDSLRSYYANWYQQKPGQAPVLVIYAKSNRPSGIADRFSGSSSGNTASLTITGAQAEDEADYYCNSRDSSGNRWVFGGGTKVTVLG

SEQ ID NO: 484 - protein, 8 aa, Artificial Sequence
  Description of Artificial Sequence Synthetic peptide
GYTVTSYG

SEQ ID NO: 485 - protein, 8 aa, Artificial Sequence
  Description of Artificial Sequence Synthetic peptide
ISAYNGDT

SEQ ID NO: 486 - protein, 16 aa, Artificial Sequence
  Description of Artificial Sequence Synthetic peptide
ARSSLPFGVVPNAFDI

SEQ ID NO: 487 - protein, 123 aa, Artificial Sequence
  Description of Artificial Sequence Synthetic polypeptide
QMQLVQSGAEVKKPGASMKVSCKASGYTVTSYGLSWVRQAPGQGLEWVGWISAYNGDTIYAQKLQGRVTMTTDTSTSTAYMELRSLRSDDTAMYYCARSSLPFGVVPNAFDIWGQGTMVTVSS

SEQ ID NO: 488 - protein, 8 aa, Artificial Sequence
  Description of Artificial Sequence Synthetic peptide
SSNIGSNT

SEQ ID NO: 489 - protein, 3 aa, Artificial Sequence
  Description of Artificial Sequence Synthetic peptide
SNN

SEQ ID NO: 490 - protein, 11 aa, Artificial Sequence
  Description of Artificial Sequence Synthetic peptide
AAWDDSLNGYV

SEQ ID NO: 491 - protein, 111 aa, Artificial Sequence
  Description of Artificial Sequence Synthetic polypeptide
SYVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGYVFGTGTKVTVLG

SEQ ID NO: 492 - protein, 8 aa, Artificial Sequence
  Description of Artificial Sequence Synthetic peptide
GYTFTDYY

SEQ ID NO: 493 - protein, 8 aa, Artificial Sequence
  Description of Artificial Sequence Synthetic peptide
INPHSGGT

SEQ ID NO: 494 - protein, 13 aa, Artificial Sequence
  Description of Artificial Sequence Synthetic peptide
AREDYSGSGSSDA

SEQ ID NO: 495 - protein, 120 aa, Artificial Sequence
  Description of Artificial Sequence Synthetic polypeptide
QMQLVQSGAEVKTTGASVRVSCKASGYTFTDYYLHWVRQAPGQGLEWMGWINPHSGGTNYAQKFQGRVTMTRDTSISTAYMELSSLRSDDTAVYYCAREDYSGSGSSDAWGQGTLVTVSS

SEQ ID NO: 496 - protein, 6 aa, Artificial Sequence
  Description of Artificial Sequence Synthetic peptide
NIGSKS

SEQ ID NO: 497 - protein, 3 aa, Artificial Sequence
  Description of Artificial Sequence Synthetic peptide
YDN

SEQ ID NO: 498 - protein, 11 aa, Artificial Sequence
  Description of Artificial Sequence Synthetic peptide
QVWNSSSDHYV

SEQ ID NO: 499 - protein, 109 aa, Artificial Sequence
  Description of Artificial Sequence Synthetic polypeptide
SYELTQPPSVSVAPGKTARITCGGNNIGSKSVNWYQQKPGQAPVLVIYYDNDRPSGIPERFSGSNSGNTATLTISRVEAGDEADYYCQVWNSSSDHYVFGTGTKVTVLG

SEQ ID NO: 500 - protein, 9 aa, Artificial Sequence
  Description of Artificial Sequence Synthetic peptide
RMFPNAPYL

SEQ ID NO: 501 - protein, 10 aa, Artificial Sequence
  Description of Artificial Sequence Synthetic peptide
GGTFSSYAIS

SEQ ID NO: 502 - protein, 17 aa, Artificial Sequence
  Description of Artificial Sequence Synthetic peptide
GIIPIFGTANYAQKFQG

SEQ ID NO: 503 - protein, 10 aa, Artificial Sequence
  Description of Artificial Sequence Synthetic peptide
RIPPYYGMDV

SEQ ID NO: 504 - protein, 119 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARRIPPYYGMDVWGQGTTVTVSS

SEQ ID NO: 505 - protein, 13 aa, Artificial Sequence
  Description of Artificial Sequence Synthetic peptide
SGSSSNIGSNYVY

SEQ ID NO: 506 - protein, 7 aa, Artificial Sequence
  Description of Artificial Sequence Synthetic peptide
RSNQRPS

SEQ ID NO: 507 - protein, 11 aa, Artificial Sequence
  Description of Artificial Sequence Synthetic peptide
AAWDDSLNGVV

SEQ ID NO: 508 - protein, 111 aa, Artificial Sequence
  Description of Artificial Sequence Synthetic polypeptide
QTVVTQPPSASGTPGQRVTISCSGSSSNIGSNYVYWYQQLPGTAPKLLIYRSNQRPSGVPDRFSGSKSGTSASLAISGPRSVDEADYYCAAWDDSLNGVVFGGGTKLTVLG

SEQ ID NO: 509 - protein, 250 aa, Artificial Sequence
  Description of Artificial Sequence Synthetic polypeptide
QTVVTQPPSASGTPGQRVTISCSGSSSNIGSNYVYWYQQLPGTAPKLLIYRSNQRPSGVPDRFSGSKSGTSASLAISGPRSVDEADYYCAAWDDSLNGVVFGGGTKLTVLGSRGGGGSGGGGSGGGSLEMAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARRIPPYYGMDVWGQGTTVTVSS

SEQ ID NO: 510 - protein, 12 aa, Artificial Sequence
  Description of Artificial Sequence Synthetic peptide
GDSVSSNSAAWN

SEQ ID NO: 511 - protein, 18 aa, Artificial Sequence
  Description of Artificial Sequence Synthetic peptide
RTYYGSKWYNDYAVSVKS

SEQ ID NO: 512 - protein, 9 aa, Artificial Sequence
  Description of Artificial Sequence Synthetic peptide
GRLGDAFDI

SEQ ID NO: 513 - protein, 121 aa, Artificial Sequence
  Description of Artificial Sequence Synthetic polypeptide
QVQLQQSGPGLVKPSQTLSLTCAISGDSVSSNSAAWNWIRQSPSRGLEWLGRTYYGSKWYNDYAVSVKSRITINPDTSKNQFSLQLNSVTPEDTAVYYCARGRLGDAFDIWGQGTMVTVSS

SEQ ID NO: 514 - protein, 11 aa, Artificial Sequence
  Description of Artificial Sequence Synthetic peptide
RASQSISSYLN

SEQ ID NO: 515 - protein, 7 aa, Artificial Sequence
  Description of Artificial Sequence Synthetic peptide
AASSLQS

SEQ ID NO: 516 - protein, 9 aa, Artificial Sequence
  Description of Artificial Sequence Synthetic peptide
QQSYSTPLT

SEQ ID NO: 517 - protein, 108 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVDIKR

SEQ ID NO: 518 - protein, 250 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVDIKRSRGGGGSGGGGSGGGGSLEMAQVQLQQSGPGLVKPSQTLSLTCAISGDSVSSNSAAWNWIRQSPSRGLEWLGRTYYGSKWYNDYAVSVKSRITINPDTSKNQFSLQLNSVTPEDTAVYYCARGRLGDAFDIWGQGTMVTVSS

SEQ ID NO: 519 - protein, 10 aa, Artificial Sequence
  Description of Artificial Sequence Synthetic peptide
GYSFTNFWIS

SEQ ID NO: 520 - protein, 17 aa, Artificial Sequence
  Description of Artificial Sequence Synthetic peptide
RVDPGYSYSTYSPSFQG

SEQ ID NO: 521 - protein, 12 aa, Artificial Sequence
  Description of Artificial Sequence Synthetic peptide
VQYSGYYDWFDP

SEQ ID NO: 522 - protein, 121 aa, Artificial Sequence
  Description of Artificial Sequence Synthetic polypeptide
QMQLVQSGAEVKEPGESLRISCKGSGYSFTNFWISWVRQMPGKGLEWMGRVDPGYSYSTYSPSFQGHVTISADKSTSTAYLQWNSLKASDTAMYYCARVQYSGYYDWFDPWGQGTLVTVSS

SEQ ID NO: 523 - protein, 13 aa, Artificial Sequence
  Description of Artificial Sequence Synthetic peptide
SGSSSNIGSNTVN

SEQ ID NO: 524 - protein, 7 aa, Artificial Sequence
  Description of Artificial Sequence Synthetic peptide
SNNQRPS

SEQ ID NO: 525 - protein, 11 aa, Artificial Sequence
  Description of Artificial Sequence Synthetic peptide
AAWDDSLNGWV

SEQ ID NO: 526 - protein, 111 aa, Artificial Sequence
  Description of Artificial Sequence Synthetic polypeptide
QAVVTQPPSASGTPGQRVTISCSGSSSNIGSNTVNWYQQVPGTAPKLLIYSNNQRPSGVPDRFSGSKSGTSASLAISGLQSEDEADYYCAAWDDSLNGWVFGGGTKLTVLG

SEQ ID NO: 527 - protein, 253 aa, Artificial Sequence
  Description of Artificial Sequence Synthetic polypeptide
QAVVTQPPSASGTPGQRVTISCSGSSSNIGSNTVNWYQQVPGTAPKLLIYSNNQRPSGVPDRFSGSKSGTSASLAISGLQSEDEADYYCAAWDDSLNGWVFGGGTKLTVLGSRGGGGSGGGGSGGGGSLEMAQMQLVQSGAEVKEPGESLRISCKGSGYSFTNFWISWVRQMPGKGLEWMGRVDPGYSYSTYSPSFQGHVTISADKSTSTAYLQWNSLKASDTAMYYCARVQYSGYYDWFDPWGQGTLVTVSS

SEQ ID NO: 528 - protein, 10 aa, Artificial Sequence
  Description of Artificial Sequence Synthetic peptide
GYNFSNKWIG

SEQ ID NO: 529 - protein, 17 aa, Artificial Sequence
  Description of Artificial Sequence Synthetic peptide
IIYPGYSDITYSPSFQG

SEQ ID NO: 530 - protein, 9 aa, Artificial Sequence
  Description of Artificial Sequence Synthetic peptide
HTALAGFDY

SEQ ID NO: 531 - protein, 118 aa, Artificial Sequence
  Description of Artificial Sequence Synthetic polypeptide
QVQLVQSGAEVKKPGESLKISCKGSGYNFSNKWIGWVRQLPGRGLEWIAIIYPGYSDITYSPSFQGRVTISADTSINTAYLHWHSLKASDTAMYYCVRHTALAGFDYWGLGTLVTVSS

SEQ ID NO: 532 - protein, 11 aa, Artificial Sequence
  Description of Artificial Sequence Synthetic peptide
RASQNINKWLA

SEQ ID NO: 533 - protein, 7 aa, Artificial Sequence
  Description of Artificial Sequence Synthetic peptide
KASSLES

SEQ ID NO: 534 - protein, 8 aa, Artificial Sequence
  Description of Artificial Sequence Synthetic peptide
QQYNSYAT

SEQ ID NO: 535 - protein, 107 aa, Artificial Sequence
  Description of Artificial Sequence Synthetic polypeptide
DIQMTQSPSTLSASVGDRVTITCRASQNINKWLAWYQQRPGKAPQLLIYKASSLESGVPSRFSGSGSGTEYTLTISSLQPDDFATYYCQQYNSYATFGQGTKVEIKR

SEQ ID NO: 536 - protein, 246 aa, Artificial Sequence
  Description of Artificial Sequence Synthetic polypeptide
DIQMTQSPSTLSASVGDRVTITCRASQNINKWLAWYQQRPGKAPQLLIYKASSLESGVPSRFSGSGSGTEYTLTISSLQPDDFATYYCQQYNSYATFGQGTKVEIKRSRGGGGSGGGGSGGGGSLEMAQVQLVQSGAEVKKPGESLKISCKGSGYNFSNKWIGWVRQLPGRGLEWIAIIYPGYSDITYSPSFQGRVTISADTSINTAYLHWHSLKASDTAMYYCVRHTALAGFDYWGLGTLVTVSS

SEQ ID NO: 537 - protein, 10 aa, Artificial Sequence
  Description of Artificial Sequence Synthetic peptide
GFTFDDYGMS

SEQ ID NO: 538 - protein, 17 aa, Artificial Sequence
  Description of Artificial Sequence Synthetic peptide
GINWNGGSTGYADSVRG

SEQ ID NO: 539 - protein, 12 aa, Artificial Sequence
  Description of Artificial Sequence Synthetic peptide
ERGYGYHDPHDY

SEQ ID NO: 540 - protein, 121 aa, Artificial Sequence
  Description of Artificial Sequence Synthetic polypeptide
EVQLVQSGGGVVRPGGSLRLSCAASGFTFDDYGMSWVRQAPGKGLEWVSGINWNGGSTGYADSVRGRFTISRDNAKNSLYLQMNSLRAEDTALYYCARERGYGYHDPHDYWGQGTLVTVSS

SEQ ID NO: 541 - protein, 11 aa, Artificial Sequence
  Description of Artificial Sequence Synthetic peptide
GRNNIGSKSVH

SEQ ID NO: 542 - protein, 7 aa, Artificial Sequence
  Description of Artificial Sequence Synthetic peptide
DDSDRPS

SEQ ID NO: 543 - protein, 11 aa, Artificial Sequence
  Description of Artificial Sequence Synthetic peptide
QVWDSSSDHVV

SEQ ID NO: 544 - protein, 109 aa, Artificial Sequence
  Description of Artificial Sequence Synthetic polypeptide
QSVVTQPPSVSVAPGKTARITCGRNNIGSKSVHWYQQKPGQAPVLVVYDDSDRPSGIPERFSGSNSGNTATLTISRVEAGDEADYYCQVWDSSSDHVVFGGGTKLTVLG

SEQ ID NO: 545 - protein, 249 aa, Artificial Sequence
  Description of Artificial Sequence Synthetic polypeptide
QSVVTQPPSVSVAPGKTARITCGRNNIGSKSVHWYQQKPGQAPVLVVYDDSDRPSGIPERFSGSNSGNTATLTISRVEAGDEADYYCQVWDSSSDHVVFGGGTKLTVLGSRGGGGSGGGGSGGSLEMAEVQLVQSGGGVVRPGGSLRLSCAASGFTFDDYGMSWVRQAPGKGLEWVSGINWNGGSTGYADSVRGRFTISRDNAKNSLYLQMNSLRAEDTALYYCARERGYGYHDPHDYWGQGTLVTVSS

SEQ ID NO: 546 - protein, 10 aa, Artificial Sequence
  Description of Artificial Sequence Synthetic peptide
GFSVSGTYMG

SEQ ID NO: 547 - protein, 16 aa, Artificial Sequence
  Description of Artificial Sequence Synthetic peptide
LLYSGGGTYHPASLQG

SEQ ID NO: 548 - protein, 10 aa, Artificial Sequence
  Description of Artificial Sequence Synthetic peptide
GGAGGGHFDS

SEQ ID NO: 549 - protein, 118 aa, Artificial Sequence
  Description of Artificial Sequence Synthetic polypeptide
EVQLVETGGGLLQPGGSLRLSCAASGFSVSGTYMGWVRQAPGKGLEWVALLYSGGGTYHPASLQGRFIVSRDSSKNMVYLQMNSLKAEDTAVYYCAKGGAGGGHFDSWGQGTLVTVSS

SEQ ID NO: 550 - protein, 14 aa, Artificial Sequence
  Description of Artificial Sequence Synthetic peptide
TGSSSNIGAGYDVH

SEQ ID NO: 551 - protein, 7 aa, Artificial Sequence
  Description of Artificial Sequence Synthetic peptide
GNSNRPS

SEQ ID NO: 552 - protein, 11 aa, Artificial Sequence
  Description of Artificial Sequence Synthetic peptide
AAWDDSLNGYV

SEQ ID NO: 553 - protein, 112 aa, Artificial Sequence
  Description of Artificial Sequence Synthetic polypeptide
QSVLTQPPSVSGAPGQRVTISCTGSSSNIGAGYDVHWYQQLPGTAPKLLIYGNSNRPSGVPDRFSGSKSGTSASLAISGLQSEDEADYYCAAWDDSLNGYVFGTGTKLTVLG

SEQ ID NO: 554 - protein, 251 aa, Artificial Sequence
  Description of Artificial Sequence Synthetic polypeptide
QSVLTQPPSVSGAPGQRVTISCTGSSSNIGAGYDVHWYQQLPGTAPKLLIYGNSNRPSGVPDRFSGSKSGTSASLAISGLQSEDEADYYCAAWDDSLNGYVFGTGTKLTVLGSRGGGGSGGGGSGGGGSLEMAEVQLVETGGGLLQPGGSLRLSCAASGFSVSGTYMGWVRQAPGKGLEWVALLYSGGGTYHPASLQGRFIVSRDSSKNMVYLQMNSLKAEDTAVYYCAKGGAGGGHFDSWGQGTLVTVSS

SEQ ID NO: 555 - protein, 10 aa, Artificial Sequence
  Description of Artificial Sequence Synthetic peptide
LTDAVKVMDL

SEQ ID NO: 556 - protein, 9 aa, Artificial Sequence
  Description of Artificial Sequence Synthetic peptide
KLQCVDLHV

SEQ ID NO: 557 - protein, 10 aa, Artificial Sequence
  Description of Artificial Sequence Synthetic peptide
VISNDVCAQV

SEQ ID NO: 558 - protein, 10 aa, Artificial Sequence
  Description of Artificial Sequence Synthetic peptide
FLTPKKLQCV

SEQ ID NO: 559 - protein, 9 aa, Artificial Sequence
  Description of Artificial Sequence Synthetic peptide
ALQCVDLHV

SEQ ID NO: 560 - protein, 9 aa, Artificial Sequence
  Description of Artificial Sequence Synthetic peptide
KLACVDLHV

SEQ ID NO: 561 - protein, 9 aa, Artificial Sequence
  Description of Artificial Sequence Synthetic peptide
KLQAVDLHV

SEQ ID NO: 562 - protein, 9 aa, Artificial Sequence
  Description of Artificial Sequence Synthetic peptide
KLQCADLHV

SEQ ID NO: 563 - protein, 9 aa, Artificial Sequence
  Description of Artificial Sequence Synthetic peptide
KLQCVALHV

SEQ ID NO: 564 - protein, 9 aa, Artificial Sequence
  Description of Artificial Sequence Synthetic peptide
KLQCVDAHV

SEQ ID NO: 565 - protein, 9 aa, Artificial Sequence
  Description of Artificial Sequence Synthetic peptide
KLQCVDLAV

SEQ ID NO: 566 - protein, 8 aa, Artificial Sequence
  Description of Artificial Sequence Synthetic peptide
GGTFSSYA

SEQ ID NO: 567 - protein, 8 aa, Artificial Sequence
  Description of Artificial Sequence Synthetic peptide
GFTFSSYA

SEQ ID NO: 568 - protein, 8 aa, Artificial Sequence
  Description of Artificial Sequence Synthetic peptide
GYNFLNYG

SEQ ID NO: 569 - protein, 8 aa, Artificial Sequence
  Description of Artificial Sequence Synthetic peptide
GYTFTGYY

SEQ ID NO: 570 - protein, 8 aa, Artificial Sequence
  Description of Artificial Sequence Synthetic peptide
GGSFSDYY

SEQ ID NO: 571 - protein, 8 aa, Artificial Sequence
  Description of Artificial Sequence Synthetic peptide
GYTFTSYG

SEQ ID NO: 572 - protein, 8 aa, Artificial Sequence
  Description of Artificial Sequence Synthetic peptide
GGTFSSYA

SEQ ID NO: 573 - protein, 8 aa, Artificial Sequence
  Description of Artificial Sequence Synthetic peptide
GYTFTSYG

SEQ ID NO: 574 - protein, 8 aa, Artificial Sequence
  Description of Artificial Sequence Synthetic peptide
GYTFTSYY

SEQ ID NO: 575 - protein, 8 aa, Artificial Sequence
  Description of Artificial Sequence Synthetic peptide
GYTFTGYF

SEQ ID NO: 576 - protein, 8 aa, Artificial Sequence
  Description of Artificial Sequence Synthetic peptide
GGTFSSYA

SEQ ID NO: 577 - protein, 8 aa, Artificial Sequence
  Description of Artificial Sequence Synthetic peptide
GYSFTSYW

SEQ ID NO: 578 - protein, 8 aa, Artificial Sequence
  Description of Artificial Sequence Synthetic peptide
GYSFTSYW

SEQ ID NO: 579 - protein, 8 aa, Artificial Sequence
  Description of Artificial Sequence Synthetic peptide
GYSFTSYR

SEQ ID NO: 580 - protein, 8 aa, Artificial Sequence
  Description of Artificial Sequence Synthetic peptide
GYTFTNYG

SEQ ID NO: 581 - protein, 8 aa, Artificial Sequence
  Description of Artificial Sequence Synthetic peptide
IIPIPGIT

SEQ ID NO: 582 - protein, 8 aa, Artificial Sequence
  Description of Artificial Sequence Synthetic peptide
ISGSGGST

SEQ ID NO: 583 - protein, 8 aa, Artificial Sequence
  Description of Artificial Sequence Synthetic peptide
ISTYTGNT

SEQ ID NO: 584 - protein, 8 aa, Artificial Sequence
  Description of Artificial Sequence Synthetic peptide
FDPEDGET

SEQ ID NO: 585 - protein, 7 aa, Artificial Sequence
  Description of Artificial Sequence Synthetic peptide
INHSGGT

SEQ ID NO: 586 - protein, 8 aa, Artificial Sequence
  Description of Artificial Sequence Synthetic peptide
ISAYNGNT

SEQ ID NO: 587 - protein, 8 aa, Artificial Sequence
  Description of Artificial Sequence Synthetic peptide
INPNSGGT

SEQ ID NO: 588 - protein, 8 aa, Artificial Sequence
  Description of Artificial Sequence Synthetic peptide
ISAYNGNT

SEQ ID NO: 589 - protein, 8 aa, Artificial Sequence
  Description of Artificial Sequence Synthetic peptide
FDPEDGET

SEQ ID NO: 590 - protein, 8 aa, Artificial Sequence
  Description of Artificial Sequence Synthetic peptide
FDPEDGET

SEQ ID NO: 591 - protein, 8 aa, Artificial Sequence
  Description of Artificial Sequence Synthetic peptide
IIPILGIA

SEQ ID NO: 592 - protein, 8 aa, Artificial Sequence
  Description of Artificial Sequence Synthetic peptide
IYPGDSDT

SEQ ID NO: 593 - protein, 8 aa, Artificial Sequence
  Description of Artificial Sequence Synthetic peptide
IYPGDSDT

SEQ ID NO: 594 - protein, 8 aa, Artificial Sequence
  Description of Artificial Sequence Synthetic peptide
IDPSDSYT

SEQ ID NO: 595 - protein, 13 aa, Artificial Sequence
  Description of Artificial Sequence Synthetic peptide
ARSYKWGSSLVDA

SEQ ID NO: 596 - protein, 13 aa, Artificial Sequence
  Description of Artificial Sequence Synthetic peptide
ARNYYSQYWMMDL

SEQ ID NO: 597 - protein, 11 aa, Artificial Sequence
  Description of Artificial Sequence Synthetic peptide
ARSSEYYTWDH

SEQ ID NO: 598 - protein, 7 aa, Artificial Sequence
  Description of Artificial Sequence Synthetic peptide
ARYGFDY

SEQ ID NO: 599 - protein, 12 aa, Artificial Sequence
  Description of Artificial Sequence Synthetic peptide
ARYNEYGSGYDK

SEQ ID NO: 600 - protein, 11 aa, Artificial Sequence
  Description of Artificial Sequence Synthetic peptide
ARSSQYYVWDS

SEQ ID NO: 601 - protein, 16 aa, Artificial Sequence
  Description of Artificial Sequence Synthetic peptide
ARWSYYYFQQFWSLDG

SEQ ID NO: 602 - protein, 10 aa, Artificial Sequence
  Description of Artificial Sequence Synthetic peptide
ARTNYNKYDI

SEQ ID NO: 603 - protein, 7 aa, Artificial Sequence
  Description of Artificial Sequence Synthetic peptide
ARYSYDY

SEQ ID NO: 604 - protein, 7 aa, Artificial Sequence
  Description of Artificial Sequence Synthetic peptide
ARYSYDL

SEQ ID NO: 605 - protein, 15 aa, Artificial Sequence
  Description of Artificial Sequence Synthetic peptide
ARVSQPVYGSSTYDI

SEQ ID NO: 606 - protein, 11 aa, Artificial Sequence
  Description of Artificial Sequence Synthetic peptide
ARLVVPDAFDI

SEQ ID NO: 607 - protein, 14 aa, Artificial Sequence
  Description of Artificial Sequence Synthetic peptide
ARWGSRGFLDAFDI

SEQ ID NO: 608 - protein, 15 aa, Artificial Sequence
  Description of Artificial Sequence Synthetic peptide
ARWGLSWDGWGVTDY

SEQ ID NO: 609 - protein, 7 aa, Artificial Sequence
  Description of Artificial Sequence Synthetic peptide
ARYNYDT

SEQ ID NO: 610 - protein, 10 aa, Artificial Sequence
  Description of Artificial Sequence Synthetic peptide
ARSFGAGYDS

SEQ ID NO: 611 - protein, 7 aa, Artificial Sequence
  Description of Artificial Sequence Synthetic peptide
ARYPWDH

SEQ ID NO: 612 - protein, 12 aa, Artificial Sequence
  Description of Artificial Sequence Synthetic peptide
ARSSYYGYLSDG

SEQ ID NO: 613 - protein, 120 aa, Artificial Sequence
  Description of Artificial Sequence Synthetic polypeptide
EVQLVQSGAEVKRPGSSVKVSCKASGGTFSSYAINWVRRAPGQGLEWMGKIIPIPGITNYAQKFQDRVTFTADTSTNIAYMELSSLRSEDTAMYYCARSYKWGSSLVDAWGQGTLVTVSS

SEQ ID NO: 614 - protein, 120 aa, Artificial Sequence
  Description of Artificial Sequence Synthetic polypeptide
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCARNYYSQYWMMDLWGQGTLVTVSS

SEQ ID NO: 615 - protein, 118 aa, Artificial Sequence
  Description of Artificial Sequence Synthetic polypeptide
QVQLVQSGAEVKKPGDSVKVSCKPSGYNFLNYGINWVRQAPGQGLEWMGWISTYTGNTNYAQKLQGRVTFTTDTSTSTAYMEMRSLRSDDTAVYYCARSSEYYTWDHWGQGTLVTVSS

SEQ ID NO: 616 - protein, 114 aa, Artificial Sequence
  Description of Artificial Sequence Synthetic polypeptide
EVQLVQSGAEVKKPGASVKVSCKASGYTFTGYYIHWVRQAPGQGLEWMGGFDPEDGETIYAQKFQDRVTMTADTSTDTAYMELSSLRSEDTAVYYCARYGFDYWGQGTLVTVSS

SEQ ID NO: 617 - protein, 118 aa, Artificial Sequence
  Description of Artificial Sequence Synthetic polypeptide
QVQLQQWGAGLLKPSETLSLTCAVKGGSFSDYYWSWIRQPPGKGLEWIGEINHSGGTNYNPSLKSRVTISVDTSKNQFSLKLLSVTAADTAVYYCARYNEYGSGYDKWGQGTLVTVSS

SEQ ID NO: 618 - protein, 118 aa, Artificial Sequence
  Description of Artificial Sequence Synthetic polypeptide
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARSSQYYVWDSWGQGTLVTVSS

SEQ ID NO: 619 - protein, 123 aa, Artificial Sequence
  Description of Artificial Sequence Synthetic polypeptide
QVQLVQSGAEVKKPGSSVKVSCKASGGTFSSYAISWVRQAPGQGLEWMGWINPNSGGTNYAQKFQGRVTMTRDTSISTAYMELSRLRSDDTAVYYCARWSYYYFQQFWSLDGWGQGTLVTVSS

SEQ ID NO: 620 - protein, 117 aa, Artificial Sequence
  Description of Artificial Sequence Synthetic polypeptide
QMQLVQSGSEVKKPGASVKVSCKASGYTFTSYGISWVRQAPGQGLEWMGWISAYNGNTDYVQKLQGRVTMTTDTSTNTAYMELGSLGSDDTAVYYCARTNYNKYDIWGQGTLVTVSS

SEQ ID NO: 621 - protein, 114 aa, Artificial Sequence
  Description of Artificial Sequence Synthetic polypeptide
QVQLVQSGAEVKKPGASVKVSCKASGYTFTSYYMHWVRQAPGQGLEWMGGFDPEDGETIYAQKFQGRVTMTEDTSTDTAYMGLSSLRSEDTAVYYCARYSYDYWGQGTLVTVSS

SEQ ID NO: 622 - protein, 114 aa, Artificial Sequence
  Description of Artificial Sequence Synthetic polypeptide
EVQLVQSGAEVKKPGASVKVSCKASGYTFTGYFMHWVRQAPGQGLEWMGGFDPEDGETIYAQKFQGRVTMTEDTSTDTAYMELSSLRSEDTAVYYCARYSYDLWGQGTLVTVSS

SEQ ID NO: 623 - protein, 122 aa, Artificial Sequence
  Description of Artificial Sequence Synthetic polypeptide
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCARVSQPVYGSSTYDIWGQGTLVTVSS

SEQ ID NO: 624 - protein, 118 aa, Artificial Sequence
  Description of Artificial Sequence Synthetic polypeptide
QMQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLVVPDAFDIWGQGTMVTVSS

SEQ ID NO: 625 - protein, 121 aa, Artificial Sequence
  Description of Artificial Sequence Synthetic polypeptide
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARWGSRGFLDAFDIWGQGTMVTVSS

SEQ ID NO: 626 - protein, 122 aa, Artificial Sequence
  Description of Artificial Sequence Synthetic polypeptide
EVQLVQSGAEVKKPGESLKISCKGSGYSFTSYRIGWVRQMPGKGLEWMGIIYPGDSDTRYSPSFQGQVTISADKSISTAYLQWSSLKASDTAMYYCARWGLSWDGWGVTDYWGQGTLVTVSS

SEQ ID NO: 627 - protein, 114 aa, Artificial Sequence
  Description of Artificial Sequence Synthetic polypeptide
QVQLVQSGAEVKKPGASVKVSCKASGYTFTSYYMHWVRQAPGKGLEWMGGFDPEDGETIYAQKFQGRVTMTEDTSTDTAYMELSSLRSEDTAVYYCARYNYDTWGQGTLVTVSS

SEQ ID NO: 628 - protein, 117 aa, Artificial Sequence
  Description of Artificial Sequence Synthetic polypeptide
EVQLVQSGAEVKKPGESLKISCKGSGYSFTSYWIGWVRQMPGKGLEWMGRIDPSDSYTNYSPSFQGHVTISADKSISTAYLQWSSLKASDTAMYYCARSFGAGYDSWGQGTLVTVSS

SEQ ID NO: 629 - protein, 114 aa, Artificial Sequence
  Description of Artificial Sequence Synthetic polypeptide
QVQLVQSGAEVKKPGASVKVSCKASGYTFTSYYMHWVRQAPGKGLEWMGGFDPEDGETIYAQKFQGRVTMTEDTSTDTAYMELSSLRSEDTAVYYCARYPWDHWGQGTLVTVSS

SEQ ID NO: 630 - protein, 119 aa, Artificial Sequence
  Description of Artificial Sequence Synthetic polypeptide
QVQLVQSGAEVKKPGASVKVSCKTSGYTFTNYGISWVRQAPGQGLEWMGWISAYNGNTNYAQNLQGRVTMTTDTSTSTAYMELRSLRSDDTAVYYCARSSYYGYLSDGWGQGTLVTVSS

SEQ ID NO: 631 - protein, 8 aa, Artificial Sequence
  Description of Artificial Sequence Synthetic peptide
NSNIGSNT

SEQ ID NO: 632 - protein, 9 aa, Artificial Sequence
  Description of Artificial Sequence Synthetic peptide
SSNFGAGYD

SEQ ID NO: 633 - protein, 9 aa, Artificial Sequence
  Description of Artificial Sequence Synthetic peptide
SSNIGAGYD

SEQ ID NO: 634 - protein, 9 aa, Artificial Sequence
  Description of Artificial Sequence Synthetic peptide
TGAVTSGYY

SEQ ID NO: 635 - protein, 8 aa, Artificial Sequence
  Description of Artificial Sequence Synthetic peptide
SYNIGNNY

SEQ ID NO: 636 - protein, 9 aa, Artificial Sequence
  Description of Artificial Sequence Synthetic peptide
SSNFGAGYD

SEQ ID NO: 637 - protein, 8 aa, Artificial Sequence
  Description of Artificial Sequence Synthetic peptide
SSNIGSNT

SEQ ID NO: 638 - protein, 9 aa, Artificial Sequence
  Description of Artificial Sequence Synthetic peptide
SSNIGAGYD

SEQ ID NO: 639 - protein, 9 aa, Artificial Sequence
  Description of Artificial Sequence Synthetic peptide
TGAVTSGYY

SEQ ID NO: 640 - protein, 9 aa, Artificial Sequence
  Description of Artificial Sequence Synthetic peptide
TGAVTSGYY

SEQ ID NO: 641 - protein, 8 aa, Artificial Sequence
  Description of Artificial Sequence Synthetic peptide
SSNLGSNS

SEQ ID NO: 642 - protein, 8 aa, Artificial Sequence
  Description of Artificial Sequence Synthetic peptide
SSNIGNNY

SEQ ID NO: 643 - protein, 8 aa, Artificial Sequence
  Description of Artificial Sequence Synthetic peptide
SSNIGNNY

SEQ ID NO: 644 - protein, 6 aa, Artificial Sequence
  Description of Artificial Sequence Synthetic peptide
QSISSY

SEQ ID NO: 645 - protein, 9 aa, Artificial Sequence
  Description of Artificial Sequence Synthetic peptide
TGTVTSTYY

SEQ ID NO: 646 - protein, 9 aa, Artificial Sequence
  Description of Artificial Sequence Synthetic peptide
SSDVGGYNY

SEQ ID NO: 647 - protein, 8 aa, Artificial Sequence
  Description of Artificial Sequence Synthetic peptide
SSNIGTNY

SEQ ID NO: 648 - protein, 3 aa, Artificial Sequence
  Description of Artificial Sequence Synthetic peptide
SNN

SEQ ID NO: 649 - protein, 3 aa, Artificial Sequence
  Description of Artificial Sequence Synthetic peptide
GDT

SEQ ID NO: 650 - protein, 3 aa, Artificial Sequence
  Description of Artificial Sequence Synthetic peptide
GNS

SEQ ID NO: 651 - protein, 3 aa, Artificial Sequence
  Description of Artificial Sequence Synthetic peptide
TTG

SEQ ID NO: 652 - protein, 3 aa, Artificial Sequence
  Description of Artificial Sequence Synthetic peptide
DNN

SEQ ID NO: 653 - protein, 3 aa, Artificial Sequence
  Description of Artificial Sequence Synthetic peptide
GNS

SEQ ID NO: 654 - protein, 3 aa, Artificial Sequence
  Description of Artificial Sequence Synthetic peptide
STS

SEQ ID NO: 655 - protein, 3 aa, Artificial Sequence
  Description of Artificial Sequence Synthetic peptide
DNH

SEQ ID NO: 656 - protein, 3 aa, Artificial Sequence
  Description of Artificial Sequence Synthetic peptide
DNY

SEQ ID NO: 657 - protein, 3 aa, Artificial Sequence
  Description of Artificial Sequence Synthetic peptide
DND

SEQ ID NO: 658 - protein, 3 aa, Artificial Sequence
  Description of Artificial Sequence Synthetic peptide
AAS

SEQ ID NO: 659 - protein, 3 aa, Artificial Sequence
  Description of Artificial Sequence Synthetic peptide
DVS

SEQ ID NO: 660 - protein, 3 aa, Artificial Sequence
  Description of Artificial Sequence Synthetic peptide
STN

SEQ ID NO: 661 - protein, 11 aa, Artificial Sequence
  Description of Artificial Sequence Synthetic peptide
ATWDDSLNGPV

SEQ ID NO: 662 - protein, 11 aa, Artificial Sequence
  Description of Artificial Sequence Synthetic peptide
QSYDTSLSGSV

SEQ ID NO: 663 - protein, 11 aa, Artificial Sequence
  Description of Artificial Sequence Synthetic peptide
QSYDSSLSGWV

SEQ ID NO: 664 - protein, 9 aa, Artificial Sequence
  Description of Artificial Sequence Synthetic peptide
LLYSGGVWV

SEQ ID NO: 665 - protein, 11 aa, Artificial Sequence
  Description of Artificial Sequence Synthetic peptide
GTWESSLSAYV

SEQ ID NO: 666 - protein, 11 aa, Artificial Sequence
  Description of Artificial Sequence Synthetic peptide
QSYDSSLSGWV

SEQ ID NO: 667 - protein, 12 aa, Artificial Sequence
  Description of Artificial Sequence Synthetic peptide
AAWDDSLNGRWV

SEQ ID NO: 668 - protein, 10 aa, Artificial Sequence
  Description of Artificial Sequence Synthetic peptide
QSYDSSLSEV

SEQ ID NO: 669 - protein, 9 aa, Artificial Sequence
  Description of Artificial Sequence Synthetic peptide
LLYYGGAYV

SEQ ID NO: 670 - protein, 10 aa, Artificial Sequence
  Description of Artificial Sequence Synthetic peptide
LLYYGGAQWV

SEQ ID NO: 671 - protein, 11 aa, Artificial Sequence
  Description of Artificial Sequence Synthetic peptide
AAWDDSLNSVV

SEQ ID NO: 672 - protein, 11 aa, Artificial Sequence
  Description of Artificial Sequence Synthetic peptide
GTWDSSLSAGV

SEQ ID NO: 673 - protein, 11 aa, Artificial Sequence
  Description of Artificial Sequence Synthetic peptide
GTWDSSLSSGV

SEQ ID NO: 674 - protein, 9 aa, Artificial Sequence
  Description of Artificial Sequence Synthetic peptide
QQSYSTPFT

SEQ ID NO: 675 - protein, 9 aa, Artificial Sequence
  Description of Artificial Sequence Synthetic peptide
LVFYGGVWV

SEQ ID NO: 676 - protein, 10 aa, Artificial Sequence
  Description of Artificial Sequence Synthetic peptide
SSYTSSSRYV

SEQ ID NO: 677 - protein, 9 aa, Artificial Sequence
  Description of Artificial Sequence Synthetic peptide
LLYYGGQGV

SEQ ID NO: 678 - protein, 12 aa, Artificial Sequence
  Description of Artificial Sequence Synthetic peptide
AAWDDSLSGLYV

SEQ ID NO: 679 - protein, 111 aa, Artificial Sequence
  Description of Artificial Sequence Synthetic polypeptide
QSVLTQPPSASGTPGQRVTLSCSGSNSNIGSNTVNWYQQLPGTNPKLLIYSNNQRPSGVPDRFSGSKSGTSASLAISGLQSEDEADYYCATWDDSLNGPVFGGGTKLTVLG

SEQ ID NO: 680 - protein, 112 aa, Artificial Sequence
  Description of Artificial Sequence Synthetic polypeptide
QSVLTQPPSVSGAPGQRVTISCTGSSSNFGAGYDVHWYQQLPGAAPKLLIYGDTNRPSGVPDRFSGSKSGTSASLAITGLQAEDEADYYCQSYDTSLSGSVFGGGTKLTVLG

SEQ ID NO: 681 - protein, 112 aa, Artificial Sequence
  Description of Artificial Sequence Synthetic polypeptide
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGWVFGGGTKLTVLG

SEQ ID NO: 682 - protein, 110 aa, Artificial Sequence
  Description of Artificial Sequence Synthetic polypeptide
QAVVTQEPSLTVSPGGTVTLTCASSTGAVTSGYYPNWFQLKPGQAPRALIYTTGKKHSWAPARFSGSLLGGKAALTLSGVQPEDEAEYYCLLYSGGVWVFGGGTKLTVLG

SEQ ID NO: 683 - protein, 111 aa, Artificial Sequence
  Description of Artificial Sequence Synthetic polypeptide
QSVLTQPPSVSAAPGQKVTISCSGSSYNIGNNYVSWYQQLPGTAPKLLIYDNNKRPSGIPDRFSGSKSGTSATLGITGLQTGDEAEYYCGTWESSLSAYVFGTGTKVTVLG

SEQ ID NO: 684 - protein, 112 aa, Artificial Sequence
  Description of Artificial Sequence Synthetic polypeptide
QSVVTQPPSVSGAPGQRVTISCTGSSSNFGAGYDVHWYQQLPGTAPKLLIYGNSNRPSGVPDRFSGSKSGTSASLAITGLQAEDEADYYCQSYDSSLSGWVFGGGTKLTVLG

SEQ ID NO: 685 - protein, 112 aa, Artificial Sequence
  Description of Artificial Sequence Synthetic polypeptide
QAVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGRWVFGGGTKLTVLG

SEQ ID NO: 686 - protein, 111 aa, Artificial Sequence
  Description of Artificial Sequence Synthetic polypeptide
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSEVFGTGTKVTVLG

SEQ ID NO: 687 - protein, 110 aa, Artificial Sequence
  Description of Artificial Sequence Synthetic polypeptide
QAVVTQEPSLTVSPGGTVTLTCASSTGAVTSGYYPNWFQQKPGQAPRALIYSTSNKHSWTPARFSGSLLGGKAALTLSGVQPEDEAEYYCLLYYGGAYVFGTGTKVTVLG

SEQ ID NO: 688 - protein, 111 aa, Artificial Sequence
  Description of Artificial Sequence Synthetic polypeptide
QTVVTQEPSLTVSPGGTVTLTCASSTGAVTSGYYPNWFQQKPGQAPRALIYSTSNKHSWTPARFSGSLLGGKAALTLSGVQPEDEAEYYCLLYYGGAQWVFGGGTKLTVLG

SEQ ID NO: 689 - protein, 111 aa, Artificial Sequence
  Description of Artificial Sequence Synthetic polypeptide
QAVLTQPPSASGTPGQRVTISCSGRSSNLGSNSVNWYQQVPGTAPKLLIFDNHQRPSGVPDRFSGSKSGTSASLAISGLRSEDETDYYCAAWDDSLNSVVFGGGTKLTVLG

SEQ ID NO: 690 - protein, 111 aa, Artificial Sequence
  Description of Artificial Sequence Synthetic polypeptide
QSVVTQPPSVSAAPGQKVTISCSGSSSNIGNNYVSWYQQLPGTAPKLLIYDNYKRPSGIPDRFSGSKSGTSATLGITGLQTGDEADYYCGTWDSSLSAGVFGGGTKLTVLG

SEQ ID NO: 691 - protein, 111 aa, Artificial Sequence
  Description of Artificial Sequence Synthetic polypeptide
QSVVTQPPSVSAAPGQRVTISCSGSSSNIGNNYVSWYQQLPGAAPRLLIYDNDKRPSGIPDRFSGSKSGTSATLGITGLQTGDEADYYCGTWDSSLSSGVFGGGTKLTVLG

SEQ ID NO: 692 - protein, 108 aa, Artificial Sequence
  Description of Artificial Sequence Synthetic polypeptide
DIQLTQSPSSLSASVGDRVTITCRASQSISSYLNWYQQKPGKAPKLLIYAASSLQSGVPSRFSGSGSGTDFTLTISSLQPEDFATYYCQQSYSTPFTFGPGTKVDIKR

SEQ ID NO: 693 - protein, 110 aa, Artificial Sequence
  Description of Artificial Sequence Synthetic polypeptide
QAVVTQEPSLTVSPGGTVTLTCASSTGTVTSTYYPNWFQQKPGQAPRALIYSTSNRHSWTPARFSGSLLGGKAALTVSGVQPDDEAEYYCLVFYGGVWVFGGGTKLTVLG

SEQ ID NO: 694 - protein, 111 aa, Artificial Sequence
  Description of Artificial Sequence Synthetic polypeptide
QSALTQPASVSGSPGQSITISCTGTSSDVGGYNYVSWYQQHPGKAPKLMIYDVSKRPSGVSNRFSGSKSGNTASLTISGLQAEDEADYYCSSYTSSSRYVFGTGTKVTVLG

SEQ ID NO: 695 - protein, 110 aa, Artificial Sequence
  Description of Artificial Sequence Synthetic polypeptide
QAVVTQEPSLTVSPGGTVTLTCASSTGAVTSGYYPNWFQQKPGQAPRPLIYSTNNKHSWTPARFSGSLLGGKAALTLSGVQPEDEAEYYCLLYYGGQGVFGGGTKLTVLG

SEQ ID NO: 696 - protein, 112 aa, Artificial Sequence
  Description of Artificial Sequence Synthetic polypeptide
LPVLTQPPSASGTPGQRVTISCSGSSSNIGTNYVYWYQQLPGTAPKLLIYSNNQRPSGVPDRFSGSKSGTSASLAISGLRSEDEADYYCAAWDDSLSGLYVFGTGTKVTVLG

SEQ ID NO: 697 - protein, 23 aa, Artificial Sequence
  Description of Artificial Sequence Synthetic peptide
KNDAPVVQEPRRLSFRSTIYGSR

SEQ ID NO: 698 - protein, 15 aa, Artificial Sequence
  Description of Artificial Sequence Synthetic peptide
AANCIRIGIPMADPI

SEQ ID NO: 699 - protein, 19 aa, Artificial Sequence
  Description of Artificial Sequence Synthetic peptide
SSTGVLFVKFGPPPTASPG

SEQ ID NO: 700 - protein, 15 aa, Artificial Sequence
  Description of Artificial Sequence Synthetic peptide
SNPMILMRLKLPNCE

SEQ ID NO: 701 - protein, 10 aa, Artificial Sequence
  Description of Artificial Sequence Synthetic peptide
GGSLSSHGVS

SEQ ID NO: 702 - protein, 17 aa, Artificial Sequence
  Description of Artificial Sequence Synthetic peptide
RIIPMFGVTDYAQKFQD

SEQ ID NO: 703 - protein, 9 aa, Artificial Sequence
  Description of Artificial Sequence Synthetic peptide
ESRGATFEY

SEQ ID NO: 704 - protein, 118 aa, Artificial Sequence
  Description of Artificial Sequence Synthetic polypeptide
QVQLVQSGTEVKKPGSSVKVSCQASGGSLSSHGVSWLRQAPGQGLEWVGRIIPMFGVTDYAQKFQDRVTITADKSTSTVYMELISLGSDDTAVYFCARESRGATFEYWGQGTLVTVSS

SEQ ID NO: 705 - protein, 12 aa, Artificial Sequence
  Description of Artificial Sequence Synthetic peptide
RASQSVSSSYLA

SEQ ID NO: 706 - protein, 7 aa, Artificial Sequence
  Description of Artificial Sequence Synthetic peptide
GASSRAT

SEQ ID NO: 707 - protein, 6 aa, Artificial Sequence
  Description of Artificial Sequence Synthetic peptide
QQYGSS

SEQ ID NO: 708 - protein, 106 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSFGPGTKVDIKR

SEQ ID NO: 709 - protein, 12 aa, Artificial Sequence
  Description of Artificial Sequence Synthetic peptide
GGSISSSSYYWG

SEQ ID NO: 710 - protein, 16 aa, Artificial Sequence
  Description of Artificial Sequence Synthetic peptide
SIYYSGSTYYNPSLKS

SEQ ID NO: 711 - protein, 9 aa, Artificial Sequence
  Description of Artificial Sequence Synthetic peptide
HDGTDAFDI

SEQ ID NO: 712 - protein, 119 aa, Artificial Sequence
  Description of Artificial Sequence Synthetic polypeptide
QLQLQESGPGLVKPSETLSLTCTVSGGSISSSSYYWGWIRQPPGKGLEWIGSIYYSGSTYYNPSLKSRVTISVDTSKNQFSLKLGSVTAADTAVYYCARHDGTDAFDIWGQGTTVTVSS

SEQ ID NO: 713 - protein, 14 aa, Artificial Sequence
  Description of Artificial Sequence Synthetic peptide
TGTSSDFGDYDYVS

SEQ ID NO: 714 - protein, 7 aa, Artificial Sequence
  Description of Artificial Sequence Synthetic peptide
DVSDRPS

SEQ ID NO: 715 - protein, 10 aa, Artificial Sequence
  Description of Artificial Sequence Synthetic peptide
SSLTTSSTLV

SEQ ID NO: 716 - protein, 111 aa, Artificial Sequence
  Description of Artificial Sequence Synthetic polypeptide
QSVLTQPASVSGSPGQSITISCTGTSSDFGDYDYVSWYQQHPGKAPKLMIYDVSDRPSGVSNRFSGSKSGNTASLTISGLQAEDEADYFCSSLTTSSTLVFGGGTKLTVLG

SEQ ID NO: 717 - protein, 27 aa, Artificial Sequence
  Description of Artificial Sequence Synthetic peptide
YPYDVPDYAYPYDVPDYAYPYDVPDYA

SEQ ID NO: 718 - protein, 30 aa, Artificial Sequence
  Description of Artificial Sequence Synthetic polypeptide
YPYDVPDYASYPYDVPDYASYPYDVPDYAS

SEQ ID NO: 719 - protein, 24 aa, Artificial Sequence
  Description of Artificial Sequence Synthetic peptide
DYKDDDDKDYKDDDDKDYKDDDDK

SEQ ID NO: 720 - protein, 30 aa, Artificial Sequence
  Description of Artificial Sequence Synthetic polypeptide
EQKLISEEDLEQKLISEEDLEQKLISEEDL